(19) United States
(12) Patent Application Publication
(10) Pub. No.: US 2026/0217843 A1
(43) Pub. Date: Jul. 30, 2026

(54) ANTIBODIES THAT BIND OX40L AND METHODS OF USE

(71) Applicant: Apogee Therapeutics, Inc., Waltham, MA (US)

(72) Inventors: Byong Ha KANG, San Francisco, CA (US); Eric Franklin ZHU, Arlington, MA (US); Hussam Hisham SHAHEEN, Vega Alta, PR (US); Jason Zee Seug OH, Shrewsbury, MA (US); Mohammad Murshid ALAM, Waltham, MA (US)

(21) Appl. No.: 19/425,386

(22) Filed: Dec. 18, 2025

Related U.S. Application Data
(63) Continuation of application No. 19/478,894, filed on Apr. 29, 2024.
(60) Provisional application No. 63/462,871, filed on Apr. 28, 2023.
(60) Provisional application No. 63/529,631, filed on Jul. 28, 2023.
(60) Provisional application No. 63/561,037, filed on Mar. 4, 2024.

(51) Int. Cl. C07K 16/28; A61K 39/00

(52) U.S. Cl. CPC C07K 16/2875; A61K 2039/505; C07K 2317/24; C07K 2317/92; C07K 2317/94

(57) ABSTRACT

Described herein are novel and improved antibodies that bind OX40L and methods of use thereof. In certain aspects, described herein are methods of inhibiting OX40L biological activity. In certain aspects, described herein are pharmaceutical compositions comprising the anti-OX40L antibodies. In certain aspects, the antibodies and methods described herein are used for treatment of an inflammatory disease or disorder associated with elevated levels of OX40L.

### CROSS-REFERENCE TO RELATED APPLICATIONS

This application is a Continuation of U.S. patent application Ser. No. 19/478,894, filed Oct. 27, 2025, pending, which application is a 35 U.S.C. § 371 national stage filing of International Application No. PCT/US24/26854, filed on Apr. 29, 2024, which claims the benefit of and priority to U.S. Provisional Application No. 63/462,871, filed Apr. 28, 2023, U.S. Provisional Application No. 63/529,631, filed Jul. 28, 2023, and U.S. Provisional Application No. 63/561,037, filed Mar. 4, 2024. The entire contents of the aforementioned applications are incorporated herein by reference.

### SEQUENCE LISTING

The instant application contains a Sequence Listing which has been submitted electronically in XML format via EFS-Web and is hereby incorporated by reference in its entirety. Said XML copy, created on Apr. 29, 2024, is named AOJ-19USCN_SL.xml and is 1,148,379 bytes in size.

### BACKGROUND

An emerging mechanism in treatments for atopic dermatitis (AD) is targeting OX40 or OX40L. OX40L is the ligand for OX40. OX40L is expressed on antigen presenting cells and its interaction with OX40 causes the accumulation of T cells by providing a survival signal. OX40L, by playing a role in activating T cells and reprogramming them into inflammatory subsets, contributes to immune overactivation in AD and other inflammatory conditions. Additionally, OX40L activation of OX40 inhibits the expression of FOXP3 and the inhibitory function of regulatory T (Treg) cells. Treg cells suppress immune response, which leads to worse symptoms in inflammatory conditions. Therefore, OX40L blockade may lead to clinical benefit in AD and other inflammatory conditions by first suppressing inflammatory T cell activation, and next by increasing the proliferation of Treg cells, which can serve to further reduce inflammatory cells. Amlitelimab, which targets OX40L, and rocatinlimab, which targets OX40, have both demonstrated promising Phase 2 data in AD.

OX40L is the ligand for OX40 expressed on antigen presenting cells. Its interaction with OX40 causes the accumulation of T cells by providing a survival signal. T cells are important types of white blood cells of the immune system that play a central role in the immune response. OX40L, by playing a role in activating T cells and reprogramming them into inflammatory subsets, contributes to immune overactivation in AD and other inflammatory conditions, such as Systemic Lupus Erythematosus. Additionally, OX40L activation of OX40 inhibits the expression of Foxp3 and the inhibitory function of regulatory T (Treg) cells. Treg cells can suppress the immune response that leads to worsening symptoms in inflammatory conditions.

Accordingly, there is a need in the art for improved therapeutics that target OX40L, such as recombinant antibodies.

### SUMMARY

In a first aspect, disclosed herein are antibodies, such as recombinant human antibodies, that specifically bind human OX40L.

In certain aspects, described herein is an isolated antibody that binds OX40L comprising a) comprising a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein: a. CDR-H1 comprises an amino acid sequence set forth in any one of SEQ ID NOs: 1-69 and 740-747; b. CDR-H2 comprises an amino acid sequence set forth in any one of SEQ ID NOs: 70-174 and 748-762; c. CDR-H3 comprises an amino acid sequence set forth in any one of SEQ ID NOs: 175-257 and 763-780, d. CDR-L1 comprises an amino acid sequence set forth in any one of SEQ ID NOs: 258-334 and 781-796, e. CDR-L2 comprises an amino acid sequence set forth in any one of SEQ ID NOs: 335-367 and 797-805; and f. CDR-L3 comprises an amino acid sequence set forth in any one of SEQ ID NOs: 268-393 and 806-811.

In some embodiments, an antibody provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 258, 286, and 311; a CDR-L2 comprising an amino acid sequence set forth in any one of SEQ ID NOs: 335 and 359; and a CDR-L3 comprising an amino acid sequence set forth in any one of SEQ ID NOs: 368 and 394.

In some embodiments, an antibody provided herein comprises a CDR-H1 comprising an amino acid sequence set forth in any one of SEQ ID NOs: 2, 23, and 45; a CDR-H2 comprising an amino acid sequence set forth in any one of SEQ ID NOs: 70, 111, and 142; a CDR-H3 comprising an amino acid sequence set forth in any one of SEQ ID NOs: 176, 204, and 224; a CDR-L1 comprising an amino acid sequence set forth in any one of SEQ ID NOs: 259, 287, and 312; a CDR-L2 comprising an amino acid sequence set forth in any one of SEQ ID NOs: 336 and 360; and a CDR-L3 comprising an amino acid sequence set forth in any one of SEQ ID NOs: 369 and 395.

In some embodiments, an antibody provided herein comprises a CDR-H1 comprising an amino acid sequence set forth in any one of SEQ ID NOs: 3, 24, and 46; a CDR-H2 comprising an amino acid sequence set forth in any one of SEQ ID NOs: 71, 112, and 143; a CDR-H3 comprising an amino acid sequence set forth in any one of SEQ ID NOs: 177, 205, and 225; a CDR-L1 comprising an amino acid sequence set forth in any one of SEQ ID NOs: 260, 288, and 313; a CDR-L2 comprising an amino acid sequence set forth in any of SEQ ID NOs: 337 and 361; and a CDR-L3 comprising an amino acid sequence set forth in any one of SEQ ID NOs: 370 and 396.

In some embodiments, an antibody provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 72, 113, and 144; a CDR-H3 comprising an amino acid sequence set forth in any one of SEQ ID NOs: 178, 206, and 226; a CDR-L1 comprising an amino acid sequence set forth in any one of SEQ ID NOs: 261, 289, and 314; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 73, 114, and 145; a CDR-H3 comprising an amino acid sequence set forth in any one of SEQ ID NOs: 179, 207, and 227; a CDR-L1 comprising an amino acid sequence set forth in any one of SEQ ID NOs: 262, 290, and 315; a CDR-L2 comprising an amino acid sequence set forth in any one of SEQ ID NOs: 338 and 362; and a CDR-L3 comprising an amino acid sequence set forth in any one of SEQ ID NOs: 372 or 398.

In some embodiments, an antibody provided herein comprises a CDR-H1 comprising an amino acid sequence set forth in any one of SEQ ID NOs: 6, 24, and 49; a CDR-H2 comprising an amino acid sequence set forth in any one of SEQ ID NOs: 74, 115, and 146; a CDR-H3 comprising an amino acid sequence set forth in any one of SEQ ID NOs: 180, 208, and 228; a CDR-L1 comprising an amino acid sequence set forth in any one of SEQ ID NOs: 263, 291, and 316; a CDR-L2 comprising an amino acid sequence set forth in any of one SEQ ID NOs: 339 and 361; and a CDR-L3 comprising an amino acid sequence set forth in any one of SEQ ID NOs: 373 and 399.

In some embodiments, an antibody provided herein comprises a CDR-H1 comprising an amino acid sequence set forth in any one of SEQ ID NOs: 7, 25, and 50; a CDR-H2 comprising an amino acid sequence set forth in any one of SEQ ID NOs: 75, 116, and 147; a CDR-H3 comprising an amino acid sequence set forth in any one of SEQ ID NOs: 181, 209, and 229; a CDR-L1 comprising an amino acid sequence set forth in any one of SEQ ID NOs: 264, 292, and 317; a CDR-L2 comprising an amino acid sequence set forth in any one of SEQ ID NOs: 340 and 363; and a CDR-L3 comprising an amino acid sequence set forth in any one of SEQ ID NOs: 374 and 400.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 182, 210, and 230; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 375 and 401.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 183, 211, and 231; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 183, 211, and 231; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 184, 212, and 232; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 184, 212, and 232; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 185, 213, and 233; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 185, 213, and 233; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, an antibody provided herein comprises a CDR-H1 comprising an amino acid sequence set forth in any one of SEQ ID NOs: 9, 27, and 52; a CDR-H2 comprising an amino acid sequence set forth in any one of SEQ ID NOs: 77, 118, and 149; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 342 and 364; and a CDR-L3 comprising an amino acid sequence set forth in any one of SEQ ID NOs: 368 and 394.

In some embodiments, an antibody provided herein comprises a CDR-H1 comprising an amino acid sequence set forth in any one of SEQ ID NOs: 1, 28, and 53; a CDR-H2 comprising an amino acid sequence set forth in any one of SEQ ID NOs: 78, 119, and 150; a CDR-H3 comprising an amino acid sequence set forth in any one of SEQ ID NOs: 175, 203, and 223; a CDR-L1 comprising an amino acid sequence set forth in any one of SEQ ID NOs: 266, 286, and 311; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 186, 203, and 235; a CDR-L1 comprising an amino acid sequence set forth in any one of SEQ ID NOs: 267, 294, and 319; a CDR-L2 comprising an amino acid sequence set forth in any one of SEQ ID NOs: 344 and 365; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 80, 120, and 151; a CDR-H3 comprising an amino acid sequence set forth in any one of SEQ ID NOs: 186, 203, and 235; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 11, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 11, 27, and 52; a CDR-H2 comprising an amino acid sequence set forth in any one of SEQ ID NOs: 81, 121, and 152; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 80, 120, and 151; a CDR-H3 comprising an amino acid sequence set forth in any one of SEQ ID NOs: 187, 203, and 236; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 187, 203, and 236; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 268, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, an antibody provided herein comprises a CDR-H1 comprising an amino acid sequence set forth in any one of SEQ ID NOs: 12, 29, and 54; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 82, 111, and 142; a CDR-H3 comprising an amino acid sequence set forth in any one of SEQ ID NOs: 189, 214, and 238; a CDR-L1 comprising an amino acid sequence set forth in any one of SEQ ID NOs: 269, 288, and 313; a CDR-L2 comprising an amino acid sequence set forth in any one of SEQ ID NOs: 345 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27 52; a CDR-H2 comprising an amino acid sequence set forth in any one of SEQ ID NOs: 83, 122, and 153; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 346 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 13, 30, and 55; a CDR-H2 comprising an amino acid sequence set forth in any one of SEQ ID NOs: 84, 111, and 142; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 347 and 365; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 345 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 85, 123, and 154; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 270, 295, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 1, 31, and 56; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 379 and 405.

In some embodiments, an antibody provided herein comprises a CDR-H1 comprising an amino acid sequence set forth in any one of SEQ ID NOs: 1, 32, and 57; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 380 and 406.

In some embodiments, an antibody provided herein comprises a CDR-H1 comprising an amino acid sequence set forth in any one of SEQ ID NOs: 14, 33, and 58; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 13, 30, and 55; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 348 and 366; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 381 and 405.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 335 and 359; and a CDR-L3 comprising an amino acid sequence set forth in any one of SEQ ID NOs: 368 and 394.

In some embodiments, an antibody provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 381 and 405.

In some embodiments, an antibody provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 78, 119, and 150; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, an antibody provided herein comprises a CDR-H1 comprising an amino acid sequence set forth in any one of SEQ ID NOs: 2, 840, and 45; a CDR-H2 comprising an amino acid sequence set forth in any one of SEQ ID NOs: 70, 111, and 142; a CDR-H3 comprising an amino acid sequence set forth in any one of SEQ ID NOs: 176, 204, and 224; a CDR-L1 comprising an amino acid sequence set forth in any one of SEQ ID NOs: 259, 287, and 312; a CDR-L2 comprising an amino acid sequence set forth in any one of SEQ ID NOs: 336 and 360; and a CDR-L3 comprising an amino acid sequence set forth in any one of SEQ ID NOs: 369 and 395.

In some embodiments, an antibody provided herein comprises a CDR-H1 comprising an amino acid sequence set forth in any one of SEQ ID NOs: 15, 34, and 59; a CDR-H2 comprising an amino acid sequence set forth in any one of SEQ ID NOs: 88, 125, and 156; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 349 and 365; and a CDR-L3 comprising an amino acid sequence set forth in any one of SEQ ID NOs: 382 and 407.

In some embodiments, an antibody provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 89, 126, and 157; a CDR-H3 comprising an amino acid sequence set forth in any one of SEQ ID NOs: 178, 206, and 226; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, an antibody provided herein comprises a CDR-H1 comprising an amino acid sequence set forth in any one of SEQ ID NOs: 4, 35, and 60; a CDR-H2 comprising an amino acid sequence set forth in any one of SEQ ID NOs: 72, 113, and 144; a CDR-H3 comprising an amino acid sequence set forth in any one of SEQ ID NOs: 178, 206, and 241; a CDR-L1 comprising an amino acid sequence set forth in any one of SEQ ID NOs: 271, 296, and 320; a CDR-L2 comprising an amino acid sequence set forth in any one of SEQ ID NOs: 350 and 361; and a CDR-L3 comprising an amino acid sequence set forth in any one of SEQ ID NOs: 371 and 397.

In some embodiments, an antibody provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 90, 127, and 158; a CDR-H3 comprising an amino acid sequence set forth in any one of SEQ ID NOs: 178, 206, and 226; a CDR-L1 comprising an amino acid sequence set forth in any one of SEQ ID NOs: 261, 289, and 314; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, an antibody provided herein comprises a CDR-H1 comprising an amino acid sequence set forth in any one of SEQ ID NOs: 16, 36, and 61; a CDR-H2 comprising an amino acid sequence set forth in any one of SEQ ID NOs: 91, 128, and 159; a CDR-H3 comprising an amino acid sequence set forth in any one of SEQ ID NOs: 191, 215, and 242; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 382 and 407.

In some embodiments, an antibody provided herein comprises a CDR-H1 comprising an amino acid sequence set forth in any one of SEQ ID NOs: 16, 36, and 61; a CDR-H2 comprising an amino acid sequence set forth in any one of SEQ ID NOs: 91, 128, and 159; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 371 and 397.

In some embodiments, an antibody provided herein comprises a CDR-H1 comprising an amino acid sequence set forth in any one of SEQ ID NOs: 15, 27, and 62; a CDR-H2 comprising an amino acid sequence set forth in any one of SEQ ID NOs: 72, 113, and 144; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 337 and 361; and a CDR-L3 comprising an amino acid sequence set forth in any one of SEQ ID NOs: 382 and 407.

In some embodiments, an antibody provided herein comprises a CDR-H1 comprising an amino acid sequence set forth in any one of SEQ ID NOs: 15, 27, and 62; a CDR-H2 comprising an amino acid sequence set forth in any one of SEQ ID NOs: 72, 113, and 144; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 383 and 408.

In some embodiments, an antibody provided herein comprises a CDR-H1 comprising an amino acid sequence set forth in any one of SEQ ID NOs: 5, 37, and 63; a CDR-H2 comprising an amino acid sequence set forth in any one of SEQ ID NOs: 92, 129, and 160; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 384 and 409.

In some embodiments, an antibody provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 93, 130, and 161; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 384 and 409.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 192, 207, and 243; a CDR-L1 comprising an amino acid sequence set forth in any one of SEQ ID NOs: 273, 298, and 322; a CDR-L2 comprising an amino acid sequence set forth in any one of SEQ ID NOs: 338 and 362; and a CDR-L3 comprising an amino acid sequence set forth in any one of SEQ ID NOs: 385 and 410.

In some embodiments, an antibody provided herein comprises a CDR-H1 comprising an amino acid sequence set forth in any one of SEQ ID NOs: 17, 38, and 64; a CDR-H2 comprising an amino acid sequence set forth in any one of SEQ ID NOs: 95, 132, and 163; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, an antibody provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 96, 130, and 164; a CDR-H3 comprising an amino acid sequence set forth in any one of SEQ ID NOs: 192, 207, and 243; a CDR-L1 comprising an amino acid sequence set forth in any one of SEQ ID NOs: 274, 299, and 323; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, an antibody provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 97, 130, and 164; a CDR-H3 comprising an amino acid sequence set forth in any one of SEQ ID NOs: 179, 207, and 227; a CDR-L1 comprising an amino acid sequence set forth in any one of SEQ ID NOs: 275, 300, and 324; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, an antibody provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 193, 216, and 244; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 193, 216, and 245; a CDR-L1 comprising an amino acid sequence set forth in any one of SEQ ID NOs: 277, 302, and 326; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 194, 217, and 246; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 194, 217, and 247; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 388 and 413.

In some embodiments, an antibody provided herein comprises a CDR-H1 comprising an amino acid sequence set forth in any one of SEQ ID NOs: 19, 40, and 66; a CDR-H2 comprising an amino acid sequence set forth in any one of SEQ ID NOs: 99, 133, and 165; a CDR-H3 comprising an amino acid sequence set forth in any one of SEQ ID NOs: 180, 208, and 248; a CDR-L1 comprising an amino acid sequence set forth in any one of SEQ ID NOs: 278, 303, and 327; a CDR-L2 comprising an amino acid sequence set forth in any one of SEQ ID NOs: 352 and 367; and a CDR-L3 comprising an amino acid sequence set forth in any one of SEQ ID NOs: 387 and 412.

In some embodiments, an antibody provided herein comprises a CDR-H1 comprising an amino acid sequence set forth in any one of SEQ ID NOs: 20, 41, and 67; a CDR-H2 comprising an amino acid sequence set forth in any one of SEQ ID NOs: 100, 134, and 166; a CDR-H3 comprising an amino acid sequence set forth in any one of SEQ ID NOs: 195, 218, and 249; a CDR-L1 comprising an amino acid sequence set forth in any one of SEQ ID NOs: 279, 304, and 328; a CDR-L2 comprising an amino acid sequence set forth in any one of SEQ ID NOs: 353 and 361; and a CDR-L3 comprising an amino acid sequence set forth in any one of SEQ ID NOs: 374 and 400.

In some embodiments, an antibody provided herein comprises a CDR-H1 comprising an amino acid sequence set forth in any one of SEQ ID NOs: 20, 840, and 48; a CDR-H2 comprising an amino acid sequence set forth in any one of SEQ ID NOs: 101, 133, and 167; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 389 and 414.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 102, 135, and 168; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 350 and 361; and a CDR-L3 comprising an amino acid sequence set forth in any one of SEQ ID NOs: 389 and 414.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 103, 136, and 169; a CDR-H3 comprising an amino acid sequence set forth in any one of SEQ ID NOs: 196, 218, and 250; a CDR-L1 comprising an amino acid sequence set forth in any one of SEQ ID NOs: 280, 305, and 329; a CDR-L2 comprising an amino acid sequence set forth in any one of SEQ ID NOs: 354 and 367; and a CDR-L3 comprising an amino acid sequence set forth in any one of SEQ ID NOs: 390 and 415.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 104, 137, and 170; a CDR-H3 comprising an amino acid sequence set forth in any one of SEQ ID NOs: 197, 208, and 251; a CDR-L1 comprising an amino acid sequence set forth in any one of SEQ ID NOs: 281, 306, and 330; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 105, 138, and 171; a CDR-H3 comprising an amino acid sequence set forth in any one of SEQ ID NOs: 197, 208, and 251; a CDR-L1 comprising an amino acid sequence set forth in any one of SEQ ID NOs: 281, 306, and 330; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 198, 219, and 252; a CDR-L1 comprising an amino acid sequence set forth in any one of SEQ ID NOs: 282, 307, and 331; a CDR-L2 comprising an amino acid sequence set forth in any one of SEQ ID NOs: 337 and 361; and a CDR-L3 comprising an amino acid sequence set forth in any one of SEQ ID NOs: 391 and 416.

In some embodiments, an antibody provided herein comprises a CDR-H1 comprising an amino acid sequence set forth in any one of SEQ ID NOs: 20, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 195, 218, and 253; a CDR-L1 comprising an amino acid sequence set forth in any one of SEQ ID NOs: 280, 305, and 329; a CDR-L2 comprising an amino acid sequence set forth in any one of SEQ ID NOs: 355 and 367; and a CDR-L3 comprising an amino acid sequence set forth in any one of SEQ ID NOs: 387 and 412.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody provided herein comprises a CDR-H1 comprising an amino acid sequence set forth in any one of SEQ ID NOs: 6, 840, and 48; a CDR-H2 comprising an amino acid sequence set forth in any one of SEQ ID NOs: 106, 137, and 170; a CDR-H3 comprising an amino acid sequence set forth in any one of SEQ ID NOs: 195, 218, and 253; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody provided herein comprises a CDR-H1 comprising an amino acid sequence set forth in any one of SEQ ID NOs: 20, 41, and 67; a CDR-H2 comprising an amino acid sequence set forth in any one of SEQ ID NOs: 107, 133, and 167; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 108, 139, and 172; a CDR-H3 comprising an amino acid sequence set forth in any one of SEQ ID NOs: 181, 209, and 229; a CDR-L1 comprising an amino acid sequence set forth in any one of SEQ ID NOs: 283, 308, and 332; a CDR-L2 comprising an amino acid sequence set forth in any one of SEQ ID NOs: 356 and 361; and a CDR-L3 comprising an amino acid sequence set forth in any one of SEQ ID NOs: 374 and 400.

In some embodiments, an antibody provided herein comprises a CDR-H1 comprising an amino acid sequence set forth in any one of SEQ ID NOs: 21, 42, and 68; a CDR-H2 comprising an amino acid sequence set forth in any one of SEQ ID NOs: 109, 140, and 173; a CDR-H3 comprising an amino acid sequence set forth in any one of SEQ ID NOs: 199, 220, and 254; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 374 and 400.

In some embodiments, an antibody provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 106, 137, and 170; a CDR-H3 comprising an amino acid sequence set forth in any one of SEQ ID NOs: 200, 221, and 255; a CDR-L1 comprising an amino acid sequence set forth in any one of SEQ ID NOs: 284, 309, and 333; a CDR-L2 comprising an amino acid sequence set forth in any one of SEQ ID NOs: 337 and 361; and a CDR-L3 comprising an amino acid sequence set forth in any one of SEQ ID NOs: 374 and 400.

In some embodiments, an antibody provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 94, 131, and 162; a CDR-H3 comprising an amino acid sequence set forth in any one of SEQ ID NOs: 201, 222, and 256; a CDR-L1 comprising an amino acid sequence set forth in any one of SEQ ID NOs: 285, 310, and 334; a CDR-L2 comprising an amino acid sequence set forth in any one of SEQ ID NOs: 357 and 361; and a CDR-L3 comprising an amino acid sequence set forth in any one of SEQ ID NOs: 392 and 417.

In some embodiments, an antibody provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 110, 141, and 174; a CDR-H3 comprising an amino acid sequence set forth in any one of SEQ ID NOs: 202, 222, and 257; a CDR-L1 comprising an amino acid sequence set forth in any one of SEQ ID NOs: 285, 310, and 334; a CDR-L2 comprising an amino acid sequence set forth in any one of SEQ ID NOs: 358 and 361; and a CDR-L3 comprising an amino acid sequence set forth in any one of SEQ ID NOs: 393 and 418.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 748, 116, and 758; a CDR-H3 comprising an amino acid sequence set forth in any one of SEQ ID NOs: 763, 769, and 775, a CDR-L1 comprising an amino acid sequence set forth in any one of SEQ ID NOs: 781, 787, and 792; a CDR-L2 comprising an amino acid sequence set forth in any one of SEQ ID NOs: 797 and 802; and a CDR-L3 comprising an amino acid sequence set forth in any one of SEQ ID NOs: 806 and 812.

In some embodiments, an antibody provided herein comprises a CDR-H1 comprising an amino acid sequence set forth in any one of SEQ ID NOs: 740, 742, and 745; a CDR-H2 comprising an amino acid sequence set forth in any one of SEQ ID NOs: 749, 754, and 759; a CDR-H3 comprising an amino acid sequence set forth in any one of SEQ ID NOs: 764, 770, and 776, a CDR-L1 comprising an amino acid sequence set forth in any one of SEQ ID NOs: 782, 788, and 793; a CDR-L2 comprising an amino acid sequence set forth in any one of SEQ ID NOs: 798 and 803; and a CDR-L3 comprising an amino acid sequence set forth in any one of SEQ ID NOs: 807 and 813.

In some embodiments, an antibody provided herein comprises a CDR-H1 comprising an amino acid sequence set forth in any one of SEQ ID NOs: 17, 743, and 746; a CDR-H2 comprising an amino acid sequence set forth in any one of SEQ ID NOs: 750, 133, and 165; a CDR-H3 comprising an amino acid sequence set forth in any one of SEQ ID NOs: 765, 771, and 777, a CDR-L1 comprising an amino acid sequence set forth in any one of SEQ ID NOs: 783, 298, and 322; a CDR-L2 comprising an amino acid sequence set forth in any one of SEQ ID NOs: 799 and 804; and a CDR-L3 comprising an amino acid sequence set forth in any one of SEQ ID NOs: 808 and 814.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 751, 755, and 760; a CDR-H3 comprising an amino acid sequence set forth in any one of SEQ ID NOs: 766, 772, and 778, a CDR-L1 comprising an amino acid sequence set forth in any one of SEQ ID NOs: 784, 789, and 794; a CDR-L2 comprising an amino acid sequence set forth in any one of SEQ ID NOs: 341 and 361; and a CDR-L3 comprising an amino acid sequence set forth in any one of SEQ ID NOs: 809 and 815.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223, a CDR-L1 comprising an amino acid sequence set forth in any one of SEQ ID NOs: 260, 288, and 313; a CDR-L2 comprising an amino acid sequence set forth in any one of SEQ ID NOs: 341 and 361; and a CDR-L3 comprising an amino acid sequence set forth in any one of SEQ ID NOs: 368 and 394.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 752, 756, and 761; a CDR-H3 comprising an amino acid sequence set forth in any one of SEQ ID NOs: 767, 773, and 779, a CDR-L1 comprising an amino acid sequence set forth in any one of SEQ ID NOs: 785, 790, and 795; a CDR-L2 comprising an amino acid sequence set forth in any one of SEQ ID NOs: 800 and 805; and a CDR-L3 comprising an amino acid sequence set forth in SEQ ID NOs: 810 and 816.

In some embodiments, an antibody provided herein comprises a CDR-H1 comprising an amino acid sequence set forth in any one of SEQ ID NOs: 741, 744, and 747; a CDR-H2 comprising an amino acid sequence set forth in any one of SEQ ID NOs: 753, 757, and 762; a CDR-H3 comprising an amino acid sequence set forth in SEQ ID NOs: 768, 774, and 780, a CDR-L1 comprising an amino acid sequence set forth in any one of SEQ ID NOs: 786, 791, and 796; a CDR-L2 comprising an amino acid sequence set forth in any one of SEQ ID NOs: 801 and 360; and a CDR-L3 comprising an amino acid sequence set forth in SEQ ID NOs: 811 and 817.

In some embodiments, wherein the antibody comprises a heavy chain variable domain (VH) sequence selected from an amino acid sequence set forth in SEQ ID NOs: 419-488 and 818-824.

In some embodiments, the antibody comprises a light chain variable domain (VL) sequence selected from an amino acid sequence set forth in any one of SEQ ID NOs: 489-544 and 825-831.

In some embodiments, the antibody comprises a VH sequence selected from an amino acid sequence set forth in any one of SEQ ID NOs: 419-488 and 818-824, and a VL sequence selected from an amino acid sequence set forth in any one of SEQ ID NOs: 489-544 and 825-831.

In certain aspects, described herein is an isolated antibody that binds OX40L, wherein the antibody comprises a VH sequence selected from an amino acid sequence set forth in any one of SEQ ID NOs: 419-488 and 818-824, and a VL sequence selected from an amino acid sequence set forth in any one of SEQ ID NOs: 489-544 and 825-831.

In certain embodiments, the antibody comprises a VH sequence set forth in SEQ ID NO: 429 and a VL sequence set forth in SEQ ID NO: 497.

In certain embodiments, the antibody comprises a VH sequence set forth in SEQ ID NO: 429 and a VL sequence set forth in SEQ ID NO: 498.

In certain embodiments, the antibody comprises a VH sequence set forth in SEQ ID NO: 430 and a VL sequence set forth in SEQ ID NO: 499.

In certain embodiments, the antibody comprises a VH sequence set forth in SEQ ID NO: 431 and a VL sequence set forth in SEQ ID NO: 500.

In certain embodiments, the antibody comprises a VH sequence set forth in SEQ ID NO: 432 and a VL sequence set forth in SEQ ID NO: 501.

In certain embodiments, the antibody comprises a VH sequence set forth in SEQ ID NO: 433 and a VL sequence set forth in SEQ ID NO: 502.

In certain embodiments, the antibody comprises a VH sequence set forth in SEQ ID NO: 434 and a VL sequence set forth in SEQ ID NO: 502.

In certain embodiments, the antibody comprises a VH sequence set forth in SEQ ID NO: 435 and a VL sequence set forth in SEQ ID NO: 502.

In certain embodiments, the antibody comprises a VH sequence set forth in SEQ ID NO: 436 and a VL sequence set forth in SEQ ID NO: 502.

In certain embodiments, the antibody comprises a VH sequence set forth in SEQ ID NO: 437 and a VL sequence set forth in SEQ ID NO: 502.

In certain embodiments, the antibody comprises a VH sequence set forth in SEQ ID NO: 438 and a VL sequence set forth in SEQ ID NO: 503.

In certain embodiments, the antibody comprises a VH sequence set forth in SEQ ID NO: 439 and a VL sequence set forth in SEQ ID NO: 502.

In certain embodiments, the antibody comprises a VH sequence set forth in SEQ ID NO: 440 and a VL sequence set forth in SEQ ID NO: 504.

In certain embodiments, the antibody comprises a VH sequence set forth in SEQ ID NO: 441 and a VL sequence set forth in SEQ ID NO: 505.

In certain embodiments, the antibody comprises a VH sequence set forth in SEQ ID NO: 442 and a VL sequence set forth in SEQ ID NO: 506.

In certain embodiments, the antibody comprises a VH sequence set forth in SEQ ID NO: 443 and a VL sequence set forth in SEQ ID NO: 507.

In certain embodiments, the antibody comprises a VH sequence set forth in SEQ ID NO: 443 and a VL sequence set forth in SEQ ID NO: 508.

In certain embodiments, the antibody comprises a VH sequence set forth in SEQ ID NO: 444 and a VL sequence set forth in SEQ ID NO: 506.

In certain embodiments, the antibody comprises a VH sequence set forth in SEQ ID NO: 445 and a VL sequence set forth in SEQ ID NO: 509.

In certain embodiments, the antibody comprises a VH sequence set forth in SEQ ID NO: 446 and a VL sequence set forth in SEQ ID NO: 510.

In certain embodiments, the antibody comprises a VH sequence set forth in SEQ ID NO: 447 and a VL sequence set forth in SEQ ID NO: 506.

In certain embodiments, the antibody comprises a VH sequence set forth in SEQ ID NO: 445 and a VL sequence set forth in SEQ ID NO: 511.

In certain embodiments, the antibody comprises a VH sequence set forth in SEQ ID NO: 448 and a VL sequence set forth in SEQ ID NO: 506.

In certain embodiments, the antibody comprises a VH sequence set forth in SEQ ID NO: 449 and a VL sequence set forth in SEQ ID NO: 502.

In certain embodiments, the antibody comprises a VH sequence set forth in SEQ ID NO: 449 and a VL sequence set forth in SEQ ID NO: 506.

In certain embodiments, the antibody comprises a VH sequence set forth in SEQ ID NO: 450 and a VL sequence set forth in SEQ ID NO: 506.

In certain embodiments, the antibody comprises a VH sequence set forth in SEQ ID NO: 445 and a VL sequence set forth in SEQ ID NO: 512.

In certain embodiments, the antibody comprises a VH sequence set forth in SEQ ID NO: 449 and a VL sequence set forth in SEQ ID NO: 513.

In certain embodiments, the antibody comprises a VH sequence set forth in SEQ ID NO: 445 and a VL sequence set forth in SEQ ID NO: 514.

In certain embodiments, the antibody comprises a VH sequence set forth in SEQ ID NO: 445 and a VL sequence set forth in SEQ ID NO: 515.

In certain embodiments, the antibody comprises a VH sequence set forth in SEQ ID NO: 451 and a VL sequence set forth in SEQ ID NO: 514.

In certain embodiments, the antibody comprises a VH sequence set forth in SEQ ID NO: 451 and a VL sequence set forth in SEQ ID NO: 506.

In certain embodiments, the antibody comprises a VH sequence set forth in SEQ ID NO: 452 and a VL sequence set forth in SEQ ID NO: 506.

In certain embodiments, the antibody comprises a VH sequence set forth in SEQ ID NO: 438 and a VL sequence set forth in SEQ ID NO: 506.

In certain embodiments, the antibody comprises a VH sequence set forth in SEQ ID NO: 453 and a VL sequence set forth in SEQ ID NO: 512.

In certain embodiments, the antibody comprises a VH sequence set forth in SEQ ID NO: 454 and a VL sequence set forth in SEQ ID NO: 490.

In certain embodiments, the antibody comprises a VH sequence set forth in SEQ ID NO: 455 and a VL sequence set forth in SEQ ID NO: 516.

In certain embodiments, the antibody comprises a VH sequence set forth in SEQ ID NO: 456 and a VL sequence set forth in SEQ ID NO: 517.

In certain embodiments, the antibody comprises a VH sequence set forth in SEQ ID NO: 457 and a VL sequence set forth in SEQ ID NO: 518.

In certain embodiments, the antibody comprises a VH sequence set forth in SEQ ID NO: 458 and a VL sequence set forth in SEQ ID NO: 492.

In certain embodiments, the antibody comprises a VH sequence set forth in SEQ ID NO: 459 and a VL sequence set forth in SEQ ID NO: 519.

In certain embodiments, the antibody comprises a VH sequence set forth in SEQ ID NO: 460 and a VL sequence set forth in SEQ ID NO: 520.

In certain embodiments, the antibody comprises a VH sequence set forth in SEQ ID NO: 461 and a VL sequence set forth in SEQ ID NO: 521.

In certain embodiments, the antibody comprises a VH sequence set forth in SEQ ID NO: 461 and a VL sequence set forth in SEQ ID NO: 522.

In certain embodiments, the antibody comprises a VH sequence set forth in SEQ ID NO: 461 and a VL sequence set forth in SEQ ID NO: 522.

In certain embodiments, the antibody comprises a VH sequence set forth in SEQ ID NO: 462 and a VL sequence set forth in SEQ ID NO: 523.

In certain embodiments, the antibody comprises a VH sequence set forth in SEQ ID NO: 463 and a VL sequence set forth in SEQ ID NO: 523.

In certain embodiments, the antibody comprises a VH sequence set forth in SEQ ID NO: 464 and a VL sequence set forth in SEQ ID NO: 524.

In certain embodiments, the antibody comprises a VH sequence set forth in SEQ ID NO: 465 and a VL sequence set forth in SEQ ID NO: 525.

In certain embodiments, the antibody comprises a VH sequence set forth in SEQ ID NO: 466 and a VL sequence set forth in SEQ ID NO: 526.

In certain embodiments, the antibody comprises a VH sequence set forth in SEQ ID NO: 467 and a VL sequence set forth in SEQ ID NO: 527.

In certain embodiments, the antibody comprises a VH sequence set forth in SEQ ID NO: 468 and a VL sequence set forth in SEQ ID NO: 528.

In certain embodiments, the antibody comprises a VH sequence set forth in SEQ ID NO: 469 and a VL sequence set forth in SEQ ID NO: 529.

In certain embodiments, the antibody comprises a VH sequence set forth in SEQ ID NO: 470 and a VL sequence set forth in SEQ ID NO: 528.

In certain embodiments, the antibody comprises a VH sequence set forth in SEQ ID NO: 471 and a VL sequence set forth in SEQ ID NO: 530.

In certain embodiments, the antibody comprises a VH sequence set forth in SEQ ID NO: 472 and a VL sequence set forth in SEQ ID NO: 531.

In certain embodiments, the antibody comprises a VH sequence set forth in SEQ ID NO: 473 and a VL sequence set forth in SEQ ID NO: 532

In certain embodiments, the antibody comprises a VH sequence set forth in SEQ ID NO: 474 and a VL sequence set forth in SEQ ID NO: 533.

In certain embodiments, the antibody comprises a VH sequence set forth in SEQ ID NO: 475 and a VL sequence set forth in SEQ ID NO: 534.

In certain embodiments, the antibody comprises a VH sequence set forth in SEQ ID NO: 476 and a VL sequence set forth in SEQ ID NO: 535.

In certain embodiments, the antibody comprises a VH sequence set forth in SEQ ID NO: 477 and a VL sequence set forth in SEQ ID NO: 536.

In certain embodiments, the antibody comprises a VH sequence set forth in SEQ ID NO: 478 and a VL sequence set forth in SEQ ID NO: 536.

In certain embodiments, the antibody comprises a VH sequence set forth in SEQ ID NO: 479 and a VL sequence set forth in SEQ ID NO: 537.

In certain embodiments, the antibody comprises a VH sequence set forth in SEQ ID NO: 480 and a VL sequence set forth in SEQ ID NO: 538.

In certain embodiments, the antibody comprises a VH sequence set forth in SEQ ID NO: 481 and a VL sequence set forth in SEQ ID NO: 539.

In certain embodiments, the antibody comprises a VH sequence set forth in SEQ ID NO: 482 and a VL sequence set forth in SEQ ID NO: 539.

In certain embodiments, the antibody comprises a VH sequence set forth in SEQ ID NO: 483 and a VL sequence set forth in SEQ ID NO: 539.

In certain embodiments, the antibody comprises a VH sequence set forth in SEQ ID NO: 484 and a VL sequence set forth in SEQ ID NO: 540.

In certain embodiments, the antibody comprises a VH sequence set forth in SEQ ID NO: 485 and a VL sequence set forth in SEQ ID NO: 541.

In certain embodiments, the antibody comprises a VH sequence set forth in SEQ ID NO: 486 and a VL sequence set forth in SEQ ID NO: 542.

In certain embodiments, the antibody comprises a VH sequence set forth in SEQ ID NO: 487 and a VL sequence set forth in SEQ ID NO: 543.

In certain embodiments, the antibody comprises a VH sequence set forth in SEQ ID NO: 488 and a VL sequence set forth in SEQ ID NO: 544.

In certain embodiments, the antibody comprises a VH sequence set forth in SEQ ID NO: 818 and a VL sequence set forth in SEQ ID NO: 825.

In certain embodiments, the antibody comprises a VH sequence set forth in SEQ ID NO: 819 and a VL sequence set forth in SEQ ID NO: 826.

In certain embodiments, the antibody comprises a VH sequence set forth in SEQ ID NO: 820 and a VL sequence set forth in SEQ ID NO: 827.

In certain embodiments, the antibody comprises a VH sequence set forth in SEQ ID NO: 821 and a VL sequence set forth in SEQ ID NO: 828.

In certain embodiments, the antibody comprises a VH sequence set forth in SEQ ID NO: 822 and a VL sequence set forth in SEQ ID NO: 829.

In certain embodiments, the antibody comprises a VH sequence set forth in SEQ ID NO: 823 and a VL sequence set forth in SEQ ID NO: 830.

In certain embodiments, the antibody comprises a VH sequence set forth in SEQ ID NO: 824 and a VL sequence set forth in SEQ ID NO: 831.

In some embodiments, the antibody is a humanized, human, or chimeric antibody.

In some embodiments, the antibody is a humanized antibody.

In some embodiments, the antibody comprises a heavy chain human constant region of a class selected from IgG, IgA, IgD, IgE, and IgM.

In some embodiments, the human Fc region comprises a human heavy chain constant region of the class IgG and a subclass selected from IgG1, IgG2, IgG3, and IgG4.

In some embodiments, the human Fc region comprises a human IgG1 Fc.

In some embodiments, the human Fc region comprises a human IgG4 Fc.

In some embodiments, the human Fc region comprises a human IgG2 Fc.

In some embodiments, the heavy chain comprises a constant heavy chain sequence selected from an amino acid sequence set forth in any one of SEQ ID NOs: 637-737, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence selected from an amino acid sequence set forth in SEQ ID NO: 651, or an amino acid sequence having at least 90%, at least 91%, at least 92%, at least 93%, at least 94%, at least 95%, at least 96%, at least 97%, at least 98%, or at least 99% sequence identity thereto.

In some embodiments, the light chain comprises a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto.

In some embodiments, the heavy chain comprises a constant heavy chain sequence selected from an amino acid sequence set forth in SEQ ID NO: 651, or an amino acid sequence having at least 90%, at least 91%, at least 92%, at least 93%, at least 94%, at least 95%, at least 96%, at least 97%, at least 98%, or at least 99% sequence identity thereto and the light chain comprises a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto.

In some embodiments, the Fc region comprises one or more amino acid substitutions, wherein the one or more substitutions result in an increase in one or more of antibody half-life, ADCC activity, ADCP activity, and/or CDC activity compared with the Fc without the one or more substitutions.

In some embodiments, the Fc region comprises one or more amino acid substitutions, wherein the one or more substitutions result in a decrease in one or more of ADCC activity, ADCP activity, and/or CDC activity compared with the Fc without the one or more substitutions.

In some embodiments, the one or more amino acid substitutions results in increased antibody half-life compared to an antibody comprising a wild-type Fc region.

In some embodiments, the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W; optionally, wherein the one or more amino acid substitutions is a combination of amino acid substitutions selected from the group consisting of M428L/N434S (LS); M252Y/S254T/T256E (YTE); T250Q/M428L; T307A/E380A/N434A; T256D/T307Q (DQ); T256D/T307W (DW); M252Y/T256D (YD); T307Q/Q311V/A378V (QVV); T256D/H285D/T307R/Q311V/A378V (DDRVV); L309D/Q311H/N434S (DHS); S228P/L235E (SPLE); L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS; D265A/LS; LALA/LS; LALAGA/LS; LALAPG/LS; N297A/DHS; D265A/DHS; LALA/DHS; LAGA/DHS; LALAGA/DHS; LALAPG/DHS; SP/YTE; SPLE/YTE; SP/LS; SPLE/LS, SP/DHS; SPLE/DHS; N297A/LA; D265A/LA, LALA/LA, LAGA/LA, LALAGA/LA, LALAPG/LA, N297A/N434A; D265A/N434A; LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

In some embodiments, the Fc region binds to Neonatal Fc receptor (FcRn).

In some embodiments, the Fc region binds an FcRn with higher affinity at pH 6.0 compared to an antibody comprising a wild-type Fc region.

In some embodiments, the Fc region binds to FcRn with a KD of <1×10−7 M at pH 6.0.

In some embodiments, the antibody is a monoclonal antibody.

In some embodiments, the antibody binds an OX40L sequence set forth in SEQ ID NO: 739.

In some embodiments, the antibody binds to an OX40L sequence set forth in SEQ ID NO: 739 with a KD of less than or equal to about 1, 2, 3, 4, 5, 6, 7, 8, or 9×10−⁹ M, as measured by surface plasmon resonance (SPR).

In some embodiments, the antibody binds to an OX40L sequence set forth in SEQ ID NO: 739 with a KD of less than or equal to about 1×10⁻¹⁰ M, as measured by surface plasmon resonance (SPR).

In some embodiments, the antibody binds to human OX40L with a KD of less than or equal to about 1×10⁻⁹ M, as measured by surface plasmon resonance (SPR).

In some embodiments, the antibody exhibits a melting temperature greater than 68° C. as measured by Differential Scanning Fluorometry (DSF).

In some embodiments, the antibody exhibits a melting temperature greater than 75° C. as measured by Differential Scanning Fluorometry (DSF).

In some embodiments, the antibody exhibits a aggregation temperature equal to or greater than 71.2° C. as measured by Differential Scanning Fluorometry (DSF).

In some embodiments, the antibody has a retention time of 15.2 minutes or less as measured by hydrophobic interaction chromatography.

In some embodiments, the antibody can inhibit OX40L dependent IFNγ release, for example, in a human cell. In some embodiments, the antibody can inhibit OX40L dependent IFNγ release, for example, in a human.

In certain embodiments, the antibody has an increased capacity to bind cell surface OX40L as compared to amlitelimab.

In some embodiments, the antibody does not have a heavy chain variable region sequence set forth in SEQ ID NO. 113.

In some embodiments, the antibody does not have a heavy chain sequence set forth in SEQ ID NO. 146.

In certain embodiments, the isolated antibody is used in the treatment of an inflammatory disorder or disease. In certain embodiments, the isolated antibody is used in the treatment of atopic dermatitis. In certain embodiments, the isolated antibody is used in the treatment of idiopathic pulmonary fibrosis. In certain embodiments, the isolated antibody is used in the treatment of alopecia areata. In certain embodiments, the isolated antibody is used in the treatment of chronic sinusitis with nasal polyps. In certain embodiments, the isolated antibody is used in the treatment of Chronic Rhinosinusitis without Nasal Polyps (CRSsNP). In certain embodiments, the isolated antibody is used in the treatment of eosinophilic esophagitis (EoE). In certain embodiments, the isolated antibody is used in the treatment of Eosinophilic gastrointestinal disorder or disease (ENID) selected from the group consisting of Eosinophilic Gastritis (EoG), Eosinophilic Enteritis (EoN), Eosinophilic Colitis (EoC), and Eosinophilic Gastroenteritis (EGE). In certain embodiments, the isolated antibody is used in the treatment of Churg-Strauss syndrome/Eosinophilic granulomatosis with polyangiitis (EGPA). In certain embodiments, the isolated antibody is used in the treatment of Prurigo Nodularis (PN). In certain embodiments, the isolated antibody is used in the treatment of Chronic Spontaneous Urticaria (CSU). In certain embodiments, the isolated antibody is used in the treatment of Chronic Pruritis of Unknown Origin (CPUO). In certain embodiments, the isolated antibody is used in the treatment of Bullous Pemphigoid (BP). In certain embodiments, the isolated antibody is used in the treatment of Cold Inducible Urticaria (ColdU). In certain embodiments, the isolated antibody is used in the treatment of Allergic Fungal Rhinosinusitis (AFRS). In certain embodiments, the isolated antibody is used in the treatment of Allergic Bronchopulmonary Aspergillosis (ABPA). In certain embodiments, the isolated antibody is used in the treatment of Chronic Obstructive Pulmonary Disease (COPD). In certain embodiments, the isolated antibody is used in the treatment of inflammatory bowel disease, such as Crohn disease or ulcerative colitis. In certain embodiments, the isolated antibody is used in the treatment of psoriasis. In certain embodiments, the isolated antibody is used in the treatment of lupus. In certain embodiments, the isolated antibody is used in the treatment of rheumatoid arthritis. In certain embodiments, the isolated antibody is used in the treatment of hidradenitis suppurativa. In certain embodiments, the isolated antibody is used in the treatment of celiac disease. In certain embodiments, the isolated antibody is used in the treatment of systemic sclerosis.

In certain aspects, disclosed herein is an isolated polynucleotide or set of polynucleotides encoding the antibody of any of the above claims, a VH thereof, a VL thereof, a light chain thereof, a heavy chain thereof, or an antigen-binding portion thereof, and optionally, wherein the polynucleotide or set of polynucleotides comprises cDNA.

In certain aspects, disclosed herein is a vector or set of vectors comprising the polynucleotide or set of polynucleotides discloses herein.

In certain aspects, disclosed herein is a host cell comprising the polynucleotide or set of polynucleotides or the vector or set of vectors disclosed herein.

In certain aspects, disclosed herein is a method of producing an antibody, the method comprising expressing the antibody with the host cell disclosed herein and isolating the expressed antibody.

In certain aspects, disclosed herein is a pharmaceutical composition comprising the antibody of any one of the embodiments disclosed herein and a pharmaceutically acceptable excipient.

In certain aspects, disclosed herein is a kit comprising the antibody of any one of the embodiments discloses herein or a pharmaceutical composition disclosed herein and instructions for use.

In certain aspects, described herein is a method for treating an inflammatory disorder or disease in a mammalian subject in need thereof, the method comprising administering to the mammalian subject a therapeutically effective amount an antibody described herein or a pharmaceutical composition described herein. In certain embodiments of the methods described herein, the inflammatory disorder or disease is atopic dermatitis. In certain embodiments, the inflammatory disorder or disease is asthma. In certain embodiments, the inflammatory disorder or disease is idiopathic pulmonary fibrosis. In certain embodiments of the methods described herein, the inflammatory disorder or disease is alopecia areata. In certain embodiments, the inflammatory disorder or disease is chronic sinusitis with nasal polyps. In certain embodiments, the inflammatory disorder or disease is Chronic Rhinosinusitis without Nasal Polyps (CRSsNP). In certain embodiments, the inflammatory disorder or disease is eosinophilic esophagitis (EoE). In certain embodiments, the inflammatory disorder or disease is an Eosinophilic gastrointestinal disorder or disease (ENID) selected from the group consisting of Eosinophilic Gastritis (EoG), Eosinophilic Enteritis (EoN), Eosinophilic Colitis (EoC), and Eosinophilic Gastroenteritis (EGE). In certain embodiments, the inflammatory disorder or disease is Churg-Strauss syndrome/Eosinophilic granulomatosis with polyangiitis (EGPA). In certain embodiments, the inflammatory disorder or disease is Prurigo Nodularis (PN). In certain embodiments, the inflammatory disorder or disease is Chronic Spontaneous Urticaria (CSU). In certain embodiments, the inflammatory disorder or disease is Chronic Pruritis of Unknown Origin (CPUO). In certain embodiments, the inflammatory disorder or disease is Bullous Pemphigoid (BP). In certain embodiments, the inflammatory disorder or disease is Cold Inducible Urticaria (ColdU). In certain embodiments, the inflammatory disorder or disease is Allergic Fungal Rhinosinusitis (AFRS). In certain embodiments, the inflammatory disorder or disease is Allergic Bronchopulmonary Aspergillosis (ABPA). In certain embodiments, the inflammatory disorder or disease is Chronic Obstructive Pulmonary Disease (COPD). In certain embodiments, the inflammatory disorder or disease is inflammatory bowel disease, such as Crohn disease or ulcerative colitis. In certain embodiments, the inflammatory disorder or disease is psoriasis. In certain embodiments, the inflammatory disorder or disease is lupus. In certain embodiments, the inflammatory disorder or disease is rheumatoid arthritis. In certain embodiments, the inflammatory disorder or disease is celiac disease. In certain embodiments of the methods described herein, the mammalian subject is a human.

In certain aspects, disclosed herein is a method for treating a pathology associated with elevated levels of OX40L in a mammalian subject in need thereof, the method comprising administering to the mammalian subject a therapeutically effective amount the antibody of any one of the embodiments disclosed herein or a pharmaceutical composition disclosed herein. In certain embodiments of the methods described herein, the mammalian subject is a human.

In certain aspects, disclosed herein is a method of reducing biological activity of OX40L in a mammalian subject in need thereof, the method comprising administering to the mammalian subject a therapeutically effective amount the antibody of any one of the embodiments disclosed herein or a pharmaceutical composition disclosed herein. In certain embodiments of the methods described herein, the mammalian subject is a human.

In certain aspects, disclosed herein is a method of inhibiting the TH2 type allergic response in a mammalian subject in need thereof, the method comprising administering to the mammalian subject a therapeutically effective amount the antibody of any one of the embodiments disclosed herein or a pharmaceutical composition disclosed herein. In certain embodiments of the methods described herein, the mammalian subject is a human.

In certain aspects, disclosed herein is a method of preventing an inflammatory disorder or disease in a mammalian subject in need thereof, the method comprising administering to the mammalian subject a therapeutically effective amount the antibody of any one of the embodiments disclosed herein or a pharmaceutical composition disclosed herein. In certain embodiments of the methods described herein, the mammalian subject is a human.

### BRIEF DESCRIPTION OF THE FIGURES

FIG. 1 is a graph showing the ability of exemplary anti-hOX40L antibodies to binding to hOX40L-expressing CHO cells.

FIG. 2 is a graph showing the OD450-540 value with increasing concentration of the indicated antibody in the presence of hOX40L to determine the ability of the indicated antibodies to block the binding of hOX40L to hOX40.

FIG. 3 is a graph showing the luminescence units (RLU) with increasing concentration of the indicated antibody in the presence of cells that express hOX40 and to determine the ability of the indicated antibodies to block the binding of hOX40L to hOX40.

FIG. 4A and FIG. 4B are graphs showing the percent inhibition of OX40L and OX40L binding by the indicated antibody compared to a positive control.

FIG. 5A-FIG. 5C are graphs showing inhibition of IFNgamma response in PBMC-CD4 T cell mixed lymphocyte reaction (MLR) with 100 nM, 20 nM and 4 nM concentrations of indicated antibodies.

FIG. 6A and FIG. 6B are a set of graphs showing the concentration of the indicated antibody over time in the serum of cynomolgus monkeys after receiving a single dose of the indicated antibody.

### DETAILED DESCRIPTION

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4^{th} ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations. A “recombinant host cell” or “host cell” refers to a cell that includes an exogenous polynucleotide, regardless of the method used for insertion, for example, direct uptake, transduction, f-mating, or other methods known in the art to create recombinant host cells.

As used herein, the term “eukaryote” refers to organisms belonging to the phylogenetic domain Eucarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

An “effective amount” or “therapeutically effective amount” as used herein refers to an amount of therapeutic compound, such as an anti-OX40L antibody, administered to an individual, either as a single dose or as part of a series of doses, which is effective to produce or contribute to a desired therapeutic effect, either alone or in combination with another therapeutic modality. Examples of a desired therapeutic effect is enhancing an immune response, slowing or delaying tumor development; stabilization of disease; amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment include preventing recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “sufficient amount” means an amount sufficient to produce a desired effect, e.g., an amount sufficient to modulate an immune response in a subject.

As used herein, the term “subject” or “individual” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an antibody provided herein. In some aspects, the disease or condition is a cancer. In some aspects, the disease or condition is a viral infection.

The term “in vitro” refers to processes that occur in a living cell growing separate from a living organism, e.g., growing in tissue culture.

The term “in vivo” refers to processes that occur in a living organism.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “co-administration”, “co-administer”, and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to the administration of a second therapeutic agent.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)=one standard deviation of that value(s).

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

For any of the structural and functional characteristics described herein, methods of determining these characteristics are known in the art.

The term “optionally” is meant, when used sequentially, to include from one to all of the enumerated combinations and contemplates all sub-combinations.

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope).

The term “kd” (sec-1), as used herein, refers to the dissociation rate constant of a particular antibody-antigen interaction. This value is also referred to as the koff value.

The term “ka” (M−1×sec-1), as used herein, refers to the association rate constant of a particular antibody-antigen interaction. This value is also referred to as the kon value.

The term “KD” (M), as used herein, refers to the dissociation equilibrium constant of a particular antibody-antigen interaction. KD=kd/ka. In some embodiments, the affinity of an antibody is described in terms of the KD for an interaction between such antibody and its antigen. For clarity, as known in the art, a smaller KD value indicates a higher affinity interaction, while a larger KD value indicates a lower affinity interaction.

The term “KA” (M−1), as used herein, refers to the association equilibrium constant of a particular antibody-antigen interaction. KA=ka/kd.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

A “anti-OX40L antibody,” “OX40L antibody,” or “OX40L specific antibody” is an antibody, as provided herein, which specifically binds to the antigen OX40L.

The term “epitope” means a portion of an antigen that specifically binds to an antibody.

The term “hypervariable region” or “HVR”, as used herein, refers to each of the regions of an antibody variable domain which are hypervariable in sequence and/or form structurally defined loops (“hypervariable loops”).

The term “antigen-binding domain” means the portion of an antibody that is capable of specifically binding to an antigen or epitope.

The term “chimeric antibody” refers to an antibody in which a portion of the heavy and/or light chain is derived from a particular source or species, while the remainder of the heavy and/or light chain is derived from a different source or species.

The term “human antibody” refers to an antibody which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

The term “humanized antibody” refers to a protein having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject.

The term “multispecific antibody” refers to an antibody that comprises two or more different antigen-binding domains that collectively specifically bind two or more different epitopes.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with β-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. Sdabs are fairly stable and easy to express as fusion partner with the Fc chain of an antibody (Harmsen M M, De Haard H J (2007). “Properties, production, and applications of camelid single-domain antibody fragments”. Appl. Microbiol Biotechnol. 77 (1): 13-22).

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “antibody fragment” refers to an antibody that comprises a portion of an intact antibody, such as the antigen-binding or variable region of an intact antibody. Antibody fragments include, for example, Fv fragments, Fab fragments, F(ab′) 2 fragments, Fab′ fragments, scFv (sFv) fragments, and scFv-Fc fragments.

The term “Fc domain” or “Fc region” herein is used to define a C-terminal region of an immunoglobulin heavy chain that contains at least a portion of the constant region. The term includes native sequence Fc regions and variant Fc regions.

The term “substantially purified” refers to a construct described herein, or variant thereof that may be substantially or essentially free of components that normally accompany or interact with the protein as found in its naturally occurring environment, i.e. a native cell, or host cell in the case of recombinantly produced heteromultimer that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It must be noted that, as used in the specification and the appended claims, the singular forms “a,” “an” and “the” include plural referents unless the context clearly dictates otherwise.

### Anti-OX40L Antibodies

### Antibody Structure

The present application provides antibodies and compositions comprising an antibody which binds OX40L.

The recognized immunoglobulin genes include the kappa, lambda, alpha, gamma, delta, epsilon and mu constant region genes, as well as the myriad immunoglobulin variable region genes. Light chains are classified as either kappa or lambda. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, β, ε, γ, and μ, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively. The IgG1 heavy chain comprises of the VH, CH1, CH2 and CH3 domains respectively from the N to C-terminus. The light chain comprises of the VL and CL domains from N to C terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain found in an antibody provided herein, is from or derived from an immunoglobulin based construct such as a diabody, or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody or any chimeric antibody.

In some embodiments, the antibodies provided herein comprise a heavy chain. In one embodiment, the heavy chain is an IgA. In one embodiment, the heavy chain is an IgD. In one embodiment, the heavy chain is an IgE. In one embodiment, the heavy chain is an IgG. In one embodiment, the heavy chain is an IgM. In one embodiment, the heavy chain is an IgG1. In one embodiment, the heavy chain is an IgG2. In one embodiment, the heavy chain is an IgG3. In one embodiment, the heavy chain is an IgG4. In one embodiment, the heavy chain is an IgA1. In one embodiment, the heavy chain is an IgA2.

In some embodiments, an antibody is an IgG1 antibody. In some embodiments, an antibody is an IgG3 antibody. In some embodiments, an antibody is an IgG2 antibody. In some embodiments, an antibody is an IgG4 antibody.

Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“aHo” numbering scheme); each of which is incorporated by reference in its entirety.

TABLE 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE 1
TABLE 1: Residues in CDRs according to
Kabat and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

One example of an antigen-binding domain is an antigen-binding domain formed by a VH-VL dimer of an antibody. Another example of an antigen-binding domain is an antigen-binding domain formed by diversification of certain loops from the tenth fibronectin type III domain of an Adnectin. An antigen-binding domain can include CDRs 1, 2, and 3 from a heavy chain in that order; and CDRs 1, 2, and 3 from a light chain in that order.

Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to OX40L variants with different point-mutations, or to chimeric OX40L variants.

To screen for antibodies which bind to an epitope on a target antigen bound by an antibody of interest (e.g., OX40L), a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art.

Chimeric antibodies are antibodies in which a portion of the heavy and/or light chain is derived from a particular source or species, while the remainder of the heavy and/or light chain is derived from a different source or species.

Human antibodies are antibodies which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297, 5,886,152 and 5,877,293. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

The two or more different epitopes may be epitopes on the same antigen (e.g., a single OX40L) or on different antigens (e.g., different OX40L molecules, or a OX40L molecule and a non-OX40L molecule). In some aspects, a multi-specific antibody binds two different epitopes (i.e., a “bispecific antibody”). In some aspects, a multi-specific antibody binds three different epitopes (i.e., a “trispecific antibody”).

Anti-OX40L antibodies can include those described herein such as the clones set forth in the drawings and/or tables. In some embodiments, the antibody comprises an alternative scaffold. In some embodiments, the antibody consists of an alternative scaffold. In some embodiments, the antibody consists essentially of an alternative scaffold. In some embodiments, the antibody comprises an antibody fragment. In some embodiments, the antibody consists of an antibody fragment. In some embodiments, the antibody consists essentially of an antibody fragment.

In some embodiments, the antibodies are monoclonal antibodies.

In some embodiments, the antibodies are polyclonal antibodies.

In some embodiments, the antibodies are produced by hybridomas. In other embodiments, the antibodies are produced by recombinant cells engineered to express the desired variable and constant domains.

In some embodiments, the antibodies may be single chain antibodies or other antibody derivatives retaining the antigen specificity and the lower hinge region or a variant thereof.

In some embodiments, the antibodies may be polyfunctional antibodies, recombinant antibodies, human antibodies, humanized antibodies, fragments or variants thereof. In particular embodiments, the antibody fragment or a derivative thereof is selected from a Fab fragment, a Fab′2 fragment, a CDR and ScFv.

In some embodiments, the antibodies are capable of forming an immune complex. For example, an immune complex can be a tumor cell covered by antibodies.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

TABLE 2
Sequences of OX40L Antibody Constructs - VH, VL, and Associated CDRs

| Identifier | VH and Heavy chain CDRs | VL and Light chain CDRs |
|---|---|---|
| 114 | VH (SEQ ID NO: 419) | VL (SEQ ID NO: 489) |
| | CDR1: Kabat (SEQ ID NO: 1) | CDR1: Kabat (SEQ ID NO: 258) |
| | Chothia (SEQ ID NO: 22) | Chothia (SEQ ID NO: 286) |
| | IMGT (SEQ ID NO: 44) | IMGT (SEQ ID NO: 311) |
| | CDR2: Kabat (SEQ ID NO: 70) | CDR2: Kabat (SEQ ID NO: 335) |
| | Chothia (SEQ ID NO: 111) | Chothia (SEQ ID NO: 359) |
| | IMGT (SEQ ID NO: 142) | IMGT (SEQ ID NO: 359) |
| | CDR3: Kabat (SEQ ID NO: 175) | CDR3: Kabat (SEQ ID NO: 368) |
| | Chothia (SEQ ID NO: 203) | Chothia (SEQ ID NO: 394) |
| | IMGT (SEQ ID NO: 223) | IMGT (SEQ ID NO: 368) |
| 117 | VH (SEQ ID NO: 420) | VL (SEQ ID NO: 490) |
| | CDR1: Kabat (SEQ ID NO: 2) | CDR1: Kabat (SEQ ID NO: 259) |
| | Chothia (SEQ ID NO: 23) | Chothia (SEQ ID NO: 287) |
| | IMGT (SEQ ID NO: 45) | IMGT (SEQ ID NO: 312) |
| | CDR2: Kabat (SEQ ID NO: 70) | CDR2: Kabat (SEQ ID NO: 336) |
| | Chothia (SEQ ID NO: 111) | Chothia (SEQ ID NO: 360) |
| | IMGT (SEQ ID NO: 142) | IMGT (SEQ ID NO: 360) |
| | CDR3: Kabat (SEQ ID NO: 176) | CDR3: Kabat (SEQ ID NO: 369) |
| | Chothia (SEQ ID NO: 204) | Chothia (SEQ ID NO: 395) |
| | IMGT (SEQ ID NO: 224) | IMGT (SEQ ID NO: 369) |
| 118 | VH (SEQ ID NO: 421) | VL (SEQ ID NO: 491) |
| | CDR1: Kabat (SEQ ID NO: 3) | CDR1: Kabat (SEQ ID NO: 260) |
| | Chothia (SEQ ID NO: 24) | Chothia (SEQ ID NO: 288) |
| | IMGT (SEQ ID NO: 46) | IMGT (SEQ ID NO: 313) |
| | CDR2: Kabat (SEQ ID NO: 71) | CDR2: Kabat (SEQ ID NO: 337) |
| | Chothia (SEQ ID NO: 112) | Chothia (SEQ ID NO: 361) |
| | IMGT (SEQ ID NO: 143) | IMGT (SEQ ID NO: 361) |
| | CDR3: Kabat (SEQ ID NO: 177) | CDR3: Kabat (SEQ ID NO: 370) |
| | Chothia (SEQ ID NO: 205) | Chothia (SEQ ID NO: 396) |
| | IMGT (SEQ ID NO: 225) | IMGT (SEQ ID NO: 370) |
| 122 | VH (SEQ ID NO: 422) | VL (SEQ ID NO: 492) |
| | CDR1: Kabat (SEQ ID NO: 4) | CDR1: Kabat (SEQ ID NO: 261) |
| | Chothia (SEQ ID NO: 22) | Chothia (SEQ ID NO: 289) |
| | IMGT (SEQ ID NO: 47) | IMGT (SEQ ID NO: 314) |
| | CDR2: Kabat (SEQ ID NO: 72) | CDR2: Kabat (SEQ ID NO: 337) |
| | Chothia (SEQ ID NO: 113) | Chothia (SEQ ID NO: 361) |
| | IMGT (SEQ ID NO: 144) | IMGT (SEQ ID NO: 361) |
| | CDR3: Kabat (SEQ ID NO: 178) | CDR3: Kabat (SEQ ID NO: 371) |
| | Chothia (SEQ ID NO: 206) | Chothia (SEQ ID NO: 397) |
| | IMGT (SEQ ID NO: 226) | IMGT (SEQ ID NO: 371) |
| 127 | VH (SEQ ID NO: 423) | VL (SEQ ID NO: 493) |
| | CDR1: Kabat (SEQ ID NO: 5) | CDR1: Kabat (SEQ ID NO: 262) |
| | Chothia (SEQ ID NO: 840) | Chothia (SEQ ID NO: 290) |
| | IMGT (SEQ ID NO: 48) | IMGT (SEQ ID NO: 315) |
| | CDR2: Kabat (SEQ ID NO: 73) | CDR2: Kabat (SEQ ID NO: 338) |
| | Chothia (SEQ ID NO: 114) | Chothia (SEQ ID NO: 362) |
| | IMGT (SEQ ID NO: 145) | IMGT (SEQ ID NO: 362) |
| | CDR3: Kabat (SEQ ID NO: 179) | CDR3: Kabat (SEQ ID NO: 372) |
| | Chothia (SEQ ID NO: 207) | Chothia (SEQ ID NO: 398) |
| | IMGT (SEQ ID NO: 227) | IMGT (SEQ ID NO: 372) |
| 128 | VH (SEQ ID NO: 424) | VL (SEQ ID NO: 494) |
| | CDR1: Kabat (SEQ ID NO: 6) | CDR1: Kabat (SEQ ID NO: 263) |
| | Chothia (SEQ ID NO: 24) | Chothia (SEQ ID NO: 291) |
| | IMGT (SEQ ID NO: 49) | IMGT (SEQ ID NO: 316) |
| | CDR2: Kabat (SEQ ID NO: 74) | |
| | Chothia (SEQ ID NO: 115) | CDR2: Kabat (SEQ ID NO: 339) |
| | IMGT (SEQ ID NO: 146) | Chothia (SEQ ID NO: 361) |
| | CDR3: Kabat (SEQ ID NO: 180) | IMGT (SEQ ID NO: 361) |
| | Chothia (SEQ ID NO: 208) | CDR3: Kabat (SEQ ID NO: 373) |
| | IMGT (SEQ ID NO: 228) | Chothia (SEQ ID NO: 399) |
| | | IMGT (SEQ ID NO: 373) |
| 130 | VH (SEQ ID NO: 425) | VL (SEQ ID NO: 495) |
| | CDR1: Kabat (SEQ ID NO: 7) | CDR1: Kabat (SEQ ID NO: 264) |
| | Chothia (SEQ ID NO: 25) | Chothia (SEQ ID NO: 292) |
| | IMGT (SEQ ID NO: 50) | IMGT (SEQ ID NO: 317) |
| | CDR2: Kabat (SEQ ID NO: 75) | |
| | Chothia (SEQ ID NO: 116) | CDR2: Kabat (SEQ ID NO: 340) |
| | IMGT (SEQ ID NO: 147) | Chothia (SEQ ID NO: 363) |
| | CDR3: Kabat (SEQ ID NO: 181) | IMGT (SEQ ID NO: 363) |
| | Chothia (SEQ ID NO: 209) | CDR3: Kabat (SEQ ID NO: 374) |
| | IMGT (SEQ ID NO: 229) | Chothia (SEQ ID NO: 400) |
| | | IMGT (SEQ ID NO: 374) |
| 327 | VH (SEQ ID NO: 426) | VL (SEQ ID NO: 496) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 182) | CDR3: Kabat (SEQ ID NO: 375) |
| | Chothia (SEQ ID NO: 210) | Chothia (SEQ ID NO: 401) |
| | IMGT (SEQ ID NO: 230) | IMGT (SEQ ID NO: 375) |
| 329 | VH (SEQ ID NO: 427) | VL (SEQ ID NO: 497) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 183) | CDR3: Kabat (SEQ ID NO: 376) |
| | Chothia (SEQ ID NO: 211) | Chothia (SEQ ID NO: 402) |
| | IMGT (SEQ ID NO: 231) | IMGT (SEQ ID NO: 376) |
| 330 | VH (SEQ ID NO: 427) | VL (SEQ ID NO: 498) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 183) | CDR3: Kabat (SEQ ID NO: 377) |
| | Chothia (SEQ ID NO: 211) | Chothia (SEQ ID NO: 403) |
| | IMGT (SEQ ID NO: 231) | IMGT (SEQ ID NO: 377) |
| 331 | VH (SEQ ID NO: 428) | VL (SEQ ID NO: 497) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 184) | CDR3: Kabat (SEQ ID NO: 376) |
| | Chothia (SEQ ID NO: 212) | Chothia (SEQ ID NO: 402) |
| | IMGT (SEQ ID NO: 232) | IMGT (SEQ ID NO: 376) |
| 332 | VH (SEQ ID NO: 428) | VL (SEQ ID NO: 498) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 184) | CDR3: Kabat (SEQ ID NO: 377) |
| | Chothia (SEQ ID NO: 212) | Chothia (SEQ ID NO: 403) |
| | IMGT (SEQ ID NO: 232) | IMGT (SEQ ID NO: 377) |
| 333 | VH (SEQ ID NO: 429) | VL (SEQ ID NO: 497) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 185) | CDR3: Kabat (SEQ ID NO: 376) |
| | Chothia (SEQ ID NO: 213) | Chothia (SEQ ID NO: 402) |
| | IMGT (SEQ ID NO: 233) | IMGT (SEQ ID NO: 376) |
| 334 | VH (SEQ ID NO: 429) | VL (SEQ ID NO: 498) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 185) | CDR3: Kabat (SEQ ID NO: 377) |
| | Chothia (SEQ ID NO: 213) | Chothia (SEQ ID NO: 403) |
| | IMGT (SEQ ID NO: 233) | IMGT (SEQ ID NO: 377) |
| 501 | VH (SEQ ID NO: 430) | VL (SEQ ID NO: 499) |
| | CDR1: Kabat (SEQ ID NO: 9) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 77) | CDR2: Kabat (SEQ ID NO: 342) |
| | Chothia (SEQ ID NO: 118) | Chothia (SEQ ID NO: 364) |
| | IMGT (SEQ ID NO: 149) | IMGT (SEQ ID NO: 364) |
| | CDR3: Kabat (SEQ ID NO: 175) | CDR3: Kabat (SEQ ID NO: 368) |
| | Chothia (SEQ ID NO: 203) | Chothia (SEQ ID NO: 394) |
| | IMGT (SEQ ID NO: 234) | IMGT (SEQ ID NO: 368) |
| 502 | VH (SEQ ID NO: 431) | VL (SEQ ID NO: 500) |
| | CDR1: Kabat (SEQ ID NO: 1) | CDR1: Kabat (SEQ ID NO: 266) |
| | Chothia (SEQ ID NO: 28) | Chothia (SEQ ID NO: 286) |
| | IMGT (SEQ ID NO: 53) | IMGT (SEQ ID NO: 311) |
| | CDR2: Kabat (SEQ ID NO: 78) | CDR2: Kabat (SEQ ID NO: 343) |
| | Chothia (SEQ ID NO: 119) | Chothia (SEQ ID NO: 365) |
| | IMGT (SEQ ID NO: 150) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 503 | VH (SEQ ID NO: 432) | VL (SEQ ID NO: 501) |
| | CDR1: Kabat (SEQ ID NO: 10) | CDR1: Kabat (SEQ ID NO: 267) |
| | Chothia (SEQ ID NO: 27) | Chothia (SEQ ID NO: 294) |
| | IMGT (SEQ ID NO: 52) | IMGT (SEQ ID NO: 319) |
| | CDR2: Kabat (SEQ ID NO: 79) | CDR2: Kabat (SEQ ID NO: 344) |
| | Chothia (SEQ ID NO: 119) | Chothia (SEQ ID NO: 365) |
| | IMGT (SEQ ID NO: 150) | IMGT (SEQ ID NO: 365) |
| | CDR3: Kabat (SEQ ID NO: 186) | CDR3: Kabat (SEQ ID NO: 378) |
| | Chothia (SEQ ID NO: 203) | Chothia (SEQ ID NO: 404) |
| | IMGT (SEQ ID NO: 235) | IMGT (SEQ ID NO: 378) |
| 504 | VH (SEQ ID NO: 433) | VL (SEQ ID NO: 502) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 80) | CDR2: Kabat (SEQ ID NO: 343) |
| | Chothia (SEQ ID NO: 120) | Chothia (SEQ ID NO: 365) |
| | IMGT (SEQ ID NO: 151) | IMGT (SEQ ID NO: 365) |
| | CDR3: Kabat (SEQ ID NO: 186) | CDR3: Kabat (SEQ ID NO: 378) |
| | Chothia (SEQ ID NO: 203) | Chothia (SEQ ID NO: 404) |
| | IMGT (SEQ ID NO: 235) | IMGT (SEQ ID NO: 378) |
| 505 | VH (SEQ ID NO: 434) | VL (SEQ ID NO: 502) |
| | CDR1: Kabat (SEQ ID NO: 11) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 79) | CDR2: Kabat (SEQ ID NO: 343) |
| | Chothia (SEQ ID NO: 119) | Chothia (SEQ ID NO: 365) |
| | IMGT (SEQ ID NO: 150) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 506 | VH (SEQ ID NO: 435) | VL (SEQ ID NO: 502) |
| | CDR1: Kabat (SEQ ID NO: 11) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 81) | CDR2: Kabat (SEQ ID NO: 343) |
| | Chothia (SEQ ID NO: 121) | Chothia (SEQ ID NO: 365) |
| | IMGT (SEQ ID NO: 152) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 507 | VH (SEQ ID NO: 436) | VL (SEQ ID NO: 502) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 80) | CDR2: Kabat (SEQ ID NO: 343) |
| | Chothia (SEQ ID NO: 120) | Chothia (SEQ ID NO: 365) |
| | IMGT (SEQ ID NO: 151) | IMGT (SEQ ID NO: 365) |
| | CDR3: Kabat (SEQ ID NO: 187) | CDR3: Kabat (SEQ ID NO: 378) |
| | Chothia (SEQ ID NO: 203) | Chothia (SEQ ID NO: 404) |
| | IMGT (SEQ ID NO: 236) | IMGT (SEQ ID NO: 378) |
| 508 | VH (SEQ ID NO: 437) | VL (SEQ ID NO: 502) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 79) | CDR2: Kabat (SEQ ID NO: 343) |
| | Chothia (SEQ ID NO: 119) | Chothia (SEQ ID NO: 365) |
| | IMGT (SEQ ID NO: 150) | IMGT (SEQ ID NO: 365) |
| | CDR3: Kabat (SEQ ID NO: 187) | CDR3: Kabat (SEQ ID NO: 378) |
| | Chothia (SEQ ID NO: 203) | Chothia (SEQ ID NO: 404) |
| | IMGT (SEQ ID NO: 236) | IMGT (SEQ ID NO: 378) |
| 509 | VH (SEQ ID NO: 438) | VL (SEQ ID NO: 503) |
| | CDR1: Kabat (SEQ ID NO: 10) | CDR1: Kabat (SEQ ID NO: 268) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 188) | CDR3: Kabat (SEQ ID NO: 368) |
| | Chothia (SEQ ID NO: 203) | Chothia (SEQ ID NO: 394) |
| | IMGT (SEQ ID NO: 237) | IMGT (SEQ ID NO: 368) |
| 510 | VH (SEQ ID NO: 439) | VL (SEQ ID NO: 502) |
| | CDR1: Kabat (SEQ ID NO: 12) | CDR1: Kabat (SEQ ID NO: 260) |
| | Chothia (SEQ ID NO: 29) | Chothia (SEQ ID NO: 288) |
| | IMGT (SEQ ID NO: 54) | IMGT (SEQ ID NO: 313) |
| | CDR2: Kabat (SEQ ID NO: 70) | CDR2: Kabat (SEQ ID NO: 343) |
| | Chothia (SEQ ID NO: 111) | Chothia (SEQ ID NO: 365) |
| | IMGT (SEQ ID NO: 142) | IMGT (SEQ ID NO: 365) |
| | CDR3: Kabat (SEQ ID NO: 188) | CDR3: Kabat (SEQ ID NO: 378) |
| | Chothia (SEQ ID NO: 203) | Chothia (SEQ ID NO: 404) |
| | IMGT (SEQ ID NO: 237) | IMGT (SEQ ID NO: 378) |
| 511 | VH (SEQ ID NO: 440) | VL (SEQ ID NO: 504) |
| | CDR1: Kabat (SEQ ID NO: 10) | CDR1: Kabat (SEQ ID NO: 269) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 82) | CDR2: Kabat (SEQ ID NO: 345) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 189) | CDR3: Kabat (SEQ ID NO: 378) |
| | Chothia (SEQ ID NO: 214) | Chothia (SEQ ID NO: 404) |
| | IMGT (SEQ ID NO: 238) | IMGT (SEQ ID NO: 378) |
| 512 | VH (SEQ ID NO: 441) | VL (SEQ ID NO: 505) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 83) | CDR2: Kabat (SEQ ID NO: 346) |
| | Chothia (SEQ ID NO: 122) | Chothia (SEQ ID NO: 361) |
| | IMGT (SEQ ID NO: 153) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 234) | IMGT (SEQ ID NO: 378) |
| 513 | VH (SEQ ID NO: 442) | VL (SEQ ID NO: 506) |
| | CDR1: Kabat (SEQ ID NO: 13) | CDR1: Kabat (SEQ ID NO: 260) |
| | Chothia (SEQ ID NO: 30) | Chothia (SEQ ID NO: 288) |
| | IMGT (SEQ ID NO: 55) | IMGT (SEQ ID NO: 313) |
| | CDR2: Kabat (SEQ ID NO: 84) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 188) | CDR3: Kabat (SEQ ID NO: 378) |
| | Chothia (SEQ ID NO: 203) | Chothia (SEQ ID NO: 404) |
| | IMGT (SEQ ID NO: 239) | IMGT (SEQ ID NO: 378) |
| 514 | VH (SEQ ID NO: 443) | VL (SEQ ID NO: 507) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 347) |
| | Chothia (SEQ ID NO: 111) | Chothia (SEQ ID NO: 365) |
| | IMGT (SEQ ID NO: 142) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 234) | IMGT (SEQ ID NO: 378) |
| 515 | VH (SEQ ID NO: 443) | VL (SEQ ID NO: 508) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 345) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 234) | IMGT (SEQ ID NO: 378) |
| 516 | VH (SEQ ID NO: 444) | VL (SEQ ID NO: 506) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 85) | CDR2: Kabat (SEQ ID NO: 337) |
| | Chothia (SEQ ID NO: 123) | Chothia (SEQ ID NO: 361) |
| | IMGT (SEQ ID NO: 154) | IMGT (SEQ ID NO: 361) |
| | CDR3: Kabat (SEQ ID NO: 188) | CDR3: Kabat (SEQ ID NO: 378) |
| | Chothia (SEQ ID NO: 203) | Chothia (SEQ ID NO: 404) |
| | IMGT (SEQ ID NO: 239) | IMGT (SEQ ID NO: 378) |
| 517 | VH (SEQ ID NO: 445) | VL (SEQ ID NO: 509) |
| | CDR1: Kabat (SEQ ID NO: 10) | CDR1: Kabat (SEQ ID NO: 270) |
| | Chothia (SEQ ID NO: 27) | Chothia (SEQ ID NO: 295) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 518 | VH (SEQ ID NO: 446) | VL (SEQ ID NO: 510) |
| | CDR1: Kabat (SEQ ID NO: 1) | CDR1: Kabat (SEQ ID NO: 260) |
| | Chothia (SEQ ID NO: 31) | Chothia (SEQ ID NO: 288) |
| | IMGT (SEQ ID NO: 56) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 379) |
| | Chothia (SEQ ID NO: 203) | Chothia (SEQ ID NO: 405) |
| | IMGT (SEQ ID NO: 223) | IMGT (SEQ ID NO: 379) |
| 519 | VH (SEQ ID NO: 447) | VL (SEQ ID NO: 506) |
| | CDR1: Kabat (SEQ ID NO: 1) | CDR1: Kabat (SEQ ID NO: 260) |
| | Chothia (SEQ ID NO: 32) | Chothia (SEQ ID NO: 288) |
| | IMGT (SEQ ID NO: 57) | IMGT (SEQ ID NO: 313) |
| | CDR2: Kabat (SEQ ID NO: 86) | CDR2: Kabat (SEQ ID NO: 337) |
| | Chothia (SEQ ID NO: 124) | Chothia (SEQ ID NO: 361) |
| | IMGT (SEQ ID NO: 155) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 520 | VH (SEQ ID NO: 445) | VL (SEQ ID NO: 511) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 380) |
| | Chothia (SEQ ID NO: 203) | Chothia (SEQ ID NO: 406) |
| | IMGT (SEQ ID NO: 223) | IMGT (SEQ ID NO: 380) |
| 521 | VH (SEQ ID NO: 448) | VL (SEQ ID NO: 506) |
| | CDR1: Kabat (SEQ ID NO: 14) | CDR1: Kabat (SEQ ID NO: 260) |
| | Chothia (SEQ ID NO: 33) | Chothia (SEQ ID NO: 288) |
| | IMGT (SEQ ID NO: 58) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 522 | VH (SEQ ID NO: 449) | VL (SEQ ID NO: 502) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 87) | CDR2: Kabat (SEQ ID NO: 343) |
| | Chothia (SEQ ID NO: 111) | Chothia (SEQ ID NO: 365) |
| | IMGT (SEQ ID NO: 142) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 523 | VH (SEQ ID NO: 449) | VL (SEQ ID NO: 506) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 87) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 524 | VH (SEQ ID NO: 450) | VL (SEQ ID NO: 506) |
| | CDR1: Kabat (SEQ ID NO: 13) | CDR1: Kabat (SEQ ID NO: 260) |
| | Chothia (SEQ ID NO: 30) | Chothia (SEQ ID NO: 288) |
| | IMGT (SEQ ID NO: 55) | IMGT (SEQ ID NO: 313) |
| | CDR2: Kabat (SEQ ID NO: 87) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 525 | VH (SEQ ID NO: 445) | VL (SEQ ID NO: 512) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | |
| | Chothia (SEQ ID NO: 111) | CDR2: Kabat (SEQ ID NO: 337) |
| | IMGT (SEQ ID NO: 142) | Chothia (SEQ ID NO: 361) |
| | CDR3: Kabat (SEQ ID NO: 175) | IMGT (SEQ ID NO: 361) |
| | Chothia (SEQ ID NO: 203) | CDR3: Kabat (SEQ ID NO: 368) |
| | IMGT (SEQ ID NO: 223) | Chothia (SEQ ID NO: 394) |
| | | IMGT (SEQ ID NO: 368) |
| 526 | VH (SEQ ID NO: 449) | VL (SEQ ID NO: 513) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 87) | CDR2: Kabat (SEQ ID NO: 348) |
| | Chothia (SEQ ID NO: 111) | Chothia (SEQ ID NO: 366) |
| | IMGT (SEQ ID NO: 142) | IMGT (SEQ ID NO: 366) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 527 | VH (SEQ ID NO: 445) | VL (SEQ ID NO: 514) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 381) |
| | Chothia (SEQ ID NO: 203) | Chothia (SEQ ID NO: 405) |
| | IMGT (SEQ ID NO: 223) | IMGT (SEQ ID NO: 381) |
| 528 | VH (SEQ ID NO: 445) | VL (SEQ ID NO: 515) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 335) |
| | Chothia (SEQ ID NO: 111) | Chothia (SEQ ID NO: 359) |
| | IMGT (SEQ ID NO: 142) | IMGT (SEQ ID NO: 359) |
| | CDR3: Kabat (SEQ ID NO: 175) | CDR3: Kabat (SEQ ID NO: 368) |
| | Chothia (SEQ ID NO: 203) | Chothia (SEQ ID NO: 394) |
| | IMGT (SEQ ID NO: 223) | IMGT (SEQ ID NO: 368) |
| 529 | VH (SEQ ID NO: 451) | VL (SEQ ID NO: 514) |
| | CDR1: Kabat (SEQ ID NO: 1) | CDR1: Kabat (SEQ ID NO: 260) |
| | Chothia (SEQ ID NO: 22) | Chothia (SEQ ID NO: 288) |
| | IMGT (SEQ ID NO: 44) | IMGT (SEQ ID NO: 313) |
| | CDR2: Kabat (SEQ ID NO: 86) | CDR2: Kabat (SEQ ID NO: 337) |
| | Chothia (SEQ ID NO: 124) | Chothia (SEQ ID NO: 361) |
| | IMGT (SEQ ID NO: 155) | IMGT (SEQ ID NO: 361) |
| | CDR3: Kabat (SEQ ID NO: 175) | CDR3: Kabat (SEQ ID NO: 381) |
| | Chothia (SEQ ID NO: 203) | Chothia (SEQ ID NO: 405) |
| | IMGT (SEQ ID NO: 223) | IMGT (SEQ ID NO: 381) |
| 530 | VH (SEQ ID NO: 451) | VL (SEQ ID NO: 506) |
| | CDR1: Kabat (SEQ ID NO: 1) | CDR1: Kabat (SEQ ID NO: 260) |
| | Chothia (SEQ ID NO: 22) | Chothia (SEQ ID NO: 288) |
| | IMGT (SEQ ID NO: 44) | IMGT (SEQ ID NO: 313) |
| | CDR2: Kabat (SEQ ID NO: 86) | CDR2: Kabat (SEQ ID NO: 337) |
| | Chothia (SEQ ID NO: 124) | Chothia (SEQ ID NO: 361) |
| | IMGT (SEQ ID NO: 155) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 531 | VH (SEQ ID NO: 452) | VL (SEQ ID NO: 506) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 188) | CDR3: Kabat (SEQ ID NO: 378) |
| | Chothia (SEQ ID NO: 203) | Chothia (SEQ ID NO: 404) |
| | IMGT (SEQ ID NO: 239) | IMGT (SEQ ID NO: 378) |
| 532 | VH (SEQ ID NO: 438) | VL (SEQ ID NO: 506) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 188) | CDR3: Kabat (SEQ ID NO: 378) |
| | Chothia (SEQ ID NO: 203) | Chothia (SEQ ID NO: 404) |
| | IMGT (SEQ ID NO: 237) | IMGT (SEQ ID NO: 378) |
| 533 | VH (SEQ ID NO: 453) | VL (SEQ ID NO: 512) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 78) | CDR2: Kabat (SEQ ID NO: 337) |
| | Chothia (SEQ ID NO: 119) | Chothia (SEQ ID NO: 361) |
| | IMGT (SEQ ID NO: 150) | IMGT (SEQ ID NO: 361) |
| | CDR3: Kabat (SEQ ID NO: 188) | CDR3: Kabat (SEQ ID NO: 368) |
| | Chothia (SEQ ID NO: 203) | Chothia (SEQ ID NO: 394) |
| | IMGT (SEQ ID NO: 237) | IMGT (SEQ ID NO: 368) |
| 534 | VH (SEQ ID NO: 454) | VL (SEQ ID NO: 490) |
| | CDR1: Kabat (SEQ ID NO: 2) | CDR1: Kabat (SEQ ID NO: 259) |
| | Chothia (SEQ ID NO: 23) | Chothia (SEQ ID NO: 287) |
| | IMGT (SEQ ID NO: 45) | IMGT (SEQ ID NO: 312) |
| | CDR2: Kabat (SEQ ID NO: 70) | CDR2: Kabat (SEQ ID NO: 336) |
| | Chothia (SEQ ID NO: 111) | Chothia (SEQ ID NO: 360) |
| | IMGT (SEQ ID NO: 142) | IMGT (SEQ ID NO: 360) |
| | CDR3: Kabat (SEQ ID NO: 176) | CDR3: Kabat (SEQ ID NO: 369) |
| | Chothia (SEQ ID NO: 204) | Chothia (SEQ ID NO: 395) |
| | IMGT (SEQ ID NO: 224) | IMGT (SEQ ID NO: 369) |
| 535 | VH (SEQ ID NO: 455) | VL (SEQ ID NO: 516) |
| | CDR1: Kabat (SEQ ID NO: 15) | CDR1: Kabat (SEQ ID NO: 269) |
| | Chothia (SEQ ID NO: 34) | Chothia (SEQ ID NO: 288) |
| | IMGT (SEQ ID NO: 59) | IMGT (SEQ ID NO: 313) |
| | CDR2: Kabat (SEQ ID NO: 88) | CDR2: Kabat (SEQ ID NO: 349) |
| | Chothia (SEQ ID NO: 125) | Chothia (SEQ ID NO: 365) |
| | IMGT (SEQ ID NO: 156) | IMGT (SEQ ID NO: 365) |
| | CDR3: Kabat (SEQ ID NO: 190) | CDR3: Kabat (SEQ ID NO: 382) |
| | Chothia (SEQ ID NO: 215) | Chothia (SEQ ID NO: 407) |
| | IMGT (SEQ ID NO: 240) | IMGT (SEQ ID NO: 382) |
| 536 | VH (SEQ ID NO: 456) | VL (SEQ ID NO: 517) |
| | CDR1: Kabat (SEQ ID NO: 4) | CDR1: Kabat (SEQ ID NO: 260) |
| | Chothia (SEQ ID NO: 22) | Chothia (SEQ ID NO: 288) |
| | IMGT (SEQ ID NO: 47) | IMGT (SEQ ID NO: 313) |
| | CDR2: Kabat (SEQ ID NO: 89) | CDR2: Kabat (SEQ ID NO: 337) |
| | Chothia (SEQ ID NO: 126) | Chothia (SEQ ID NO: 361) |
| | IMGT (SEQ ID NO: 157) | IMGT (SEQ ID NO: 361) |
| | CDR3: Kabat (SEQ ID NO: 178) | CDR3: Kabat (SEQ ID NO: 371) |
| | Chothia (SEQ ID NO: 206) | Chothia (SEQ ID NO: 397) |
| | IMGT (SEQ ID NO: 226) | IMGT (SEQ ID NO: 371) |
| 537 | VH (SEQ ID NO: 457) | VL (SEQ ID NO: 518) |
| | CDR1: Kabat (SEQ ID NO: 4) | CDR1: Kabat (SEQ ID NO: 271) |
| | Chothia (SEQ ID NO: 35) | Chothia (SEQ ID NO: 296) |
| | IMGT (SEQ ID NO: 60) | IMGT (SEQ ID NO: 320) |
| | CDR2: Kabat (SEQ ID NO: 72) | CDR2: Kabat (SEQ ID NO: 350) |
| | Chothia (SEQ ID NO: 113) | Chothia (SEQ ID NO: 361) |
| | IMGT (SEQ ID NO: 144) | IMGT (SEQ ID NO: 361) |
| | CDR3: Kabat (SEQ ID NO: 178) | CDR3: Kabat (SEQ ID NO: 371) |
| | Chothia (SEQ ID NO: 206) | Chothia (SEQ ID NO: 397) |
| | IMGT (SEQ ID NO: 241) | IMGT (SEQ ID NO: 371) |
| 538 | VH (SEQ ID NO: 458) | VL (SEQ ID NO: 492) |
| | CDR1: Kabat (SEQ ID NO: 4) | CDR1: Kabat (SEQ ID NO: 261) |
| | Chothia (SEQ ID NO: 22) | Chothia (SEQ ID NO: 289) |
| | IMGT (SEQ ID NO: 47) | IMGT (SEQ ID NO: 314) |
| | CDR2: Kabat (SEQ ID NO: 90) | CDR2: Kabat (SEQ ID NO: 337) |
| | Chothia (SEQ ID NO: 127) | Chothia (SEQ ID NO: 361) |
| | IMGT (SEQ ID NO: 158) | IMGT (SEQ ID NO: 361) |
| | CDR3: Kabat (SEQ ID NO: 178) | CDR3: Kabat (SEQ ID NO: 371) |
| | Chothia (SEQ ID NO: 206) | Chothia (SEQ ID NO: 397) |
| | IMGT (SEQ ID NO: 226) | IMGT (SEQ ID NO: 371) |
| 539 | VH (SEQ ID NO: 459) | VL (SEQ ID NO: 519) |
| | CDR1: Kabat (SEQ ID NO: 16) | CDR1: Kabat (SEQ ID NO: 269) |
| | Chothia (SEQ ID NO: 36) | Chothia (SEQ ID NO: 288) |
| | IMGT (SEQ ID NO: 61) | IMGT (SEQ ID NO: 313) |
| | CDR2: Kabat (SEQ ID NO: 91) | CDR2: Kabat (SEQ ID NO: 351) |
| | Chothia (SEQ ID NO: 128) | Chothia (SEQ ID NO: 361) |
| | IMGT (SEQ ID NO: 159) | IMGT (SEQ ID NO: 361) |
| | CDR3: Kabat (SEQ ID NO: 191) | CDR3: Kabat (SEQ ID NO: 382) |
| | Chothia (SEQ ID NO: 215) | Chothia (SEQ ID NO: 407) |
| | IMGT (SEQ ID NO: 242) | IMGT (SEQ ID NO: 382) |
| 540 | VH (SEQ ID NO: 460) | VL (SEQ ID NO: 520) |
| | CDR1: Kabat (SEQ ID NO: 16) | CDR1: Kabat (SEQ ID NO: 269) |
| | Chothia (SEQ ID NO: 36) | Chothia (SEQ ID NO: 288) |
| | IMGT (SEQ ID NO: 61) | IMGT (SEQ ID NO: 313) |
| | CDR2: Kabat (SEQ ID NO: 91) | CDR2: Kabat (SEQ ID NO: 351) |
| | Chothia (SEQ ID NO: 128) | Chothia (SEQ ID NO: 361) |
| | IMGT (SEQ ID NO: 159) | IMGT (SEQ ID NO: 361) |
| | CDR3: Kabat (SEQ ID NO: 190) | CDR3: Kabat (SEQ ID NO: 371) |
| | Chothia (SEQ ID NO: 215) | Chothia (SEQ ID NO: 397) |
| | IMGT (SEQ ID NO: 240) | IMGT (SEQ ID NO: 371) |
| 541 | VH (SEQ ID NO: 461) | VL (SEQ ID NO: 521) |
| | CDR1: Kabat (SEQ ID NO: 15) | CDR1: Kabat (SEQ ID NO: 269) |
| | Chothia (SEQ ID NO: 27) | Chothia (SEQ ID NO: 288) |
| | IMGT (SEQ ID NO: 62) | IMGT (SEQ ID NO: 313) |
| | CDR2: Kabat (SEQ ID NO: 72) | CDR2: Kabat (SEQ ID NO: 337) |
| | Chothia (SEQ ID NO: 113) | Chothia (SEQ ID NO: 361) |
| | IMGT (SEQ ID NO: 144) | IMGT (SEQ ID NO: 361) |
| | CDR3: Kabat (SEQ ID NO: 190) | CDR3: Kabat (SEQ ID NO: 382) |
| | Chothia (SEQ ID NO: 215) | Chothia (SEQ ID NO: 407) |
| | IMGT (SEQ ID NO: 240) | IMGT (SEQ ID NO: 382) |
| 542 | VH (SEQ ID NO: 461) | VL (SEQ ID NO: 522) |
| | CDR1: Kabat (SEQ ID NO: 15) | CDR1: Kabat (SEQ ID NO: 269) |
| | Chothia (SEQ ID NO: 27) | Chothia (SEQ ID NO: 288) |
| | IMGT (SEQ ID NO: 62) | IMGT (SEQ ID NO: 313) |
| | CDR2: Kabat (SEQ ID NO: 72) | CDR2: Kabat (SEQ ID NO: 351) |
| | Chothia (SEQ ID NO: 113) | Chothia (SEQ ID NO: 361) |
| | IMGT (SEQ ID NO: 144) | IMGT (SEQ ID NO: 361) |
| | CDR3: Kabat (SEQ ID NO: 190) | CDR3: Kabat (SEQ ID NO: 383) |
| | Chothia (SEQ ID NO: 215) | Chothia (SEQ ID NO: 408) |
| | IMGT (SEQ ID NO: 240) | IMGT (SEQ ID NO: 383) |
| 543 | VH (SEQ ID NO: 462) | VL (SEQ ID NO: 523) |
| | CDR1: Kabat (SEQ ID NO: 5) | CDR1: Kabat (SEQ ID NO: 272) |
| | Chothia (SEQ ID NO: 37) | Chothia (SEQ ID NO: 297) |
| | IMGT (SEQ ID NO: 63) | IMGT (SEQ ID NO: 321) |
| | CDR2: Kabat (SEQ ID NO: 92) | CDR2: Kabat (SEQ ID NO: 338) |
| | Chothia (SEQ ID NO: 129) | Chothia (SEQ ID NO: 362) |
| | IMGT (SEQ ID NO: 160) | IMGT (SEQ ID NO: 362) |
| | CDR3: Kabat (SEQ ID NO: 179) | CDR3: Kabat (SEQ ID NO: 384) |
| | Chothia (SEQ ID NO: 207) | Chothia (SEQ ID NO: 409) |
| | IMGT (SEQ ID NO: 227) | IMGT (SEQ ID NO: 384) |
| 544 | VH (SEQ ID NO: 463) | VL (SEQ ID NO: 523) |
| | CDR1: Kabat (SEQ ID NO: 5) | CDR1: Kabat (SEQ ID NO: 272) |
| | Chothia (SEQ ID NO: 24) | Chothia (SEQ ID NO: 297) |
| | IMGT (SEQ ID NO: 49) | IMGT (SEQ ID NO: 321) |
| | CDR2: Kabat (SEQ ID NO: 93) | CDR2: Kabat (SEQ ID NO: 338) |
| | Chothia (SEQ ID NO: 130) | Chothia (SEQ ID NO: 362) |
| | IMGT (SEQ ID NO: 161) | IMGT (SEQ ID NO: 362) |
| | CDR3: Kabat (SEQ ID NO: 179) | CDR3: Kabat (SEQ ID NO: 384) |
| | Chothia (SEQ ID NO: 207) | Chothia (SEQ ID NO: 409) |
| | IMGT (SEQ ID NO: 227) | IMGT (SEQ ID NO: 384) |
| 545 | VH (SEQ ID NO: 464) | VL (SEQ ID NO: 524) |
| | CDR1: Kabat (SEQ ID NO: 5) | CDR1: Kabat (SEQ ID NO: 273) |
| | Chothia (SEQ ID NO: 840) | Chothia (SEQ ID NO: 298) |
| | IMGT (SEQ ID NO: 48) | IMGT (SEQ ID NO: 322) |
| | CDR2: Kabat (SEQ ID NO: 94) | CDR2: Kabat (SEQ ID NO: 338) |
| | Chothia (SEQ ID NO: 131) | Chothia (SEQ ID NO: 362) |
| | IMGT (SEQ ID NO: 162) | IMGT (SEQ ID NO: 362) |
| | CDR3: Kabat (SEQ ID NO: 192) | CDR3: Kabat (SEQ ID NO: 385) |
| | Chothia (SEQ ID NO: 207) | Chothia (SEQ ID NO: 410) |
| | IMGT (SEQ ID NO: 243) | IMGT (SEQ ID NO: 385) |
| 546 | VH (SEQ ID NO: 465) | VL (SEQ ID NO: 525) |
| | CDR1: Kabat (SEQ ID NO: 17) | CDR1: Kabat (SEQ ID NO: 272) |
| | Chothia (SEQ ID NO: 38) | Chothia (SEQ ID NO: 297) |
| | IMGT (SEQ ID NO: 64) | IMGT (SEQ ID NO: 321) |
| | CDR2: Kabat (SEQ ID NO: 95) | CDR2: Kabat (SEQ ID NO: 338) |
| | Chothia (SEQ ID NO: 132) | Chothia (SEQ ID NO: 362) |
| | IMGT (SEQ ID NO: 163) | IMGT (SEQ ID NO: 362) |
| | CDR3: Kabat (SEQ ID NO: 179) | CDR3: Kabat (SEQ ID NO: 386) |
| | Chothia (SEQ ID NO: 207) | Chothia (SEQ ID NO: 411) |
| | IMGT (SEQ ID NO: 227) | IMGT (SEQ ID NO: 386) |
| 547 | VH (SEQ ID NO: 466) | VL (SEQ ID NO: 526) |
| | CDR1: Kabat (SEQ ID NO: 5) | CDR1: Kabat (SEQ ID NO: 274) |
| | Chothia (SEQ ID NO: 24) | Chothia (SEQ ID NO: 299) |
| | IMGT (SEQ ID NO: 49) | IMGT (SEQ ID NO: 323) |
| | CDR2: Kabat (SEQ ID NO: 96) | CDR2: Kabat (SEQ ID NO: 338) |
| | Chothia (SEQ ID NO: 130) | Chothia (SEQ ID NO: 362) |
| | IMGT (SEQ ID NO: 164) | IMGT (SEQ ID NO: 362) |
| | CDR3: Kabat (SEQ ID NO: 192) | CDR3: Kabat (SEQ ID NO: 386) |
| | Chothia (SEQ ID NO: 207) | Chothia (SEQ ID NO: 411) |
| | IMGT (SEQ ID NO: 243) | IMGT (SEQ ID NO: 386) |
| 548 | VH (SEQ ID NO: 467) | VL (SEQ ID NO: 527) |
| | CDR1: Kabat (SEQ ID NO: 5) | CDR1: Kabat (SEQ ID NO: 275) |
| | Chothia (SEQ ID NO: 24) | Chothia (SEQ ID NO: 300) |
| | IMGT (SEQ ID NO: 49) | IMGT (SEQ ID NO: 324) |
| | CDR2: Kabat (SEQ ID NO: 97) | CDR2: Kabat (SEQ ID NO: 338) |
| | Chothia (SEQ ID NO: 130) | Chothia (SEQ ID NO: 362) |
| | IMGT (SEQ ID NO: 164) | IMGT (SEQ ID NO: 362) |
| | CDR3: Kabat (SEQ ID NO: 179) | CDR3: Kabat (SEQ ID NO: 386) |
| | Chothia (SEQ ID NO: 207) | Chothia (SEQ ID NO: 411) |
| | IMGT (SEQ ID NO: 227) | IMGT (SEQ ID NO: 386) |
| 549 | VH (SEQ ID NO: 468) | VL (SEQ ID NO: 528) |
| | CDR1: Kabat (SEQ ID NO: 18) | CDR1: Kabat (SEQ ID NO: 276) |
| | Chothia (SEQ ID NO: 39) | Chothia (SEQ ID NO: 301) |
| | IMGT (SEQ ID NO: 65) | IMGT (SEQ ID NO: 325) |
| | CDR2: Kabat (SEQ ID NO: 98) | CDR2: Kabat (SEQ ID NO: 337) |
| | Chothia (SEQ ID NO: 116) | Chothia (SEQ ID NO: 361) |
| | IMGT (SEQ ID NO: 147) | IMGT (SEQ ID NO: 361) |
| | CDR3: Kabat (SEQ ID NO: 193) | CDR3: Kabat (SEQ ID NO: 387) |
| | Chothia (SEQ ID NO: 216) | Chothia (SEQ ID NO: 412) |
| | IMGT (SEQ ID NO: 244) | IMGT (SEQ ID NO: 387) |
| 550 | VH (SEQ ID NO: 469) | VL (SEQ ID NO: 529) |
| | CDR1: Kabat (SEQ ID NO: 18) | CDR1: Kabat (SEQ ID NO: 277) |
| | Chothia (SEQ ID NO: 39) | Chothia (SEQ ID NO: 302) |
| | IMGT (SEQ ID NO: 65) | IMGT (SEQ ID NO: 326) |
| | CDR2: Kabat (SEQ ID NO: 98) | CDR2: Kabat (SEQ ID NO: 337) |
| | Chothia (SEQ ID NO: 116) | Chothia (SEQ ID NO: 361) |
| | IMGT (SEQ ID NO: 147) | IMGT (SEQ ID NO: 361) |
| | CDR3: Kabat (SEQ ID NO: 193) | CDR3: Kabat (SEQ ID NO: 387) |
| | Chothia (SEQ ID NO: 216) | Chothia (SEQ ID NO: 412) |
| | IMGT (SEQ ID NO: 245) | IMGT (SEQ ID NO: 387) |
| 551 | VH (SEQ ID NO: 470) | VL (SEQ ID NO: 528) |
| | CDR1: Kabat (SEQ ID NO: 18) | CDR1: Kabat (SEQ ID NO: 276) |
| | Chothia (SEQ ID NO: 39) | Chothia (SEQ ID NO: 301) |
| | IMGT (SEQ ID NO: 65) | IMGT (SEQ ID NO: 325) |
| | CDR2: Kabat (SEQ ID NO: 98) | CDR2: Kabat (SEQ ID NO: 337) |
| | Chothia (SEQ ID NO: 116) | Chothia (SEQ ID NO: 361) |
| | IMGT (SEQ ID NO: 147) | IMGT (SEQ ID NO: 361) |
| | CDR3: Kabat (SEQ ID NO: 194) | CDR3: Kabat (SEQ ID NO: 387) |
| | Chothia (SEQ ID NO: 217) | Chothia (SEQ ID NO: 412) |
| | IMGT (SEQ ID NO: 246) | IMGT (SEQ ID NO: 387) |
| 552 | VH (SEQ ID NO: 471) | VL (SEQ ID NO: 530) |
| | CDR1: Kabat (SEQ ID NO: 18) | CDR1: Kabat (SEQ ID NO: 276) |
| | Chothia (SEQ ID NO: 39) | Chothia (SEQ ID NO: 301) |
| | IMGT (SEQ ID NO: 65) | IMGT (SEQ ID NO: 325) |
| | CDR2: Kabat (SEQ ID NO: 98) | CDR2: Kabat (SEQ ID NO: 337) |
| | Chothia (SEQ ID NO: 116) | Chothia (SEQ ID NO: 361) |
| | IMGT (SEQ ID NO: 147) | IMGT (SEQ ID NO: 361) |
| | CDR3: Kabat (SEQ ID NO: 194) | CDR3: Kabat (SEQ ID NO: 388) |
| | Chothia (SEQ ID NO: 217) | Chothia (SEQ ID NO: 413) |
| | IMGT (SEQ ID NO: 247) | IMGT (SEQ ID NO: 388) |
| 553 | VH (SEQ ID NO: 472) | VL (SEQ ID NO: 531) |
| | CDR1: Kabat (SEQ ID NO: 19) | CDR1: Kabat (SEQ ID NO: 278) |
| | Chothia (SEQ ID NO: 40) | Chothia (SEQ ID NO: 303) |
| | IMGT (SEQ ID NO: 66) | IMGT (SEQ ID NO: 327) |
| | CDR2: Kabat (SEQ ID NO: 99) | CDR2: Kabat (SEQ ID NO: 352) |
| | Chothia (SEQ ID NO: 133) | Chothia (SEQ ID NO: 367) |
| | IMGT (SEQ ID NO: 165) | IMGT (SEQ ID NO: 367) |
| | CDR3: Kabat (SEQ ID NO: 180) | CDR3: Kabat (SEQ ID NO: 387) |
| | Chothia (SEQ ID NO: 208) | Chothia (SEQ ID NO: 412) |
| | IMGT (SEQ ID NO: 248) | IMGT (SEQ ID NO: 387) |
| 554 | VH (SEQ ID NO: 473) | VL (SEQ ID NO: 532) |
| | CDR1: Kabat (SEQ ID NO: 20) | CDR1: Kabat (SEQ ID NO: 279) |
| | Chothia (SEQ ID NO: 41) | Chothia (SEQ ID NO: 304) |
| | IMGT (SEQ ID NO: 67) | IMGT (SEQ ID NO: 328) |
| | CDR2: Kabat (SEQ ID NO: 100) | CDR2: Kabat (SEQ ID NO: 353) |
| | Chothia (SEQ ID NO: 134) | Chothia (SEQ ID NO: 361) |
| | IMGT (SEQ ID NO: 166) | IMGT (SEQ ID NO: 361) |
| | CDR3: Kabat (SEQ ID NO: 195) | CDR3: Kabat (SEQ ID NO: 374) |
| | Chothia (SEQ ID NO: 218) | Chothia (SEQ ID NO: 400) |
| | IMGT (SEQ ID NO: 249) | IMGT (SEQ ID NO: 374) |
| 555 | VH (SEQ ID NO: 474) | VL (SEQ ID NO: 533) |
| | CDR1: Kabat (SEQ ID NO: 20) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 101) | CDR2: Kabat (SEQ ID NO: 337) |
| | Chothia (SEQ ID NO: 133) | Chothia (SEQ ID NO: 361) |
| | IMGT (SEQ ID NO: 167) | IMGT (SEQ ID NO: 361) |
| | CDR3: Kabat (SEQ ID NO: 180) | CDR3: Kabat (SEQ ID NO: 389) |
| | Chothia (SEQ ID NO: 208) | Chothia (SEQ ID NO: 414) |
| | IMGT (SEQ ID NO: 248) | IMGT (SEQ ID NO: 389) |
| 556 | VH (SEQ ID NO: 475) | VL (SEQ ID NO: 534) |
| | CDR1: Kabat (SEQ ID NO: 5) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 102) | CDR2: Kabat (SEQ ID NO: 350) |
| | Chothia (SEQ ID NO: 135) | Chothia (SEQ ID NO: 361) |
| | IMGT (SEQ ID NO: 168) | IMGT (SEQ ID NO: 361) |
| | CDR3: Kabat (SEQ ID NO: 180) | CDR3: Kabat (SEQ ID NO: 389) |
| | Chothia (SEQ ID NO: 208) | Chothia (SEQ ID NO: 414) |
| | IMGT (SEQ ID NO: 248) | IMGT (SEQ ID NO: 389) |
| 557 | VH (SEQ ID NO: 476) | VL (SEQ ID NO: 535) |
| | CDR1: Kabat (SEQ ID NO: 5) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 103) | CDR2: Kabat (SEQ ID NO: 354) |
| | Chothia (SEQ ID NO: 136) | Chothia (SEQ ID NO: 367) |
| | IMGT (SEQ ID NO: 169) | IMGT (SEQ ID NO: 367) |
| | CDR3: Kabat (SEQ ID NO: 196) | CDR3: Kabat (SEQ ID NO: 390) |
| | Chothia (SEQ ID NO: 218) | Chothia (SEQ ID NO: 415) |
| | IMGT (SEQ ID NO: 250) | IMGT (SEQ ID NO: 390) |
| 558 | VH (SEQ ID NO: 477) | VL (SEQ ID NO: 536) |
| | CDR1: Kabat (SEQ ID NO: 5) | CDR1: Kabat (SEQ ID NO: 281) |
| | Chothia (SEQ ID NO: 840) | Chothia (SEQ ID NO: 306) |
| | IMGT (SEQ ID NO: 48) | IMGT (SEQ ID NO: 330) |
| | CDR2: Kabat (SEQ ID NO: 104) | CDR2: Kabat (SEQ ID NO: 337) |
| | Chothia (SEQ ID NO: 137) | Chothia (SEQ ID NO: 361) |
| | IMGT (SEQ ID NO: 170) | IMGT (SEQ ID NO: 361) |
| | CDR3: Kabat (SEQ ID NO: 197) | CDR3: Kabat (SEQ ID NO: 387) |
| | Chothia (SEQ ID NO: 208) | Chothia (SEQ ID NO: 412) |
| | IMGT (SEQ ID NO: 251) | IMGT (SEQ ID NO: 387) |
| 559 | VH (SEQ ID NO: 478) | VL (SEQ ID NO: 536) |
| | CDR1: Kabat (SEQ ID NO: 5) | CDR1: Kabat (SEQ ID NO: 281) |
| | Chothia (SEQ ID NO: 840) | Chothia (SEQ ID NO: 306) |
| | IMGT (SEQ ID NO: 48) | IMGT (SEQ ID NO: 330) |
| | CDR2: Kabat (SEQ ID NO: 105) | CDR2: Kabat (SEQ ID NO: 337) |
| | Chothia (SEQ ID NO: 138) | Chothia (SEQ ID NO: 361) |
| | IMGT (SEQ ID NO: 171) | IMGT (SEQ ID NO: 361) |
| | CDR3: Kabat (SEQ ID NO: 197) | CDR3: Kabat (SEQ ID NO: 387) |
| | Chothia (SEQ ID NO: 208) | Chothia (SEQ ID NO: 412) |
| | IMGT (SEQ ID NO: 251) | IMGT (SEQ ID NO: 387) |
| 560 | VH (SEQ ID NO: 479) | VL (SEQ ID NO: 537) |
| | CDR1: Kabat (SEQ ID NO: 5) | CDR1: Kabat (SEQ ID NO: 282) |
| | Chothia (SEQ ID NO: 840) | Chothia (SEQ ID NO: 307) |
| | IMGT (SEQ ID NO: 48) | IMGT (SEQ ID NO: 331) |
| | CDR2: Kabat (SEQ ID NO: 94) | CDR2: Kabat (SEQ ID NO: 337) |
| | Chothia (SEQ ID NO: 131) | Chothia (SEQ ID NO: 361) |
| | IMGT (SEQ ID NO: 162) | IMGT (SEQ ID NO: 361) |
| | CDR3: Kabat (SEQ ID NO: 198) | CDR3: Kabat (SEQ ID NO: 391) |
| | Chothia (SEQ ID NO: 219) | Chothia (SEQ ID NO: 416) |
| | IMGT (SEQ ID NO: 252) | IMGT (SEQ ID NO: 391) |
| 561 | VH (SEQ ID NO: 480) | VL (SEQ ID NO: 538) |
| | CDR1: Kabat (SEQ ID NO: 20) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 94) | CDR2: Kabat (SEQ ID NO: 355) |
| | Chothia (SEQ ID NO: 131) | Chothia (SEQ ID NO: 367) |
| | IMGT (SEQ ID NO: 162) | IMGT (SEQ ID NO: 367) |
| | CDR3: Kabat (SEQ ID NO: 195) | CDR3: Kabat (SEQ ID NO: 387) |
| | Chothia (SEQ ID NO: 218) | Chothia (SEQ ID NO: 412) |
| | IMGT (SEQ ID NO: 253) | IMGT (SEQ ID NO: 387) |
| 562 | VH (SEQ ID NO: 481) | VL (SEQ ID NO: 539) |
| | CDR1: Kabat (SEQ ID NO: 5) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 94) | CDR2: Kabat (SEQ ID NO: 337) |
| | Chothia (SEQ ID NO: 131) | Chothia (SEQ ID NO: 361) |
| | IMGT (SEQ ID NO: 162) | IMGT (SEQ ID NO: 361) |
| | CDR3: Kabat (SEQ ID NO: 180) | CDR3: Kabat (SEQ ID NO: 387) |
| | Chothia (SEQ ID NO: 208) | Chothia (SEQ ID NO: 412) |
| | IMGT (SEQ ID NO: 248) | IMGT (SEQ ID NO: 387) |
| 563 | VH (SEQ ID NO: 482) | VL (SEQ ID NO: 539) |
| | CDR1: Kabat (SEQ ID NO: 6) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 106) | CDR2: Kabat (SEQ ID NO: 337) |
| | Chothia (SEQ ID NO: 137) | Chothia (SEQ ID NO: 361) |
| | IMGT (SEQ ID NO: 170) | IMGT (SEQ ID NO: 361) |
| | CDR3: Kabat (SEQ ID NO: 195) | CDR3: Kabat (SEQ ID NO: 387) |
| | Chothia (SEQ ID NO: 218) | Chothia (SEQ ID NO: 412) |
| | IMGT (SEQ ID NO: 253) | IMGT (SEQ ID NO: 387) |
| 564 | VH (SEQ ID NO: 483) | VL (SEQ ID NO: 539) |
| | CDR1: Kabat (SEQ ID NO: 20) | CDR1: Kabat (SEQ ID NO: 280) |
| | Chothia (SEQ ID NO: 41) | Chothia (SEQ ID NO: 305) |
| | IMGT (SEQ ID NO: 67) | IMGT (SEQ ID NO: 329) |
| | CDR2: Kabat (SEQ ID NO: 107) | CDR2: Kabat (SEQ ID NO: 337) |
| | Chothia (SEQ ID NO: 133) | Chothia (SEQ ID NO: 361) |
| | IMGT (SEQ ID NO: 167) | IMGT (SEQ ID NO: 361) |
| | CDR3: Kabat (SEQ ID NO: 180) | CDR3: Kabat (SEQ ID NO: 387) |
| | Chothia (SEQ ID NO: 208) | Chothia (SEQ ID NO: 412) |
| | IMGT (SEQ ID NO: 248) | IMGT (SEQ ID NO: 387) |
| 565 | VH (SEQ ID NO: 484) | VL (SEQ ID NO: 540) |
| | CDR1: Kabat (SEQ ID NO: 5) | CDR1: Kabat (SEQ ID NO: 283) |
| | Chothia (SEQ ID NO: 840) | Chothia (SEQ ID NO: 308) |
| | IMGT (SEQ ID NO: 48) | IMGT (SEQ ID NO: 332) |
| | CDR2: Kabat (SEQ ID NO: 108) | CDR2: Kabat (SEQ ID NO: 356) |
| | Chothia (SEQ ID NO: 139) | Chothia (SEQ ID NO: 361) |
| | IMGT (SEQ ID NO: 172) | IMGT (SEQ ID NO: 361) |
| | CDR3: Kabat (SEQ ID NO: 181) | CDR3: Kabat (SEQ ID NO: 374) |
| | Chothia (SEQ ID NO: 209) | Chothia (SEQ ID NO: 400) |
| | IMGT (SEQ ID NO: 229) | IMGT (SEQ ID NO: 374) |
| 566 | VH (SEQ ID NO: 485) | VL (SEQ ID NO: 541) |
| | CDR1: Kabat (SEQ ID NO: 21) | CDR1: Kabat (SEQ ID NO: 280) |
| | Chothia (SEQ ID NO: 42) | Chothia (SEQ ID NO: 305) |
| | IMGT (SEQ ID NO: 68) | IMGT (SEQ ID NO: 329) |
| | CDR2: Kabat (SEQ ID NO: 109) | CDR2: Kabat (SEQ ID NO: 337) |
| | Chothia (SEQ ID NO: 140) | Chothia (SEQ ID NO: 361) |
| | IMGT (SEQ ID NO: 173) | IMGT (SEQ ID NO: 361) |
| | CDR3: Kabat (SEQ ID NO: 199) | CDR3: Kabat (SEQ ID NO: 374) |
| | Chothia (SEQ ID NO: 220) | Chothia (SEQ ID NO: 400) |
| | IMGT (SEQ ID NO: 254) | IMGT (SEQ ID NO: 374) |
| 567 | VH (SEQ ID NO: 486) | VL (SEQ ID NO: 542) |
| | CDR1: Kabat (SEQ ID NO: 5) | CDR1: Kabat (SEQ ID NO: 284) |
| | Chothia (SEQ ID NO: 43) | Chothia (SEQ ID NO: 309) |
| | IMGT (SEQ ID NO: 69) | IMGT (SEQ ID NO: 333) |
| | CDR2: Kabat (SEQ ID NO: 106) | CDR2: Kabat (SEQ ID NO: 337) |
| | Chothia (SEQ ID NO: 137) | Chothia (SEQ ID NO: 361) |
| | IMGT (SEQ ID NO: 170) | IMGT (SEQ ID NO: 361) |
| | CDR3: Kabat (SEQ ID NO: 200) | CDR3: Kabat (SEQ ID NO: 374) |
| | Chothia (SEQ ID NO: 221) | Chothia (SEQ ID NO: 400) |
| | IMGT (SEQ ID NO: 255) | IMGT (SEQ ID NO: 374) |
| 568 | VH (SEQ ID NO: 487) | VL (SEQ ID NO: 543) |
| | CDR1: Kabat (SEQ ID NO: 5) | CDR1: Kabat (SEQ ID NO: 285) |
| | Chothia (SEQ ID NO: 43) | Chothia (SEQ ID NO: 310) |
| | IMGT (SEQ ID NO: 69) | IMGT (SEQ ID NO: 334) |
| | CDR2: Kabat (SEQ ID NO: 94) | CDR2: Kabat (SEQ ID NO: 357) |
| | Chothia (SEQ ID NO: 131) | Chothia (SEQ ID NO: 361) |
| | IMGT (SEQ ID NO: 162) | IMGT (SEQ ID NO: 361) |
| | CDR3: Kabat (SEQ ID NO: 201) | CDR3: Kabat (SEQ ID NO: 392) |
| | Chothia (SEQ ID NO: 222) | Chothia (SEQ ID NO: 417) |
| | IMGT (SEQ ID NO: 256) | IMGT (SEQ ID NO: 392) |
| 569 | VH (SEQ ID NO: 488) | VL (SEQ ID NO: 544) |
| | CDR1: Kabat (SEQ ID NO: 5) | CDR1: Kabat (SEQ ID NO: 285) |
| | Chothia (SEQ ID NO: 43) | Chothia (SEQ ID NO: 310) |
| | IMGT (SEQ ID NO: 69) | IMGT (SEQ ID NO: 334) |
| | CDR2: Kabat (SEQ ID NO: 110) | CDR2: Kabat (SEQ ID NO: 358) |
| | Chothia (SEQ ID NO: 141) | Chothia (SEQ ID NO: 361) |
| | IMGT (SEQ ID NO: 174) | IMGT (SEQ ID NO: 361) |
| | CDR3: Kabat (SEQ ID NO: 202) | CDR3: Kabat (SEQ ID NO: 393) |
| | Chothia (SEQ ID NO: 222) | Chothia (SEQ ID NO: 418) |
| | IMGT (SEQ ID NO: 257) | IMGT (SEQ ID NO: 393) |
| 104 | VH (SEQ ID NO: 818) | VL (SEQ ID NO: 825) |
| | CDR1: Kabat (SEQ ID NO: 5) | CDR1: Kabat (SEQ ID NO: 781) |
| | Chothia (SEQ ID NO: 840) | Chothia (SEQ ID NO: 787) |
| | IMGT (SEQ ID NO: 48) | IMGT (SEQ ID NO: 792) |
| | CDR2: Kabat (SEQ ID NO: 748) | CDR2: Kabat (SEQ ID NO: 797) |
| | Chothia (SEQ ID NO: 116) | Chothia (SEQ ID NO: 802) |
| | IMGT (SEQ ID NO: 758) | IMGT (SEQ ID NO: 802) |
| | CDR3: Kabat (SEQ ID NO: 763) | CDR3: Kabat (SEQ ID NO: 806) |
| | Chothia (SEQ ID NO: 769) | Chothia (SEQ ID NO: 812) |
| | IMGT (SEQ ID NO: 775) | IMGT (SEQ ID NO: 806) |
| 111 | VH (SEQ ID NO: 819) | VL (SEQ ID NO: 826) |
| | CDR1: Kabat (SEQ ID NO: 740) | CDR1: Kabat (SEQ ID NO: 782) |
| | Chothia (SEQ ID NO: 742) | Chothia (SEQ ID NO: 788) |
| | IMGT (SEQ ID NO: 745) | IMGT (SEQ ID NO: 793) |
| | CDR2: Kabat (SEQ ID NO: 749) | CDR2: Kabat (SEQ ID NO: 798) |
| | Chothia (SEQ ID NO: 754) | Chothia (SEQ ID NO: 803) |
| | IMGT (SEQ ID NO: 759) | IMGT (SEQ ID NO: 803) |
| | CDR3: Kabat (SEQ ID NO: 764) | CDR3: Kabat (SEQ ID NO: 807) |
| | Chothia (SEQ ID NO: 770) | Chothia (SEQ ID NO: 813) |
| | IMGT (SEQ ID NO: 776) | IMGT (SEQ ID NO: 807) |
| 115 | VH (SEQ ID NO: 820) | VL (SEQ ID NO: 827) |
| | CDR1: Kabat (SEQ ID NO: 17) | CDR1: Kabat (SEQ ID NO: 783) |
| | Chothia (SEQ ID NO: 743) | Chothia (SEQ ID NO: 298) |
| | IMGT (SEQ ID NO: 746) | IMGT (SEQ ID NO: 322) |
| | CDR2: Kabat (SEQ ID NO: 750) | CDR2: Kabat (SEQ ID NO: 799) |
| | Chothia (SEQ ID NO: 133) | Chothia (SEQ ID NO: 804) |
| | IMGT (SEQ ID NO: 165) | IMGT (SEQ ID NO: 804) |
| | CDR3: Kabat (SEQ ID NO: 765) | CDR3: Kabat (SEQ ID NO: 808) |
| | Chothia (SEQ ID NO: 771) | Chothia (SEQ ID NO: 814) |
| | IMGT (SEQ ID NO: 777) | IMGT (SEQ ID NO: 808) |
| 119 | VH (SEQ ID NO: 821) | VL (SEQ ID NO: 828) |
| | CDR1: Kabat (SEQ ID NO: 5) | CDR1: Kabat (SEQ ID NO: 784) |
| | Chothia (SEQ ID NO: 840) | Chothia (SEQ ID NO: 789) |
| | IMGT (SEQ ID NO: 48) | IMGT (SEQ ID NO: 794) |
| | CDR2: Kabat (SEQ ID NO: 751) | CDR2: Kabat (SEQ ID NO: 341) |
| | Chothia (SEQ ID NO: 755) | Chothia (SEQ ID NO: 361) |
| | IMGT (SEQ ID NO: 760) | IMGT (SEQ ID NO: 361) |
| | CDR3: Kabat (SEQ ID NO: 766) | CDR3: Kabat (SEQ ID NO: 809) |
| | Chothia (SEQ ID NO: 772) | Chothia (SEQ ID NO: 815) |
| | IMGT (SEQ ID NO: 778) | IMGT (SEQ ID NO: 809) |
| 216 | VH (SEQ ID NO: 822) | VL (SEQ ID NO: 829) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 368) |
| | Chothia (SEQ ID NO: 203) | Chothia (SEQ ID NO: 394) |
| | IMGT (SEQ ID NO: 223) | IMGT (SEQ ID NO: 368) |
| 231 | VH (SEQ ID NO: 823) | VL (SEQ ID NO: 830) |
| | CDR1: Kabat (SEQ ID NO: 8) | CDR1: Kabat (SEQ ID NO: 785) |
| | Chothia (SEQ ID NO: 26) | Chothia (SEQ ID NO: 790) |
| | IMGT (SEQ ID NO: 51) | IMGT (SEQ ID NO: 795) |
| | CDR2: Kabat (SEQ ID NO: 752) | CDR2: Kabat (SEQ ID NO: 800) |
| | Chothia (SEQ ID NO: 756) | Chothia (SEQ ID NO: 805) |
| | IMGT (SEQ ID NO: 761) | IMGT (SEQ ID NO: 805) |
| | CDR3: Kabat (SEQ ID NO: 767) | CDR3: Kabat (SEQ ID NO: 810) |
| | Chothia (SEQ ID NO: 773) | Chothia (SEQ ID NO: 816) |
| | IMGT (SEQ ID NO: 779) | IMGT (SEQ ID NO: 810) |
| 328 | VH (SEQ ID NO: 824) | VL (SEQ ID NO: 831) |
| | CDR1: Kabat (SEQ ID NO: 741) | CDR1: Kabat (SEQ ID NO: 786) |
| | Chothia (SEQ ID NO: 744) | Chothia (SEQ ID NO: 791) |
| | IMGT (SEQ ID NO: 747) | IMGT (SEQ ID NO: 796) |
| | CDR2: Kabat (SEQ ID NO: 753) | CDR2: Kabat (SEQ ID NO: 801) |
| | Chothia (SEQ ID NO: 757) | Chothia (SEQ ID NO: 360) |
| | IMGT (SEQ ID NO: 762) | IMGT (SEQ ID NO: 360) |
| | CDR3: Kabat (SEQ ID NO: 768) | CDR3: Kabat (SEQ ID NO: 811) |
| | Chothia (SEQ ID NO: 774) | Chothia (SEQ ID NO: 817) |
| | IMGT (SEQ ID NO: 780) | IMGT (SEQ ID NO: 811) |
| 105 | VH (SEQ ID NO: 841) | VL (SEQ ID NO: 842) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 347) |
| | Chothia (SEQ ID NO: 111) | Chothia (SEQ ID NO: 365) |
| | IMGT (SEQ ID NO: 142) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 234) | IMGT (SEQ ID NO: 378) |

### V_{H} Domains

In some embodiments, an antibody provided herein comprises a VH sequence selected from any one of SEQ ID NOs: 419-488, 818-824, and 841.

In some embodiments, an antibody provided herein comprises a VH sequence having at least about 80%, 90%, 91%, 92%, 93%, 94%, 95%, 96%, 97%, 98% or 99% identity to an illustrative VH sequence selected from any one of SEQ ID NOs: 419-488, 818-824, and 841. In some embodiments, an antibody provided herein comprises a VH sequence selected from any one of SEQ ID NOs: 419-488, 818-824, and 841, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### V_{L} Domains

In some embodiments, an antibody provided herein comprises a VL sequence selected from any one of SEQ ID NOs: 489-544, 825-831, and 842.

In some embodiments, an antibody provided herein comprises a VL sequence having at least about 80%, 90%, 91%, 92%, 93%, 94%, 95%, 96%, 97%, 98% or 99% identity to a VL sequence selected from any one of SEQ ID NOs: 489-544, 825-831, and 842. In some embodiments, an antibody provided herein comprises a VL sequence selected from any one of SEQ ID NOs: 489-544, 825-831, and 842, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VH-VL Combinations

In some embodiments, an antibody provided herein comprises a VH sequence selected from any one of SEQ ID NOs: 419-488, 818-824, and 841; and a VL sequence selected from any one of SEQ ID NOs: 489-544, 825-831, and 842.

In certain aspects, a VH sequence selected from any one of SEQ ID NOs: 419-488, 818-824, and 841 can be combined with a VL sequence selected from any one of SEQ ID NOs: 489-544, 825-831, and 842.

In certain aspects, an antibody provided herein comprises a VH sequence and a VL sequence of a construct provided in TABLE 2.

In some embodiments, an antibody provided herein comprises a VH sequence having at least about 80%, 90%, 91%, 92%, 93%, 94%, 95%, 96%, 97%, 98% or 99% identity to a VH sequence selected from any one of SEQ ID NOs: 419-488, 818-824, and 841; and a VL sequence having at least about 80%, 90%, 91%, 92%, 93%, 94%, 95%, 96%, 97%, 98% or 99% identity to a VL sequence selected from any one of SEQ ID NOs: 489-544, 825-831, and 842. In some embodiments, an antibody provided herein comprises a VH sequence selected from any one of SEQ ID NOs: 419-488, 818-824, and 841, with up to 1, 2, 3, 4, 5, 6, 7, 8, 9, 10, 11, 12, 13, 14, 15, 16, 17, 18, 19, or 20 amino acid substitutions, and a VL sequence selected from any one of SEQ ID NOs: 489-544, 825-831, and 842, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In certain embodiments, the antibody comprises a VH sequence set forth in SEQ ID NO: 419 and a VL sequence set forth in SEQ ID NO: 489. In certain embodiments, the antibody comprises a VH sequence set forth in SEQ ID NO: 420 and a VL sequence set forth in SEQ ID NO: 490. In certain embodiments, the antibody comprises a VH sequence set forth in SEQ ID NO: 421 and a VL sequence set forth in SEQ ID NO: 491 In certain embodiments, the antibody comprises a VH sequence set forth in SEQ ID NO: 422 and a VL sequence set forth in SEQ ID NO: 492. In certain embodiments, the antibody comprises a VH sequence set forth in SEQ ID NO: 423 and a VL sequence set forth in SEQ ID NO: 493.

In certain embodiments, the antibody comprises a VH sequence set forth in SEQ ID NO: 424 and a VL sequence set forth in SEQ ID NO: 494. In certain embodiments, the antibody comprises a VH sequence set forth in SEQ ID NO: 425 and a VL sequence set forth in SEQ ID NO: 495. In certain embodiments, the antibody comprises a VH sequence set forth in SEQ ID NO: 426 and a VL sequence set forth in SEQ ID NO: 496. In certain embodiments, the antibody comprises a VH sequence set forth in SEQ ID NO: 427 and a VL sequence set forth in SEQ ID NO: 497. In certain embodiments, the antibody comprises a VH sequence set forth in SEQ ID NO: 427 and a VL sequence set forth in SEQ ID NO: 498. In certain embodiments, the antibody comprises a VH sequence set forth in SEQ ID NO: 428 and a VL sequence set forth in SEQ ID NO: 497. In certain embodiments, the antibody comprises a VH sequence set forth in SEQ ID NO: 428 and a VL sequence set forth in SEQ ID NO: 498.

In certain embodiments, the antibody comprises a VH sequence set forth in SEQ ID NO: 429 and a VL sequence set forth in SEQ ID NO: 497.

In certain embodiments, the antibody comprises a VH sequence set forth in SEQ ID NO: 429 and a VL sequence set forth in SEQ ID NO: 498.

In certain embodiments, the antibody comprises a VH sequence set forth in SEQ ID NO: 430 and a VL sequence set forth in SEQ ID NO: 499.

In certain embodiments, the antibody comprises a VH sequence set forth in SEQ ID NO: 431 and a VL sequence set forth in SEQ ID NO: 500.

In certain embodiments, the antibody comprises a VH sequence set forth in SEQ ID NO: 432 and a VL sequence set forth in SEQ ID NO: 501.

In certain embodiments, the antibody comprises a VH sequence set forth in SEQ ID NO: 433 and a VL sequence set forth in SEQ ID NO: 502.

In certain embodiments, the antibody comprises a VH sequence set forth in SEQ ID NO: 434 and a VL sequence set forth in SEQ ID NO: 502.

In certain embodiments, the antibody comprises a VH sequence set forth in SEQ ID NO: 435 and a VL sequence set forth in SEQ ID NO: 502.

In certain embodiments, the antibody comprises a VH sequence set forth in SEQ ID NO: 436 and a VL sequence set forth in SEQ ID NO: 502.

In certain embodiments, the antibody comprises a VH sequence set forth in SEQ ID NO: 437 and a VL sequence set forth in SEQ ID NO: 502.

In certain embodiments, the antibody comprises a VH sequence set forth in SEQ ID NO: 438 and a VL sequence set forth in SEQ ID NO: 503.

In certain embodiments, the antibody comprises a VH sequence set forth in SEQ ID NO: 439 and a VL sequence set forth in SEQ ID NO: 502.

In certain embodiments, the antibody comprises a VH sequence set forth in SEQ ID NO: 440 and a VL sequence set forth in SEQ ID NO: 504.

In certain embodiments, the antibody comprises a VH sequence set forth in SEQ ID NO: 441 and a VL sequence set forth in SEQ ID NO: 505.

In certain embodiments, the antibody comprises a VH sequence set forth in SEQ ID NO: 442 and a VL sequence set forth in SEQ ID NO: 506.

In certain embodiments, the antibody comprises a VH sequence set forth in SEQ ID NO: 443 and a VL sequence set forth in SEQ ID NO: 507.

In certain embodiments, the antibody comprises a VH sequence set forth in SEQ ID NO: 443 and a VL sequence set forth in SEQ ID NO: 508.

In certain embodiments, the antibody comprises a VH sequence set forth in SEQ ID NO: 444 and a VL sequence set forth in SEQ ID NO: 506.

In certain embodiments, the antibody comprises a VH sequence set forth in SEQ ID NO: 445 and a VL sequence set forth in SEQ ID NO: 509.

In certain embodiments, the antibody comprises a VH sequence set forth in SEQ ID NO: 446 and a VL sequence set forth in SEQ ID NO: 510.

In certain embodiments, the antibody comprises a VH sequence set forth in SEQ ID NO: 447 and a VL sequence set forth in SEQ ID NO: 506.

In certain embodiments, the antibody comprises a VH sequence set forth in SEQ ID NO: 445 and a VL sequence set forth in SEQ ID NO: 511.

In certain embodiments, the antibody comprises a VH sequence set forth in SEQ ID NO: 448 and a VL sequence set forth in SEQ ID NO: 506.

In certain embodiments, the antibody comprises a VH sequence set forth in SEQ ID NO: 449 and a VL sequence set forth in SEQ ID NO: 502.

In certain embodiments, the antibody comprises a VH sequence set forth in SEQ ID NO: 449 and a VL sequence set forth in SEQ ID NO: 506.

In certain embodiments, the antibody comprises a VH sequence set forth in SEQ ID NO: 450 and a VL sequence set forth in SEQ ID NO: 506.

In certain embodiments, the antibody comprises a VH sequence set forth in SEQ ID NO: 445 and a VL sequence set forth in SEQ ID NO: 512.

In certain embodiments, the antibody comprises a VH sequence set forth in SEQ ID NO: 449 and a VL sequence set forth in SEQ ID NO: 513.

In certain embodiments, the antibody comprises a VH sequence set forth in SEQ ID NO: 445 and a VL sequence set forth in SEQ ID NO: 514.

In certain embodiments, the antibody comprises a VH sequence set forth in SEQ ID NO: 445 and a VL sequence set forth in SEQ ID NO: 515.

In certain embodiments, the antibody comprises a VH sequence set forth in SEQ ID NO: 451 and a VL sequence set forth in SEQ ID NO: 514.

In certain embodiments, the antibody comprises a VH sequence set forth in SEQ ID NO: 451 and a VL sequence set forth in SEQ ID NO: 506.

In certain embodiments, the antibody comprises a VH sequence set forth in SEQ ID NO: 452 and a VL sequence set forth in SEQ ID NO: 506.

In certain embodiments, the antibody comprises a VH sequence set forth in SEQ ID NO: 438 and a VL sequence set forth in SEQ ID NO: 506.

In certain embodiments, the antibody comprises a VH sequence set forth in SEQ ID NO: 453 and a VL sequence set forth in SEQ ID NO: 512.

In certain embodiments, the antibody comprises a VH sequence set forth in SEQ ID NO: 454 and a VL sequence set forth in SEQ ID NO: 490.

In certain embodiments, the antibody comprises a VH sequence set forth in SEQ ID NO: 455 and a VL sequence set forth in SEQ ID NO: 516.

In certain embodiments, the antibody comprises a VH sequence set forth in SEQ ID NO: 456 and a VL sequence set forth in SEQ ID NO: 517.

In certain embodiments, the antibody comprises a VH sequence set forth in SEQ ID NO: 457 and a VL sequence set forth in SEQ ID NO: 518.

In certain embodiments, the antibody comprises a VH sequence set forth in SEQ ID NO: 458 and a VL sequence set forth in SEQ ID NO: 492.

In certain embodiments, the antibody comprises a VH sequence set forth in SEQ ID NO: 459 and a VL sequence set forth in SEQ ID NO: 519.

In certain embodiments, the antibody comprises a VH sequence set forth in SEQ ID NO: 460 and a VL sequence set forth in SEQ ID NO: 520.

In certain embodiments, the antibody comprises a VH sequence set forth in SEQ ID NO: 461 and a VL sequence set forth in SEQ ID NO: 521.

In certain embodiments, the antibody comprises a VH sequence set forth in SEQ ID NO: 461 and a VL sequence set forth in SEQ ID NO: 522.

In certain embodiments, the antibody comprises a VH sequence set forth in SEQ ID NO: 461 and a VL sequence set forth in SEQ ID NO: 522.

In certain embodiments, the antibody comprises a VH sequence set forth in SEQ ID NO: 462 and a VL sequence set forth in SEQ ID NO: 523.

In certain embodiments, the antibody comprises a VH sequence set forth in SEQ ID NO: 463 and a VL sequence set forth in SEQ ID NO: 523.

In certain embodiments, the antibody comprises a VH sequence set forth in SEQ ID NO: 464 and a VL sequence set forth in SEQ ID NO: 524.

In certain embodiments, the antibody comprises a VH sequence set forth in SEQ ID NO: 465 and a VL sequence set forth in SEQ ID NO: 525.

In certain embodiments, the antibody comprises a VH sequence set forth in SEQ ID NO: 466 and a VL sequence set forth in SEQ ID NO: 526.

In certain embodiments, the antibody comprises a VH sequence set forth in SEQ ID NO: 467 and a VL sequence set forth in SEQ ID NO: 527.

In certain embodiments, the antibody comprises a VH sequence set forth in SEQ ID NO: 468 and a VL sequence set forth in SEQ ID NO: 528.

In certain embodiments, the antibody comprises a VH sequence set forth in SEQ ID NO: 469 and a VL sequence set forth in SEQ ID NO: 529.

In certain embodiments, the antibody comprises a VH sequence set forth in SEQ ID NO: 470 and a VL sequence set forth in SEQ ID NO: 528.

In certain embodiments, the antibody comprises a VH sequence set forth in SEQ ID NO: 471 and a VL sequence set forth in SEQ ID NO: 530.

In certain embodiments, the antibody comprises a VH sequence set forth in SEQ ID NO: 472 and a VL sequence set forth in SEQ ID NO: 531.

In certain embodiments, the antibody comprises a VH sequence set forth in SEQ ID NO: 473 and a VL sequence set forth in SEQ ID NO: 532

In certain embodiments, the antibody comprises a VH sequence set forth in SEQ ID NO: 474 and a VL sequence set forth in SEQ ID NO: 533.

In certain embodiments, the antibody comprises a VH sequence set forth in SEQ ID NO: 475 and a VL sequence set forth in SEQ ID NO: 534.

In certain embodiments, the antibody comprises a VH sequence set forth in SEQ ID NO: 476 and a VL sequence set forth in SEQ ID NO: 535.

In certain embodiments, the antibody comprises a VH sequence set forth in SEQ ID NO: 477 and a VL sequence set forth in SEQ ID NO: 536.

In certain embodiments, the antibody comprises a VH sequence set forth in SEQ ID NO: 478 and a VL sequence set forth in SEQ ID NO: 536.

In certain embodiments, the antibody comprises a VH sequence set forth in SEQ ID NO: 479 and a VL sequence set forth in SEQ ID NO: 537.

In certain embodiments, the antibody comprises a VH sequence set forth in SEQ ID NO: 480 and a VL sequence set forth in SEQ ID NO: 538.

In certain embodiments, the antibody comprises a VH sequence set forth in SEQ ID NO: 481 and a VL sequence set forth in SEQ ID NO: 539.

In certain embodiments, the antibody comprises a VH sequence set forth in SEQ ID NO: 482 and a VL sequence set forth in SEQ ID NO: 539.

In certain embodiments, the antibody comprises a VH sequence set forth in SEQ ID NO: 483 and a VL sequence set forth in SEQ ID NO: 539.

In certain embodiments, the antibody comprises a VH sequence set forth in SEQ ID NO: 484 and a VL sequence set forth in SEQ ID NO: 540.

In certain embodiments, the antibody comprises a VH sequence set forth in SEQ ID NO: 485 and a VL sequence set forth in SEQ ID NO: 541.

In certain embodiments, the antibody comprises a VH sequence set forth in SEQ ID NO: 486 and a VL sequence set forth in SEQ ID NO: 542.

In certain embodiments, the antibody comprises a VH sequence set forth in SEQ ID NO: 487 and a VL sequence set forth in SEQ ID NO: 543.

In certain embodiments, the antibody comprises a VH sequence set forth in SEQ ID NO: 488 and a VL sequence set forth in SEQ ID NO: 544.

In certain embodiments, the antibody comprises a VH sequence set forth in SEQ ID NO: 818 and a VL sequence set forth in SEQ ID NO: 825.

In certain embodiments, the antibody comprises a VH sequence set forth in SEQ ID NO: 819 and a VL sequence set forth in SEQ ID NO: 826.

In certain embodiments, the antibody comprises a VH sequence set forth in SEQ ID NO: 820 and a VL sequence set forth in SEQ ID NO: 827.

In certain embodiments, the antibody comprises a VH sequence set forth in SEQ ID NO: 821 and a VL sequence set forth in SEQ ID NO: 828.

In certain embodiments, the antibody comprises a VH sequence set forth in SEQ ID NO: 822 and a VL sequence set forth in SEQ ID NO: 829.

In certain embodiments, the antibody comprises a VH sequence set forth in SEQ ID NO: 823 and a VL sequence set forth in SEQ ID NO: 830.

In certain embodiments, the antibody comprises a VH sequence set forth in SEQ ID NO: 824 and a VL sequence set forth in SEQ ID NO: 831.

In certain embodiments, the antibody comprises a VH sequence set forth in SEQ ID NO: 841 and a VL sequence set forth in SEQ ID NO: 842.

In certain embodiments, an antibody comprises any of the VH and VL combinations described above and further comprises a heavy chain comprising a human IgG sequence selected from a sequence set forth in SEQ ID NOs: 637-737 or a sequence having at least about 80%, 90%, 91%, 92%, 93%, 94%, 95%, 96%, 97%, 98% or 99% identity thereto. In certain embodiments, an antibody comprises any of the VH and VL combinations described above and further comprises a heavy chain comprising a heavy chain sequence set forth in SEQ ID NO: 651 or a sequence having at least about 80%, 90%, 91%, 92%, 93%, 94%, 95%, 96%, 97%, 98% or 99% identity thereto. In certain embodiments, an antibody comprises any of the VH and VL combinations described above and further comprises a human light chain sequence set forth in SEQ ID NO: 738 or a sequence having at least about 80%, 90%, 91%, 92%, 93%, 94%, 95%, 96%, 97%, 98% or 99% identity thereto. In certain embodiments, an antibody comprises any of the VH and VL combinations described above and further comprises a heavy chain sequence set forth in SEQ ID NO: 651 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In some embodiments, an antibody provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 258, 286, and 311; a CDR-L2 comprising an amino acid sequence set forth in any one of SEQ ID NOs: 335 and 359; a CDR-L3 comprising the sequence set forth in any one of SEQ ID NOs: 368 and 394, a heavy chain sequence set forth in SEQ ID NO: 651 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In certain embodiments, an antibody comprises a VH comprising SEQ ID NO: 419 or a sequence having at least about 80%, 90%, 91%, 92%, 93%, 94%, 95%, 96%, 97%, 98% or 99% identity thereto, a VL comprising SEQ ID NO: 489 or a sequence having at least about 80%, 90%, 91%, 92%, 93%, 94%, 95%, 96%, 97%, 98% or 99% identity thereto, a heavy chain sequence set forth in SEQ ID NO: 651 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any of SEQ ID NOs: 70, 111, and 142; a CDR-H3 comprising an amino acid sequence set forth in any one of SEQ ID NOs: 175, 203, and 234, a CDR-L1 comprising an amino acid sequence set forth in any of SEQ ID NOs: 260, 288, 313; a CDR-L2 comprising an amino acid sequence set forth in any of SEQ ID NOs: 347 and 365; a CDR-L3 comprising an amino acid sequence set forth in any one of SEQ ID NOs: 378 and 404, a heavy chain sequence set forth in SEQ ID NO: 651 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In certain embodiments, an antibody comprises a VH comprising SEQ ID NO: 841 or a sequence having at least about 80%, 90%, 91%, 92%, 93%, 94%, 95%, 96%, 97%, 98% or 99% identity thereto, a VL comprising SEQ ID NO: 842 or a sequence having at least about 80%, 90%, 91%, 92%, 93%, 94%, 95%, 96%, 97%, 98% or 99% identity thereto, a heavy chain sequence set forth in SEQ ID NO: 651 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

### CDRs

In some embodiments, disclosed herein is an antibody comprising 1, 2, 3, 4, 5, or 6 of the CDRs of TABLE 2 or TABLE 3. In some embodiments, disclosed herein is an antibody comprising 6 of the Kabat CDRs of TABLE 2 or TABLE 3 of the Chothia CDRs of TABLE 2 or TABLE 3 of the IMGT CDRs of TABLE 2 or TABLE 3.

In some embodiments, an antibody provided herein comprises one to three CDRs of a VH domain selected from SEQ ID NOs: 419-488, 818-824, and 840. In some embodiments, an antibody provided herein comprises two to three CDRs of a VH domain selected from SEQ ID NOs: 419-488, 818-824, and 840. In some embodiments, an antibody provided herein comprises three CDRs of a VH domain selected from SEQ ID NOs: 419-488, 818-824, and 840. In some aspects, the CDRs are exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity with a CDR-H1, CDR-H2, or CDR-H3 selected from SEQ ID NOs: 1-257 and 740-780. In some embodiments, the CDR-H1 is a CDR-H1 of a VH domain selected from SEQ ID NOs: 419-488, 818-824, and 840, with up to 1, 2, 3, 4, or 5 amino acid substitutions. In some embodiments, the CDR-H2 is a CDR-H2 of a VH domain selected from SEQ ID NOs: 419-488, 818-824, and 840, with up to 1, 2, 3, 4, 5, 6, 7, or 8 amino acid substitutions. In some embodiments, the CDR-H3 is a CDR-H3 of a VH domain selected from SEQ ID NOs: 419-488, 818-824, and 840, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a VL domain selected from SEQ ID NOs: 489-544 and 825-831. In some embodiments, an antibody provided herein comprises two to three CDRs of a VL domain selected from SEQ ID NOs: 489-544 and 825-831. In some embodiments, an antibody provided herein comprises three CDRs of a VL domain selected from SEQ ID NOs: 489-544 and 825-831. In some aspects, the CDRs are exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity with a CDR-L1, CDR-L2, or CDR-L3 selected from SEQ ID NOs: 258-393 and 781-811, In some embodiments, the CDR-L1 is a CDR-L1 of a VL domain selected from SEQ ID NOs: 489-544 and 825-831, with up to 1, 2, 3, 4, or 5 amino acid substitutions. In some embodiments, the CDR-L2 is a CDR-L2 of a VL domain selected from SEQ ID NOs: 489-544 and 825-831, with up to 1, 2, 3, 4, 5, 6, 7, or 8 amino acid substitutions. In some embodiments, the CDR-L3 is a CDR-L3 of a VL domain selected from SEQ ID NOs: 489-544 and 825-831, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises one to three CDRs of a VH domain selected from SEQ ID NOs: 419-488, 818-824, and 840 and one to three CDRs of a VL domain selected from SEQ ID NOs: 489-544 and 825-831. In some embodiments, an antibody provided herein comprises two to three CDRs of a VH domain selected from SEQ ID NOs: 419-488, 818-824, and 840 and two to three CDRs of a VL domain selected from SEQ ID NOs: 489-544 and 825-831. In some embodiments, an antibody provided herein comprises three CDRs of a VH domain selected from SEQ ID NOs: 419-488, 818-824, and 840 and three CDRs of a VL domain selected from SEQ ID NOs: 489-544 and 825-831. In some aspects, the CDRs are exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, an antibody provided herein comprises a CDR-H3 selected from SEQ ID NOs: 175-257 and 763-780. In some aspects, the CDR-H3 has at least about 80%, 90%, 91%, 92%, 93%, 94%, 95%, 96%, 97%, 98% or 99% identity with a CDR-H3 selected from SEQ ID NOs: 175-257 and 763-780. In some embodiments, the CDR-H3 is a CDR-H3 selected from SEQ ID NOs: 175-257 and 763-780, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H1 selected from SEQ ID NOs: 1-69, 740-747, and 840. In some aspects, the CDR-H1 has at least about 80%, 90%, 91%, 92%, 93%, 94%, 95%, 96%, 97%, 98% or 99% identity with a CDR-H1 selected from SEQ ID NOs: 1-69, 740-747, and 840. In some embodiments, the CDR-H1 is a CDR-H1 selected from SEQ ID NOs: 1-69, 740-747, and 840, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H2 selected from SEQ ID NOs: 70-174 and 748-762. In some aspects, the CDR-H2 has at least about 80%, 90%, 91%, 92%, 93%, 94%, 95%, 96%, 97%, 98% or 99% identity with a CDR-H2 H2 selected from SEQ ID NOs: 70-174 and 748-762. In some embodiments, the CDR-H2 is a CDR-H2 selected from SEQ ID NOs: 70-174 and 748-762, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L3 selected from SEQ ID NOs: 268-393 and 806-811. In some aspects, the CDR-L3 has at least about 80%, 90%, 91%, 92%, 93%, 94%, 95%, 96%, 97%, 98% or 99% identity with a CDR-L3 selected from SEQ ID NOs: 268-393 and 806-811. In some embodiments, the CDR-L3 is a CDR-L3 selected from SEQ ID NOs: 268-393 and 806-811, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L2 selected from SEQ ID NOs: 335-367 and 797-805. In some aspects, the CDR-L2 has at least about 80%, 90%, 91%, 92%, 93%, 94%, 95%, 96%, 97%, 98% or 99% identity with a CDR-L2 selected from SEQ ID NOs: 335-367 and 797-805. In some embodiments, the CDR-L2 is a CDR-L2 selected from SEQ ID NOs: 335-367 and 797-805, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-L1 selected from SEQ ID NOs: 258-334 and 781-796. In some aspects, the CDR-L1 has at least about 80%, 90%, 91%, 92%, 93%, 94%, 95%, 96%, 97%, 98% or 99% identity with a CDR-L1 selected from SEQ ID NOs: 258-334 and 781-796. In some embodiments, the CDR-L1 is a CDR-L1 selected from SEQ ID NOs: 258-334 and 781-796, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody provided herein comprises a CDR-H3 selected from SEQ ID NOs: 175-257 and 763-780, a CDR-H2 selected from SEQ ID NOs: 70-174 and 748-762, a CDR-H1 selected from SEQ ID NOs: 1-69 and 740-747, a CDR-L3 selected from SEQ ID NOs: 268-393 and 806-811, a CDR-L2 selected from SEQ ID NOs: 335-367 and 797-805, and a CDR-L1 selected from SEQ ID NOs: 258-334 and 781-796. In some embodiments, the CDR-H3 has at least about 80%, 90%, 91%, 92%, 93%, 94%, 95%, 96%, 97%, 98% or 99% identity with a CDR-H3 selected from SEQ ID NOs: 175-257 and 763-780, the CDR-H2 has at least about 80%, 90%, 91%, 92%, 93%, 94%, 95%, 96%, 97%, 98% or 99% identity with a CDR-H2 selected from SEQ ID NOs: 70-174 and 748-762, the CDR-H1 has at least about 80%, 90%, 91%, 92%, 93%, 94%, 95%, 96%, 97%, 98% or 99% identity with a CDR-H1 selected from SEQ ID NOs: 1-69 and 740-747, the CDR-L3 has at least about 80%, 90%, 91%, 92%, 93%, 94%, 95%, 96%, 97%, 98% or 99% identity with a CDR-L3 selected from SEQ ID NOs: 268-393 and 806-811, the CDR-L2 has at least about 80%, 90%, 91%, 92%, 93%, 94%, 95%, 96%, 97%, 98% or 99% identity with a CDR-L2 selected from SEQ ID NOs: 335-367 and 797-805, and the CDR-L1 has at least about 80%, 90%, 91%, 92%, 93%, 94%, 95%, 96%, 97%, 98% or 99% identity with a CDR-L1 selected from SEQ ID NOs: 258-334 and 781-796. In some embodiments, the CDR-H3 is a CDR-H3 selected from SEQ ID NOs: 175-257 and 763-780, with up to 1, 2, 3, 4, 5, 6, 7, or 8 amino acid substitutions; the CDR-H2 is a CDR-H2 selected from SEQ ID NOs: 70-174 and 748-762, with up to 1, 2, 3, 4, 5, 6, 7, or 8 amino acid substitutions; the CDR-H1 is a CDR-H1 selected from SEQ ID NOs: 1-69 and 740-747, with up to 1, 2, 3, 4, or 5 amino acid substitutions; the CDR-L3 is a CDR-L3 selected from SEQ ID NOs: 268-393 and 806-811, with up to 1, 2, 3, 4, or 5 amino acid substitutions; the CDR-L2 is a CDR-L2 selected from SEQ ID NOs: 335-367 and 797-805, with up to 1, 2, 3, or 4 amino acid substitutions; and the CDR-L1 is a CDR-L1 selected from SEQ ID NOs: 258-334 and 781-796, with up to 1, 2, 3, 4, 5, or 6 amino acid substitutions.

In some embodiments, an antibody provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 258, 286, and 311; a CDR-L2 comprising an amino acid sequence set forth in any one of SEQ ID NOs: 335 and 359; and a CDR-L3 comprising the sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity with a CDR-H3 selected from SEQ ID NOs: 17 and 91, the CDR-H2 has at least about 80%, 90%, 91%, 92%, 93%, 94%, 95%, 96%, 97%, 98% or 99% identity with a CDR-H2 selected from SEQ ID NOs: 5, 71, and 79, the CDR-H1 has at least about 80%, 90%, 91%, 92%, 93%, 94%, 95%, 96%, 97%, 98% or 99% identity with a CDR-H1 selected from SEQ ID NOs: 1, 67, and 187, the CDR-L3 has at least about 80%, 90%, 91%, 92%, 93%, 94%, 95%, 96%, 97%, 98% or 99% identity with a CDR-L3 set forth in SEQ ID NO: 53, the CDR-L2 has at least about 80%, 90%, 91%, 92%, 93%, 94%, 95%, 96%, 97%, 98% or 99% identity with a CDR-L2 selected from SEQ ID NOs: 41 and 108, and the CDR-L1 has at least about 80%, 90%, 91%, 92%, 93%, 94%, 95%, 96%, 97%, 98% or 99% identity with a CDR-L1 selected from SEQ ID NOs: 26 and 100. In some embodiments, the CDR-H3 is a CDR-H3 selected from SEQ ID NOs: 17 and 91, with up to 1, 2, 3, 4, 5, 6, 7, and 8 amino acid substitutions; the CDR-H2 is a CDR-H2 selected from SEQ ID NOs: 5, 71, and 79, with up to 1, 2, 3, 4, 5, 6, 7, and 8 amino acid substitutions; the CDR-H1 is a CDR-H1 selected from SEQ ID NOs: 1, 67, and 187, with up to 1, 2, 3, 4, and 5 amino acid substitutions; the CDR-L3 is a CDR-L3 set forth in SEQ ID NO: 53 with up to 1, 2, 3, 4, and 5 amino acid substitutions; the CDR-L2 is a CDR-L2 selected from SEQ ID NOs: 41 and 108, with up to 1, 2, 3, and 4 amino acid substitutions; and the CDR-L1 is a CDR-L1 selected from SEQ ID NOs: 26 and 100, with up to 1, 2, 3, 4, 5, and 6 amino acid substitutions.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 1, 22, and 44, the CDR-L3 has at least about 80%, 90%, 91%, 92%, 93%, 94%, 95%, 96%, 97%, 98% or 99% identity with a CDR-L3 selected from SEQ ID NOs: 368 and 394, the CDR-L2 has at least about 80%, 90%, 91%, 92%, 93%, 94%, 95%, 96%, 97%, 98% or 99% identity with a CDR-L2 selected from SEQ ID NOs: 335 and 359, and the CDR-L1 has at least about 80%, 90%, 91%, 92%, 93%, 94%, 95%, 96%, 97%, 98% or 99% identity with a CDR-L1 selected from SEQ ID NOs: 258, 286, and 311. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NOs: 1, 22, and 44, with up to 1, 2, 3, 4, or 5 amino acid substitutions; the CDR-L3 is a CDR-L3 selected from SEQ ID NOs: 368 and 394, with up to 1, 2, 3, 4, or 5 amino acid substitutions; the CDR-L2 is a CDR-L2 selected from SEQ ID NOs: 335 and 359, with up to 1, 2, 3, or 4 amino acid substitutions; and the CDR-L1 is a CDR-L1 selected from SEQ ID NOs: 258, 286, and 311, with up to 1, 2, 3, 4, 5, or 6 amino acid substitutions.

In some embodiments, an antibody provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 258, 286, and 311; a CDR-L2 comprising an amino acid sequence set forth in any one of SEQ ID NOs: 335 and 359; and a CDR-L3 comprising an amino acid sequence set forth in any one of SEQ ID NOs: 368 and 394.

In some embodiments, the CDR-H3 has at least about 80%, 90%, 91%, 92% 93%, 94%, 95%, 96%, 97%, 98% or 99% identity with a CDR-H3 selected from SEQ ID NOs: 176, 204, and 224, the CDR-H2 has at least about 80%, 90%, 91%, 92%, 93%, 94%, 95%, 96%, 97%, 98% or 99% identity with a CDR-H2 selected from SEQ ID NO: 70, 111, and 142, the CDR-H1 has at least about 80%, 90%, 91%, 92%, 93%, 94%, 95%, 96%, 97%, 98% or 99% identity with a CDR-H1 selected from SEQ ID NO: 2, 23, and 45, the CDR-L3 has at least about 80%, 90%, 91%, 92%, 93%, 94%, 95%, 96%, 97%, 98% or 99% identity with a CDR-L3 selected from SEQ ID NO: 369 or 395, the CDR-L2 has at least about 80%, 90%, 91%, 92%, 93%, 94%, 95%, 96%, 97%, 98% or 99% identity with a CDR-L2 selected from SEQ ID NO: 336 or 360, and the CDR-L1 has at least about 80%, 90%, 91%, 92%, 93%, 94%, 95%, 96%, 97%, 98% or 99% identity with a CDR-L1 selected from SEQ ID NO: 259, 287, and 312. In some embodiments, the CDR-H3 is a CDR-H3 selected from SEQ ID NOs: 176, 204, and 224, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NOs: 2, 23, and 45, with up to 1, 2, 3, 4, or 5 amino acid substitutions; the CDR-L3 is a CDR-L3 selected from SEQ ID NOs: 369 and 395, with up to 1, 2, 3, 4, and 5 amino acid substitutions; the CDR-L2 is a CDR-L2 selected from SEQ ID NOs: 336 and 360, with up to 1, 2, 3, and 4 amino acid substitutions; and the CDR-L1 is a CDR-L1 selected from SEQ ID NOs: 259, 287, and 312.

In some embodiments, an antibody provided herein comprises a CDR-H1 comprising an amino acid sequence set forth in any one of SEQ ID NOs: 2, 23, and 45; a CDR-H2 comprising an amino acid sequence set forth in any one of SEQ ID NOs: 70, 111, and 142; a CDR-H3 comprising an amino acid sequence set forth in any one of SEQ ID NOs: 176, 204, and 224; a CDR-L1 comprising an amino acid sequence set forth in any one of SEQ ID NOs: 259, 287, and 312; a CDR-L2 comprising an amino acid sequence set forth in any one of SEQ ID NOs: 336 and 360; and a CDR-L3 comprising an amino acid sequence set forth in any one of SEQ ID NOs: 369 and 395.

In some embodiments, the CDR-H3 has at least about 80%, 90%, 91%, 92%, 93%, 94%, 95%, 96%, 97%, 98% or 99% identity with a CDR-H3 selected from SEQ ID NOs: 177, 205, and 225, the CDR-H2 has at least about 80%, 90%, 91%, 92%, 93%, 94%, 95%, 96%, 97%, 98% or 99% identity with a CDR-H2 selected from SEQ ID NO: 71, 112, and 143, the CDR-H1 has at least about 80%, 90%, 91%, 92%, 93%, 94%, 95%, 96%, 97%, 98% or 99% identity with a CDR-H1 selected from SEQ ID NOs: 3, 24, and 46, the CDR-L3 has at least about 80%, 90%, 91%, 92%, 93%, 94%, 95%, 96%, 97%, 98% or 99% identity with a CDR-L3 selected from SEQ ID NOs: 370 and 396, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 177, 205, and 225, with up to 1, 2, 3, 4, 5, 6, 7, or 8 amino acid substitutions; the CDR-H2 is a CDR-H2 selected from SEQ ID NOs: 71, 112, and 143, with up to 1, 2, 3, 4, 5, 6, 7, or 8 amino acid substitutions; the CDR-H1 is a CDR-H1 selected from SEQ ID NOs: 3, 24, and 46, with up to 1, 2, 3, 4, or 5 amino acid substitutions; the CDR-L3 is a CDR-L3 selected from SEQ ID NOs: 370 and 396, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequences set forth in any one of SEQ ID NOs: 3, 24, and 46; a CDR-H2 comprising an amino acid sequence set forth in any one of SEQ ID NOs: 71, 112, and 143; a CDR-H3 comprising an amino acid sequence set forth in any one of SEQ ID NOs: 177, 205, and 225; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 370 and 396.

In some embodiments, the CDR-H3 has at least about 80%, 90%, 91%, 92%, 93%, 94%, 95%, 96%, 97%, 98% or 99% identity with a CDR-H3 selected from SEQ ID NO: 178, 206, and 226, the CDR-H2 has at least about 80%, 90%, 91%, 92%, 93%, 94%, 95%, 96%, 97%, 98% or 99% identity with a CDR-H2 selected from SEQ ID NO: 72, 113, and 144, the CDR-H1 has at least about 80%, 90%, 91%, 92%, 93%, 94%, 95%, 96%, 97%, 98% or 99% identity with a CDR-H1 selected from SEQ ID NO: 4, 22, and 47, the CDR-L3 has at least about 80%, 90%, 91%, 92%, 93%, 94%, 95%, 96%, 97%, 98% or 99% identity with a CDR-L3 selected from SEQ ID NO: 371 and 397, the CDR-L2 has at least about 80%, 90%, 91%, 92%, 93%, 94%, 95%, 96%, 97%, 98% or 99% identity with a CDR-L2 selected from SEQ ID NO: 337 and 361, and the CDR-L1 has at least about 80%, 90%, 91%, 92%, 93%, 94%, 95%, 96%, 97%, 98% or 99% identity with a CDR-L1 selected from SEQ ID NO: 261, 289, and 314. In some embodiments, the CDR-H3 is a CDR-H3 selected from SEQ ID NO: 178, 206, and 226, with up to 1, 2, 3, 4, 5, 6, 7, or 8 amino acid substitutions; the CDR-H2 is a CDR-H2 selected from SEQ ID NOs: 72, 113, and 144, with up to 1, 2, 3, 4, 5, 6, 7, or 8 amino acid substitutions; the CDR-H1 is a CDR-H1 selected from SEQ ID NO: 4, 22, and 47, with up to 1, 2, 3, 4, or 5 amino acid substitutions; the CDR-L3 is a CDR-L3 selected from SEQ ID NOs: 371 and 397, with up to 1, 2, 3, 4, or 5 amino acid substitutions; the CDR-L2 is a CDR-L2 selected from SEQ ID NO: 337 and 361, with up to 1, 2, 3, or 4 amino acid substitutions; and the CDR-L1 is a CDR-L1 selected from SEQ ID NO: 261, 289, and 314.

In some embodiments, an antibody provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 72, 113, and 144; a CDR-H3 comprising an amino acid sequence set forth in any one of SEQ ID NOs: 178, 206, and 226; a CDR-L1 comprising an amino acid sequence set forth in any one of SEQ ID NOs: 261, 289, and 314; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, the CDR-H3 has at least about 80%, 90%, 91%, 92%, 93%, 94%, 95%, 96%, 97%, 98% or 99% identity with a CDR-H3 selected from SEQ ID NOs: 179, 207, and 227, the CDR-H2 has at least about 80%, 90%, 91%, 92%, 93%, 94%, 95%, 96%, 97%, 98% or 99% identity with a CDR-H2 selected from SEQ ID NOs: 73, 114, and 145, the CDR-H1 has at least about 80%, 90%, 91%, 92%, 93%, 94%, 95%, 96%, 97%, 98% or 99% identity with a CDR-H1 selected from SEQ ID NOs: 5, 840, and 48, the CDR-L3 has at least about 80%, 90%, 91%, 92%, 93%, 94%, 95%, 96%, 97%, 98% or 99% identity with a CDR-L3 selected from SEQ ID NOs: 372 and 398, the CDR-L2 has at least about 80%, 90%, 91%, 92%, 93%, 94%, 95%, 96%, 97%, 98% or 99% identity with a CDR-L2 selected from SEQ ID NOs: 338 and 362, and the CDR-L1 has at least about 80%, 90%, 91%, 92%, 93%, 94%, 95%, 96%, 97%, 98% or 99% identity with a CDR-L1 selected from SEQ ID NOs: 262, 290, and 315. In some embodiments, the CDR-H3 is a CDR-H3 selected from SEQ ID NOs: 179, 207, and 227, with up to 1, 2, 3, 4, 5, 6, 7, and 8 amino acid substitutions; the CDR-H2 is a CDR-H2 selected from SEQ ID NOs: 73, 114, and 145, with up to 1, 2, 3, 4, 5, 6, 7 or 8 amino acid substitutions; the CDR-H1 is a CDR-H1 selected from SEQ ID NO: 5, 840, and 48, with up to 1, 2, 3, 4, or 5 amino acid substitutions; the CDR-L3 is a CDR-L3 selected from SEQ ID NOs: 372 and 398, with up to 1, 2, 3, 4, or 5 amino acid substitutions; the CDR-L2 is a CDR-L2 selected from SEQ ID NOs: 338 and 362, with up to 1, 2, 3, or 4 amino acid substitutions; and the CDR-L1 is a CDR-L1 selected from SEQ ID NOs: 262, 290, and 315.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 73, 114, and 145; a CDR-H3 comprising an amino acid sequence set forth in any one of SEQ ID NOs: 179, 207, and 227; a CDR-L1 comprising an amino acid sequence set forth in any one of SEQ ID NOs: 262, 290, and 315; a CDR-L2 comprising an amino acid sequence set forth in any one of SEQ ID NOs: 338 and 362; and a CDR-L3 comprising an amino acid sequence set forth in any one of SEQ ID NOs: 372 and 398.

In some embodiments, the CDR-H3 has at least about 80%, 90%, 91%, 92%, 93%, 94%, 95%, 96%, 97%, 98% or 99% identity with a CDR-H3 selected from SEQ ID NOs: 180, 208, and 228, the CDR-H2 has at least about 80%, 90%, 91%, 92%, 93%, 94%, 95%, 96%, 97%, 98% or 99% identity with a CDR-H2 selected from SEQ ID NOs: 74, 115, and 146, the CDR-H1 has at least about 80%, 90%, 91%, 92%, 93%, 94%, 95%, 96%, 97%, 98% or 99% identity with a CDR-H1 selected from SEQ ID NOs: 6, 24, and 49, the CDR-L3 has at least about 80%, 90%, 91%, 92%, 93%, 94%, 95%, 96%, 97%, 98% or 99% identity with a CDR-L3 selected from SEQ ID NOs: 373 and 399, the CDR-L2 has at least about 80%, 90%, 91%, 92%, 93%, 94%, 95%, 96%, 97%, 98% or 99% identity with a CDR-L2 selected from SEQ ID NOs: 339 and 361, and the CDR-L1 has at least about 80%, 90%, 91%, 92%, 93%, 94%, 95%, 96%, 97%, 98% or 99% identity with a CDR-L1 selected from SEQ ID NOs: 263, 291, and 316. In some embodiments, the CDR-H3 is a CDR-H3 selected from SEQ ID NOs: 180, 208, and 228, with up to 1, 2, 3, 4, 5, 6, 7, and 8 amino acid substitutions; the CDR-H2 is a CDR-H2 selected from SEQ ID NOs: 74, 115, and 146, with up to 1, 2, 3, 4, 5, 6, 7, and 8 amino acid substitutions; the CDR-H1 is a CDR-H1 selected from SEQ ID NO: 6, 24, and 49, with up to 1, 2, 3, 4, and 5 amino acid substitutions; the CDR-L3 is a CDR-L3 selected from SEQ ID NOs: 373 and 399, with up to 1, 2, 3, 4 or 5 amino acid substitutions; the CDR-L2 is a CDR-L2 selected from SEQ ID NOs: 339 and 361, with up to 1, 2, 3, or 4 amino acid substitutions; and the CDR-L1 is a CDR-L1 selected from SEQ ID NOs: 263, 291, and 316.

In some embodiments, an antibody provided herein comprises a CDR-H1 comprising an amino acid sequence set forth in any one of SEQ ID NOs: 6, 24, and 49; a CDR-H2 comprising an amino acid sequence set forth in any one of SEQ ID NOs: 74, 115, and 146; a CDR-H3 comprising an amino acid sequence set forth in any one of SEQ ID NOs: 180, 208, and 228; a CDR-L1 comprising an amino acid sequence set forth in any one of SEQ ID NOs: 263, 291, and 316; a CDR-L2 comprising an amino acid sequence set forth in any one of SEQ ID NOs: 339 and 361; and a CDR-L3 comprising an amino acid sequence set forth in any one of SEQ ID NOs: 373 and 399.

In some embodiments, the CDR-H3 has at least about 80%, 90%, 91%, 92%, 93%, 94%, 95%, 96%, 97%, 98% or 99% identity with a CDR-H3 selected from SEQ ID NOs: 181, 209, and 229, the CDR-H2 has at least about 80%, 90%, 91%, 92%, 93%, 94%, 95%, 96%, 97%, 98% or 99% identity with a CDR-H2 selected from SEQ ID NOs: 75, 116, and 147, the CDR-H1 has at least about 80%, 90%, 91%, 92%, 93%, 94%, 95%, 96%, 97%, 98% or 99% identity with a CDR-H1 selected from SEQ ID NOs: 7, 25, and 50, the CDR-L3 has at least about 80%, 90%, 91%, 92%, 93%, 94%, 95%, 96%, 97%, 98% or 99% identity with a CDR-L3 selected from SEQ ID NOs: 374 and 400, the CDR-L2 has at least about 80%, 90%, 91%, 92%, 93%, 94%, 95%, 96%, 97%, 98% or 99% identity with a CDR-L2 selected from SEQ ID NOs: 340 and 363, and the CDR-L1 has at least about 80%, 90%, 91%, 92%, 93%, 94%, 95%, 96%, 97%, 98% or 99% identity with a CDR-L1 selected from SEQ ID NOs: 264, 292, and 317. In some embodiments, the CDR-H3 is a CDR-H3 selected from SEQ ID NOs: 181, 209, and 229, with up to 1, 2, 3, 4, 5, 6, 7, and 8 amino acid substitutions; the CDR-H2 is a CDR-H2 selected from SEQ ID NOs: 75, 116, and 147, with up to 1, 2, 3, 4, 5, 6, 7, or 8 amino acid substitutions; the CDR-H1 is a CDR-H1 selected from SEQ ID NO: 7, 25, and 50, with up to 1, 2, 3, 4, or 5 amino acid substitutions; the CDR-L3 is a CDR-L3 selected from SEQ ID NOs: 374 and 400, with up to 1, 2, 3, 4, or 5 amino acid substitutions; the CDR-L2 is a CDR-L2 selected from SEQ ID NOs: 340 and 363, with up to 1, 2, 3, or 4 amino acid substitutions; and the CDR-L1 is a CDR-L1 selected from SEQ ID NOs: 264, 292, and 317.

In some embodiments, an antibody provided herein comprises a CDR-H1 comprising an amino acid sequence set forth in any one of SEQ ID NOs: 7, 25, and 50; a CDR-H2 comprising an amino acid sequence set forth in any one of SEQ ID NOs: 75, 116, and 147; a CDR-H3 comprising an amino acid sequence set forth in any one of SEQ ID NOs: 181, 209, and 229; a CDR-L1 comprising an amino acid sequence set forth in any one of SEQ ID NOs: 264, 292, and 317; a CDR-L2 comprising an amino acid sequence set forth in any one of SEQ ID NOs: 340 and 363; and a CDR-L3 comprising an amino acid sequence set forth in any one of SEQ ID NOs: 374 and 400.

In some embodiments, the CDR-H3 has at least about 80%, 90%, 91%, 92%, 93%, 94%, 95%, 96%, 97%, 98% or 99% identity with a CDR-H3 selected from SEQ ID NOs: 182, 210, and 230, the CDR-H2 has at least about 80%, 90%, 91%, 92%, 93%, 94%, 95%, 96%, 97%, 98% or 99% identity with a CDR-H2 selected from SEQ ID NOs: 76, 117, and 148, the CDR-H1 has at least about 80%, 90%, 91%, 92%, 93%, 94%, 95%, 96%, 97%, 98% or 99% identity with a CDR-H1 selected from SEQ ID NOs: 8, 26, and 51, the CDR-L3 has at least about 80%, 90%, 91%, 92%, 93%, 94%, 95%, 96%, 97%, 98% or 99% identity with a CDR-L3 selected from SEQ ID NOs: 375 and 401, the CDR-L2 has at least about 80%, 90%, 91%, 92%, 93%, 94%, 95%, 96%, 97%, 98% or 99% identity with a CDR-L2 selected from SEQ ID NOs: 341 and 361, and the CDR-L1 has at least about 80%, 90%, 91%, 92%, 93%, 94%, 95%, 96%, 97%, 98% or 99% identity with a CDR-L1 selected from SEQ ID NOs: 265, 293, and 318. In some embodiments, the CDR-H3 is a CDR-H3 selected from SEQ ID NOs: 182, 210, and 230, with up to 1, 2, 3, 4, 5, 6, 7, or 8 amino acid substitutions; the CDR-H2 is a CDR-H2 selected from SEQ ID NOs: 76, 117, and 148, with up to 1, 2, 3, 4, 5, 6, 7, or 8 amino acid substitutions; the CDR-H1 is a CDR-H1 selected from SEQ ID NO: 8, 26, and 51, with up to 1, 2, 3, 4, or 5 amino acid substitutions; the CDR-L3 is a CDR-L3 selected from SEQ ID NOs: 375 and 401, with up to 1, 2, 3, 4, or 5 amino acid substitutions; the CDR-L2 is a CDR-L2 selected from SEQ ID NOs: 341 and 361, with up to 1, 2, 3, or 4 amino acid substitutions; and the CDR-L1 is a CDR-L1 selected from SEQ ID NOs: 265, 293, and 318.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOS: 182, 210, and 230; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 375 and 401.

In some embodiments, the CDR-H3 has at least about 80%, 90%, 91%, 92%, 93%, 94%, 95%, 96%, 97%, 98% or 99% identity with a CDR-H3 selected from SEQ ID NOs: 183, 211, and 231, the CDR-H2 has at least about 80%, 90%, 91%, 92%, 93%, 94%, 95%, 96%, 97%, 98% or 99% identity with a CDR-H2 selected from SEQ ID NOs: 76, 117, and 148, the CDR-H1 has at least about 80%, 90%, 91%, 92%, 93%, 94%, 95%, 96%, 97%, 98% or 99% identity with a CDR-H1 selected from SEQ ID NOs: 8, 26, and 51, the CDR-L3 has at least about 80%, 90%, 91%, 92%, 93%, 94%, 95%, 96%, 97%, 98% or 99% identity with a CDR-L3 selected from SEQ ID NOs: 376 and 402, the CDR-L2 has at least about 80%, 90%, 91%, 92%, 93%, 94%, 95%, 96%, 97%, 98% or 99% identity with a CDR-L2 selected from SEQ ID NOs: 341 and 361, and the CDR-L1 has at least about 80%, 90%, 91%, 92%, 93%, 94%, 95%, 96%, 97%, 98% or 99% identity with a CDR-L1 selected from SEQ ID NOs: 265, 293, and 318. In some embodiments, the CDR-H3 is a CDR-H3 selected from SEQ ID NOs: 183, 211, and 231, with up to 1, 2, 3, 4, 5, 6, 7, or 8 amino acid substitutions; the CDR-H2 is a CDR-H2 selected from SEQ ID NOs: 76, 117, and 148, with up to 1, 2, 3, 4, 5, 6, 7, or 8 amino acid substitutions; the CDR-H1 is a CDR-H1 selected from SEQ ID NO: 8, 26, and 51, with up to 1, 2, 3, 4, or 5 amino acid substitutions; the CDR-L3 is a CDR-L3 selected from SEQ ID NOs: 376 and 402, with up to 1, 2, 3, 4, or 5 amino acid substitutions; the CDR-L2 is a CDR-L2 selected from SEQ ID NOs: 341 and 361, with up to 1, 2, 3, or 4 amino acid substitutions; and the CDR-L1 is a CDR-L1 selected from SEQ ID NOs: 265, 293, and 318.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 183, 211, and 231; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, the CDR-H3 has at least about 80%, 90%, 91%, 92%, 93%, 94%, 95%, 96%, 97%, 98% or 99% identity with a CDR-H3 selected from SEQ ID NOs: 183, 211, and 231, the CDR-H2 has at least about 80%, 90%, 91%, 92%, 93%, 94%, 95%, 96%, 97%, 98% or 99% identity with a CDR-H2 selected from SEQ ID NOs: 76, 117, and 148, the CDR-H1 has at least about 80%, 90%, 91%, 92%, 93%, 94%, 95%, 96%, 97%, 98% or 99% identity with a CDR-H1 selected from SEQ ID NOs: 8, 26, and 51, the CDR-L3 has at least about 80%, 90%, 91%, 92%, 93%, 94%, 95%, 96%, 97%, 98% or 99% identity with a CDR-L3 selected from SEQ ID NOs: 377 and 403, the CDR-L2 has at least about 80%, 90%, 91%, 92%, 93%, 94%, 95%, 96%, 97%, 98% or 99% identity with a CDR-L2 selected from SEQ ID NOs: 341 and 361, and the CDR-L1 has at least about 80%, 90%, 91%, 92%, 93%, 94%, 95%, 96%, 97%, 98% or 99% identity with a CDR-L1 selected from SEQ ID NOs: 265, 293, and 318. In some embodiments, the CDR-H3 is a CDR-H3 selected from SEQ ID NOs: 183, 211, or 231, with up to 1, 2, 3, 4, 5, 6, 7, or 8 amino acid substitutions; the CDR-H2 is a CDR-H2 selected from SEQ ID NOs: 76, 117, and 148, with up to 1, 2, 3, 4, 5, 6, 7, or 8 amino acid substitutions; the CDR-H1 is a CDR-H1 selected from SEQ ID NO: 8, 26, and 51, with up to 1, 2, 3, 4, or 5 amino acid substitutions; the CDR-L3 is a CDR-L3 selected from SEQ ID NOs: 377 and 403, with up to 1, 2, 3, 4, or 5 amino acid substitutions; the CDR-L2 is a CDR-L2 selected from SEQ ID NOs: 341 and 361, with up to 1, 2, 3, or 4 amino acid substitutions; and the CDR-L1 is a CDR-L1 selected from SEQ ID NOs: 265, 293, and 318.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 183, 211, and 231; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, the CDR-H3 has at least about 80%, 90%, 91%, 92%, 93%, 94%, 95%, 96%, 97%, 98% or 99% identity with a CDR-H3 selected from SEQ ID NOs: 184, 212, and 232, the CDR-H2 has at least about 80%, 90%, 91%, 92%, 93%, 94%, 95%, 96%, 97%, 98% or 99% identity with a CDR-H2 selected from SEQ ID NOs: 76, 117, and 148, the CDR-H1 has at least about 80%, 90%, 91%, 92%, 93%, 94%, 95%, 96%, 97%, 98% or 99% identity with a CDR-H1 selected from SEQ ID NOs: 8, 26, and 51, the CDR-L3 has at least about 80%, 90%, 91%, 92%, 93%, 94%, 95%, 96%, 97%, 98% or 99% identity with a CDR-L3 selected from SEQ ID NOs: 376 and 402, the CDR-L2 has at least about 80%, 90%, 91%, 92%, 93%, 94%, 95%, 96%, 97%, 98% or 99% identity with a CDR-L2 selected from SEQ ID NOs: 341 and 361, and the CDR-L1 has at least about 80%, 90%, 91%, 92%, 93%, 94%, 95%, 96%, 97%, 98% or 99% identity with a CDR-L1 selected from SEQ ID NOs: 265, 293, and 318. In some embodiments, the CDR-H3 is a CDR-H3 selected from SEQ ID NOs: 184, 212, and 232, with up to 1, 2, 3, 4, 5, 6, 7, or 8 amino acid substitutions; the CDR-H2 is a CDR-H2 selected from SEQ ID NOs: 76, 117, and 148, with up to 1, 2, 3, 4, 5, 6, 7, or 8 amino acid substitutions; the CDR-H1 is a CDR-H1 selected from SEQ ID NO: 8, 26, and 51, with up to 1, 2, 3, 4, or 5 amino acid substitutions; the CDR-L3 is a CDR-L3 selected from SEQ ID NOs: 376 and 402, with up to 1, 2, 3, 4, or 5 amino acid substitutions; the CDR-L2 is a CDR-L2 selected from SEQ ID NOs: 341 and 361, with up to 1, 2, 3, or 4 amino acid substitutions; and the CDR-L1 is a CDR-L1 selected from SEQ ID NOs: 265, 293, and 318.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 184, 212, and 232; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, the CDR-H3 has at least about 80%, 90%, 91%, 92%, 93%, 94%, 95%, 96%, 97%, 98% or 99% identity with a CDR-H3 selected from SEQ ID NOs: 184, 212, and 232, the CDR-H2 has at least about 80%, 90%, 91%, 92%, 93%, 94%, 95%, 96%, 97%, 98% or 99% identity with a CDR-H2 selected from SEQ ID NOs: 76, 117, and 148, the CDR-H1 has at least about 80%, 90%, 91%, 92%, 93%, 94%, 95%, 96%, 97%, 98% or 99% identity with a CDR-H1 selected from SEQ ID NOs: 8, 26, and 51, the CDR-L3 has at least about 80%, 90%, 91%, 92%, 93%, 94%, 95%, 96%, 97%, 98% or 99% identity with a CDR-L3 selected from SEQ ID NOs: 377 and 403, the CDR-L2 has at least about 80%, 90%, 91%, 92%, 93%, 94%, 95%, 96%, 97%, 98% or 99% identity with a CDR-L2 selected from SEQ ID NOs: 341 or 361, and the CDR-L1 has at least about 80%, 90%, 91%, 92%, 93%, 94%, 95%, 96%, 97%, 98% or 99% identity with a CDR-L1 selected from SEQ ID NOs: 265, 293, and 318. In some embodiments, the CDR-H3 is a CDR-H3 selected from SEQ ID NOs: 184, 212, and 232, with up to 1, 2, 3, 4, 5, 6, 7, or 8 amino acid substitutions; the CDR-H2 is a CDR-H2 selected from SEQ ID NOs: 76, 117, and 148, with up to 1, 2, 3, 4, 5, 6, 7, or 8 amino acid substitutions; the CDR-H1 is a CDR-H1 selected from SEQ ID NO: 8, 26, and 51, with up to 1, 2, 3, 4, or 5 amino acid substitutions; the CDR-L3 is a CDR-L3 selected from SEQ ID NOs: 377 and 403, with up to 1, 2, 3, 4, or 5 amino acid substitutions; the CDR-L2 is a CDR-L2 selected from SEQ ID NOs: 341 and 361, with up to 1, 2, 3, or 4 amino acid substitutions; and the CDR-L1 is a CDR-L1 selected from SEQ ID NOs: 265, 293, and 318.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 184, 212, and 232; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, the CDR-H3 has at least about 80%, 90%, 91%, 92%, 93%, 94%, 95%, 96%, 97%, 98% or 99% identity with a CDR-H3 selected from SEQ ID NOs: 185, 213, and 233, the CDR-H2 has at least about 80%, 90%, 91%, 92%, 93%, 94%, 95%, 96%, 97%, 98% or 99% identity with a CDR-H2 selected from SEQ ID NOs: 76, 117, and 148, the CDR-H1 has at least about 80%, 90%, 91%, 92%, 93%, 94%, 95%, 96%, 97%, 98% or 99% identity with a CDR-H1 selected from SEQ ID NOs: 8, 26, and 51, the CDR-L3 has at least about 80%, 90%, 91%, 92%, 93%, 94%, 95%, 96%, 97%, 98% or 99% identity with a CDR-L3 selected from SEQ ID NOs: 376 and 402, the CDR-L2 has at least about 80%, 90%, 91%, 92%, 93%, 94%, 95%, 96%, 97%, 98% or 99% identity with a CDR-L2 selected from SEQ ID NOs: 341 or 361, and the CDR-L1 has at least about 80%, 90%, 91%, 92%, 93%, 94%, 95%, 96%, 97%, 98% or 99% identity with a CDR-L1 selected from SEQ ID NOs: 265, 293, and 318. In some embodiments, the CDR-H3 is a CDR-H3 selected from SEQ ID NOs: 185, 213, and 233, with up to 1, 2, 3, 4, 5, 6, 7, or 8 amino acid substitutions; the CDR-H2 is a CDR-H2 selected from SEQ ID NOs: 76, 117, and 148, with up to 1, 2, 3, 4, 5, 6, 7, or 8 amino acid substitutions; the CDR-H1 is a CDR-H1 selected from SEQ ID NO: 8, 26, and 51, with up to 1, 2, 3, 4, or 5 amino acid substitutions; the CDR-L3 is a CDR-L3 selected from SEQ ID NOs: 376 and 402, with up to 1, 2, 3, 4, or 5 amino acid substitutions; the CDR-L2 is a CDR-L2 selected from SEQ ID NOs: 341 and 361, with up to 1, 2, 3, or 4 amino acid substitutions; and the CDR-L1 is a CDR-L1 selected from SEQ ID NOs: 265, 293, and 318.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 185, 213, and 233; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, the CDR-H3 has at least about 80%, 90%, 91%, 92%, 93%, 94%, 95%, 96%, 97%, 98% or 99% identity with a CDR-H3 selected from SEQ ID NOs: 185, 213, and 233, the CDR-H2 has at least about 80%, 90%, 91%, 92%, 93%, 94%, 95%, 96%, 97%, 98% or 99% identity with a CDR-H2 selected from SEQ ID NOs: 76, 117, and 148, the CDR-H1 has at least about 80%, 90%, 91%, 92%, 93%, 94%, 95%, 96%, 97%, 98% or 99% identity with a CDR-H1 selected from SEQ ID NOs: 8, 26, and 51, the CDR-L3 has at least about 80%, 90%, 91%, 92%, 93%, 94%, 95%, 96%, 97%, 98% or 99% identity with a CDR-L3 selected from SEQ ID NOs: 377 and 403, the CDR-L2 has at least about 80%, 90%, 91%, 92%, 93%, 94%, 95%, 96%, 97%, 98% or 99% identity with a CDR-L2 selected from SEQ ID NOs: 341 or 361, and the CDR-L1 has at least about 80%, 90%, 91%, 92%, 93%, 94%, 95%, 96%, 97%, 98% or 99% identity with a CDR-L1 selected from SEQ ID NOs: 265, 293, and 318. In some embodiments, the CDR-H3 is a CDR-H3 selected from SEQ ID NOs: 185, 213, and 233, with up to 1, 2, 3, 4, 5, 6, 7, or 8 amino acid substitutions; the CDR-H2 is a CDR-H2 selected from SEQ ID NOs: 76, 117, and 148, with up to 1, 2, 3, 4, 5, 6, 7, or 8 amino acid substitutions; the CDR-H1 is a CDR-H1 selected from SEQ ID NOs: 8, 26, and 51, with up to 1, 2, 3, 4, or 5 amino acid substitutions; the CDR-L3 is a CDR-L3 selected from SEQ ID NOs: 377 and 403, with up to 1, 2, 3, 4, or 5 amino acid substitutions; the CDR-L2 is a CDR-L2 selected from SEQ ID NOs: 341 and 361, with up to 1, 2, 3, or 4 amino acid substitutions; and the CDR-L1 is a CDR-L1 selected from SEQ ID NOs: 265, 293, and 318.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 185, 213, and 233; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 234, the CDR-H2 has at least about 80%, 90%, 91%, 92%, 93%, 94%, 95%, 96%, 97%, 98% or 99% identity with a CDR-H2 selected from SEQ ID NOs: 77, 118, and 149, the CDR-H1 has at least about 80%, 90%, 91%, 92%, 93%, 94%, 95%, 96%, 97%, 98% or 99% identity with a CDR-H1 selected from SEQ ID NOs: 9, 27, and 52, the CDR-L3 has at least about 80%, 90%, 91%, 92%, 93%, 94%, 95%, 96%, 97%, 98% or 99% identity with a CDR-L3 selected from SEQ ID NOs: 368 and 394, the CDR-L2 has at least about 80%, 90%, 91%, 92%, 93%, 94%, 95%, 96%, 97%, 98% or 99% identity with a CDR-L2 selected from SEQ ID NOs: 342 and 364, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 234, with up to 1, 2, 3, 4, 5, 6, 7, or 8 amino acid substitutions; the CDR-H2 is a CDR-H2 selected from SEQ ID NOs: 77, 118, and 149, with up to 1, 2, 3, 4, 5, 6, 7, or 8 amino acid substitutions; the CDR-H1 is a CDR-H1 selected from SEQ ID NOs: 9, 27, and 52, with up to 1, 2, 3, 4, or 5 amino acid substitutions; the CDR-L3 is a CDR-L3 selected from SEQ ID NOs: 368 and 394, with up to 1, 2, 3, 4, or 5 amino acid substitutions; the CDR-L2 is a CDR-L2 selected from SEQ ID NOs: 342 and 364, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 9, 27, and 52; a CDR-H2 comprising an amino acid sequence set forth in any one of SEQ ID NOs: 77, 118, and 149; a CDR-H3 comprising an amino acid sequence set forth in any one of SEQ ID NOS: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 342 and 364; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 78, 119, and 150, the CDR-H1 has at least about 80%, 90%, 91%, 92%, 93%, 94%, 95%, 96%, 97%, 98% or 99% identity with a CDR-H1 selected from SEQ ID NOs: 1, 28, and 53, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 343 and 365, and the CDR-L1 has at least about 80%, 90%, 91%, 92%, 93%, 94%, 95%, 96%, 97%, 98% or 99% identity with a CDR-L1 selected from SEQ ID NOs: 266, 286, and 311. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 78, 119, and 150, with up to 1, 2, 3, 4, 5, 6, 7, or 8 amino acid substitutions; the CDR-H1 is a CDR-H1 selected from SEQ ID NO: 1, 28, and 53, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 343 and 365, with up to 1, 2, 3, or 4 amino acid substitutions; and the CDR-L1 is a CDR-L1 selected from SEQ ID NOs: 266, 286, and 311.

In some embodiments, an antibody provided herein comprises a CDR-H1 comprising an amino acid sequence set forth in any one of SEQ ID NOs: 1, 28, and 53; a CDR-H2 comprising an amino acid sequence set forth in any one of SEQ ID NOs: 78, 119, and 150; a CDR-H3 comprising an amino acid sequence set forth in any one of SEQ ID NOs: 175, 203, and 223; a CDR-L1 comprising an amino acid sequence set forth in any one of SEQ ID NOs: 266, 286, and 311; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 186, 203, and 235, the CDR-H2 has at least about 80%, 90%, 91%, 92%, 93%, 94%, 95%, 96%, 97%, 98% or 99% identity with a CDR-H2 selected from SEQ ID NOs: 79, 119, and 150, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 344 and 365, and the CDR-L1 has at least about 80%, 90%, 91%, 92%, 93%, 94%, 95%, 96%, 97%, 98% or 99% identity with a CDR-L1 selected from SEQ ID NOs: 267, 294, and 319. In some embodiments, the CDR-H3 is a CDR-H3 selected from SEQ ID NOs: 186, 203, and 235, with up to 1, 2, 3, 4, 5, 6, 7, or 8 amino acid substitutions; the CDR-H2 is a CDR-H2 selected from SEQ ID NOs: 79, 119, and 150, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 344 and 365, with up to 1, 2, 3, or 4 amino acid substitutions; and the CDR-L1 is a CDR-L1 selected from SEQ ID NOs: 267, 294, and 319.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 186, 203, and 235; a CDR-L1 comprising an amino acid sequence set forth in any one of SEQ ID NOs: 267, 294, and 319; a CDR-L2 comprising an amino acid sequence set forth in any one of SEQ ID NOs: 344 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 186, 203, and 235, the CDR-H2 has at least about 80%, 90%, 91%, 92%, 93%, 94%, 95%, 96%, 97%, 98% or 99% identity with a CDR-H2 selected from SEQ ID NOs: 80, 120, and 151, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 343 and 365, and the CDR-L1 has at least about 80%, 90%, SEQ ID NOs: 260, 288, and 313. In some embodiments, the CDR-H3 is a CDR-H3 selected from SEQ ID NOs: 186, 203, and 235, with up to 1, 2, 3, 4, 5, 6, 7, or 8 amino acid substitutions; the CDR-H2 is a CDR-H2 selected from SEQ ID NOs: 80, 120, and 151, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 343 and 365, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 80, 120, and 151; a CDR-H3 comprising an amino acid sequence set forth in any one of SEQ ID NOs: 186, 203, and 235; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 79, 119, and 150, the CDR-H1 has at least about 80%, 90%, 91%, 92%, 93%, 94%, 95%, 96%, 97%, 98% or 99% identity with a CDR-H1 selected from SEQ ID NOs: 11, 27, or 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 343 and 365, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 79, 119, and 150, with SEQ ID NO: 11, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 343 and 365, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 11, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 81, 121, and 152, the CDR-H1 has at least about 80%, 90%, 91%, 92%, 93%, 94%, 95%, 96%, 97%, 98% or 99% identity with a CDR-H1 selected from SEQ ID NOs: 11, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 343 and 365, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 81, 121, and 152, with up to 1, 2, 3, 4, 5, 6, 7, or 8 amino acid substitutions; the CDR-H1 is a CDR-H1 selected from SEQ ID NO: 11, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 343 and 365, with up to NOs: 260, 288, and 313.

In some embodiments, an antibody provided herein comprises a CDR-H1 comprising an amino acid sequence set forth in any one of SEQ ID NOs: 11, 27, and 52; a CDR-H2 comprising an amino acid sequence set forth in any one of SEQ ID NOs: 81, 121, and 152; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 187, 203, and 236, the CDR-H2 has at least about 80%, 90%, 91%, 92%, 93%, 94%, 95%, 96%, 97%, 98% or 99% identity with a CDR-H2 selected from SEQ ID NOs: 80, 120, and 151, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NO: 343 and 365, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 187, 203, and 236, with up to 1, 2, 3, 4, 5, 6, 7, or 8 amino acid substitutions; the CDR-H2 is a CDR-H2 selected from SEQ ID NOs: 80, 120, and 151, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 343 and 365, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 80, 120, and 151; a CDR-H3 comprising an amino acid sequence set forth in any one of SEQ ID NOs: 187, 203, and 236; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 187, 203, and 236, the CDR-H2 has at least about 80%, 90%, 91%, 92%, 93%, 94%, 95%, 96%, 97%, 98% or 99% identity with a CDR-H2 selected from SEQ ID NOs: 79, 119, and 150, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 343 and 365, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 187, 203, and 236, with up to 1, 2, 3, 4, 5, 6, 7, or 8 amino acid substitutions; the CDR-H2 is a CDR-H2 selected from SEQ ID NOs: 79, 119, and 150, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 343 and 365, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 187, 203, and 236; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 188, 203, and 237, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 368 and 394, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 268, 288, and 313. In some embodiments, the CDR-H3 is a CDR-H3 selected from SEQ ID NOs: 188, 203, and 237, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 368 and 394, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 268, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 268, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity with a CDR-H3 selected from SEQ ID NOs: 188, 203, and 237, the CDR-H2 has at least about 80%, 90%, 91%, 92%, 93%, 94%, 95%, 96%, 97%, 98% or 99% identity with a CDR-H2 selected from SEQ ID NOs: 70, 111, 98% or 99% identity with a CDR-H1 selected from SEQ ID NOs: 12, 29, and 54, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 343 and 365, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 188, 203, and 237, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 12, 29, and 54, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 343 and 365, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 12, 29, and 54; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 189, 214, and 238, the CDR-H2 has at least about 80%, 90%, 91%, 92%, 93%, 94%, 95%, 96%, 97%, 98% or 99% identity with a CDR-H2 selected from SEQ ID NOs: 82, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 345 and 361, and the CDR-L1 has at least about 80%, 90%, 91%, 92%, 93%, 94%, 95%, 96%, 97%, 98% or 99% identity with a CDR-L1 selected from SEQ ID NOs: 269, 288, and 313. In some embodiments, the CDR-H3 is a CDR-H3 selected from SEQ ID NOs: 189, 214, and 238, with up to 1, 2, 3, 4, 5, 6, 7, or 8 amino acid substitutions; the CDR-H2 is a CDR-H2 selected from SEQ ID NOs: 82, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 345 and 361, with up to 1, 2, 3, or 4 amino acid substitutions; and the CDR-L1 is a CDR-L1 selected from SEQ ID NOs: 269, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 82, 111, and 142; a CDR-H3 comprising an amino acid sequence set forth in any one of SEQ ID NOs: 189, 214, and 238; a CDR-L1 comprising an amino acid sequence set forth in any one of SEQ ID NOs: 269, 288, and 313; a CDR-L2 comprising an amino acid sequence set forth in any one of SEQ ID NOs: 345 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 234, the CDR-H2 has at least about 80%, 90%, 91%, 92%, 93%, 94%, 95%, 96%, 97%, 98% or 99% identity with a CDR-H2 selected from SEQ ID NOs: 83, 122, and 153, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 346 or 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 234, with up to 1, 2, 3, 4, 5, 6, 7, or 8 amino acid substitutions; the CDR-H2 is a CDR-H2 selected from SEQ ID NOs: 83, 122, and 153, with SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 346 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 83, 122, and 153; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 346 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 188, 203, and 239, the CDR-H2 has at least about 80%, 90%, 91%, 92%, 93%, 94%, 95%, 96%, 97%, 98% or 99% identity with a CDR-H2 selected from SEQ ID NOs: 84, 111, and 142, the CDR-H1 has at least about 80%, 90%, 91%, 92%, 93%, 94%, 95%, 96%, 97%, 98% or 99% identity with a CDR-H1 selected from SEQ ID NOs: 13, 30, and 55, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 337 or 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 188, 203, and 239, with up to 1, 2, 3, 4, 5, 6, 7, or 8 amino acid substitutions; the CDR-H2 is a CDR-H2 selected from SEQ ID NOs: 84, 111, and 142, with up to 1, 2, 3, 4, 5, 6, 7, or 8 amino acid substitutions; the CDR-H1 is a CDR-H1 selected from SEQ ID NO: 13, 30, and 55, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 337 and 361, with up to NOs: 260, 288, and 313.

In some embodiments, an antibody provided herein comprises a CDR-H1 comprising an amino acid sequence set forth in any one of SEQ ID NOs: 13, 30, and 55; a CDR-H2 comprising an amino acid sequence set forth in any one of SEQ ID NOs: 84, 111, and 142; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 234, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 347 and 365, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 234, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 347 and 365, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 347 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 234, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, CDR-L2 has at least about 80%, 90%, 91%, 92%, 93%, 94%, 95%, 96%, 97%, 98% or 99% identity with a CDR-L2 selected from SEQ ID NOs: 345 or 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 234, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 345 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 345 and 361; and a CDR-L3 comprising an amino acid sequence set forth in any one of SEQ ID NOS: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 188, 203, and 239, the CDR-H2 has at least about 80%, 90%, 91%, 92%, 93%, 94%, 95%, 96%, 97%, 98% or 99% identity with a CDR-H2 selected from SEQ ID NOs: 85, 123, and 154, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 337 or 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 188, 203, and 239, with up to 1, 2, 3, 4, 5, 6, 7, or 8 amino acid substitutions; the CDR-H2 is a CDR-H2 selected from SEQ ID NOs: 85, 123, and 154, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 85, 123, and 154; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, and CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 270, 295, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 270, 295, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 270, 295, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, or 223, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 1, 31, and 56, the CDR-L3 has at least about 80%, 90%, 91%, 92%, 93%, 94%, 95%, 96%, 97%, 98% or 99% identity with a CDR-L3 selected from SEQ ID NOs: 379 and 405, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 1, 31, and 56, with up to 1, 2, 3, 4, or 5 amino acid substitutions; the CDR-L3 is a CDR-L3 selected from SEQ ID NOs: 379 and 405, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 1, 31, and 56; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 379 and 405.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 86, 124, and 155, the CDR-H1 has at least about 80%, 90%, 91%, 92%, 93%, 94%, 95%, 96%, 97%, 98% or 99% identity with a CDR-H1 selected from SEQ ID NOs: 1, 32, and 57, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 86, 124, and 155, with up to 1, 2, 3, 4, 5, 6, 7, or 8 amino acid substitutions; the CDR-H1 is a CDR-H1 selected from SEQ ID NO: 1, 32, and 57, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 1, 32, and 57; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 380 and 406, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 380 and 406, with up to 1, 2, 3, 4, or 5 amino acid substitutions; the CDR-L2 is a CDR-L2 selected from SEQ ID NOs: 337 and 361, with up to NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 380 and 406.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 14, 33, and 58, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 14, 33, and 58, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 14, 33, and 58; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 87, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 343 and 365, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 87, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 343 and 365, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 87, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 87, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 87, 111, 98% or 99% identity with a CDR-H1 selected from SEQ ID NOs: 13, 30, and 55, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 87, 111, and 142, with up to 1, 2, 3, 4, 5, 6, 7, or 8 amino acid substitutions; the CDR-H1 is a CDR-H1 selected from SEQ ID NO: 13, 30, and 55, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 13, 30, and 55; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 368 and 394, the CDR-L2 has at least about 80%, 90%, 91%, 92%, 93%, 94%, 95%, 96%, 97%, 98% or 99% identity with a CDR-91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 368 and 394, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 87, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 348 and 366, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 87, 111, and 142, with SEQ ID NO: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 348 and 366, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 348 and 366; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 381 and 405, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NOs: 10, 27, and 52, with up to 1, 2, 3, 4, or 5 amino acid substitutions; the CDR-L3 is a CDR-L3 selected from SEQ ID NOs: 381 and 405, with up to 1, 2, 3, 4, or 5 amino acid substitutions; the CDR-L2 is a CDR-L2 selected from SEQ ID NOs: 337 and 361, with up to NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 381 and 405.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 368 and 394, the CDR-L2 has at least about 80%, 90%, 91%, 92%, 93%, 94%, 95%, 96%, 97%, 98% or 99% identity with a CDR-L2 selected from SEQ ID NOs: 335 and 359, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from SEQ ID NOs: 368 and 394, with up to 1, 2, 3, 4, or 5 amino acid substitutions; the CDR-L2 is a CDR-L2 selected from SEQ ID NOs: 335 and 359, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 335 and 359; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 86, 124, and 155, the CDR-H1 has at least about 80%, 90%, 91%, 92%, 93%, 94%, 95%, 96%, 97%, 98% or 99% identity with a CDR-H1 selected from SEQ ID NOs: 1, 22, and 44, the CDR-L3 has at least about 80%, 90%, 91%, 92%, 93%, 94%, 95%, 96%, 97%, 98% or 99% identity with a CDR-L3 selected from SEQ ID NOs: 381 and 405, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 86, 124, and 155, with up to 1, 2, 3, 4, 5, 6, 7, or 8 amino acid substitutions; the CDR-H1 is a CDR-H1 selected from SEQ ID NO: 1, 22, and 44, with up to 1, 2, 3, 4, or 5 amino acid substitutions; the CDR-L3 is a CDR-L3 selected from SEQ ID NOs: 381 and 405, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 86, 124, and 155; a CDR-H3 comprising an amino acid sequence set forth in any one of SEQ ID NOS: 175, 203, and 223; a CDR-L1 comprising an amino acid sequence set forth in any one of SEQ ID NOs: 260, 288, or 313; a CDR-L2 comprising an amino acid sequence set forth in any one of SEQ ID NOs: 337 and 361; and a CDR-L3 comprising an amino acid sequence set forth in any one of SEQ ID NOs: 381 and 405.

93%, 94%, 95%, 96%, 97%, 98% or 99% identity with a CDR-H3 selected from SEQ ID NOs: 175, 203, and 223, the CDR-H2 has at least about 80%, 90%, 91%, 92%, 93%, 94%, 95%, 96%, 97%, 98% or 99% identity with a CDR-H2 selected from SEQ ID NOs: 86, 124, and 155, the CDR-H1 has at least about 80%, 90%, 91%, 92%, 93%, 94%, 95%, 96%, 97%, 98% or 99% identity with a CDR-H1 selected from SEQ ID NOs: 1, 22, and 44, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 175, 203, and 223, with up to 1, 2, 3, 4, 5, 6, 7, or 8 amino acid substitutions; the CDR-H2 is a CDR-H2 selected from SEQ ID NOs: 86, 124, and 155, with up to 1, 2, 3, 4, 5, 6, 7, or 8 amino acid substitutions; the CDR-H1 is a CDR-H1 selected from SEQ ID NO: 1, 22, and 44, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 188, 203, and 239, the CDR-H2 has at least about 80%, 90%, 91%, 92%, 93%, 94%, 95%, 96%, 97%, 98% or 99% identity with a CDR-H2 selected from SEQ ID NO: 70, 111, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NO: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 188, 203, and 239, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NOs: 10, 27, and 52, with up to 1, 2, 3, 4, or 5 amino acid substitutions; the CDR-L3 is a CDR-L3 selected from SEQ ID NO: 378 and 404, with up to 1, 2, 3, 4, or 5 amino acid substitutions; the CDR-L2 is a CDR-L2 selected from SEQ ID NO: 337 and 361, with up to 1, 2, 3, or 4 amino acid substitutions; and the CDR-L1 is a CDR-L1 selected from SEQ ID NO: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 188, 203, and 237, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 378 and 404, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 188, 203, and 237, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NOs: 10, 27, and 52, with up to 1, 2, 3, 4, or 5 amino acid substitutions; the CDR-L3 is a CDR-L3 selected from SEQ ID NOs: 378 and 404, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity with a CDR-H3 selected from SEQ ID NOs: 188, 203, and 237, the CDR-H2 has at least about 80%, 90%, 91%, 92%, 93%, 94%, 95%, 96%, 97%, 98% or 99% identity with a CDR-H2 selected from SEQ ID NOs: 78, 119, and 150, the CDR-H1 has at least about 80%, 90%, 91%, 92%, 93%, 94%, 95%, 96%, 97%, 98% or 99% identity with a CDR-H1 selected from SEQ ID NOs: 10, 27, and 52, the CDR-L3 has at least about 80%, 90%, 91%, 92%, 93%, 94%, 95%, 96%, 97%, 98% or 99% identity with a CDR-L3 selected from SEQ ID NOs: 368 and 394, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 188, 203, and 237, with up to 1, 2, 3, 4, 5, 6, 7, or 8 amino acid substitutions; the CDR-H2 is a CDR-H2 selected from SEQ ID NOs: 78, 119, and 150, with up to 1, 2, 3, 4, 5, 6, 7, or 8, amino acid substitutions; the CDR-H1 is a CDR-H1 selected from SEQ ID NOs: 10, 27, and 52, with up to 1, 2, 3, 4, or 5 amino acid substitutions; the CDR-L3 is a CDR-L3 selected from SEQ ID NOs: 368 and 394, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 78, 119, and 150; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity with a CDR-H3 selected from SEQ ID NOs: 176, 204, and 224, the CDR-H2 has at least about 80%, 90%, 91%, 92%, 93%, 94%, 95%, 96%, 97%, 98% or 99% identity with a CDR-H2 selected from SEQ ID NOs: 70, 111, and 142, the CDR-H1 has at least about 80%, 90%, 91%, 92%, 93%, 94%, 95%, 96%, 97%, 98% or 99% identity with a CDR-H1 selected from SEQ ID NOs: 2, 23, and 45, the CDR-L3 has at least about 80%, 90%, 91%, 92%, 93%, 94%, 95%, 96%, 97%, 98% or 99% identity with a CDR-L3 selected from SEQ ID NOs: 369 and 395, the CDR-L2 has at least about 80%, 90%, 91%, 92%, 93%, 94%, 95%, 96%, 97%, 98% or 99% identity with a CDR-L2 selected from SEQ ID NOs: 336 and 360, and the CDR-L1 has at least about 80%, 90%, 91%, 92%, 93%, 94%, 95%, 96%, 97%, 98% or 99% identity with a CDR-L1 selected from SEQ ID NOs: 259, 287, and 312. In some embodiments, the CDR-H3 is a CDR-H3 selected from SEQ ID NOs: 176, 204, and 224, with up to 1, 2, 3, 4, 5, 6, 7, or 8 amino acid substitutions; the CDR-H2 is a CDR-H2 selected from SEQ ID NOs: 70, 111, and 142, with up to 1, 2, 3, 4, 5, 6, 7, or 8 amino acid substitutions; the CDR-H1 is a CDR-H1 selected from SEQ ID NO: 2, 23, and 45, with up to 1, 2, 3, 4, or 5 amino acid substitutions; the CDR-L3 is a CDR-L3 selected from SEQ ID NOs: 369 and 395, with up to 1, 2, 3, 4, or 5 amino acid substitutions; the CDR-L2 is a CDR-L2 selected from SEQ ID NOs: 336 and 360, with up to NOs: 259, 287, and 312.

In some embodiments, an antibody provided herein comprises a CDR-H1 comprising an amino acid sequence set forth in any one of SEQ ID NOs: 2, 23, and 45; a CDR-H2 comprising an amino acid sequence set forth in any one of SEQ ID NOs: 70, 111, and 142; a CDR-H3 comprising an amino acid sequence set forth in any one of SEQ ID NOs: 176, 204, and 224; a CDR-L1 comprising an amino acid sequence set forth in any one of SEQ ID NOs: 259, 287, and 312; a CDR-L2 comprising an amino acid sequence set forth in any one of SEQ ID NOs: 336 and 360; and a CDR-L3 comprising an amino acid sequence set forth in any one of SEQ ID NOs: 369 and 395.

In some embodiments, the CDR-H3 has at least about 80%, 90%, 91%, 92%, 93%, 94%, 95%, 96%, 97%, 98% or 99% identity with a CDR-H3 selected from SEQ ID NOs: 190, 215, and 240, the CDR-H2 has at least about 80%, 90%, 91%, 92%, 93%, 94%, 95%, 96%, 97%, 98% or 99% identity with a CDR-H2 selected from SEQ ID NOs: 88, 125, and 156, the CDR-H1 has at least about 80%, 90%, 91%, 92%, 93%, 94%, 95%, 96%, 97%, 98% or 99% identity with a CDR-H1 selected from SEQ ID NOs: 15, 34, and 59, the CDR-L3 has at least about 80%, 90%, 91%, 92%, 93%, 94%, 95%, 96%, 97%, 98% or 99% identity with a CDR-L3 selected from SEQ ID NOs: 382 and 407, the CDR-L2 has at least about 80%, 90%, 91%, 92%, 93%, 94%, 95%, 96%, 97%, 98% or 99% identity with a CDR-L2 selected from SEQ ID NOs: 349 and 365, and the CDR-L1 has at least about 80%, 90%, 91%, 92%, 93%, 94%, 95%, 96%, 97%, 98% or 99% identity with a CDR-L1 selected from SEQ ID NOs: 269, 288, and 313. In some embodiments, the CDR-H3 is a CDR-H3 selected from SEQ ID NOs: 190, 215, and 240, with up to 1, 2, 3, 4, 5, 6, 7, or 8 amino acid substitutions; the CDR-H2 is a CDR-H2 selected from SEQ ID NOs: 88, 125, and 156, with up to 1, 2, 3, 4, 5, 6, 7, or 8 amino acid substitutions; the CDR-H1 is a CDR-H1 selected from SEQ ID NOs: 15, 34, and 59, with up to 1, 2, 3, 4, or 5 amino acid substitutions; the CDR-L3 is a CDR-L3 selected from SEQ ID NOs: 382 and 407, with up to 1, 2, 3, 4, or 5 amino acid substitutions; the CDR-L2 is a CDR-L2 selected from SEQ ID NOs: 349 and 365, with up to 1, 2, 3, or 4 amino acid substitutions; and the CDR-L1 is a CDR-L1 selected from SEQ ID NOs: 269, 288, and 313.

In some embodiments, an antibody provided herein comprises a CDR-H1 comprising an amino acid sequence set forth in any one of SEQ ID NOs: 15, 34, and 59; a CDR-H2 comprising an amino acid sequence set forth in any one of SEQ ID NOs: 88, 125, and 156; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 349 and 365; and a CDR-L3 comprising an amino acid sequence set forth in any one of SEQ ID NOs: 382 and 407.

In some embodiments, the CDR-H3 has at least about 80%, 90%, 91%, 92%, 93%, 94%, 95%, 96%, 97%, 98% or 99% identity with a CDR-H3 selected from SEQ ID NOs: 178, 206, and 226, the CDR-H2 has at least about 80%, 90%, 91%, 92%, 93%, 94%, 95%, 96%, 97%, 98% or 99% identity with a CDR-H2 selected from SEQ ID NOs: 89, 126, and 157, the CDR-H1 has at least about 80%, 90%, 91%, 92%, 93%, 94%, 95%, 96%, 97%, 98% or 99% identity with a CDR-H1 selected from SEQ ID NOs: 4, 22, and 47, the CDR-L3 has at least about 80%, 90%, 91%, 92%, 93%, 94%, 95%, 96%, 97%, 98% or 99% identity with a CDR-L3 selected from SEQ ID NOs: 371 and 397, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 260, 288, and 313. In some embodiments, the CDR-H3 is a CDR-H3 selected from SEQ ID NOs: 178, 206, and 226, with up to 1, 2, 3, 4, 5, 6, 7, or 8 amino acid substitutions; the CDR-H2 is a CDR-H2 selected from SEQ ID NOs: 89, 126, and 157, with up to 1, 2, 3, 4, 5, 6, 7, or 8 amino acid substitutions; the CDR-H1 is a CDR-H1 selected from SEQ ID NO: 4, 22, and 47, with up to 1, 2, 3, 4, or 5 amino acid substitutions; the CDR-L3 is a CDR-L3 selected from SEQ ID NOs: 371 and 397, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 260, 288, and 313.

In some embodiments, an antibody provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 89, 126, and 157; a CDR-H3 comprising an amino acid sequence set forth in any one of SEQ ID NOS: 178, 206, and 226; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, the CDR-H3 has at least about 80%, 90%, 91%, 92%, 93%, 94%, 95%, 96%, 97%, 98% or 99% identity with a CDR-H3 selected from SEQ ID NOs: 178, 206, and 241, the CDR-H2 has at least about 80%, 90%, 91%, 92%, 93%, 94%, 95%, 96%, 97%, 98% or 99% identity with a CDR-H2 selected from SEQ ID NOs: 72, 113, and 144, the CDR-H1 has at least about 80%, 90%, 91%, 92%, 93%, 94%, 95%, 96%, 97%, 98% or 99% identity with a CDR-H1 selected from SEQ ID NOs: 4, 35, and 60, the CDR-L3 has at least about 80%, 90%, 91%, 92%, 93%, 94%, 95%, 96%, 97%, 98% or 99% identity with a CDR-L3 selected from SEQ ID NOs: 371 and 397, the CDR-L2 has at least about 80%, 90%, 91%, 92%, 93%, 94%, 95%, 96%, 97%, 98% or 99% identity with a CDR-L2 selected from SEQ ID NOs: 350 and 361, and the CDR-L1 has at least about 80%, 90%, 91%, 92%, 93%, 94%, 95%, 96%, 97%, 98% or 99% identity with a CDR-L1 selected from SEQ ID NOs: 271, 296, and 320. In some embodiments, the CDR-H3 is a CDR-H3 selected from SEQ ID NOs: 178, 206, and 241, with up to 1, 2, 3, 4, 5, 6, 7, or 8 amino acid substitutions; the CDR-H2 is a CDR-H2 selected from SEQ ID NOs: 72, 113, and 144, with up to 1, 2, 3, 4, 5, 6, 7, or 8 amino acid substitutions; the CDR-H1 is a CDR-H1 selected from SEQ ID NOs: 4, 35, and 60, with up to 1, 2, 3, 4, or 5 amino acid substitutions; the CDR-L3 is a CDR-L3 selected from SEQ ID NOs: 371 and 397, with up to 1, 2, 3, 4, or 5 amino acid substitutions; the CDR-L2 is a CDR-L2 selected from SEQ ID NOs: 350 and 361, with up to 1, 2, 3, or 4 amino acid substitutions; and the CDR-L1 is a CDR-L1 selected from SEQ ID NOs: 271, 296, and 320.

In some embodiments, an antibody provided herein comprises a CDR-H1 comprising an amino acid sequence set forth in any one of SEQ ID NOs: 4, 35, and 60; a CDR-H2 comprising an amino acid sequence set forth in any one of SEQ ID NOs: 72, 113, and 144; a CDR-H3 comprising an amino acid sequence set forth in any one of SEQ ID NOs: 178, 206, and 241; a CDR-L1 comprising an amino acid sequence set forth in any one of SEQ ID NOs: 271, 296, and 320; a CDR-L2 comprising an amino acid sequence set forth in any one of SEQ ID NOs: 350 and 361; and a CDR-L3 comprising an amino acid sequence set forth in any one of SEQ ID NOs: 371 and 397.

In some embodiments, the CDR-H3 has at least about 80%, 90%, 91%, 92%, 93%, 94%, 95%, 96%, 97%, 98% or 99% identity with a CDR-H3 selected from SEQ ID NOs: 178, 206, and 226, the CDR-H2 has at least about 80%, 90%, 91%, 92%, 93%, 94%, 95%, 96%, 97%, 98% or 99% identity with a CDR-H2 selected from SEQ ID NOs: 90, 127, and 158, the CDR-H1 has at least about 80%, 90%, 91%, 92%, 93%, 94%, 95%, 96%, 97%, 98% or 99% identity with a CDR-H1 selected from SEQ ID NOs: 4, 22, and 47, the CDR-L3 has at least about 80%, 90%, 91%, 92%, 93%, 94%, 95%, 96%, 97%, 98% or 99% identity with a CDR-L3 selected from SEQ ID NOs: 371 or 397, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 261, 289, and 314. In some embodiments, the CDR-H3 is a CDR-H3 selected from SEQ ID NOs: 178, 206, and 226, with up to 1, 2, 3, 4, 5, 6, 7, or 8 amino acid substitutions; the CDR-H2 is a CDR-H2 selected from SEQ ID NOs: 90, 127, and 158, with up to 1, 2, 3, 4, 5, 6, 7, or 8 amino acid substitutions; the CDR-H1 is a CDR-H1 selected from SEQ ID NO: 4, 22, and 47, with up to 1, 2, 3, 4, or 5 amino acid substitutions; the CDR-L3 is a CDR-L3 selected from SEQ ID NOs: 371 and 397, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 261, 289, and 314.

In some embodiments, an antibody provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 90, 127, and 158; a CDR-H3 comprising an amino acid sequence set forth in any one of SEQ ID NOs: 178, 206, and 226; a CDR-L1 comprising an amino acid sequence set forth in any one of SEQ ID NOs: 261, 289, and 314; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, the CDR-H3 has at least about 80%, 90%, 91%, 92%, 93%, 94%, 95%, 96%, 97%, 98% or 99% identity with a CDR-H3 selected from SEQ ID NOs: 191, 215, and 242, the CDR-H2 has at least about 80%, 90%, 91%, 92%, 93%, 94%, 95%, 96%, 97%, 98% or 99% identity with a CDR-H2 selected from SEQ ID NOs: 91, 128, and 159, the CDR-H1 has at least about 80%, 90%, 91%, 92%, 93%, 94%, 95%, 96%, 97%, 98% or 99% identity with a CDR-H1 selected from SEQ ID NOs: 16, 36, and 61, the CDR-L3 has at least about 80%, 90%, 91%, 92%, 93%, 94%, 95%, 96%, 97%, 98% or 99% identity with a CDR-L3 selected from SEQ ID NOs: 382 and 407, the CDR-L2 has at least about 80%, 90%, 91%, 92%, 93%, 94%, 95%, 96%, 97%, 98% or 99% identity with a CDR-L2 selected from SEQ ID NOs: 351 or 361, and the CDR-L1 has at least about 80%, 90%, 91%, 92%, 93%, 94%, 95%, 96%, 97%, 98% or 99% identity with a CDR-L1 selected from SEQ ID NOs: 269, 288, and 313. In some embodiments, the CDR-H3 is a CDR-H3 selected from SEQ ID NOs: 191, 215, and 242, with up to 1, 2, 3, 4, 5, 6, 7, or 8 amino acid substitutions; the CDR-H2 is a CDR-H2 selected from SEQ ID NOs: 91, 128, and 159, with up to 1, 2, 3, 4, 5, 6, 7, or 8 amino acid substitutions; the CDR-H1 is a CDR-H1 selected from SEQ ID NOs: 16, 36, and 61, with up to 1, 2, 3, 4, or 5 amino acid substitutions; the CDR-L3 is a CDR-L3 selected from SEQ ID NOs: 382 and 407, with up to 1, 2, 3, 4, or 5 amino acid substitutions; the CDR-L2 is a CDR-L2 selected from SEQ ID NOs: 351 and 361, with up to 1, 2, 3, or 4 amino acid substitutions; and the CDR-L1 is a CDR-L1 selected from SEQ ID NOs: 269, 288, and 313.

In some embodiments, an antibody provided herein comprises a CDR-H1 comprising an amino acid sequence set forth in any one of SEQ ID NOs: 16, 36, and 61; a CDR-H2 comprising an amino acid sequence set forth in any one of SEQ ID NOs: 91, 128, and 159; a CDR-H3 comprising an amino acid sequence set forth in any one of SEQ ID NOs: 191, 215, and 242; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 382 and 407.

In some embodiments, the CDR-H3 has at least about 80%, 90%, 91%, 92%, 93%, 94%, 95%, 96%, 97%, 98% or 99% identity with a CDR-H3 selected from SEQ ID NOs: 190, 215, and 240, the CDR-H2 has at least about 80%, 90%, 91%, 92%, 93%, 94%, 95%, 96%, 97%, 98% or 99% identity with a CDR-H2 selected from SEQ ID NOs: 91, 128, and 159, the CDR-H1 has at least about 80%, 90%, 91%, 92%, 93%, 94%, 95%, 96%, 97%, 98% or 99% identity with a CDR-H1 selected from SEQ ID NOs: 16, 36, and 61, the CDR-L3 has at least about 80%, 90%, 91%, 92%, 93%, 94%, 95%, 96%, 97%, 98% or 99% identity with a CDR-L3 selected from SEQ ID NOs: 371 and 397, the CDR-L2 has at least about 80%, 90%, 91%, 92%, 93%, 94%, 95%, 96%, 97%, 98% or 99% identity with a CDR-L2 selected from SEQ ID NOs: 351 and 361, and the CDR-L1 has at least about 80%, 90%, 91%, 92%, 93%, 94%, 95%, 96%, 97%, 98% or 99% identity with a CDR-L1 selected from SEQ ID NOs: 269, 288, and 313. In some embodiments, the CDR-H3 is a CDR-H3 selected from SEQ ID NOs: 190, 215, and 240, with up to 1, 2, 3, 4, 5, 6, 7, or 8 amino acid substitutions; the CDR-H2 is a CDR-H2 selected from SEQ ID NOs: 91, 128, and 159, with up to 1, 2, 3, 4, 5, 6, 7, or 8, amino acid substitutions; the CDR-H1 is a CDR-H1 selected from SEQ ID NOs: 16, 36, and 61, with up to 1, 2, 3, 4, or 5 amino acid substitutions; the CDR-L3 is a CDR-L3 selected from SEQ ID NOs: 371 and 397, with up to 1, 2, 3, 4, or 5 amino acid substitutions; the CDR-L2 is a CDR-L2 selected from SEQ ID NOs: 351 and 361, with up to 1, 2, 3, or 4 amino acid substitutions; and the CDR-L1 is a CDR-L1 selected from SEQ ID NOs: 269, 288, and 313.

In some embodiments, an antibody provided herein comprises a CDR-H1 comprising an amino acid sequence set forth in any one of SEQ ID NOs: 16, 36, and 61; a CDR-H2 comprising an amino acid sequence set forth in any one of SEQ ID NOs: 91, 128, and 159; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 371 and 397.

In some embodiments, the CDR-H3 has at least about 80%, 90%, 91%, 92%, 93%, 94%, 95%, 96%, 97%, 98% or 99% identity with a CDR-H3 selected from SEQ ID NOs: 190, 215, and 240, the CDR-H2 has at least about 80%, 90%, 91%, 92%, 93%, 94%, 95%, 96%, 97%, 98% or 99% identity with a CDR-H2 selected from SEQ ID NOs: 72, 113, and 144, the CDR-H1 has at least about 80%, 90%, 91%, 92%, 93%, 94%, 95%, 96%, 97%, 98% or 99% identity with a CDR-H1 selected from SEQ ID NOs: 15, 27, and 62, the CDR-L3 has at least about 80%, 90%, 91%, 92%, 93%, 94%, 95%, 96%, 97%, 98% or 99% identity with a CDR-L3 selected from SEQ ID NOs: 382 and 407, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 269, 288, and 313. In some embodiments, the CDR-H3 is a CDR-H3 selected from SEQ ID NOs: 190, 215, and 240, with up to 1, 2, 3, 4, 5, 6, 7, or 8 amino acid substitutions; the CDR-H2 is a CDR-H2 selected from SEQ ID NOs: 72, 113, and 144, with up to 1, 2, 3, 4, 5, 6, 7, or 8 amino acid substitutions; the CDR-H1 is a CDR-H1 selected from SEQ ID NOs: 15, 27, and 62, with up to 1, 2, 3, 4, or 5 amino acid substitutions; the CDR-L3 is a CDR-L3 selected from SEQ ID NOs: 382 and 407, with up to 1, 2, 3, 4, or 5 amino acid substitutions; the CDR-L2 is a CDR-L2 selected from SEQ ID NOs: 337 and 361, with up to NOs: 269, 288, and 313.

In some embodiments, an antibody provided herein comprises a CDR-H1 comprising an amino acid sequence set forth in any one of SEQ ID NOs: 15, 27, and 62; a CDR-H2 comprising an amino acid sequence set forth in any one of SEQ ID NOs: 72, 113, and 144; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 337 and 361; and a CDR-L3 comprising an amino acid sequence set forth in any one of SEQ ID NOs: 382 and 407.

In some embodiments, the CDR-H3 has at least about 80%, 90%, 91%, 92%, 93%, 94%, 95%, 96%, 97%, 98% or 99% identity with a CDR-H3 selected from SEQ ID NO: 190, 215, and 240, the CDR-H2 has at least about 80%, 90%, 91%, 92%, 93%, 94%, 95%, 96%, 97%, 98% or 99% identity with a CDR-H2 selected from SEQ ID NO: 72, 113, and 144, the CDR-H1 has at least about 80%, 90%, 91%, 92%, 93%, 94%, 95%, 96%, 97%, 98% or 99% identity with a CDR-H1 selected from SEQ ID NOs: 15, 27, and 62, the CDR-L3 has at least about 80%, 90%, 91%, 92%, 93%, 94%, 95%, 96%, 97%, 98% or 99% identity with a CDR-L3 selected from SEQ ID NOs: 383 and 408, the CDR-L2 has at least about 80%, 90%, 91%, 92%, 93%, 94%, 95%, 96%, 97%, 98% or 99% identity with a CDR-L2 selected from SEQ ID NOs: 351 and 361, and the CDR-L1 has at least about 80%, 90%, 91%, 92%, 93%, 94%, 95%, 96%, 97%, 98% or 99% identity with a CDR-L1 selected from SEQ ID NOs: 269, 288, and 313. In some embodiments, the CDR-H3 is a CDR-H3 selected from SEQ ID NOs: 190, 215, and 240, with up to 1, 2, 3, 4, 5, 6, 7, or 8 amino acid substitutions; the CDR-H2 is a CDR-H2 selected from SEQ ID NO: 72, 113, and 144, with up to 1, 2, 3, 4, 5, 6, 7, or 8 amino acid substitutions; the CDR-H1 is a CDR-H1 selected from SEQ ID NOs: 15, 27, and 62, with up to 1, 2, 3, 4, or 5 amino acid substitutions; the CDR-L3 is a CDR-L3 selected from SEQ ID NOs: 383 and 408, with up to 1, 2, 3, 4, or 5 amino acid substitutions; the CDR-L2 is a CDR-L2 selected from SEQ ID NOs: 351 and 361, with up to 1, 2, 3, or 4 amino acid substitutions; and the CDR-L1 is a CDR-L1 selected from SEQ ID NOs: 269, 288, and 313.

In some embodiments, an antibody provided herein comprises a CDR-H1 comprising an amino acid sequence set forth in any one of SEQ ID NOs: 15, 27, and 62; a CDR-H2 comprising an amino acid sequence set forth in any one of SEQ ID NOs: 72, 113, and 144; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 383 and 408.

In some embodiments, the CDR-H3 has at least about 80%, 90%, 91%, 92%, 93%, 94%, 95%, 96%, 97%, 98% or 99% identity with a CDR-H3 selected from SEQ ID NOs: 179, 207, and 227, the CDR-H2 has at least about 80%, 90%, 91%, 92%, 93%, 94%, 95%, 96%, 97%, 98% or 99% identity with a CDR-H2 selected from SEQ ID NOs: 92, 129, and 160, the CDR-H1 has at least about 80%, 90%, 91%, 92%, 93%, 94%, 95%, 96%, 97%, 98% or 99% identity with a CDR-H1 selected from SEQ ID NOs: 5, 37, and 63, the CDR-L3 has at least about 80%, 90%, 91%, 92%, 93%, 94%, 95%, 96%, 97%, 98% or 99% identity with a CDR-L3 selected from SEQ ID NOs: 384 and 409, the CDR-L2 has at least about 80%, 90%, 91%, 92%, 93%, 94%, 95%, 96%, 97%, 98% or 99% identity with a CDR-L2 selected from SEQ ID NOs: 338 and 362, and the CDR-L1 has at least about 80%, 90%, 91%, 92%, 93%, 94%, 95%, 96%, 97%, 98% or 99% identity with a CDR-L1 selected from SEQ ID NOs: 272, 297, and 321. In some embodiments, the CDR-H3 is a CDR-H3 selected from SEQ ID NOs: 179, 207, and 227, with up to 1, 2, 3, 4, 5, 6, 7, or 8 amino acid substitutions; the CDR-H2 is a CDR-H2 selected from SEQ ID NOs: 92, 129, and 160, with up to 1, 2, 3, 4, 5, 6, 7, or 8 amino acid substitutions; the CDR-H1 is a CDR-H1 selected from SEQ ID NO: 5, 37, and 63, with up to 1, 2, 3, 4, or 5 amino acid substitutions; the CDR-L3 is a CDR-L3 selected from SEQ ID NOs: 384 and 409, with up to 1, 2, 3, 4, or 5 amino acid substitutions; the CDR-L2 is a CDR-L2 selected from SEQ ID NOs: 338 and 362, with up to 1, 2, 3, or 4 amino acid substitutions; and the CDR-L1 is a CDR-L1 selected from SEQ ID NOs: 272, 297, and 321.

In some embodiments, an antibody provided herein comprises a CDR-H1 comprising an amino acid sequence set forth in any one of SEQ ID NOs: 5, 37, and 63; a CDR-H2 comprising an amino acid sequence set forth in any one of SEQ ID NOs: 92, 129, and 160; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 384 and 409.

In some embodiments, the CDR-H3 has at least about 80%, 90%, 91%, 92%, 93%, 94%, 95%, 96%, 97%, 98% or 99% identity with a CDR-H3 selected from SEQ ID NOs: 179, 207, and 227, the CDR-H2 has at least about 80%, 90%, 91%, 92%, 93%, 94%, 95%, 96%, 97%, 98% or 99% identity with a CDR-H2 selected from SEQ ID NOs: 93, 130, and 161, the CDR-H1 has at least about 80%, 90%, 91%, 92%, 93%, 94%, 95%, 96%, 97%, 98% or 99% identity with a CDR-H1 selected from SEQ ID NOs: 5, 24, and 49, the CDR-L3 has at least about 80%, 90%, 91%, 92%, 93%, 94%, 95%, 96%, 97%, 98% or 99% identity with a CDR-L3 selected from SEQ ID NOs: 384 and 409, the CDR-L2 has at least about 80%, 90%, 91%, 92%, 93%, 94%, 95%, 96%, 97%, 98% or 99% identity with a CDR-L2 selected from SEQ ID NOs: 338 and 362, and the CDR-L1 has at least about 80%, 90%, 91%, 92%, 93%, 94%, 95%, 96%, 97%, 98% or 99% identity with a CDR-L1 selected from SEQ ID NOs: 272, 297, and 321. In some embodiments, the CDR-H3 is a CDR-H3 selected from SEQ ID NOs: 179, 207, and 227, with up to 1, 2, 3, 4, 5, 6, 7, or 8 amino acid substitutions; the CDR-H2 is a CDR-H2 selected from SEQ ID NOs: 93, 130, and 161, with up to 1, 2, 3, 4, 5, 6, 7, or 8 amino acid substitutions; the CDR-H1 is a CDR-H1 selected from SEQ ID NO: 5, 24, and 49, with up to 1, 2, 3, 4, or 5 amino acid substitutions; the CDR-L3 is a CDR-L3 selected from SEQ ID NOs: 384 and 409, with up to 1, 2, 3, 4, or 5 amino acid substitutions; the CDR-L2 is a CDR-L2 selected from SEQ ID NOs: 338 and 362, with up to 1, 2, 3, or 4 amino acid substitutions; and the CDR-L1 is a CDR-L1 selected from SEQ ID NOs: 272, 297, and 321.

In some embodiments, an antibody provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 93, 130, and 161; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 384 and 409.

In some embodiments, the CDR-H3 has at least about 80%, 90%, 91%, 92%, 93%, 94%, 95%, 96%, 97%, 98% or 99% identity with a CDR-H3 selected from SEQ ID NOs: 192, 207, and 243, the CDR-H2 has at least about 80%, 90%, 91%, 92%, 93%, 94%, 95%, 96%, 97%, 98% or 99% identity with a CDR-H2 selected from SEQ ID NOs: 94, 131, and 162, the CDR-H1 has at least about 80%, 90%, 91%, 92%, 93%, 94%, 95%, 96%, 97%, 98% or 99% identity with a CDR-H1 selected from SEQ ID NOs: 5, 840, and 48, the CDR-L3 has at least about 80%, 90%, 91%, 92%, 93%, 94%, 95%, 96%, 97%, 98% or 99% identity with a CDR-L3 selected from SEQ ID NOs: 385 and 410, the CDR-L2 has at least about 80%, 90%, 91%, 92%, 93%, 94%, 95%, 96%, 97%, 98% or 99% identity with a CDR-L2 selected from SEQ ID NOs: 338 and 362, and the CDR-L1 has at least about 80%, 90%, 91%, 92%, 93%, 94%, 95%, 96%, 97%, 98% or 99% identity with a CDR-L1 selected from SEQ ID NOs: 273, 298, and 322. In some embodiments, the CDR-H3 is a CDR-H3 selected from SEQ ID NOs: 192, 207, and 243, with up to 1, 2, 3, 4, 5, 6, 7, or 8 amino acid substitutions; the CDR-H2 is a CDR-H2 selected from SEQ ID NOs: 94, 131, and 162, with up to 1, 2, 3, 4, 5, 6, 7, or 8 amino acid substitutions; the CDR-H1 is a CDR-H1 selected from SEQ ID NOs: 5, 840, and 48, with up to 1, 2, 3, 4, or 5 amino acid substitutions; the CDR-L3 is a CDR-L3 selected from SEQ ID NOs: 385 and 410, with up to 1, 2, 3, 4, or 5 amino acid substitutions; the CDR-L2 is a CDR-L2 selected from SEQ ID NOs: 338 and 362, with up to 1, 2, 3, or 4 amino acid substitutions; and the CDR-L1 is a CDR-L1 selected from SEQ ID NOs: 273, 298, and 322.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 192, 207, and 243; a CDR-L1 comprising an amino acid sequence set forth in any one of SEQ ID NOs: 273, 298, and 322; a CDR-L2 comprising an amino acid sequence set forth in any one of SEQ ID NOs: 338 and 362; and a CDR-L3 comprising an amino acid sequence set forth in any one of SEQ ID NOs: 385 and 410.

In some embodiments, the CDR-H3 has at least about 80%, 90%, 91%, 92%, 93%, 94%, 95%, 96%, 97%, 98% or 99% identity with a CDR-H3 selected from SEQ ID NOs: 179, 207, and 227, the CDR-H2 has at least about 80%, 90%, 91%, 92%, 93%, 94%, 95%, 96%, 97%, 98% or 99% identity with a CDR-H2 selected from SEQ ID NOs: 95, 132, and 163, the CDR-H1 has at least about 80%, 90%, 91%, 92%, 93%, 94%, 95%, 96%, 97%, 98% or 99% identity with a CDR-H1 selected from SEQ ID NOs: 17, 38, and 64, the CDR-L3 has at least about 80%, 90%, 91%, 92%, 93%, 94%, 95%, 96%, 97%, 98% or 99% identity with a CDR-L3 selected from SEQ ID NOs: 386 and 411, the CDR-L2 has at least about 80%, 90%, 91%, 92%, 93%, 94%, 95%, 96%, 97%, 98% or 99% identity with a CDR-L2 selected from SEQ ID NOs: 338 and 362, and the CDR-L1 has at least about 80%, 90%, SEQ ID NOs: 272, 297, and 321. In some embodiments, the CDR-H3 is a CDR-H3 selected from SEQ ID NOs: 179, 207, and 227, with up to 1, 2, 3, 4, 5, 6, 7, or 8 amino acid substitutions; the CDR-H2 is a CDR-H2 selected from SEQ ID NOs: 95, 132, and 163, with up to 1, 2, 3, 4, 5, 6, 7, or 8 amino acid substitutions; the CDR-H1 is a CDR-H1 selected from SEQ ID NOs: 17, 38, and 64, with up to 1, 2, 3, 4, or 5 amino acid substitutions; the CDR-L3 is a CDR-L3 selected from SEQ ID NOs: 386 and 411, with up to 1, 2, 3, 4, or 5 amino acid substitutions; the CDR-L2 is a CDR-L2 selected from SEQ ID NOs: 338 and 362, with up to 1, 2, 3, or 4 amino acid substitutions; and the CDR-L1 is a CDR-L1 selected from SEQ ID NOs: 272, 297, and 321.

In some embodiments, an antibody provided herein comprises a CDR-H1 comprising an amino acid sequence set forth in any one of SEQ ID NOs: 17, 38, and 64; a CDR-H2 comprising an amino acid sequence set forth in any one of SEQ ID NOs: 95, 132, and 163; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, the CDR-H3 has at least about 80%, 90%, 91%, 92%, 93%, 94%, 95%, 96%, 97%, 98% or 99% identity with a CDR-H3 selected from SEQ ID NOs: 192, 207, and 243, the CDR-H2 has at least about 80%, 90%, 91%, 92%, 93%, 94%, 95%, 96%, 97%, 98% or 99% identity with a CDR-H2 selected from SEQ ID NOs: 96, 130, and 164, the CDR-H1 has at least about 80%, 90%, 91%, 92%, 93%, 94%, 95%, 96%, 97%, 98% or 99% identity with a CDR-H1 selected from SEQ ID NOs: 5, 24, and 49, the CDR-L3 has at least about 80%, 90%, 91%, 92%, 93%, 94%, 95%, 96%, 97%, 98% or 99% identity with a CDR-L3 selected from SEQ ID NOs: 386 and 411, the CDR-L2 has at least about 80%, 90%, 91%, 92%, 93%, 94%, 95%, 96%, 97%, 98% or 99% identity with a CDR-L2 selected from SEQ ID NOs: 338 and 362, and the CDR-L1 has at least about 80%, 90%, 91%, 92%, 93%, 94%, 95%, 96%, 97%, 98% or 99% identity with a CDR-L1 selected from SEQ ID NOs: 274, 299, and 323. In some embodiments, the CDR-H3 is a CDR-H3 selected from SEQ ID NOs: 192, 207, and 243, with up to 1, 2, 3, 4, 5, 6, 7, or 8 amino acid substitutions; the CDR-H2 is a CDR-H2 selected from SEQ ID NOs: 96, 130, and 164, with up to 1, 2, 3, 4, 5, 6, 7, or 8 amino acid substitutions; the CDR-H1 is a CDR-H1 selected from SEQ ID NO: 5, 24, and 49, with up to 1, 2, 3, 4, or 5 amino acid substitutions; the CDR-L3 is a CDR-L3 selected from SEQ ID NOs: 386 and 411, with up to 1, 2, 3, 4, or 5 amino acid substitutions; the CDR-L2 is a CDR-L2 selected from SEQ ID NOs: 338 and 362, with up to 1, 2, 3, or 4 amino acid substitutions; and the CDR-L1 is a CDR-L1 selected from SEQ ID NOs: 274, 299, and 323.

In some embodiments, an antibody provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 96, 130, and 164; a CDR-H3 comprising an amino acid sequence set forth in any one of SEQ ID NOs: 192, 207, and 243; a CDR-L1 comprising an amino acid sequence set forth in any one of SEQ ID NOs: 274, 299, and 323; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, the CDR-H3 has at least about 80%, 90%, 91%, 92%, 93%, 94%, 95%, 96%, 97%, 98% or 99% identity with a CDR-H3 selected from SEQ ID NOs: 179, 207, and 227, the CDR-H2 has at least about 80%, 90%, 91%, 92%, 93%, 94%, 95%, 96%, 97%, 98% or 99% identity with a CDR-H2 selected from SEQ ID NOs: 97, 130, and 164, the CDR-H1 has at least about 80%, 90%, 91%, 92%, 93%, 94%, 95%, 96%, 97%, 98% or 99% identity with a CDR-H1 selected from SEQ ID NOs: 5, 24, and 49, the CDR-L3 has at least about 80%, 90%, 91%, 92%, 93%, 94%, 95%, 96%, 97%, 98% or 99% identity with a CDR-L3 selected from SEQ ID NOs: 386 and 411, the CDR-L2 has at least about 80%, 90%, 91%, 92%, 93%, 94%, 95%, 96%, 97%, 98% or 99% identity with a CDR-L2 selected from SEQ ID NOs: 338 and 362, and the CDR-L1 has at least about 80%, 90%, 91%, 92%, 93%, 94%, 95%, 96%, 97%, 98% or 99% identity with a CDR-L1 selected from SEQ ID NOs: 275, 300, and 324. In some embodiments, the CDR-H3 is a CDR-H3 selected from SEQ ID NOs: 179, 207, and 227, with up to 1, 2, 3, 4, 5, 6, 7, or 8 amino acid substitutions; the CDR-H2 is a CDR-H2 selected from SEQ ID NOs: 97, 130, and 164, with up to 1, 2, 3, 4, 5, 6, 7, or 8 amino acid substitutions; the CDR-H1 is a CDR-H1 selected from SEQ ID NO: 5, 24, and 49, with up to 1, 2, 3, 4, or 5 amino acid substitutions; the CDR-L3 is a CDR-L3 selected from SEQ ID NOs: 386 and 411, with up to 1, 2, 3, 4, or 5 amino acid substitutions; the CDR-L2 is a CDR-L2 selected from SEQ ID NOs: 338 and 362, with up to NOs: 275, 300, and 324.

In some embodiments, an antibody provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 97, 130, and 164; a CDR-H3 comprising an amino acid sequence set forth in any one of SEQ ID NOs: 179, 207, and 227; a CDR-L1 comprising an amino acid sequence set forth in any one of SEQ ID NOs: 275, 300, and 324; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, the CDR-H3 has at least about 80%, 90%, 91%, 92%, 93%, 94%, 95%, 96%, 97%, 98% or 99% identity with a CDR-H3 selected from SEQ ID NOs: 193, 216, and 244, the CDR-H2 has at least about 80%, 90%, 91%, 92%, 93%, 94%, 95%, 96%, 97%, 98% or 99% identity with a CDR-H2 selected from SEQ ID NOs: 98, 116, and 147, the CDR-H1 has at least about 80%, 90%, 91%, 92%, 93%, 94%, 95%, 96%, 97%, 98% or 99% identity with a CDR-H1 selected from SEQ ID NOs: 18, 39, and 65, the CDR-L3 has at least about 80%, 90%, 91%, 92%, 93%, 94%, 95%, 96%, 97%, 98% or 99% identity with a CDR-L3 selected from SEQ ID NOs: 387 and 412, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 276, 301, and 325. In some embodiments, the CDR-H3 is a CDR-H3 selected from SEQ ID NOs: 193, 216, and 244, with up to 1, 2, 3, 4, 5, 6, 7, or 8 amino acid substitutions; the CDR-H2 is a CDR-H2 selected from SEQ ID NOs: 98, 116, and 147, with up to 1, 2, 3, 4, 5, 6, 7, or 8 amino acid substitutions; the CDR-H1 is a CDR-H1 selected from SEQ ID NO: 18, 39, and 65, with up to 1, 2, 3, 4, or 5 amino acid substitutions; the CDR-L3 is a CDR-L3 selected from SEQ ID NOs: 387 and 412, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 276, 301, and 325.

In some embodiments, an antibody provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 193, 216, and 244; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity with a CDR-H3 selected from SEQ ID NOs: 193, 216, and 245, the CDR-H2 has at least about 80%, 90%, 91%, 92%, 93%, 94%, 95%, 96%, 97%, 98% or 99% identity with a CDR-H2 selected from SEQ ID NOs: 98, 116, and 147, the CDR-H1 has at least about 80%, 90%, 91%, 92%, 93%, 94%, 95%, 96%, 97%, 98% or 99% identity with a CDR-H1 selected from SEQ ID NOs: 18, 39, and 65, the CDR-L3 has at least about 80%, 90%, 91%, 92%, 93%, 94%, 95%, 96%, 97%, 98% or 99% identity with a CDR-L3 selected from SEQ ID NOs: 387 and 412, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 277, 302, and 326. In some embodiments, the CDR-H3 is a CDR-H3 selected from SEQ ID NOs: 193, 216, and 245, with up to 1, 2, 3, 4, 5, 6, 7, or 8 amino acid substitutions; the CDR-H2 is a CDR-H2 selected from SEQ ID NOs: 98, 116, and 147, with up to 1, 2, 3, 4, 5, 6, 7, or 8 amino acid substitutions; the CDR-H1 is a CDR-H1 selected from SEQ ID NOs: 18, 39, and 65, with up to 1, 2, 3, 4, or 5 amino acid substitutions; the CDR-L3 is a CDR-L3 selected from SEQ ID NOs: 387 and 412, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 277, 302, and 326.

In some embodiments, an antibody provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 193, 216, and 245; a CDR-L1 comprising an amino acid sequence set forth in any one of SEQ ID NOs: 277, 302, and 326; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity with a CDR-H3 selected from SEQ ID NOs: 194, 217, and 246, the CDR-H2 has at least about 80%, 90%, 91%, 92%, 93%, 94%, 95%, 96%, 97%, 98% or 99% identity with a CDR-H2 selected from SEQ ID NOs: 98, 116, and 147, the CDR-H1 has at least about 80%, 90%, 91%, 92%, 93%, 94%, 95%, 96%, 97%, 98% or 99% identity with a CDR-H1 selected from SEQ ID NOs: 18, 39, and 65, the CDR-L3 has at least about 80%, 90%, 91%, 92%, 93%, 94%, 95%, 96%, 97%, 98% or 99% identity with a CDR-L3 selected from SEQ ID NOs: 387 and 412, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 276, 301, and 325. In some embodiments, the CDR-H3 is a CDR-H3 selected from SEQ ID NOs: 194, 217, and 246, with up to 1, 2, 3, 4, 5, 6, 7, or 8 amino acid substitutions; the CDR-H2 is a CDR-H2 selected from SEQ ID NOs: 98, 116, and 147, with up to 1, 2, 3, 4, 5, 6, 7, or 8 amino acid substitutions; the CDR-H1 is a CDR-H1 selected from SEQ ID NOs: 18, 39, and 65, with up to 1, 2, 3, 4, or 5 amino acid substitutions; the CDR-L3 is a CDR-L3 selected from SEQ ID NOs: 387 and 412, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 276, 301, and 325.

In some embodiments, an antibody provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 194, 217, and 246; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity with a CDR-H3 selected from SEQ ID NOs: 194, 217, and 247, the CDR-H2 has at least about 80%, 90%, 91%, 92%, 93%, 94%, 95%, 96%, 97%, 98% or 99% identity with a CDR-H2 selected from SEQ ID NOs: 98, 116, and 147, the CDR-H1 has at least about 80%, 90%, 91%, 92%, 93%, 94%, 95%, 96%, 97%, 98% or 99% identity with a CDR-H1 selected from SEQ ID NOs: 18, 39, and 65, the CDR-L3 has at least about 80%, 90%, 91%, 92%, 93%, 94%, 95%, 96%, 97%, 98% or 99% identity with a CDR-L3 selected from SEQ ID NOs: 388 and 413, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 276, 301, and 325. In some embodiments, the CDR-H3 is a CDR-H3 selected from SEQ ID NOs: 194, 217, and 247, with up to 1, 2, 3, 4, 5, 6, 7, or 8 amino acid substitutions; the CDR-H2 is a CDR-H2 selected from SEQ ID NOs: 98, 116, and 147, with up to 1, 2, 3, 4, 5, 6, 7, or 8 amino acid substitutions; the CDR-H1 is a CDR-H1 selected from SEQ ID NO: 18, 39, and 65, with up to 1, 2, 3, 4, or 5 amino acid substitutions; the CDR-L3 is a CDR-L3 selected from SEQ ID NOs: 388 and 413, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 276, 301, and 325.

In some embodiments, an antibody provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 194, 217, and 247; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 388 and 413.

In some embodiments, the CDR-H3 has at least about 80%, 90%, 91%, 92%, 93%, 94%, 95%, 96%, 97%, 98% or 99% identity with a CDR-H3 selected from SEQ ID NOs: 180, 208, and 248, the CDR-H2 has at least about 80%, 90%, 91%, 92%, 93%, 94%, 95%, 96%, 97%, 98% or 99% identity with a CDR-H2 selected from SEQ ID NOs: 99, 133, and 165, the CDR-H1 has at least about 80%, 90%, 91%, 92%, 93%, 94%, 95%, 96%, 97%, 98% or 99% identity with a CDR-H1 selected from SEQ ID NOs: 19, 40, and 66, the CDR-L3 has at least about 80%, 90%, 91%, 92%, 93%, 94%, 95%, 96%, 97%, 98% or 99% identity with a CDR-L3 selected from SEQ ID NOs: 387 and 412, the CDR-L2 has at least about 80%, 90%, 91%, 92%, 93%, 94%, 95%, 96%, 97%, 98% or 99% identity with a CDR-L2 selected from SEQ ID NOs: 352 and 367, and the CDR-L1 has at least about 80%, 90%, 91%, 92%, 93%, 94%, 95%, 96%, 97%, 98% or 99% identity with a CDR-L1 selected from SEQ ID NOs: 278, 303, and 327. In some embodiments, the CDR-H3 is a CDR-H3 selected from SEQ ID NOs: 180, 208, and 248, with up to 1, 2, 3, 4, 5, 6, 7, or 8 amino acid substitutions; the CDR-H2 is a CDR-H2 selected from SEQ ID NOs: 99, 133, and 165, with up to 1, 2, 3, 4, 5, 6, 7, or 8 amino acid substitutions; the CDR-H1 is a CDR-H1 selected from SEQ ID NOs: 19, 40, and 66, with up to 1, 2, 3, 4, or 5 amino acid substitutions; the CDR-L3 is a CDR-L3 selected from SEQ ID NOs: 387 and 412, with up to 1, 2, 3, 4, or 5 amino acid substitutions; the CDR-L2 is a CDR-L2 selected from SEQ ID NOs: 352 and 367, with up to 1, 2, 3, or 4 amino acid substitutions; and the CDR-L1 is a CDR-L1 selected from SEQ ID NOs: 278, 303, and 327.

In some embodiments, an antibody provided herein comprises a CDR-H1 comprising an amino acid sequence set forth in any one of SEQ ID NOs: 19, 40, and 66; a CDR-H2 comprising an amino acid sequence set forth in any one of SEQ ID NOs: 99, 133, and 165; a CDR-H3 comprising an amino acid sequence set forth in any one of SEQ ID NOs: 180, 208, and 248; a CDR-L1 comprising an amino acid sequence set forth in any one of SEQ ID NOs: 278, 303, and 327; a CDR-L2 comprising an amino acid sequence set forth in any one of SEQ ID NOs: 352 and 367; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity with a CDR-H3 selected from SEQ ID NOs: 195, 218, and 249, the CDR-H2 has at least about 80%, 90%, 91%, 92%, 93%, 94%, 95%, 96%, 97%, 98% or 99% identity with a CDR-H2 selected from SEQ ID NOs: 100, 134, and 166, the CDR-H1 has at least about 80%, 90%, 91%, 92%, 93%, 94%, 95%, 96%, 97%, 98% or 99% identity with a CDR-H1 selected from SEQ ID NOs: 20, 41, and 67, the CDR-L3 has at least about 80%, 90%, 91%, 92%, 93%, 94%, 95%, 96%, 97%, 98% or 99% identity with a CDR-L3 selected from SEQ ID NOs: 374 and 400, the CDR-L2 has at least about 80%, 90%, 91%, 92%, 93%, 94%, 95%, 96%, 97%, 98% or 99% identity with a CDR-L2 selected from SEQ ID NOs: 353 and 361, and the CDR-L1 has at least about 80%, 90%, 91%, 92%, 93%, 94%, 95%, 96%, 97%, 98% or 99% identity with a CDR-L1 selected from SEQ ID NOs: 279, 304, and 328. In some embodiments, the CDR-H3 is a CDR-H3 selected from SEQ ID NOs: 195, 218, and 249, with up to 1, 2, 3, 4, 5, 6, 7, or 8 amino acid substitutions; the CDR-H2 is a CDR-H2 selected from SEQ ID NOs: 100, 134, and 166, with up to 1, 2, 3, 4, 5, 6, 7, or 8 amino acid substitutions; the CDR-H1 is a CDR-H1 selected from SEQ ID NOs: 20, 41, and 67, with up to 1, 2, 3, 4, or 5 amino acid substitutions; the CDR-L3 is a CDR-L3 selected from SEQ ID NOs: 374 and 400, with up to 1, 2, 3, 4, or 5 amino acid substitutions; the CDR-L2 is a CDR-L2 selected from SEQ ID NOs: 353 and 361, with up to 1, 2, 3, or 4 amino acid substitutions; and the CDR-L1 is a CDR-L1 selected from SEQ ID NOs: 279, 304, and 328.

In some embodiments, an antibody provided herein comprises a CDR-H1 comprising an amino acid sequence set forth in any one of SEQ ID NOs: 20, 41, and 67; a CDR-H2 comprising an amino acid sequence set forth in any one of SEQ ID NOs: 100, 134, and 166; a CDR-H3 comprising an amino acid sequence set forth in any one of SEQ ID NOs: 195, 218, and 249; a CDR-L1 comprising an amino acid sequence set forth in any one of SEQ ID NOs: 279, 304, and 328; a CDR-L2 comprising an amino acid sequence set forth in any one of SEQ ID NOs: 353 and 361; and a CDR-L3 comprising an amino acid sequence set forth in any one of SEQ ID NOs: 374 and 400.

In some embodiments, the CDR-H3 has at least about 80%, 90%, 91%, 92%, 93%, 94%, 95%, 96%, 97%, 98% or 99% identity with a CDR-H3 selected from SEQ ID NOs: 180, 208, and 248, the CDR-H2 has at least about 80%, 90%, 91%, 92%, 93%, 94%, 95%, 96%, 97%, 98% or 99% identity with a CDR-H2 selected from SEQ ID NOs: 101, 133, and 167, the CDR-H1 has at least about 80%, 90%, 91%, 92%, 93%, 94%, 95%, 96%, 97%, 98% or 99% identity with a CDR-H1 selected from SEQ ID NOs: 20, 840, and 48, the CDR-L3 has at least about 80%, 90%, 91%, 92%, 93%, 94%, 95%, 96%, 97%, 98% or 99% identity with a CDR-L3 selected from SEQ ID NOs: 389 and 414, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 280, 305, and 329. In some embodiments, the CDR-H3 is a CDR-H3 selected from SEQ ID NOs: 180, 208, and 248, with up to 1, 2, 3, 4, 5, 6, 7, or 8 amino acid substitutions; the CDR-H2 is a CDR-H2 selected from SEQ ID NOs: 101, 133, and 167, with up to 1, 2, 3, 4, 5, 6, 7, or 8 amino acid substitutions; the CDR-H1 is a CDR-H1 selected from SEQ ID NO: 20, 840, and 48, with up to 1, 2, 3, 4, or 5 amino acid substitutions; the CDR-L3 is a CDR-L3 selected from SEQ ID NOs: 389 and 414, with up to 1, 2, 3, 4, or 5 amino acid substitutions; the CDR-L2 is a CDR-L2 selected from SEQ ID NOs: 337 and 361, with up to NOs: 280, 305, and 329.

In some embodiments, an antibody provided herein comprises a CDR-H1 comprising an amino acid sequence set forth in any one of SEQ ID NOs: 20, 840, and 48; a CDR-H2 comprising an amino acid sequence set forth in any one of SEQ ID NOs: 101, 133, and 167; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 389 and 414.

In some embodiments, the CDR-H3 has at least about 80%, 90%, 91%, 92%, 93%, 94%, 95%, 96%, 97%, 98% or 99% identity with a CDR-H3 selected from SEQ ID NOs: 180, 208, and 248, the CDR-H2 has at least about 80%, 90%, 91%, 92%, 93%, 94%, 95%, 96%, 97%, 98% or 99% identity with a CDR-H2 selected from SEQ ID NOs: 102, 135, and 168, the CDR-H1 has at least about 80%, 90%, 91%, 92%, 93%, 94%, 95%, 96%, 97%, 98% or 99% identity with a CDR-H1 selected from SEQ ID NOs: 5, 840, and 48, the CDR-L3 has at least about 80%, 90%, 91%, 92%, 93%, 94%, 95%, 96%, 97%, 98% or 99% identity with a CDR-L3 selected from SEQ ID NOs: 389 and 414, the CDR-L2 has at least about 80%, 90%, 91%, 92%, 93%, 94%, 95%, 96%, 97%, 98% or 99% identity with a CDR-L2 selected from SEQ ID NOs: 350 and 361, and the CDR-L1 has at least about 80%, 90%, 91%, 92%, 93%, 94%, 95%, 96%, 97%, 98% or 99% identity with a CDR-L1 selected from SEQ ID NOs: 280, 305, and 329. In some embodiments, the CDR-H3 is a CDR-H3 selected from SEQ ID NOs: 180, 208, and 248, with up to 1, 2, 3, 4, 5, 6, 7, or 8 amino acid substitutions; the CDR-H2 is a CDR-H2 selected from SEQ ID NOs: 102, 135, and 168, with up to 1, 2, 3, 4, 5, 6, 7, or 8 amino acid substitutions; the CDR-H1 is a CDR-H1 selected from SEQ ID NOs: 5, 840, and 48, with up to 1, 2, 3, 4, or 5 amino acid substitutions; the CDR-L3 is a CDR-L3 selected from SEQ ID NOs: 389 and 414, with up to 1, 2, 3, 4, or 5 amino acid substitutions; the CDR-L2 is a CDR-L2 selected from SEQ ID NOs: 350 and 361, with up to 1, 2, 3, or 4 amino acid substitutions; and the CDR-L1 is a CDR-L1 selected from SEQ ID NOs: 280, 305, and 329.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 102, 135, and 168; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 350 and 361; and a CDR-L3 comprising an amino acid sequence set forth in any one of SEQ ID NOs: 389 and 414.

In some embodiments, the CDR-H3 has at least about 80%, 90%, 91%, 92%, 93%, 94%, 95%, 96%, 97%, 98% or 99% identity with a CDR-H3 selected from SEQ ID NOs: 196, 218, and 250, the CDR-H2 has at least about 80%, 90%, 91%, 92%, 93%, 94%, 95%, 96%, 97%, 98% or 99% identity with a CDR-H2 selected from SEQ ID NOs: 103, 136, and 169, the CDR-H1 has at least about 80%, 90%, 91%, 92%, 93%, 94%, 95%, 96%, 97%, 98% or 99% identity with a CDR-H1 selected from SEQ ID NOs: 5, 840, and 48, the CDR-L3 has at least about 80%, 90%, 91%, 92%, 93%, 94%, 95%, 96%, 97%, 98% or 99% identity with a CDR-L3 selected from SEQ ID NOs: 390 and 415, the CDR-L2 has at least about 80%, 90%, 91%, 92%, 93%, 94%, 95%, 96%, 97%, 98% or 99% identity with a CDR-L2 selected from SEQ ID NOs: 354 and 367, and the CDR-L1 has at least about 80%, 90%, 91%, 92%, 93%, 94%, 95%, 96%, 97%, 98% or 99% identity with a CDR-L1 selected from SEQ ID NOs: 280, 305, and 329. In some embodiments, the CDR-H3 is a CDR-H3 selected from SEQ ID NOs: 196, 218, and 250, with up to 1, 2, 3, 4, 5, 6, 7, or 8 amino acid substitutions; the CDR-H2 is a CDR-H2 selected from SEQ ID NOs: 103, 136, and 169, with up to 1, 2, 3, 4, 5, 6, 7, or 8 amino acid substitutions; the CDR-H1 is a CDR-H1 selected from SEQ ID NO: 5, 840, and 48, with up to 1, 2, 3, 4, or 5 amino acid substitutions; the CDR-L3 is a CDR-L3 selected from SEQ ID NOs: 390 and 415, with up to 1, 2, 3, 4, or 5 amino acid substitutions; the CDR-L2 is a CDR-L2 selected from SEQ ID NOs: 354 and 367, with up to 1, 2, 3, or 4 amino acid substitutions; and the CDR-L1 is a CDR-L1 selected from SEQ ID NOs: 280, 305, and 329.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 103, 136, and 169; a CDR-H3 comprising an amino acid sequence set forth in any one of SEQ ID NOs: 196, 218, and 250; a CDR-L1 comprising an amino acid sequence set forth in any one of SEQ ID NOs: 280, 305, and 329; a CDR-L2 comprising an amino acid sequence set forth in any one of SEQ ID NOs: 354 and 367; and a CDR-L3 comprising an amino acid sequence set forth in any one of SEQ ID NOs: 390 and 415.

In some embodiments, the CDR-H3 has at least about 80%, 90%, 91%, 92%, 93%, 94%, 95%, 96%, 97%, 98% or 99% identity with a CDR-H3 selected from SEQ ID NOs: 197, 208, and 251, the CDR-H2 has at least about 80%, 90%, 91%, 92%, 93%, 94%, 95%, 96%, 97%, 98% or 99% identity with a CDR-H2 selected from SEQ ID NOs: 104, 137, and 170, the CDR-H1 has at least about 80%, 90%, 91%, 92%, 93%, 94%, 95%, 96%, 97%, 98% or 99% identity with a CDR-H1 selected from SEQ ID NOs: 5, 840, and 48, the CDR-L3 has at least about 80%, 90%, 91%, 92%, 93%, 94%, 95%, 96%, 97%, 98% or 99% identity with a CDR-L3 selected from SEQ ID NOs: 387 and 412, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 281, 306, and 330. In some embodiments, the CDR-H3 is a CDR-H3 selected from SEQ ID NOs: 197, 208, and 251, with up to 1, 2, 3, 4, 5, 6, 7, or 8 amino acid substitutions; the CDR-H2 is a CDR-H2 selected from SEQ ID NOs: 104, 137, and 170, with up to 1, 2, 3, 4, 5, 6, 7, or 8 amino acid substitutions; the CDR-H1 is a CDR-H1 selected from SEQ ID NO: 5, 840, and 48, with up to 1, 2, 3, 4, or 5 amino acid substitutions; the CDR-L3 is a CDR-L3 selected from SEQ ID NOs: 387 and 412, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 281, 306, and 330.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 104, 137, and 170; a CDR-H3 comprising an amino acid sequence set forth in any one of SEQ ID NOs: 197, 208, and 251; a CDR-L1 comprising an amino acid sequence set forth in any one of SEQ ID NOs: 281, 306, and 330; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity with a CDR-H3 selected from SEQ ID NOs: 197, 208, and 251, the CDR-H2 has at least about 80%, 90%, 91%, 92%, 93%, 94%, 95%, 96%, 97%, 98% or 99% identity with a CDR-H2 selected from SEQ ID NOs: 105, 138, and 171, the CDR-H1 has at least about 80%, 90%, 91%, 92%, 93%, 94%, 95%, 96%, 97%, 98% or 99% identity with a CDR-H1 selected from SEQ ID NOs: 5, 840, and 48, the CDR-L3 has at least about 80%, 90%, 91%, 92%, 93%, 94%, 95%, 96%, 97%, 98% or 99% identity with a CDR-L3 selected from SEQ ID NOs: 387 and 412, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 281, 306, and 330. In some embodiments, the CDR-H3 is a CDR-H3 selected from SEQ ID NOs: 197, 208, and 251, with up to 1, 2, 3, 4, 5, 6, 7, or 8 amino acid substitutions; the CDR-H2 is a CDR-H2 selected from SEQ ID NOs: 105, 138, and 171, with up to 1, 2, 3, 4, 5, 6, 7, or 8 amino acid substitutions; the CDR-H1 is a CDR-H1 selected from SEQ ID NOs: 5, 840, and 48, with up to 1, 2, 3, 4, or 5 amino acid substitutions; the CDR-L3 is a CDR-L3 selected from SEQ ID NOs: 387 and 412, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 281, 306, and 330.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 105, 138, and 171; a CDR-H3 comprising an amino acid sequence set forth in any one of SEQ ID NOs: 197, 208, and 251; a CDR-L1 comprising an amino acid sequence set forth in any one of SEQ ID NOs: 281, 306, and 330; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity with a CDR-H3 selected from SEQ ID NOs: 198, 219, and 252, the CDR-H2 has at least about 80%, 90%, 91%, 92%, 93%, 94%, 95%, 96%, 97%, 98% or 99% identity with a CDR-H2 selected from SEQ ID NOs: 94, 131, and 162, the CDR-H1 has at least about 80%, 90%, 91%, 92%, 93%, 94%, 95%, 96%, 97%, 98% or 99% identity with a CDR-H1 selected from SEQ ID NOs: 5, 840, and 48, the CDR-L3 has at least about 80%, 90%, 91%, 92%, 93%, 94%, 95%, 96%, 97%, 98% or 99% identity with a CDR-L3 selected from SEQ ID NOs: 391 and 416, the CDR-L2 has at least about 80%, 90%, 91%, 92%, 93%, 94%, 95%, 96%, 97%, 98% or 99% identity with a CDR-SEQ ID NOs: 282, 307, and 331. In some embodiments, the CDR-H3 is a CDR-H3 selected from SEQ ID NOs: 198, 219, and 252, with up to 1, 2, 3, 4, 5, 6, 7, or 8 amino acid substitutions; the CDR-H2 is a CDR-H2 selected from SEQ ID NOs: 94, 131, and 162, with up to 1, 2, 3, 4, 5, 6, 7, or 8 amino acid substitutions; the CDR-H1 is a CDR-H1 selected from SEQ ID NOs: 5, 840, and 48, with up to 1, 2, 3, 4, or 5 amino acid substitutions; the CDR-L3 is a CDR-L3 selected from SEQ ID NOs: 391 and 416, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 282, 307, and 331.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 198, 219, and 252; a CDR-L1 comprising an amino acid sequence set forth in any one of SEQ ID NOs: 282, 307, and 331; a CDR-L2 comprising an amino acid sequence set forth in any one of SEQ ID NOs: 337 and 361; and a CDR-L3 comprising an amino acid sequence set forth in any one of SEQ ID NOs: 391 and 416.

In some embodiments, the CDR-H3 has at least about 80%, 90%, 91%, 92%, 93%, 94%, 95%, 96%, 97%, 98% or 99% identity with a CDR-H3 selected from SEQ ID NOs: 195, 218, and 253, the CDR-H2 has at least about 80%, 90%, 91%, 92%, 93%, 94%, 95%, 96%, 97%, 98% or 99% identity with a CDR-H2 selected from SEQ ID NOs: 94, 131, and 162, the CDR-H1 has at least about 80%, 90%, 91%, 92%, 93%, 94%, 95%, 96%, 97%, 98% or 99% identity with a CDR-H1 selected from SEQ ID NOs: 20, 840, and 48, the CDR-L3 has at least about 80%, 90%, 91%, 92%, 93%, 94%, 95%, 96%, 97%, 98% or 99% identity with a CDR-L3 selected from SEQ ID NOs: 387 and 412, the CDR-L2 has at least about 80%, 90%, 91%, 92%, 93%, 94%, 95%, 96%, 97%, 98% or 99% identity with a CDR-L2 selected from SEQ ID NOs: 355 and 367, and the CDR-L1 has at least about 80%, 90%, 91%, 92%, 93%, 94%, 95%, 96%, 97%, 98% or 99% identity with a CDR-L1 selected from SEQ ID NOs: 280, 305, and 329. In some embodiments, the CDR-H3 is a CDR-H3 selected from SEQ ID NOs: 195, 218, and 253, with up to 1, 2, 3, 4, 5, 6, 7, or 8 amino acid substitutions; the CDR-H2 is a CDR-H2 selected from SEQ ID NOs: 94, 131, and 162, with up to 1, 2, 3, 4, 5, 6, 7, or 8 amino acid substitutions; the CDR-H1 is a CDR-H1 selected from SEQ ID NOs: 20, 840, and 48, with up to 1, 2, 3, 4, or 5 amino acid substitutions; the CDR-L3 is a CDR-L3 selected from SEQ ID NOs: 387 and 412, with up to 1, 2, 3, 4, or 5 amino acid substitutions; the CDR-L2 is a CDR-L2 selected from SEQ ID NOs: 355 and 367, with up to 1, 2, 3, or 4 amino acid substitutions; and the CDR-L1 is a CDR-L1 selected from SEQ ID NOs: 280, 305, and 329.

In some embodiments, an antibody provided herein comprises a CDR-H1 comprising an amino acid sequence set forth in any one of SEQ ID NOs: 20, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 195, 218, and 253; a CDR-L1 comprising an amino acid sequence set forth in any one of SEQ ID NOs: 280, 305, and 329; a CDR-L2 comprising an amino acid sequence set forth in any one of SEQ ID NOs: 355 and 367; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity with a CDR-H3 selected from SEQ ID NOs: 180, 208, and 248, the CDR-H2 has at least about 80%, 90%, 91%, 92%, 93%, 94%, 95%, 96%, 97%, 98% or 99% identity with a CDR-H2 selected from SEQ ID NOs: 94, 131, and 162, the CDR-H1 has at least about 80%, 90%, 91%, 92%, 93%, 94%, 95%, 96%, 97%, 98% or 99% identity with a CDR-H1 selected from SEQ ID NOs: 5, 840, and 48, the CDR-L3 has at least about 80%, 90%, 91%, 92%, 93%, 94%, 95%, 96%, 97%, 98% or 99% identity with a CDR-L3 selected from SEQ ID NOs: 387 and 412, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 280, 305, and 329. In some embodiments, the CDR-H3 is a CDR-H3 selected from SEQ ID NOs: 180, 208, and 248, with up to 1, 2, 3, 4, 5, 6, 7, or 8 amino acid substitutions; the CDR-H2 is a CDR-H2 selected from SEQ ID NOs: 94, 131, and 162, with up to 1, 2, 3, 4, 5, 6, 7, or 8 amino acid substitutions; the CDR-H1 is a CDR-H1 selected from SEQ ID NOs: 5, 840, and 48, with up to 1, 2, 3, 4, or 5 amino acid substitutions; the CDR-L3 is a CDR-L3 selected from SEQ ID NOs: 387 and 412, with up to 1, 2, 3, 4, or 5 amino acid substitutions; the CDR-L2 is a CDR-L2 selected from SEQ ID NOs: 337 and 361, with up to NOs: 280, 305, and 329.

In some embodiments, an antibody provided herein comprises a CDR-H1 comprising an amino acid sequence set forth in any one of SEQ ID NOs: 5, 840, or 48; a CDR-H2 comprising an amino acid sequence set forth in any one of SEQ ID NOs: 94, 131, and 162; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity with a CDR-H3 selected from SEQ ID NOs: 195, 218, and 253, the CDR-H2 has at least about 80%, 90%, 91%, 92%, 93%, 94%, 95%, 96%, 97%, 98% or 99% identity with a CDR-H2 selected from SEQ ID NOs: 106, 137, and 170, the CDR-H1 has at least about 80%, 90%, 91%, 92%, 93%, 94%, 95%, 96%, 97%, 98% or 99% identity with a CDR-H1 selected from SEQ ID NOs: 6, 840, and 48, the CDR-L3 has at least about 80%, 90%, 91%, 92%, 93%, 94%, 95%, 96%, 97%, 98% or 99% identity with a CDR-L3 selected from SEQ ID NOs: 387 and 412, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 280, 305, and 329. In some embodiments, the CDR-H3 is a CDR-H3 selected from SEQ ID NOs: 195, 218, and 253, with up to 1, 2, 3, 4, 5, 6, 7, or 8 amino acid substitutions; the CDR-H2 is a CDR-H2 selected from SEQ ID NOs: 106, 137, and 170, with up to 1, 2, 3, 4, 5, 6, 7, or 8 amino acid substitutions; the CDR-H1 is a CDR-H1 selected from SEQ ID NO: 6, 840, and 48, with up to 1, 2, 3, 4, or 5 amino acid substitutions; the CDR-L3 is a CDR-L3 selected from SEQ ID NOs: 387 and 412, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 280, 305, and 329.

In some embodiments, an antibody provided herein comprises a CDR-H1 comprising an amino acid sequence set forth in any one of SEQ ID NOs: 6, 840, and 48; a CDR-H2 comprising an amino acid sequence set forth in any one of SEQ ID NOs: 106, 137, and 170; a CDR-H3 comprising an amino acid sequence set forth in any one of SEQ ID NOs: 195, 218, and 253; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity with a CDR-H3 selected from SEQ ID NOs: 180, 208, and 248, the CDR-H2 has at least about 80%, 90%, 91%, 92%, 93%, 94%, 95%, 96%, 97%, 98% or 99% identity with a CDR-H2 selected from SEQ ID NOs: 107, 133, and 167, the CDR-H1 has at least about 80%, 90%, 91%, 92%, 93%, 94%, 95%, 96%, 97%, 98% or 99% identity with a CDR-H1 selected from SEQ ID NOs: 20, 41, and 67, the CDR-L3 has at least about 80%, 90%, 91%, 92%, 93%, 94%, 95%, 96%, 97%, 98% or 99% identity with a CDR-L3 selected from SEQ ID NOs: 387 and 412, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 280, 305, and 329. In some embodiments, the CDR-H3 is a CDR-H3 selected from SEQ ID NOs: 180, 208, or 248, with up to 1, 2, 3, 4, 5, 6, 7, or 8 amino acid substitutions; the CDR-H2 is a CDR-H2 selected from SEQ ID NOs: 107, 133, and 167, with up to 1, 2, 3, 4, 5, 6, 7, or 8 amino acid substitutions; the CDR-H1 is a CDR-H1 selected from SEQ ID NOs: 20, 41, and 67, with up to 1, 2, 3, 4, or 5 amino acid substitutions; the CDR-L3 is a CDR-L3 selected from SEQ ID NOs: 387 and 412, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 280, 305, and 329.

In some embodiments, an antibody provided herein comprises a CDR-H1 comprising an amino acid sequence set forth in any one of SEQ ID NOs: 20, 41, and 67; a CDR-H2 comprising an amino acid sequence set forth in any one of SEQ ID NOs: 107, 133, and 167; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity with a CDR-H3 selected from SEQ ID NOs: 181, 209, and 229, the CDR-H2 has at least about 80%, 90%, 91%, 92%, 93%, 94%, 95%, 96%, 97%, 98% or 99% identity with a CDR-H2 selected from SEQ ID NOs: 108, 139, and 172, the CDR-H1 has at least about 80%, 90%, 91%, 92%, 93%, 94%, 95%, 96%, 97%, 98% or 99% identity with a CDR-H1 selected from SEQ ID NOs: 5, 840, and 48, the CDR-L3 has at least about 80%, 90%, 91%, 92%, 93%, 94%, 95%, 96%, 97%, 98% or 99% identity with a CDR-L3 selected from SEQ ID NOs: 374 and 400, the CDR-L2 has at least about 80%, 90%, 91%, 92%, 93%, 94%, 95%, 96%, 97%, 98% or 99% identity with a CDR-L2 selected from SEQ ID NOs: 356 and 361, and the CDR-L1 has at least about 80%, 90%, 91%, 92%, 93%, 94%, 95%, 96%, 97%, 98% or 99% identity with a CDR-L1 selected from SEQ ID NOs: 283, 308, and 332. In some embodiments, the CDR-H3 is a CDR-H3 selected from SEQ ID NOs: 181, 209, and 229, with up to 1, 2, 3, 4, 5, 6, 7, or 8 amino acid substitutions; the CDR-H2 is a CDR-H2 selected from SEQ ID NOs: 108, 139, and 172, with up to 1, 2, 3, 4, 5, 6, 7, or 8 amino acid substitutions; the CDR-H1 is a CDR-H1 selected from SEQ ID NOs: 5, 840, and 48, with up to 1, 2, 3, 4, or 5 amino acid substitutions; the CDR-L3 is a CDR-L3 selected from SEQ ID NOs: 374 and 400, with up to 1, 2, 3, 4, or 5 amino acid substitutions; the CDR-L2 is a CDR-L2 selected from SEQ ID NOs: 356 and 361, with up to 1, 2, 3, or 4 amino acid substitutions; and the CDR-L1 is a CDR-L1 selected from SEQ ID NOs: 283, 308, and 332.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 108, 139, and 172; a CDR-H3 comprising an amino acid sequence set forth in any one of SEQ ID NOs: 181, 209, and 229; a CDR-L1 comprising an amino acid sequence set forth in any one of SEQ ID NOs: 283, 308, and 332; a CDR-L2 comprising an amino acid sequence set forth in any one of SEQ ID NOs: 356 and 361; and a CDR-L3 comprising an amino acid sequence set forth in any one of SEQ ID NOs: 374 and 400.

In some embodiments, the CDR-H3 has at least about 80%, 90%, 91%, 92%, 93%, 94%, 95%, 96%, 97%, 98% or 99% identity with a CDR-H3 selected from SEQ ID NOs: 199, 220, and 254, the CDR-H2 has at least about 80%, 90%, 91%, 92%, 93%, 94%, 95%, 96%, 97%, 98% or 99% identity with a CDR-H2 selected from SEQ ID NOs: 109, 140, and 173, the CDR-H1 has at least about 80%, 90%, 91%, 92%, 93%, 94%, 95%, 96%, 97%, 98% or 99% identity with a CDR-H1 selected from SEQ ID NOs: 21, 42, and 68, the CDR-L3 has at least about 80%, 90%, 91%, 92%, 93%, 94%, 95%, 96%, 97%, 98% or 99% identity with a CDR-L3 selected from SEQ ID NOs: 374 and 400, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 280, 305, and 329. In some embodiments, the CDR-H3 is a CDR-H3 selected from SEQ ID NOs: 199, 220, and 254, with up to 1, 2, 3, 4, 5, 6, 7, or 8 amino acid substitutions; the CDR-H2 is a CDR-H2 selected from SEQ ID NOs: 109, 140, and 173, with up to 1, 2, 3, 4, 5, 6, 7, or 8 amino acid substitutions; the CDR-H1 is a CDR-H1 selected from SEQ ID NO: 21, 42, and 68, with up to 1, 2, 3, 4, or 5 amino acid substitutions; the CDR-L3 is a CDR-L3 selected from SEQ ID NOs: 374 and 400, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 280, 305, and 329.

In some embodiments, an antibody provided herein comprises a CDR-H1 comprising an amino acid sequence set forth in any one of SEQ ID NOs: 21, 42, and 68; a CDR-H2 comprising an amino acid sequence set forth in any one of SEQ ID NOs: 109, 140, and 173; a CDR-H3 comprising an amino acid sequence set forth in any one of SEQ ID NOs: 199, 220, and 254; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 374 and 400.

In some embodiments, the CDR-H3 has at least about 80%, 90%, 91%, 92%, 93%, 94%, 95%, 96%, 97%, 98% or 99% identity with a CDR-H3 selected from SEQ ID NOs: 200, 221, and 255, the CDR-H2 has at least about 80%, 90%, 91%, 92%, 93%, 94%, 95%, 96%, 97%, 98% or 99% identity with a CDR-H2 selected from SEQ ID NOs: 106, 137, and 170, the CDR-H1 has at least about 80%, 90%, 91%, 92%, 93%, 94%, 95%, 96%, 97%, 98% or 99% identity with a CDR-H1 selected from SEQ ID NOs: 5, 43, and 69, the CDR-L3 has at least about 80%, 90%, 91%, 92%, 93%, 94%, 95%, 96%, 97%, 98% or 99% identity with a CDR-L3 selected from SEQ ID NOs: 374 and 400, the CDR-L2 has at least about 80%, 90%, 91%, 92%, 93%, 94%, 95%, 96%, 97%, 98% or 99% identity with a CDR-L2 selected from SEQ ID NOs: 337 and 361, and the CDR-L1 has at least about 80%, 90%, 91%, 92%, 93%, 94%, 95%, 96%, 97%, 98% or 99% identity with a CDR-L1 selected from SEQ ID NOs: 284, 309, and 333. In some embodiments, the CDR-H3 is a CDR-H3 selected from SEQ ID NOs: 200, 221, and 255, with up to 1, 2, 3, 4, 5, 6, 7, or 8 amino acid substitutions; the CDR-H2 is a CDR-H2 selected from SEQ ID NOs: 106, 137, and 170, with up to 1, 2, 3, 4, 5, 6, 7, or 8 amino acid substitutions; the CDR-H1 is a CDR-H1 selected from SEQ ID NOs: 5, 43, and 69, with up to 1, 2, 3, 4, or 5 amino acid substitutions; the CDR-L3 is a CDR-L3 selected from SEQ ID NOs: 374 or 400, with up to 1, 2, 3, 4, or 5 amino acid substitutions; the CDR-L2 is a CDR-L2 selected from SEQ ID NOs: 337 and 361, with up to 1, 2, 3, or 4 amino acid substitutions; and the CDR-L1 is a CDR-L1 selected from SEQ ID NOs: 284, 309, and 333.

In some embodiments, an antibody provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 106, 137, and 170; a CDR-H3 comprising an amino acid sequence set forth in any one of SEQ ID NOs: 200, 221, and 255; a CDR-L1 comprising an amino acid sequence set forth in any one of SEQ ID NOs: 284, 309, and 333; a CDR-L2 comprising an amino acid sequence set forth in any one of SEQ ID NOs: 337 and 361; and a CDR-L3 comprising an amino acid sequence set forth in any one of SEQ ID NOs: 374 and 400.

In some embodiments, the CDR-H3 has at least about 80%, 90%, 91%, 92%, 93%, 94%, 95%, 96%, 97%, 98% or 99% identity with a CDR-H3 selected from SEQ ID NOs: 201, 222, and 256, the CDR-H2 has at least about 80%, 90%, 91%, 92%, 93%, 94%, 95%, 96%, 97%, 98% or 99% identity with a CDR-H2 selected from SEQ ID NOs: 94, 131, and 162, the CDR-H1 has at least about 80%, 90%, 91%, 92%, 93%, 94%, 95%, 96%, 97%, 98% or 99% identity with a CDR-H1 selected from SEQ ID NOs: 5, 43, and 69, the CDR-L3 has at least about 80%, 90%, 91%, 92%, 93%, 94%, 95%, 96%, 97%, 98% or 99% identity with a CDR-L3 selected from SEQ ID NOs: 392 and 417, the CDR-L2 has at least about 80%, 90%, 91%, 92%, 93%, 94%, 95%, 96%, 97%, 98% or 99% identity with a CDR-L2 selected from SEQ ID NOs: 357 or 361, and the CDR-L1 has at least about 80%, 90%, 91%, 92%, 93%, 94%, 95%, 96%, 97%, 98% or 99% identity with a CDR-L1 selected from SEQ ID NOs: 285, 310, and 334. In some embodiments, the CDR-H3 is a CDR-H3 selected from SEQ ID NOs: 201, 222, or 256, with up to 1, 2, 3, 4, 5, 6, 7, or 8 amino acid substitutions; the CDR-H2 is a CDR-H2 selected from SEQ ID NOs: 94, 131, and 162, with up to 1, 2, 3, 4, 5, 6, 7, or 8 amino acid substitutions; the CDR-H1 is a CDR-H1 selected from SEQ ID NOs: 5, 43, and 69, with up to 1, 2, 3, 4, or 5 amino acid substitutions; the CDR-L3 is a CDR-L3 selected from SEQ ID NOs: 392 and 417, with up to 1, 2, 3, 4, or 5 amino acid substitutions; the CDR-L2 is a CDR-L2 selected from SEQ ID NOs: 357 and 361, with up to 1, 2, 3, or 4 amino acid substitutions; and the CDR-L1 is a CDR-L1 selected from SEQ ID NOs: 285, 310, and 334.

In some embodiments, an antibody provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 94, 131, and 162; a CDR-H3 comprising an amino acid sequence set forth in any one of SEQ ID NOs: 201, 222, and 256; a CDR-L1 comprising an amino acid sequence set forth in any one of SEQ ID NOs: 285, 310, and 334; a CDR-L2 comprising an amino acid sequence set forth in any one of SEQ ID NOs: 357 and 361; and a CDR-L3 comprising an amino acid sequence set forth in any one of SEQ ID NOs: 392 and 417.

In some embodiments, the CDR-H3 has at least about 80%, 90%, 91%, 92%, 93%, 94%, 95%, 96%, 97%, 98% or 99% identity with a CDR-H3 selected from SEQ ID NOs: 202, 222, and 257, the CDR-H2 has at least about 80%, 90%, 91%, 92%, 93%, 94%, 95%, 96%, 97%, 98% or 99% identity with a CDR-H2 selected from SEQ ID NOs: 110, 141, and 174, the CDR-H1 has at least about 80%, 90%, 91%, 92%, 93%, 94%, 95%, 96%, 97%, 98% or 99% identity with a CDR-H1 selected from SEQ ID NOs: 5, 43, and 69, the CDR-L3 has at least about 80%, 90%, 91%, 92%, 93%, 94%, 95%, 96%, 97%, 98% or 99% identity with a CDR-L3 selected from SEQ ID NOs: 393 and 418, the CDR-L2 has at least about 80%, 90%, 91%, 92%, 93%, 94%, 95%, 96%, 97%, 98% or 99% identity with a CDR-L2 selected from SEQ ID NOs: 358 and 361, and the CDR-L1 has at least about 80%, 90%, 91%, 92%, 93%, 94%, 95%, 96%, 97%, 98% or 99% identity with a CDR-L1 selected from SEQ ID NOs: 285, 310, and 334. In some embodiments, the CDR-H3 is a CDR-H3 selected from SEQ ID NOs: 202, 222, and 257, with up to 1, 2, 3, 4, 5, 6, 7, or 8 amino acid substitutions; the CDR-H2 is a CDR-H2 selected from SEQ ID NOs: 110, 141, or 174, with up to 1, 2, 3, 4, 5, 6, 7, or 8 amino acid substitutions; the CDR-H1 is a CDR-H1 selected from SEQ ID NOs: 5, 43, and 69, with up to 1, 2, 3, 4, or 5 amino acid substitutions; the CDR-L3 is a CDR-L3 selected from SEQ ID NOs: 393 or 418, with up to 1, 2, 3, 4, or 5 amino acid substitutions; the CDR-L2 is a CDR-L2 selected from SEQ ID NOs: 358 and 361, with up to NOs: 285, 310, and 334.

In some embodiments, an antibody provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 110, 141, and 174; a CDR-H3 comprising an amino acid sequence set forth in any one of SEQ ID NOs: 202, 222, and 257; a CDR-L1 comprising an amino acid sequence set forth in any one of SEQ ID NOs: 285, 310, and 334; a CDR-L2 comprising an amino acid sequence set forth in any one of SEQ ID NOs: 358 and 361; and a CDR-L3 comprising an amino acid sequence set forth in any one of SEQ ID NOs: 393 and 418.

In some embodiments, an antibody provided herein comprises a CDR-H3 comprising an amino acid sequence set forth in any one of SEQ ID NOs: 545, 546, and 547; and a CDR-L3 comprising an amino acid sequence set forth in any one of SEQ ID NOs: 548, 549, and 550.

In some embodiments, an antibody provided herein comprises a CDR-H1 comprising an amino acid sequence set forth in any one of SEQ ID NOs: 551, 557, and 563; a CDR-H2 comprising an amino acid sequence set forth in any one of SEQ ID NOs: 552, 558, and 564; a CDR-H3 comprising an amino acid sequence set forth in any one of SEQ ID NOs: 553, 559, and 565; a CDR-L1 comprising an amino acid sequence set forth in any one of SEQ ID NOs: 554, 560, and 566; a CDR-L2 comprising an amino acid sequence set forth in any one of SEQ ID NOs: 555, 561, and 567; and a CDR-L3 comprising an amino acid sequence set forth in any one of SEQ ID NOs: 556, 562, and 568.

In some embodiments, an antibody provided herein comprises a CDR-H1 comprising an amino acid sequence set forth in any one of SEQ ID NOs: 569, 575, and 581; a CDR-H2 comprising an amino acid sequence set forth in any one of SEQ ID NOs: 570, 576, and 582; a CDR-H3 comprising an amino acid sequence set forth in any one of SEQ ID NOs: 571, 577, and 583; a CDR-L1 comprising an amino acid sequence set forth in any one of SEQ ID NOs: 572, 578, and 584; a CDR-L2 comprising an amino acid sequence set forth in any one of SEQ ID NOs: 573, 579, and 585; and a CDR-L3 comprising an amino acid sequence set forth in any one of SEQ ID NOs: 574, 580, and 586.

In some embodiments, an antibody provided herein comprises a CDR-H1 comprising an amino acid sequence set forth in any one of SEQ ID NOs: 587, 592, and 596; a CDR-H2 comprising an amino acid sequence set forth in any one of SEQ ID NOs: 588, 593, and 597; a CDR-H3 comprising an amino acid sequence set forth in any one of SEQ ID NOs: 589, 207, and 598; a CDR-L1 comprising an amino acid sequence set forth in any one of SEQ ID NOs: 590, 594, and 599; a CDR-L2 comprising an amino acid sequence set forth in any one of SEQ ID NOs: 338, 362, and 362; and a CDR-L3 comprising an amino acid sequence set forth in any one of SEQ ID NOs: 591, 595, and 600.

In some embodiments, an antibody provided herein comprises a CDR-H1 comprising an amino acid sequence set forth in any one of SEQ ID NOs: 601, 607, and 613; a CDR-H2 comprising an amino acid sequence set forth in any one of SEQ ID NOs: 602, 608, and 614; a CDR-H3 comprising an amino acid sequence set forth in any one of SEQ ID NOs: 603, 609, and 615; a CDR-L1 comprising an amino acid sequence set forth in any one of SEQ ID NOs: 604, 610, and 616; a CDR-L2 comprising an amino acid sequence set forth in any one of SEQ ID NOs: 605, 611, and 617; and a CDR-L3 comprising an amino acid sequence set forth in any one of SEQ ID NOs: 606, 612, and 618.

In some embodiments, an antibody provided herein comprises a CDR-H1 comprising an amino acid sequence set forth in any one of SEQ ID NOs: 619, 626, and 631; a CDR-H2 comprising an amino acid sequence set forth in any one of SEQ ID NOs: 620, 626, and 632; a CDR-H3 comprising an amino acid sequence set forth in any one of SEQ ID NOs: 621, 627, and 633; a CDR-L1 comprising an amino acid sequence set forth in any one of SEQ ID NOs: 622, 628, and 634; a CDR-L2 comprising an amino acid sequence set forth in any one of SEQ ID NOs: 623, 629, and 635; and a CDR-L3 comprising an amino acid sequence set forth in any one of SEQ ID NOs: 624, 630, and 636.

In some embodiments, the CDR-H3 has at least about 80%, 90%, 91%, 92%, 93%, 94%, 95%, 96%, 97%, 98% or 99% identity with a CDR-H3 selected from SEQ ID NOs: 763, 769, and 775, the CDR-H2 has at least about 80%, 90%, 91%, 92%, 93%, 94%, 95%, 96%, 97%, 98% or 99% identity with a CDR-H2 selected from SEQ ID NOs: 748, 116, and 758, the CDR-H1 has at least about 80%, 90%, 91%, 92%, 93%, 94%, 95%, 96%, 97%, 98% or 99% identity with a CDR-H1 selected from SEQ ID NOs: 5, 840, and 48, the CDR-L3 has at least about 80%, 90%, 91%, 92%, 93%, 94%, 95%, 96%, 97%, 98% or 99% identity with a CDR-L3 selected from SEQ ID NOs: 806 and 812, the CDR-L2 has at least about 80%, 90%, 91%, 92%, 93%, 94%, 95%, 96%, 97%, 98% or 99% identity with a CDR-L2 selected from SEQ ID NOs: 797, and 802, and the CDR-L1 has at least about 80%, 90%, 91%, 92%, 93%, 94%, 95%, 96%, 97%, 98% or 99% identity with a CDR-L1 selected from SEQ ID NOs: 781, 787, and 792. In some embodiments, the CDR-H3 is a CDR-H3 selected from SEQ ID NOs: 763, 769, and 775, with up to 1, 2, 3, 4, 5, 6, 7, and 8 amino acid substitutions; the CDR-H2 is a CDR-H2 selected from SEQ ID NOs: 748, 116, and 758, with up to 1, 2, 3, 4, 5, 6, 7, and 8 amino acid substitutions; the CDR-H1 is a CDR-H1 selected from SEQ ID NOs: 5, 840, and 48, with up to 1, 2, 3, 4, and 5 amino acid substitutions; the CDR-L3 is a CDR-L3 selected from SEQ ID NOs: 806 and 812, with up to 1, 2, 3, 4, and 5 amino acid substitutions; the CDR-L2 is a CDR-L2 selected from SEQ ID NOs: 797 and 802, with up to 1, 2, 3, and 4 amino acid substitutions; and the CDR-L1 is a CDR-L1 selected from SEQ ID NOs: 781, 787, and 792.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 748, 116, and 758; a CDR-H3 comprising an amino acid sequence set forth in any one of SEQ ID NOs: 763, 769, and 775, a CDR-L1 comprising an amino acid sequence set forth in any one of SEQ ID NOs: 781, 787, and 792; a CDR-L2 comprising an amino acid sequence set forth in any one of SEQ ID NOs: 797 and 802; and a CDR-L3 comprising an amino acid sequence set forth in any one of SEQ ID NOs: 806 and 812.

In some embodiments, an antibody provided herein comprises a CDR-H1 comprising an amino acid sequence set forth in any one of SEQ ID NOs: 740, 742, and 745; a CDR-H2 comprising an amino acid sequence set forth in any one of SEQ ID NOs: 749, 754, and 759; a CDR-H3 comprising an amino acid sequence set forth in any one of SEQ ID NOs: 764, 770, and 776, a CDR-L1 comprising an amino acid sequence set forth in any one of SEQ ID NOs: 782, 788, and 793; a CDR-L2 comprising an amino acid sequence set forth in any one of SEQ ID NOs: 798 and 803; and a CDR-L3 comprising an amino acid sequence set forth in any one of SEQ ID NOs: 807 and 813.

In some embodiments, an antibody provided herein comprises a CDR-H1 comprising an amino acid sequence set forth in any one of SEQ ID NOs: 17, 743, and 746; a CDR-H2 comprising an amino acid sequence set forth in any one of SEQ ID NOs: 750, 133, and 165; a CDR-H3 comprising an amino acid sequence set forth in any one of SEQ ID NOs: 765, 771, and 777, a CDR-L1 comprising an amino acid sequence set forth in any one of SEQ ID NOs: 783, 298, and 322; a CDR-L2 comprising an amino acid sequence set forth in any one of SEQ ID NOs: 799 and 804; and a CDR-L3 comprising an amino acid sequence set forth in any one of SEQ ID NOs: 808 and 814.

In some embodiments, an antibody provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 751, 755, and 760; a CDR-H3 comprising an amino acid sequence set forth in any one of SEQ ID NOs: 766, 772, and 778, a CDR-L1 comprising an amino acid sequence set forth in any one of SEQ ID NOs: 784, 789, and 794; a CDR-L2 comprising an amino acid sequence set forth in any one of SEQ ID NOs: 341 and 361; and a CDR-L3 comprising an amino acid sequence set forth in any one of SEQ ID NOs: 809 and 815.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223, a CDR-L1 comprising an amino acid sequence set forth in any one of SEQ ID NOs: 260, 288, and 313; a CDR-L2 comprising an amino acid sequence set forth in any one of SEQ ID NOs: 341 and 361; and a CDR-L3 comprising an amino acid sequence set forth in any one of SEQ ID NOs: 368 and 394.

In some embodiments, an antibody provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 752, 756, and 761; a CDR-H3 comprising an amino acid sequence set forth in any one of SEQ ID NOs: 767, 773, and 779, a CDR-L1 comprising an amino acid sequence set forth in any one of SEQ ID NOs: 785, 790, and 795; a CDR-L2 comprising an amino acid sequence set forth in any one of SEQ ID NOs: 800 and 805; and a CDR-L3 comprising an amino acid sequence set forth in any one of SEQ ID NOs: 810 and 816.

In some embodiments, an antibody provided herein comprises a CDR-H1 comprising an amino acid sequence set forth in any one of SEQ ID NOs: 741, 744, and 747; a CDR-H2 comprising an amino acid sequence set forth in any of SEQ ID NOs: 753, 757, and 762; a CDR-H3 comprising an amino acid sequence set forth in any one of SEQ ID NOs: 768, 774, and 780, a CDR-L1 comprising an amino acid sequence set forth in any of SEQ ID NOs: 786, 791, and 796; a CDR-L2 comprising an amino acid sequence set forth in any of SEQ ID NOs: 801 and 360; and a CDR-L3 comprising an amino acid sequence set forth in any one of SEQ ID NOs: 811 and 817.

In some embodiments, an antibody provided herein comprises a CDR-H1 comprising an amino acid sequence set forth in any one of SEQ ID NOs: 10, 27, and 52; a CDR-H2 comprising an amino acid sequence set forth in any of SEQ ID NOs: 70, 111, and 142; a CDR-H3 comprising an amino acid sequence set forth in any one of SEQ ID NOs: 175, 203, and 234, a CDR-L1 comprising an amino acid sequence set forth in any of SEQ ID NOs: 260, 288, 313; a CDR-L2 comprising an amino acid sequence set forth in any of SEQ ID NOs: 347 and 365; and a CDR-L3 comprising an amino acid sequence set forth in any one of SEQ ID NOs: 378 and 404.

TABLE 3
Consensus sequences of clones

| | |
|---|---|
| Consensus Sequence | SEQ ID NO: |

Variants of 327

| | |
|---|---|
| CDRH3 (Kabat) EGYDLWSGX₁SSNWLDP, where X₁ = D, E, H, or Y | 545 |
| CDRH3 (Chothia) GYDLWSGX₁SSNWLD, where X₁ = D, E, H, or Y | 546 |
| CDRH3 (IMGT) AREGYDLWSGX₁SSNWLDP, where X₁ = D, E, H, or Y | 547 |
| CDRL3 (Kabat) QQLX₁X₂YPRT, where X₁ = N or Q, and X₂ = S or N | 548 |
| CDRL3 (Chothia) LX₁X₂YPR, where X₁ = N or Q, and X₂ = S or N | 549 |
| CDRL3 (IMGT) QQLX₁X₂YPRT, where X₁ = N or Q, and X₂ = S or N | 550 |

Variants of 114

| | |
|---|---|
| CDRH1 (Kabat) X₁X₂AMX₃, where X₁ = N, S, T, or I, X₂ = Y or H, and X₃ = T, Y, or S | 551 |
| CDRH2 (Kabat) LIX₁GX₂GX₃X₄TX₅YADSVX₆G, where X₁ = S or T, X₂ = S or G, | 552 |
| X₃ = G, A, or S, X₄ = L, S, I, or V, X₅ = K, H, N, or Y, and X₆ = K or R | |
| CDRH3 (Kabat) X₁EGLTX₂GEX₃, where X₁ = D or E, X₂ = T or S, and X₃ = Y, F, or S | 553 |
| CDRL1 (Kabat) RX₁SQX₂IRNX₃LX₄, where X₁ = A or T, X₂ = D, G, or A, | 554 |
| X₃ = D or N, and X₄ = A, G, or D | |
| CDRL2 (Kabat) X₁X₂SX₃X₄X₅S, where X₁ = A or V, X₂ = S, V, T, or A, | 555 |
| X₃ = S or N, X₄ = L or F, and X₅ = Q or K | |
| CDRL3 (Kabat) LQHX₁X₂YPFX₃, where X₁ = N or S, X₂ = N, S, or T, and X₃ = T or S | 556 |
| CDRH1 (Chothia) GFTFX₁X₂X₃, where X₁ = S, R, N, G, or T, X₂ = N, S, T, or I, | 557 |
| and X₃ = Y or H | |
| CDRH2 (Chothia) X₁GX₂GX₃X₄, where X₁ = S or T, X₂ = S or G, X₃ = G, A, or S, | 558 |
| and X₄ = L, S, I, or V | |
| CDRH3 (Chothia) EGLTX₁GE, where X₁ = T or S | 559 |
| CDRL1 (Chothia) SQX₁IRNX₂, where X₁ = D, G, or A, and X₂ = D or N | 560 |
| CDRL2 (Chothia) X₁X₂S, where X₁ = A or V, and X₂ = S, V, T, or A | 561 |
| CDRL3 (Chothia) HX₁X₂YPF, where X₁ = N or S, and X₂ = N, S, or T | 562 |
| CDRH1 (IMGT) GFTFX₁X₂X₃A, where X₁ = S, R, N, G, or T, X₂ = N, S, T, or I, and | 563 |
| X₃ = Y or H | |
| CDRH2 (IMGT) IX₁GX₂GX₃X₄T, where X₁ = S or T, X₂ = S or G, X₃ = G, A, or S, | 564 |
| and X₄ = L, S, I, or V | |
| CDRH3 (IMGT) X₁KX₂EGLTX₃GEX₄, where X₁ = V or A, X₂ = D or E, X₃ = T or S, | 565 |
| and X₄ = Y, F, or S | |
| CDRL1 (IMGT) QX₁IRNX₂, where X₁ = D, G, or A, and X₂ = D or N | 566 |
| CDRL2 (IMGT) X₁X₂S, where X₁ = A or V, and X₂ = S, V, T, or A | 567 |
| CDRL3 (IMGT) LQHX₁X₂YPFX₃, where X₁ = N or S, X₂ = N, S, or T, and X₃ = T or S | 568 |

Variants of 122

| | |
|---|---|
| CDRH1 (Kabat) X₁X₂WMH, where X₁ = N or S, and X₂ = Y or F | 569 |
| CDRH2 (Kabat) RIX₁X₂X₃GX₄X₅TRYADSVKG, where X₁ = N, D, or S, X₂ = S or N, | 570 |
| X₃ = D or N, X₄ = I or F, and X₅ = S or N | |
| CDRH3 (Kabat) DFQLELX₁DX₂, where X₁ = P or R, and X₂ = Y or F | 571 |
| CDRL1 (Kabat) RASQX₁IRX₂DLX₃, where X₁ = G or A, X₂ = D or N, and X₃ = A or G | 572 |
| CDRL2 (Kabat) AX₁SX₂LQX₃, where X₁ = A or T, X₂ = S or I, and X₃ = S or N | 573 |
| CDRL3 (Kabat) LQHX₁SNPFT, where X₁ = K, N, or Y | 574 |
| CDRH1 (Chothia) GX₁TFX₂X₃X₄, where X₁ = F or L, X₂ = S or R, and X₃ = N or S, and | 575 |
| X₄ = Y or F | |
| CDRH2 (Chothia) X₁X₂X₃GX₄X₅, where X₁ = N, D, or S, X₂ = S or N, X₃ = D or N, | 576 |
| X₄ = I or F, and X₅ = S or N | |
| CDRH3 (Chothia) FQLELX₁D, where X₁ = P or R | 577 |
| CDRL1 (Chothia) SQX₁IRX₂D, where X₁ = G or A, and X₂ = D or N | 578 |
| CDRL2 (Chothia) AX₁S, where X₁ = A or T | 579 |
| CDRL3 (Chothia) HX₁SNPF, where X₁ = K, N, or Y | 580 |
| CDRH1 (IMGT) GX₁TFX₂X₃X₄W, where X₁ = F or L, X₂ = S or R, and X₃ = N or S, and | 581 |
| X₄ = Y or F | |
| CDRH2 (IMGT) IX₁X₂X₃GX₄X₅T, where X₁ = N, D, or S, X₂ = S or N, X₃ = D or N, | 582 |
| X₄ = I or F, and X₅ = S or N | |
| CDRH3 (IMGT) VX₁DFQLELX₂DX₃, where X₁ = R or S, X₂ = P or R, and X₃ = Y or F | 583 |
| CDRL1 (IMGT) QX₁IRX₂D, where X₁ = G or A, X₂ = D or N | 584 |
| CDRL2 (IMGT) AXIS, where X₁ = A or T | 585 |
| CDRL3 (IMGT) LQHX₁SNPFT, where X₁ = K, N, or Y | 586 |

Variants of 127

| | |
|---|---|
| CDRH1 (Kabat) X₁AWMS, where X₁ = N or S | 587 |
| CDRH2 (Kabat) RX₁KX₂X₃X₄X₅GGTX₆DX₇X₈APX₉X₁₀G, where X₁ = I or V, X₂ = S or G, X₃ = N, | 588 |
| V, or K, X₄ = S or T, X₅ = Y, A, D, or T, X₆ = T or A, X₇ = Y or F, X₈ = A or P, | |
| X₉ = A or V, and X₁₀ = I or K | |
| CDRH3 (Kabat) DHPKFGFDX₁, where X₁ = F or Y | 589 |
| CDRL1 (Kabat) KSSQSVX₁YX₂SX₃X₄KX₅YLX₆, where X₁ = F or L, X₂ = S or T, | 590 |
| and X₃ = N or S, X₄ = N or S, X₅ = N or H, and X₆ = A or N | |
| CDRL2 (Kabat) WASTRES | 338 |
| CDRL3 (Kabat) QQX₁YX₂X₃PRT, where X₁ = H or Y, X₂ = D or S, and X₃ = I or T | 591 |
| CDRH1 (Chothia) GFTFX₁X₂A, where X₁ = S, H, or N, X₂ = N or S | 592 |
| CDRH2 (Chothia) KX₁X₂X₃X₄GGT, where X₁ = S or G, X₂ = N, V, or K, X₃ = S or T, | 593 |
| and X₄ = Y, A, D, or T | |
| CDRH3 (Chothia) HPKFGFD | 207 |
| CDRL1 (Chothia) SQSVX₁YX₂SX₃X₄KX₅Y, where X₁ = F or L, X₂ = S or T, and | 594 |
| X₃ = N or S, X₄ = N or S, and X₅ = N or H | |
| CDRL2 (Chothia) WAS | 362 |
| CDRL3 (Chothia) X₁YX₂X₃PR, where X₁ = H or Y, X₂ = D or S, and X₃ = I or T | 595 |
| CDRH1 (IMGT) GFTFX₁X₂AW, where X₁ = S, H, or N, X₂ = N or S | 596 |
| CDRH2 (IMGT) X₁KX₂X₃X₄X₅GGTX₆, where X₁ = I or V, X₂ = S or G, X₃ = N, V, | 597 |
| or K, X₄ = S or T, X₅ = Y, A, D, or T, X₆ = T or A | |
| CDRH3 (IMGT) TTDHPKFGFDX₁, where X₁ = F or Y | 598 |
| CDRL1 (IMGT) QSVX₁YX₂SX₃X₄KX₅Y, where X₁ = For L, X₂ = S or T, and X₃ = N or | 599 |
| S, X₄ = N or S, X₅ = N or H | |
| CDRL2 (IMGT) WAS | 362 |
| CDRL3 (IMGT) QQX₁YX₂X₃PRT, where X₁ = H or Y, X₂ = D or S, and X₃ = I or T | 600 |

Variants of 128

| | |
|---|---|
| CDRH1 (Kabat) X₁AWMX₂, where X₁ = N or Y, and X₂ = T, R, N, or S | 601 |
| CDRH2 (Kabat) X₁IKX₂X₃X₄X₅GGTX₆X₇X₈X₉APVX₁0G, where X₁ = R or H, | 602 |
| X₂ = S, R, or T, X₃ = I, E, K, L, N, or Q, X₄ = S or T, X₅ = Y or | |
| D, X₆ = T or R, X₇ = D or E, X₈ = S or Y, X₉ = A or D, and X₁₀ = K or Q | |
| CDRH3 (Kabat)GX₁VWX₂YGGSLDX₃, where X₁ = S or G, X₂ = K, N, or R, and X₃ = F or Y | 603 |
| CDRL1 (Kabat) RASQX₁IX₂RX₃LN, where X₁ = G, S, F, N, or I, X₂ = S or T, and | 604 |
| X₃ = F, Y, or S | |
| CDRL2 (Kabat) X₁ASX₂LX₃X₄, where X₁ = A or G, X₂ = T, S, or R, X₃ = Q or E, | 605 |
| and X₄ = N or S | |
| CDRL3 (Kabat) QQX₁YX₂X₃X₄X₅T, where X₁ = T or S, X₂ = S, N, R, or G, X₃ = T or S, | 606 |
| X₄ = P or S, and X₅ = L or V | |
| CDRH1 (Chothia) GFX₁FX₂X₃A, where X₁ = T or I, X₂ = N, I, or S, and X₃ = N, or Y | 607 |
| CDRH2 (Chothia) KX₁X₂X₃X₄GGT, where X₁ = S, R, or T, X₂ = I, E, K, L, N, or | 608 |
| Q, X₃ = S or T, and X₄ = Y or D | |
| CDRH3 (Chothia) X₁VWX₂YGGSLD, where X₁ = S or G, and X₂ = K, N, or R | 609 |
| CDRL1 (Chothia) SQX₁IX₂RX₃, where X₁ = G, S, F, N, or I, X₂ = S or T, and | 610 |
| X₃ = F, Y, or S | |
| CDRL2 (Chothia) X₁AS, where X₁ = A or G | 611 |
| CDRL3 (Chothia) X₁YX₂X₃X₄X₅, where X₁ = T or S, X₂ = S, N, R, or G, X₃ = T or S, | 612 |
| X₄ = P or S, and X₅ = L or V | |
| CDRH1 (IMGT) GFX₁FX₂X₃AW, where X₁ = T or I, X₂ = N, I, or S, and X₃ = N, or Y | 613 |
| CDRH2 (IMGT) IKX₁X₂X₃X₄GGTX₅, where X₁ = S, R, or T, X₂ = I, E, K, L, N, or Q, | 614 |
| X₃ = S or T, X₄ = Y or D, and X₅ = T or R | |
| CDRH3 (IMGT) X₁TGX₂VWX₃YGGSLDX₄, where X₁ = S, T, I, or A, X₂ = S or G, | 615 |
| X₃ = K, N, or R, and X₄ = F or Y | |
| CDRL1 (IMGT) QX₁IX₂RX₃, where X₁ = G, S, F, N, or I, X₂ = S or T, and X₃ = F, Y, | 616 |
| or S | |
| CDRL2 (IMGT) X₁AS, where X₁ = A or G | 617 |
| CDRL3 (IMGT) QQX₁YX₂X₃X₄X₅T, where X₁ = T or S, X₂ = S, N, R, or G, X₃ = T or S, | 618 |
| X₄ = P or S, and X₅ = L or V | |

Variants of 130

| | |
|---|---|
| CDRH1 (Kabat) X₁AWMS, where X₁ = Y, N, or T | 619 |
| CDRH2 (Kabat) RIKX₁X₂X₃X₄GGTTDYAX₅PVKG, where X₁ = S or R, X₂ = E or K, | 620 |
| X₃ = T, S, or I, X₄ = D or Y, and X₅ = A or S | |
| CDRH3 (Kabat) GSSWX₁X₂YX₃FDX₄, where X₁ = N, H, S or Y, X₂ = V or L, X₃ = F or Y, | 621 |
| and X₄ = F or Y | |
| CDRL1 (Kabat) RASQSX₁SX₂X₃LN, where X₁ = I or M, X₂ = N, S, R or T, and X₃ = Y | 622 |
| or H | |
| CDRL2 (Kabat) AAX₁X₂LX₃X₄, where X₁ = Y or S, X₂ = R, S, or N, X₃ = Q or E, | 623 |
| and X₄ = T or S | |
| CDRL3 (Kabat) QQSYX₁X₂PX₃T, where X₁ = S or R, X₂ = S or T, and X₃ = L or F | 624 |
| CDRH1 (Chothia) GFX₁FSX₂A, where X₁ = T or I, and X₂ = Y, N, or T | 625 |
| CDRH2 (Chothia) KX₁X₂X₃X₄GGT, where X₁ = S or R, X₂ = E or K, X₃ = T, S, or I, | 626 |
| X₄ = D or Y | |
| CDRH3 (Chothia) SSWX₁X₂YX₃FD, X₁ = N, H, S or Y, X₂ = V or L, and X₃ = F or Y | 627 |
| CDRL1 (Chothia) SQSX₁SX₂X₃, where X₁ = I or M, X₂ = N, S, R or T, and X₃ = Y or H | 628 |
| CDRL2 (Chothia) AAX₁, where X₁ = Y or S | 629 |
| CDRL3 (Chothia) SYX₁X₂PX₃, where X₁ = S or R, X₂ = S or T, and X₃ = L or F | 630 |
| CDRH1 (IMGT) GFX₁FSX₂AW, where X₁ = T or I, and X₂ = Y, N, or T | 631 |
| CDRH2 (IMGT) IKX₁X₂X₃X₄GGTT, where X₁ = S or R, X₂ = E or K, X₃ = T, S, or I, | 632 |
| and X₄ = D or Y | |
| CDRH3 (IMGT) TTGSSWX₁X₂YX₃FDX₄, where X₁ = N, H, S or Y, X₂ = V or L, X₃ = F or Y, | 633 |
| and X₄ = F or Y | |
| CDRL1 (IMGT) QSXISX₂X₃, where X₁ = I or M, X₂ = N, S, R or T, and X₃ = Y or H | 634 |
| CDRL2 (IMGT) AAX₁, where X₁ = Y or S | 635 |
| CDRL3 (IMGT) QQSYX₁X₂PX₃T, where X₁ = S or R, X₂ = S or T, and X₃ = L or F | 636 |

In certain embodiments, any of the antibodies described above further comprises a heavy chain comprising a human IgG sequence selected from a sequence set forth in SEQ ID NOs: 637-737 or a sequence having at least about 80%, 90%, 91%, 92%, 93%, 94%, 95%, 96%, 97%, 98% or 99% identity thereto. In certain embodiments, any of the antibodies described above further comprises a heavy chain sequence set forth in SEQ ID NO: 651 or a sequence having at least about 80%, 90%, 91%, 92%, 93%, 94%, 95%, 96%, 97%, 98% or 99% identity thereto. In certain embodiments, any of the antibodies described above further comprises a human light chain sequence set forth in SEQ ID NO: 738 or a sequence having at least about 80%, 90%, 91%, 92%, 93%, 94%, 95%, 96%, 97%, 98% or 99% identity thereto. In certain embodiments, any of the antibodies described above further comprises a heavy chain sequence set forth in SEQ ID NO: 651 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In some aspects, the amino acid substitutions are conservative amino acid substitutions. In some embodiments, the antibodies described in this disclosure are referred to herein as “variants” or “clones”. In some embodiments, such variants or clones are derived from a sequence provided herein, for example, by affinity maturation, site directed mutagenesis, random mutagenesis, or any other method known in the art or described herein. In some embodiments, such variants or cones are not derived from a sequence provided herein and may, for example, be isolated de novo according to the methods provided herein for obtaining antibodies.

### Fc Region

The structures of the Fc regions of various immunoglobulins, and the glycosylation sites contained therein, are known in the art. See Schroeder et al. (2010) Allergy Clin. Immunol. 125: S41-52, incorporated by reference in its entirety. The Fc region may be a naturally occurring Fc region, or an Fc region modified as described in the art or elsewhere in this disclosure.

Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM, and several of these may be further divided into subclasses (isotypes), e.g., IgG1, IgG2, IgG3, IgG4, IgA1, and IgA2.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

Modifications in the CH2 domain can affect the binding of FcRs to the Fc. A number of amino acid modifications in the Fc region are known in the art for selectively altering the affinity of the Fc for different Fcgamma receptors. In some aspects, the Fc comprises one or more modifications to promote selective binding of Fc-gamma receptors.

Exemplary mutations that alter the binding of FcRs to the Fc are listed below:
S298A/E333A/K334A, S298A/E333A/K334A/K326A (Lu Y, Vernes J M, Chiang N, et al. J Immunol Methods. 2011 Feb. 28; 365 (1-2): 132-41);
F243L/R292P/Y300L/V305I/P396L, F243L/R292P/Y300L/L235V/P396L (Stavenhagen J B, Gorlatov S, Tuaillon N, et al. Cancer Res. 2007 Sep. 15; 67 (18): 8882-90; Nordstrom J L, Gorlatov S, Zhang W, et al. Breast Cancer Res. 2011 Nov. 30; 13 (6): R123);
F243L (Stewart R, Thom G, Levens M, et al. Protein Eng Des Sel. 2011 September; 24 (9): 671-8.), S298A/E333A/K334A (Shields R L, Namenuk A K, Hong K, et al. J Biol Chem. 2001 Mar. 2; 276 (9): 6591-604);
S239D/1332E/A330L, S239D/1332E (Lazar et al. (2006) Proc Natl Acad Sci USA. 103 (11): 4005-10);
S239D/S267E, S267E/L328F (Chu et al. (2008) Mol Immunol. 45 (15): 3926-33);
S239D/D265S/S298A/I332E, S239E/S298A/K326A/A327H, G237F/S298A/A330L/133 2E, S239D/1332E/S298A, S239D/K326E/A330L/1332E/S298A, G236A/S239D/D270 L/1332E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments, the heavy chain comprises a constant heavy chain sequence selected from an amino acid sequence set forth in any one of SEQ ID NOs: 637-737, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence set forth in SEQ ID NO: 651, or an amino acid sequence having at least 90%, at least 91%, at least 92%, at least 93%, at least 94%, at least 95%, at least 96%, at least 97%, at least 98%, or at least 99% sequence identity thereto. In some embodiments, the light chain comprises a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence set forth in SEQ ID NO: 651, or an amino acid sequence having at least 90%, at least 91%, at least 92%, at least 93%, at least 94%, at least 95%, at least 96%, at least 97%, at least 98%, or at least 99% sequence identity thereto and the light chain comprises a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto.

In some embodiments, an antibody described herein includes modifications to improve its ability to mediate effector function. Such modifications are known in the art and include afucosylation, or engineering of the affinity of the Fc towards an activating receptor, mainly FCGR3a for ADCC, and towards C1q for CDC. The following TABLE 4 summarizes various designs reported in the literature for effector function engineering.

Methods of producing antibodies with little or no fucose on the Fc glycosylation site (Asn 297 EU numbering) without altering the amino acid sequence are well known in the art. The GlymaX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 20 (12): 1607-18. Another approach to obtaining antibodies with lowered levels of fucosylation can be found in U.S. Pat. No. 8,409,572, which teaches selecting cell lines for antibody production for their ability to yield lower levels of fucosylation on antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

Thus, in one embodiment, an antibody described herein can include a dimeric Fc that comprises one or more amino acid modifications as noted in TABLE 4 that confer improved effector function. In another embodiment, the antibody can be afucosylated to improve effector function.

TABLE 4
TABLE 4: CH2 domains and effector function engineering

| Reference | Mutations | Effect |
|---|---|---|
| Lu (2011), supra; | Afucosylated | Increased |
| Ferrara( 2011), | | ADCC |
| supra; Mizushima | | |
| et al. (2011) | | |
| Genes Cells. | | |
| 16(11): 1071-1080. | | |
| Lu (2011), supra | S298A/E333A/K334A | Increased |
| | | ADCC |
| Lu (2011), supra | S298A/E333A/K334A/K326A | Increased |
| | | ADCC |
| Stavenhagen (2007), | F243L/R292P/Y300L/V305I/P396L | Increased |
| supra | | ADCC |
| Nordstrom (2011), | F243L/R292P/Y300L/L235V/P396L | Increased |
| supra | | ADCC |
| Stewart (2011), supra | F243L | Increased |
| | | ADCC |
| Shields (2001), supra | S298A/E333A/K334A | Increased |
| | | ADCC |
| Lazar (2006), supra | S239D/I332E/A330L | Increased |
| | | ADCC |
| Lazar (2006), supra | S239D/I332E | Increased |
| | | ADCC |
| Bowles et al. (2006) | AME-D, not specified mutations | Increased |
| Blood 108(8): 2648-54 | | ADCC |
| Heider et al. (2011) | 37.1, mutations not disclosed | Increased |
| Blood 118(15): | | ADCC |
| 4159-68 | | |
| Moore et al. (2010) | S267E/H268F/S324T | Increased |
| MAbs 2(2): 181-9 | | CDC |

Fc modifications reducing FcgR and/or complement binding and/or effector function are known in the art. Recent publications describe strategies that have been used to engineer antibodies with reduced or silenced effector activity (see Strohl, WR (2009) Curr Opin Biotech 20:685-691, and Strohl, WR and Strohl LM, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, U.S. Patent Publication No. 2011/0212087 (Strohl), International Patent Publication No. WO 2006/105338 (Xencor), U.S. Patent Publication No. 2012/0225058 (Xencor), U.S. Patent Publication No. 2012/0251531 (Genentech), and Strop et al ((2012) J. Mol. Biol. 420:204-219) describe specific modifications to reduce FcgR or complement binding to the Fc.

Specific, non-limiting examples of known amino acid modifications to reduce FcgR or complement binding to the Fc include those identified in the following TABLE 5:

TABLE 5
Modifications to reduce FcgR or complement binding to the Fc
Mutations

N297A
L234A/L235A
IGG2 V234A/G237A
IGG4 L235A/G237A/E318A
IGG4 S228P/L236E
IGG2/IGG4
IGG2 H268Q/V309L/A330S/A331S
C220S/C226S/C229S/P238S
C226S/C229S/E3233P/L235V/L235A
E. coli production, non glyco
L234F/L235E/P331S
Hinge mutant, e.g., C226S/P230S
D265A
L234A/G237A
L234A/L235A/G237A
L234A/L235A/P329G

Methods of producing antibodies with little or no fucose on the Fc glycosylation site (Asn 297 EU numbering) without altering the amino acid sequence are well known in the art. The GlymaxX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) supra). Examples of cell lines capable of producing defucosylated antibody include CHO-DG44 with stable overexpression of the bacterial oxidoreductase GDP-6-deoxy-D-lyxo-4-hexylose reductase (RMD) (see von Horsten et al. (2010) supra) or Lec13 CHO cells, which are deficient in protein fucosylation (see Ripka et al. (1986) Arch. Biochem. Biophys. 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al. (2004) Biotech. Bioeng. 87:614-622; Kanda et al. (2006) Biotechnol. Bioeng. 94:680-688; and WO 2003/085107; each of which is incorporated by reference in its entirety). Another approach to obtaining antibodies with lowered levels of fucosylation can be found in U.S. Pat. No. 8,409,572, which teaches selecting cell lines for antibody production for their ability to yield lower levels of fucosylation on antibodies

Antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

In some aspects, an antibody provided herein comprises an IgG1 domain with reduced fucose content at position Asn 297 compared to a naturally occurring IgG1 domain. Such Fc domains are known to have improved ADCC. See Shields et al. (2002) J. Biol. Chem. 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

In certain embodiments, an antibody provided herein comprises an Fc region with one or more amino acid substitutions which improve ADCC, such as a substitution at one or more of positions 298, 333, and 334 of the Fc region. In some embodiments, an antibody provided herein comprises an Fc region with one or more amino acid substitutions at positions 239, 332, and 330, as described in Lazar et al. (2006) Proc. Natl. Acad. Sci. USA 103:4005-4010, incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, 2004/0109865; International Pat. Pub. Nos. 2000/61739, 2001/29246, 2003/085119, 2003/084570, 2005/035586, 2005/035778; 2005/053742, 2002/031140; Okazaki et al., J. Mol. Biol., 2004, 336:1239-1249; and Yamane-Ohnuki et al. (2004) supra; each of which is incorporated by reference in its entirety.

In some embodiments, an antibody provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which his incorporated by reference in its entirety.

In some embodiments, an antibody provided herein comprises one or more alterations that improves or diminishes C1q binding and/or CDC. See U.S. Pat. No. 6,194,551; WO 99/51642; and Idusogie et al. (2000) J. Immunol. 164:4178-4184; each of which is incorporated by reference in its entirety.

In certain embodiments, the heavy chain comprises a constant heavy chain sequence selected from the sequences set forth in SEQ ID NOs: 637-737.

In certain embodiments, an antibody provided herein comprises a VH sequence and a VL sequence with a heavy chain constant region provided in TABLE 6. In certain embodiments, an antibody provided herein comprises a VH sequence and a VL sequence with a heavy chain constant region and a light chain constant region provided in TABLE 6.

In certain embodiments, the Fc region comprises one or more amino acid substitutions, wherein the one or more substitutions result in an increase in one or more of antibody half-life, ADCC activity, ADCP activity, or CDC activity compared with the Fc without the one or more substitutions. In certain embodiments, the one or more amino acid substitutions results in increased antibody half-life at pH 6.0 compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to amlitelimab.

In certain embodiments, the Fc region comprises one or more amino acid substitutions, wherein the one or more substitutions result in a decrease in one or more of ADCC activity, ADCP activity, or CDC activity compared with the Fc without the one or more substitutions.

In certain embodiments, the one or more amino acid substitutions is selected from the group consisting of S228P (SP), M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W. In certain embodiments, the one or more amino acid substitutions comprises a specific combination of amino acid substitutions selected from the group consisting of M428L/N434S (LS); M252Y/S254T/T256E (YTE); T250Q/M428L; T307A/E380A/N434A; T256D/T307Q (DQ); T256D/T307W (DW); M252Y/T256D (YD); T307Q/Q311V/A378V (QVV); T256D/H285D/T307R/Q311V/A378V (DDRVV); L309D/Q311H/N434S (DHS); S228P/L235E (SPLE); L234A/L235A (LALA), M428L/N434A (LA), L235A/G237A (LAGA), L234A/L235A/G237A (LALAGA), L234A/L235A/P329G, D265A/YTE, LALA/YTE, LAGA/YTE, LALAGA/YTE, LALAPG/YTE, N297A/LS; D265A/LS; LALA/LS; LALAGA/LS; LALAPG/LS; N297A/DHS; D265A/DHS; LALA/DHS; LAGA/DHS; LALAGA/DHS; LALAPG/DHS; SP/YTE; SPLE/YTE; SP/LS; SPLE/LS, SP/DHS; SPLE/DHS; N297A/LA; D265A/LA, LALA/LA, LAGA/LA, LALAGA/LA, LALAPG/LA, N297A/N434A; D265A/N434A; LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

In certain embodiments, the Fc region binds an Fcγ Receptor selected from the group consisting of: FcγRI, FcγRIIa, FcγRIIb, FcγRIIc, FcγRIIIa, and FcγRIIIb. In certain embodiments, the Fc region binds an Fcγ Receptor with higher affinity at pH 6.0 compared to an antibody comprising a wild-type Fc region.

In some embodiments, an antibody disclosed herein comprises a VH and/or VL sequence from TABLE 6. In some embodiments, an antibody containing a VH and/or VL sequence of TABLE 6 also comprises one or more amino acid substitutions, e.g., substitutions in the Fc region, disclosed herein. In some embodiments, an antibody containing a VH and/or VL sequence of TABLE 6 comprises a constant heavy chain sequence selected from the sequences set forth in SEQ ID NOs: 637-737. In some embodiments, an antibody comprising a VH and/or VL sequence of TABLE 6 comprises one or more amino acid substitutions selected from the group consisting of S228P (SP), M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W. In certain embodiments, an antibody containing a VH and/or VL sequence of TABLE 6 comprises a specific combination of amino acid substitutions selected from the group consisting of M428L/N434S (LS); M252Y/S254T/T256E (YTE); T250Q/M428L; T307A/E380A/N434A; T256D/T307Q (DQ); T256D/T307W (DW); M252Y/T256D (YD); T307Q/Q311V/A378V (QVV); T256D/H285D/T307R/Q311V/A378V (DDRVV); L309D/Q311H/N434S (DHS); S228P/L235E (SPLE); L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS; D265A/LS; LALA/LS; LALAGA/LS; LALAPG/LS; N297A/DHS; D265A/DHS; LALA/DHS; LAGA/DHS; LALAGA/DHS; LALAPG/DHS; SP/YTE; SPLE/YTE; SP/LS; SPLE/LS, SP/DHS; SPLE/DHS; N297A/LA; D265A/LA, LALA/LA, LAGA/LA, LALAGA/LA, LALAPG/LA, N297A/N434A; D265A/N434A; LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

TABLE 6
VH and VL sequences of anti-OX40L antibodies

| Antibody | VH | VL |
|---|---|---|
| Amlitelimab | EVQLVESGGGLVQPGG | DIQMTQSPSSLSASVGD |
| | SLRLSCAASGFTFSNYA | RVTITCRASQSISSYLN |
| | MNWVRQAPGKGLEWV | WYQQKPGKAPNLLIYA |
| | STISGSGGATRYADSVK | ASSLQSGVPSRFSGSGS |
| | GRFTISRDNSRNTVYLQ | ETDFTLTISSLQPEDFAT |
| | MNSLRVEDTAVFYCTK | YYCQQSHSVSFTFGPGT |
| | DRLIMATVRGPYYYGM | KVDIK |
| | DVWGQGTTVTVSS | SEQ ID NO: 833 |
| | SEQ ID NO: 832 | |
| LC.001 | EVQLLESGGGLVQPGG | DIQMTQSPSSLSASVGD |
| (Oxelumab) | SLRLSCAASGFTFNSYA | RVTITCRASQGISSWLA |
| | MSWVRQAPGKGLEWV | WYQQKPEKAPKSLIYA |
| | SIISGSGGFTYYADSVK | ASSLQSGVPSRFSGSGS |
| | GRFTISRDNSRTTLYLQ | GTDFTLTISSLQPEDFAT |
| | MNSLRAEDTAVYYCA | YYCQQYNSYPYTFGQG |
| | KDRLVAPGTFDYWGQ | TKLEIK |
| | GALVTVSS | SEQ ID NO: 835 |
| | SEQ ID NO: 834 | |
| LC.005 | QVQLVESGGGVVQPGR | EIVLTQSPGTLSLSPGER |
| | SLRLSCAASGFTFSNFG | ATLSCRASQSVSSNYLA |
| | MHWVRQAPGKGLEWV | WYQQKPGQAPRLLIYG |
| | AAIWYDGHDKYYSYY | ASSRATGIPDRFSGSGS |
| | VKGRFTISRDNSKNTLF | GTDFTLTISRLEPEDFA |
| | LQMNSLRAEDTAVYYC | VYYCQQYGSSFTFGPG |
| | ARDSSSWYRYFDYWG | TKVDIK |
| | QGTLVTVSS | SEQ ID NO: 837 |
| | SEQ ID NO: 836 | |
| LC.019 | QKQLVEFGGGVVQPGR | EIVLTQSPATLSLSPGER |
| | SLRLSCAASGFTFSNYG | ATLSCRASQGVSRYLA |
| | MHWVRQAPGKGLWV | WYQQKPGQAPRLLIYD |
| | AVIWNDGSNKYYVDSV | ASNRATGIPARVSGSGP |
| | KGRFIISRDNSKNTLYL | GTDFTLTISSLEPEDFAV |
| | QMNSLRAEDTAVYYC | DYCQQRSNWQYTFGQ |
| | ARDRMGIYYYGMDVW | GTKLEIK |
| | GQGTTVTVSS | SEQ ID NO: 839 |
| | SEQ ID NO: 838 | |

### Binding

The affinity of a molecule X for its partner Y can be represented by the dissociation equilibrium constant (KD). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule (i.e., OX40L) and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some embodiments, the affinity of an anti-OX40L antibody for a non-target molecule is less than about 50% of the affinity for OX40L. In some embodiments, the affinity of an anti-OX40L antibody for a non-target molecule is less than about 40% of the affinity for OX40L. In some embodiments, the affinity of an anti-OX40L antibody for a non-target molecule is less than about 30% of the affinity for OX40L. In some embodiments, the affinity of an anti-OX40L antibody for a non-target molecule is less than about 20% of the affinity for OX40L. In some embodiments, the affinity of an anti-OX40L antibody for a non-target molecule is less than about 10% of the affinity for OX40L. In some embodiments, the affinity of an anti-OX40L antibody for a non-target molecule is less than about 1% of the affinity for OX40L. In some embodiments, the affinity of an anti-OX40L antibody for a non-target molecule is less than about 0.1% of the affinity for OX40L.

When used herein in the context of two or more antibodies, the term “competes with” or “cross-competes with” indicates that the two or more antibodies compete for binding to an antigen (e.g., OX40L). In one exemplary assay, OX40L is coated on a surface and contacted with a first anti-OX40L antibody, after which a second anti-OX40L antibody is added. In another exemplary assay, a first anti-OX40L antibody is coated on a surface and contacted with OX40L, and then a second anti-OX40L antibody is added. If the presence of the first anti-OX40L antibody reduces binding of the second anti-OX40L antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, at least 95%, or at least 99% as measured in a competitive binding assay. A skilled artisan can select the concentrations of the antibodies used in the competition assays based on the affinities of the antibodies for OX-40L and the valency of the antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (ncbi.nlm.nih.gov/books/NBK92434/; accessed Sep. 29, 2015); Silman et al. (2001) Cytometry 44:30-37; and Finco et al. (2011) J. Pharm. Biomed. Anal. 54:351-358; each of which is incorporated by reference in its entirety.

A test antibody competes with a reference antibody if an excess of a test antibody (e.g., at least 2×, 5×, 10×, 20×, or 100×) inhibits or blocks binding of the reference antibody by, e.g., at least 50%, 60%, 70%, 75%, 80%, 85%, 90%, 95%, or 99% as measured in a competitive binding assay. Antibodies identified by competition assay (competing antibody) include antibodies binding to the same epitope as the reference antibody and antibodies binding to an adjacent epitope sufficiently proximal to the epitope bound by the reference antibody for steric hindrance to occur. For example, a second, competing antibody can be identified that competes for binding to OX40L with a first antibody described herein. In certain instances, the second antibody can block or inhibit binding of the first antibody by, e.g., at least 50%, 60%, 70%, 75%, 80%, 85%, 90%, 95%, or 99% as measured in a competitive binding assay. In certain instances, the second antibody can displace the first antibody by greater than 50%, 60%, 70%, 75%, 80%, 85%, 90%, 95%, or 99%.

In certain embodiments, the antibody binds an OX40L sequence set forth in SEQ ID NO: 739.

In certain embodiments, the antibody binds to an OX40L sequence set forth in SEQ ID NO: 739 with a K_{D} of less than or equal to about 1, 2, 3, 4, 5, 6, 7, 8, 9×10⁻⁹ M, as measured by surface plasmon resonance (SPR). In certain embodiments, the antibody binds to an OX40L sequence set forth in SEQ ID NO: 739 with a K_{D} of less than or equal to about 1×10⁻¹⁰ M, as measured by surface plasmon resonance (SPR). In certain embodiments, the antibody binds to human OX40L with a K_{D} of less than or equal to about 1×10⁻⁹ M, as measured by surface plasmon resonance (SPR).

In some embodiments, an antibody provided herein binds OX40L with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁸ M, as measured by ELISA or any other suitable method known in the art. In some embodiments, an antibody provided herein binds OX40L with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁹ M, as measured by ELISA or any other suitable method known in the art.

In some embodiments, the K_{D} of the antibody provided herein for the binding of OX40L is between about 0.001-0.01, 0.01-0.1, 0.01-0.05, 0.05-0.1, 0.1-0.5, 0.5-1, 0.25-0.75, 0.25-0.5, 0.5-0.75, 0.75-1, 0.75-2, 1.1-1.2, 1.2-1.3, 1.3-1.4, 1.4-1.5, 1.5-1.6, 1.6-1.7, 1.7-1.8, 1.8-1.9, 1.9-2, 1-2, 1-5, 2-7, 3-8, 3-5, 4-6, 5-7, 6-8, 7-9, 7-10, or 5-10×10⁻⁸ M, as measured by ELISA or any other suitable method known in the art. In some embodiments, an antibody provided herein binds OX40L with a K_{D} of less than or equal to about 1×10⁻⁸ M, or less than or equal to above 1×10⁻⁹ M as measured by ELISA or any other suitable method known in the art.

In some embodiments, the antibody provided herein binds OX40L with a KD of less than or equal to about 10, 9, 8, 7, 6, 5, 4.5, 4, 3.5, 3, 2.5, 2, 1.98, 1.95, 1.9, 1.85, 1.8, 1.75, 1.7, 1.65, 1.6, 1.55, 1.50, 1.45, 1.4, 1.3, 1.2, 1.1, 1, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0005, or 0.0001×10⁻⁸ M, or less, as measured by ELISA or any other suitable method known in the art. In some embodiments, the antibody provided herein binds OX40L with a K_{D} between 5-3, 4-2, 3-1, 1.9-1.8, 1.8-1.7, 1.7-1.6, 1.6-1.5, 1.9-1.5, 1.5-1, 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

In some embodiments, the antibody provided herein binds FcRn with an affinity at pH 7.4 compared to pH 6.0 at a ratio (pH 7.4/pH 6.0) of about 10,000, 1,000, 500, 100, 50, 20, 10, 9, 8, 7, 6, 5, 4.5, 4, 3.5, 3, 2.5, 2, 1.95, 1.9, 1.85, 1.8, 1.75, 1.7, 1.65, 1.6, 1.55, 1.50, 1.45, 1.4, 1.3, 1.2, 1.1, or 1.05, as measured by ELISA or any other suitable method known in the art. In some embodiments, the antibody provided herein binds FcRn with an affinity at pH 6.0 compared to pH 7.4 at a ratio (pH 6.0/pH 7.4) of about 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

### Function

“Effector functions” refer to those biological activities mediated by the Fc region of an antibody, which activities may vary depending on the antibody isotype. Examples of antibody effector functions include receptor ligand blocking, agonism, or antagonism, C1q binding to activate complement dependent cytotoxicity (CDC), Fc receptor binding to activate antibody-dependent cellular cytotoxicity (ADCC), and antibody dependent cellular phagocytosis (ADCP).

### Pharmaceutical Compositions

The present application provides compositions comprising the antibodies including pharmaceutical compositions comprising any one or more of the antibodies described herein with one or more pharmaceutically acceptable excipients. In some embodiments the composition is sterile. The pharmaceutical compositions generally comprise an effective amount of an antibody.

These compositions can comprise, in addition to one or more of the antibodies disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

The anti-OX40L antibody that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Methods

### Methods of Preparation

Antibodies described herein can be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody) or an amino acid sequence comprising the VHH of a single domain antibody. In a further embodiment, one or more vectors (e.g., expression vectors) comprising such nucleic acid are provided. In one embodiment, the nucleic acid is provided in a multicistronic vector. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antigen-binding polypeptide construct, or (2) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In one embodiment, the host cell is eukaryotic, e.g. a Chinese Hamster Ovary (CHO) cell, or human embryonic kidney (HEK) cell, or lymphoid cell (e.g., YO, NSO, Sp20 cell). In one embodiment, a method of making an antibody is provided, wherein the method comprises culturing a host cell comprising nucleic acid encoding the antibody, as provided above, under conditions suitable for expression of the antibody, and optionally recovering the antibody from the host cell (or host cell culture medium).

For recombinant production of the antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

When a heteromultimer or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the heteromultimer or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, “substantially purified” heteromultimer produced by the methods described herein, has a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of antibody-encoding vectors include prokaryotic or eukaryotic cells described herein.

Recombinant host cells or host cells are cells that include an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome. Host cells can include CHO, derivatives of CHO, NS0, Sp20, CV-1, VERO-76, HeLa, HepG2, Per.C6, or BHK.

For example, antibody may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. (1980) Proc. Natl. Acad. Sci. USA 77:4216); and myeloma cell lines such as Y0, NSO and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

In one embodiment, the antibodies described herein are produced in stable mammalian cells, by a method comprising: transfecting at least one stable mammalian cell with: nucleic acid encoding the antibody, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antibody in the expressed product.

In some embodiments, is the method of producing an antibody in stable mammalian cells as described herein wherein the expression product of the at least one stable mammalian cell comprises a larger percentage of the desired glycosylated antibody as compared to the monomeric heavy or light chain polypeptides, or other antibodies.

In some embodiments, is the method of producing a glycosylated antibody in stable mammalian cells described herein, said method comprising identifying and purifying the desired glycosylated antibody. In some embodiments, the said identification is by one or both of liquid chromatography and mass spectrometry.

If required, the antibodies can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of antibodies. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed or immobilized anti-flag antibody if a flag-tag is used. For general guidance in suitable purification techniques, see, e.g. incorporated entirely by reference Protein Purification: Principles and Practice, 3rd Ed., Scopes, Springer-Verlag, NY, 1994, incorporated entirely by reference. The degree of purification necessary will vary depending on the use of the antibodies. In some instances, no purification is necessary.

In certain embodiments, the antibodies are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments, the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antibodies described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Non-classical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, alanine, fluoro-amino acids, designer amino acids such as methyl amino acids, C-methyl amino acids, N-methyl amino acids, and amino acid analogs in general. Furthermore, the amino acid can be D (dextrorotary) or L (levorotary).

### Methods of Use

In an aspect, the present application provides methods of contacting OX40L with an anti-OX40L antibody, such as a human or humanized antibody, which results in inhibition of OX40 binding to OX40L expressed on antigen presenting cells.

In an aspect, the present application provides methods of using the isolated anti-OX40L antibodies described herein for treatment of a disorder or disease in a subject. In certain aspects, described herein is a method for treating a subject in need thereof with an anti-OX40L antibody, the method comprising administering to a mammalian subject a therapeutically effective amount of an anti-OX40L antibody or pharmaceutical composition comprising an anti-OX40L antibody described herein. In certain embodiments, the present application provides methods of treating a disorder or disease associated with elevated levels of OX40L and/or IgE in a subject.

In certain aspects, described herein are methods for treating a pathology associated with OX40L activity, the method comprising administering to a mammalian subject a therapeutically effective amount an isolated anti-OX40L antibody or a pharmaceutical composition comprising an isolated anti-OX40L antibody described herein.

In certain embodiments, the isolated antibody is used in the treatment of an inflammatory disorder or disease. In certain embodiments, the isolated antibody is used in the treatment of atopic dermatitis. In certain embodiments, the treatment reduces disease severity in a patient and wherein disease severity is assessed by an Atopic Dermatitis Disease Severity Outcome Measure. In certain embodiments, the isolated antibody is used in the treatment of idiopathic pulmonary fibrosis. In certain embodiments, the isolated antibody is used in the treatment of alopecia areata. In certain embodiments, the isolated antibody is used in the treatment of chronic sinusitis with nasal polyps. In certain embodiments, the isolated antibody is used in the treatment of Chronic Rhinosinusitis without Nasal Polyps (CRSsNP). In certain embodiments, the isolated antibody is used in the treatment of eosinophilic esophagitis (EoE). In certain embodiments, the isolated antibody is used in the treatment of Eosinophilic gastrointestinal disorder or disease (ENID) selected from the group consisting of Eosinophilic Gastritis (EoG), Eosinophilic Enteritis (EoN), Eosinophilic Colitis (EoC), and Eosinophilic Gastroenteritis (EGE). In certain embodiments, the isolated antibody is used in the treatment of Churg-Strauss syndrome/Eosinophilic granulomatosis with polyangiitis (EGPA). In certain embodiments, the isolated antibody is used in the treatment of Prurigo Nodularis (PN). In certain embodiments, the isolated antibody is used in the treatment of Chronic Spontaneous Urticaria (CSU). In certain embodiments, the isolated antibody is used in the treatment of Chronic Pruritis of Unknown Origin (CPUO). In certain embodiments, the isolated antibody is used in the treatment of Bullous Pemphigoid (BP). In certain embodiments, the isolated antibody is used in the treatment of Cold Inducible Urticaria (ColdU). In certain embodiments, the isolated antibody is used in the treatment of Allergic Fungal Rhinosinusitis (AFRS). In certain embodiments, the isolated antibody is used in the treatment of Allergic Bronchopulmonary Aspergillosis (ABPA). In certain embodiments, the isolated antibody is used in the treatment of Chronic Obstructive Pulmonary Disease (COPD). In certain embodiments, the isolated antibody is used in the treatment of inflammatory bowel disease, such as Crohn disease or ulcerative colitis. In certain embodiments, the isolated antibody is used in the treatment of psoriasis. In certain embodiments, the isolated antibody is used in the treatment of lupus. In certain embodiments, the isolated antibody is used in the treatment of rheumatoid arthritis. In certain embodiments, the isolated antibody is used in the treatment of hidradenitis suppurativa. In certain embodiments, the isolated antibody is used in the treatment of celiac disease. In certain embodiments, the isolated antibody is used in the treatment of systemic sclerosis.

In certain aspects, described herein is a method for treating an inflammatory disorder or disease in a mammalian subject in need thereof, the method comprising administering to the mammalian subject a therapeutically effective amount an antibody described herein or a pharmaceutical composition described herein. In certain embodiments of the methods described herein, the inflammatory disorder or disease is atopic dermatitis. In certain embodiments, the inflammatory disorder or disease is asthma. In certain embodiments, the inflammatory disorder or disease is idiopathic pulmonary fibrosis. In certain embodiments of the methods described herein, the inflammatory disorder or disease is alopecia areata. In certain embodiments, the inflammatory disorder or disease is chronic sinusitis with nasal polyps. In certain embodiments, the inflammatory disorder or disease is Chronic Rhinosinusitis without Nasal Polyps (CRSsNP). In certain embodiments, the inflammatory disorder or disease is eosinophilic esophagitis (EoE). In certain embodiments, the inflammatory disorder or disease is an Eosinophilic gastrointestinal disorder or disease (ENID) selected from the group consisting of Eosinophilic Gastritis (EoG), Eosinophilic Enteritis (EoN), Eosinophilic Colitis (EoC), and Eosinophilic Gastroenteritis (EGE). In certain embodiments, the inflammatory disorder or disease is Churg-Strauss syndrome/Eosinophilic granulomatosis with polyangiitis (EGPA). In certain embodiments, the inflammatory disorder or disease is Prurigo Nodularis (PN). In certain embodiments, the inflammatory disorder or disease is Chronic Spontaneous Urticaria (CSU). In certain embodiments, the inflammatory disorder or disease is Chronic Pruritis of Unknown Origin (CPUO). In certain embodiments, the inflammatory disorder or disease is Bullous Pemphigoid (BP). In certain embodiments, the inflammatory disorder or disease is Cold Inducible Urticaria (ColdU). In certain embodiments, the inflammatory disorder or disease is Allergic Fungal Rhinosinusitis (AFRS). In certain embodiments, the inflammatory disorder or disease is Allergic Bronchopulmonary Aspergillosis (ABPA). In certain embodiments, the inflammatory disorder or disease is Chronic Obstructive Pulmonary Disease (COPD). In certain embodiments, the inflammatory disorder or disease is inflammatory bowel disease, such as Crohn disease or ulcerative colitis. In certain embodiments, the inflammatory disorder or disease is psoriasis. In certain embodiments, the inflammatory disorder or disease is lupus. In certain embodiments, the inflammatory disorder or disease is rheumatoid arthritis. In certain embodiments, the inflammatory disorder or disease is celiac disease.

In certain aspects, described herein are methods for treating a pathology associated with elevated levels of OX40L in a mammalian subject in need thereof, the method comprising administering to the mammalian subject a therapeutically effective amount an antibody or a pharmaceutical composition described herein.

In certain aspects, described herein are methods of reducing biological activity of OX40L in a mammalian subject in need thereof, the method comprising administering to the mammalian subject a therapeutically effective amount an antibody or a pharmaceutical composition described herein.

In certain aspects, described herein are methods of preventing an inflammatory disorder or disease in a mammalian subject in need thereof, the method comprising administering to the mammalian subject a therapeutically effective amount an antibody or a pharmaceutical composition described herein.

### Methods of Administration

In some embodiments, the methods provided herein are useful for the treatment of a disease or disorder in an individual. In an embodiment, the individual is a human and the antibody is an anti-OX40L antibody described herein.

In some embodiments, an antibody is administered intravenously, intramuscularly, subcutaneously, topically, orally, transdermally, intraperitoneally, intraorbitally, by implantation, by inhalation, intrathecally, intraventricularly, or intranasally. An effective amount of an anti-OX40L antibody may be administered for the treatment of a disease or disorder. The appropriate dosage of the anti-OX40L antibody may be determined based on the type of disease or disorder to be treated, the type of the anti-OX40L antibody, the severity and course of the disease or disorder, the clinical condition of the individual, the individual's clinical history and response to the treatment, and the discretion of the attending physician.

In some embodiments, an antibody provided herein is administered with at least one additional therapeutic agent. Any suitable additional therapeutic or immunotherapeutic agent may be administered with an antibody provided herein. Additional therapeutic agents include agents that are used to treat or prevent a disease or disorder such as, but not limited to, an inflammatory disease or disorder associated with elevated levels of OX40L and/or IgE.

The additional therapeutic agent can be administered by any suitable means. In some embodiments, an antibody provided herein and the additional therapeutic agent are included in the same pharmaceutical composition. In some embodiments, an antibody provided herein and the additional therapeutic agent are included in different pharmaceutical compositions.

In embodiments where an antibody provided herein and the additional therapeutic agent are included in different pharmaceutical compositions, administration of the antibody can occur prior to, simultaneously, and/or following, administration of the additional therapeutic agent. In some embodiments, administration of an antibody provided herein and the additional therapeutic agent occur within about one month of each other. In some embodiments, administration of an antibody provided herein and the additional therapeutic agent occur within about one week of each other. In some embodiments, administration of an antibody provided herein and the additional therapeutic agent occur within about one day of each other. In some embodiments, administration of an antibody provided herein and the additional therapeutic agent occur within about twelve hours of each other. In some embodiments, administration of an antibody provided herein and the additional therapeutic agent occur within about one hour of each other.

### Kits and Articles of Manufacture

The present application provides kits comprising any one or more of the antibody compositions described herein and instructions for use. In some embodiments, the kits further contain a component selected from any of secondary antibodies, reagents for immunohistochemistry analysis, pharmaceutically acceptable excipient and instruction manual and any combination thereof. In one specific embodiment, the kit comprises a pharmaceutical composition comprising any one or more of the antibody compositions described herein, with one or more pharmaceutically acceptable excipients.

The present application also provides articles of manufacture comprising any one of the antibody compositions or kits described herein. Examples of an article of manufacture include vials (including sealed vials).

### Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Generation of anti-hOX40L antibodies

Alloy ATX mice expressing chimeric antibodies with fully-human variable regions were immunized with recombinant human OX40L (hOX40L) protein and/or DNA encoding hOX40L. During immunizations, the serum was used to measure titers against hOX40L by ELISA.

Splenic and lymph node cells from immunized mice were fused with fusion partner cells to generate hybridomas. Upon expansion, the supernatants were screened for binding to recombinant hOX40L protein by ELISA and hOX40L-expressing CHO cells by flow cytometry.

Alternatively, plasma cells from immunized mice were cultured as single cells and supernatants were screened for binding to recombinant hOX40L protein to identify anti-hOX40L-antibody producing cells.

Total RNA was collected from hybridomas and plasma cells of interest to generate cDNA and heavy and light variable regions were PCR amplified and sequenced.

The coding sequences for HC and LC of the antibody were generated by DNA synthesis and PCR, subsequently subcloned into an expression vector for protein expression in mammalian cell system. The gene sequences in the expression vectors were confirmed by DNA sequencing.

### Example 2: Production and Purification of Recombinant Anti-hOX40L Antibodies

To produce fully-human anti-hOX40L antibodies, DNA sequences encoding heavy and light variable regions of hybridoma and plasma cells of interest were recombinantly assembled into separate mammalian cell expression vectors with the mutant human IgG1 constant region comprising LALA/YTE mutations (SEQ ID NO: 651) and wild-type human IgK constant region (SEQ ID NO: 738), respectively. ExpiCHO cells were transiently transfected and supernatants containing antibodies were harvested after up to 14 days.

Protein purification by affinity chromatography, and ion exchange chromatography was performed using an AKTA pure instrument (GE Lifesciences). Conditional medium expressing target antibody was harvested by centrifugation at 4000 rpm, 50 min, and filtered with a 0.22 μm filter. The harvested supernatants were loaded to a column of HiTrap Mabselect™ SuRe™ Protein A and polished by size-exclusion chromatography with HiLoad™ 26/200 Superdex™ 200 μg. Alternatively after washing column with Buffer A (PBS, PH 7.4), the protein was eluted with Buffer B (1 M Glycine, pH 2.7), and immediately neutralized with 1/10 volume of Buffer D (1 M sodium citrate, pH 6.0). The affinity purified antibody was then buffer exchanged into 20 mM sodium acetate pH 5.5.

### Example 3: Determination of Antibody Affinity to hOX40L

### Measuring Antibody-OX-40L Binding Kinetics Using Surface Plasmon Resonance

A Biacore 8K SPR system (GE HealthCare) equipped with Series S Sensor Chip Protein G (Cytiva, Cat. 29179315) was used to determine the binding kinetic rate and affinity constants at 25° C. and in a running buffer of HBS-EP+ (10 mM HEPES pH 7.4, 150 mM NaCl, 3 mM EDTA, 0.05% Surfactant P20). Following a stabilization period in running buffer, the anti-OX40L mAb constructs (diluted to 1 μg/mL were captured onto flow cell 2 (active) for 60 sec at a flow rate of 10 uL/min. Recombinant Human OX40L Protein, His Tag (Acro Cat. IL3-H52H4) was prepared at concentrations of 0, 0.39, 0.78, 1.56, 3.13, 6.25, 12.5 and 0 nM and injected over flow cell 1 (reference) and flow cell 2 (active) for 180 sec at a flow rate of 30 μL/min. Recombinant Cynomolgus OX40L Protein, His Tag (SINO BIOLOGICAL, Cat. 11057-C07H) was prepared at concentrations of 0, 0.39, 0.78, 1.56, 3.13, 6.25, 12.5, 25 and 0 nM and injected over flow cell 1 (reference) and flow cell 2 (active) for 180 sec at a flow rate of 30 μL/min. Samples were injected in a multi-cycle manner over freshly captured mAb, by regenerating the capture surfaces with injection of glycine pH 1.5 for 30 sec at a flow rate of 30 μL/min. The data was processed and analyzed with Biacore Insight Evaluation Software Version 2.0.15.12933 (GE Healthcare) as follows. Responses from flow cell 1 (reference) were subtracted from the responses from flow cell 2 (active). The responses from the two buffer blank injections were then subtracted from the reference subtracted data (2-1) to yield double-referenced data, which were fit to an 1:1 binding model to determine the apparent association (ka) and dissociation rate constants (kd). Their ratio provided the apparent equilibrium dissociation constant or affinity constant (KD=kd/ka).

Binding affinity (KD) of antibodies to hOX40L was determined by surface plasmon resonance using Biacore 8K+ (TABLE 7). Briefly, a CM5 Series S sensor chip functionalized with anti-human IgG Fc or anti-mouse IgG Fc antibody by amine coupling was used to capture antibodies at 20 nM. Subsequently, concentrations of hOX40L ranging from 500 nM to 31.25 nM were injected at 30 μL/min. The chip was regenerated with 10 mM glycine pH 1.5 between runs. Results are summarized in TABLE 7.

TABLE 7

| | Human OX40L | | | Cynomolgus OX40L | | |
|---|---|---|---|---|---|---|
| Clone | kₒₙ (M⁻¹ × s⁻¹) | k_{off} (s⁻¹) | K_{D} (M) | kₒₙ (M⁻¹ × s⁻¹) | k_{off} (s⁻¹) | K_{D} (M) |
| 105 | 8.86 × 10⁵ | 1.25 × 10⁻⁴ | 1.41 × 10⁻¹⁰ | 1.64 × 10⁶ | 8.09 × 10⁻⁵ | 4.94 × 10⁻¹¹ |
| 114 | 1.07 × 10⁶ | 1.14 × 10⁻⁴ | 1.06 × 10⁻¹⁰ | 2.30 × 10⁶ | 7.73 × 10⁻⁵ | 3.36 × 10⁻¹¹ |
| 118 | 1.55 × 10⁵ | 2.82 × 10⁻⁵ | 1.82 × 10⁻¹⁰ | n.d. | n.d. | n.d. |
| 122 | 1.51 × 10⁵ | <1.00 × 10⁻⁶ | <6.62 × 10⁻¹² | n.d. | n.d. | n.d. |
| 127 | 1.89 × 10⁵ | 2.10 × 10⁻⁴ | 1.11 × 10⁻⁹ | n.d. | n.d. | n.d. |
| 128 | 1.12 × 10⁵ | <1.00 × 10⁻⁶ | <8.93 × 10⁻¹² | n.d. | n.d. | n.d. |
| 130 | 1.31 × 10⁵ | 1.14 × 10⁻⁵ | 8.73 × 10⁻¹¹ | n.d. | n.d. | n.d. |
| 231 | 9.43 × 10⁵ | 1.30 × 10⁻⁴ | 1.37 × 10⁻¹⁰ | 1.47 × 10⁶ | 1.05 × 10⁻⁴ | 7.14 × 10⁻¹¹ |
| 328 | 2.00 × 10⁶ | 2.71 × 10⁻⁴ | 1.36 × 10⁻¹⁰ | 4.80 × 10⁶ | 1.52 × 10⁻⁴ | 3.16 × 10⁻¹¹ |
| Pos. | 1.58 × 10⁶ | 1.12 × 10⁻⁴ | 7.10 × 10⁻¹¹ | 2.62 × 10⁶ | 1.19 × 10⁻⁴ | 4.55 × 10⁻¹¹ |
| Ctrl. | | | | | | |

n.d.: No data.

### Example 4. Binding of hOX40L-Expressing CHO Cells by Anti-hOX40L Antibodies

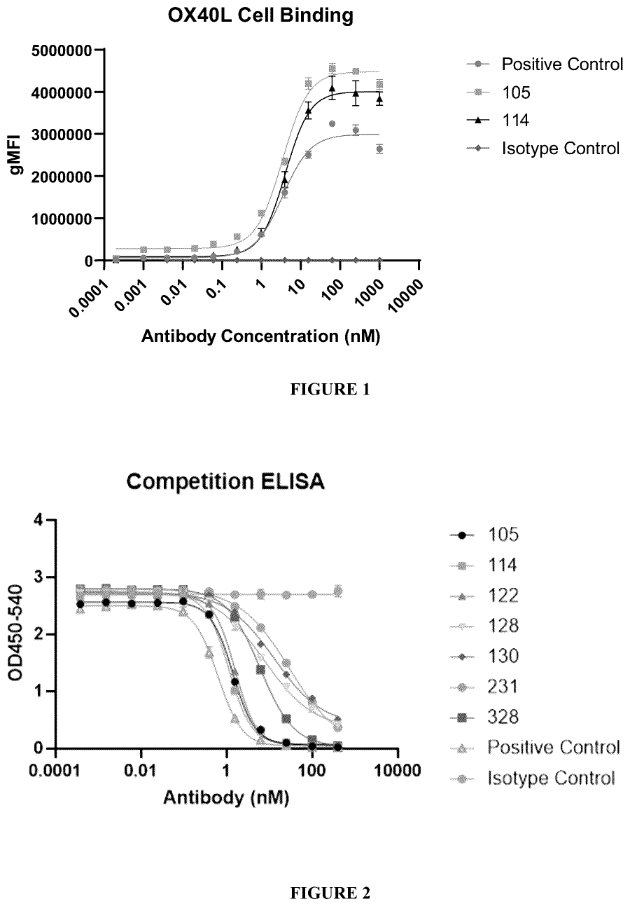

Anti-hOX40L antibodies were assessed for their ability to bind hOX40L-expressing CHO cells. Briefly, CHO cells were transduced to generate a pool of cells overexpressing hOX40L. Single clones were isolated and expanded to select a clone stably expressing hOX40L. A positive control based on the sequences of the anti-hOX40L antibody amlitelimab (called “positive control” throughout the Examples and Figures) was also generated. The cells were incubated with titrated anti-hOX40L antibodies, then with fluorophore-labeled goat anti-human IgG secondary antibody to detect bound antibodies by flow cytometry. Nonlinear regression was performed on geometric mean fluorescence intensity (gMFI) values to determine EC50 values (FIG. 1, TABLE 8). The exemplary anti-hOX40L antibodies bound hOX40L-expressing CHO cells with an EC50 less than 5 nM. Furthermore, clones 105 and 114 exhibited higher gMFI at maximum concentration tested suggesting an increased capacity to bind cell surface OX40L compared to positive control (FIG. 1).

TABLE 8

| Clone | EC₅₀ (nM) |
|---|---|
| 105 | 3.43 |
| 114 | 4.04 |
| Positive Control | 3.49 |

### Example 5: Blocking Activity of Anti-hOX40L Antibodies

Anti-hOX40L antibodies were assessed for their ability to block hOX40L-hOX40 interaction by ELISA. Plates were coated with hOX40-Fc and biotinylated hOX40L preincubated with titrated anti-hOX40L antibodies added to the plates. The binding of hOX40L to hOX40-Fc was detected with Extravidin-HRP. An isotype control was included as a control for no blocking of the binding of hOX40L to hOX40-Fc. Nonlinear regression was performed on optical density (OD) at A450 to determine the IC50 value, which was found to be less than 30 nM for all the tested clones (FIG. 2, TABLE 9).

TABLE 9

| Clone | IC₅₀ (nM) |
|---|---|
| 105 | 1.41 |
| 114 | 1.12 |
| 122 | 1.57 |
| 128 | 7.52 |
| 130 | 12.99 |
| 231 | 28.5 |
| 328 | 5.99 |
| Pos. Ctrl. | 0.62 |
| Iso. Ctrl. | N/A |

N/A: No data.

### Example 6: Functional Activity of Anti-hOX40L Antibodies in hOX40 Reporter Assay

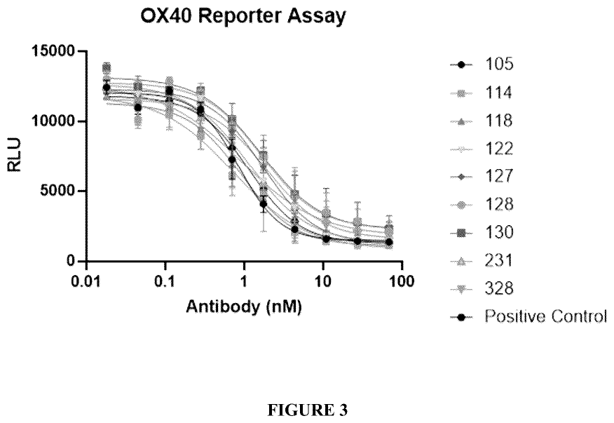

Anti-hOX40L antibodies were assessed for their functional blockade of the OX40-OX40L interaction using an OX40 bioassay. hOX40 cells are commercial cells that overexpress OX40 and have a downstream element that drives luciferase expression under hOX40L stimulation. These cells were used to determine the ability of anti-hOX40L antibodies to block hOX40-mediated signaling. hOX40 cells were incubated with 50 ng/mL hOX40L and titrated anti-hOX40L antibodies for 3-5 hours, and Bio-Glo reagent was added to quantify luminescence (RLU). Nonlinear regression was performed on the luminescence values to determine the IC50 value (FIG. 3, TABLE 10), which were calculated to be less than 5 nM for all the tested clones.

TABLE 10

| Clone | IC₅₀ (nM) |
|---|---|
| 105 | 0.852 |
| 114 | 0.732 |
| 118 | 0.9123 |
| 122 | 1.141 |
| 127 | 1.715 |
| 128 | 1.656 |
| 130 | 1.647 |
| 231 | 0.9087 |
| 328 | 1.898 |
| Pos. Ctrl. | 1.171 |

### Example 7: Functional Activity of Anti-hOX40L Antibodies in Human CD4+ T Cell IL-2 Release Assay

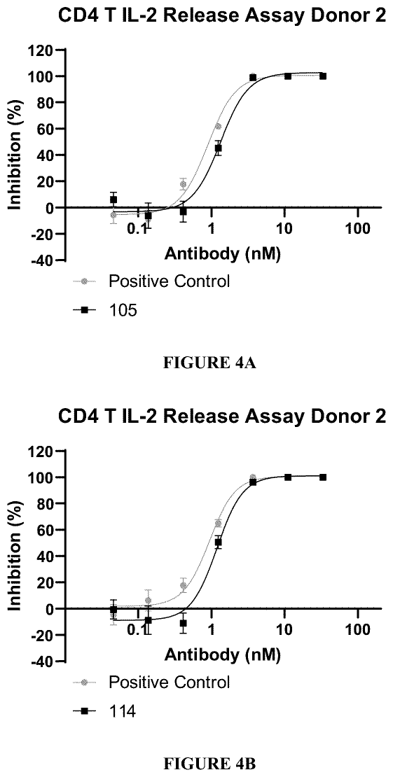

Anti-hOX40L antibodies were assessed for their ability to block the interaction between exogenous OX40L and OX40L expressed on activated CD4+ T cells, thereby inhibiting the release of cytokine interleukin-2 (IL-2). CD4+ T cells were isolated from PBMCs from 3 different donors using an isolation kit, such as EasySep™ Human CD4+ T Cell Isolation Kit. CD4+ T cells were cultured with 2 μg/mL PHA-L and 20 IU/mL recombinant human IL-2 in complete RPMI culture medium for 3 days. Microplates were coated with OKT3 (5 μg/mL) at 4° C. overnight and pre-activated CD4+ T cells were seeded at 3×10⁴ cells per well. Serially-diluted antibodies with human OX40L at final concentration of 20 ng/ml were added and the cells were cultured at 37° C. for 3 days. Microplates were centrifuged and supernatant was collected to measure the amount of IL-2 by ELISA. Nonlinear regression was performed to determine the IC50 values (FIGS. 4A-4B, TABLE 11).

TABLE 11

| | Donor 1 IC₅₀ | Donor 2 IC₅₀ | Donor 3 IC₅₀ |
|---|---|---|---|
| Clone | (nM) | (nM) | (nM) |
| 105 | 1.37 | 1.33 | 1.00 |
| 114 | 1.25 | 1.19 | 1.18 |
| Pos. Ctrl. | 0.96-1.02 | 0.94-0.89 | 0.70-0.49 |

### Example 8. Determination of Antibody Affinity to Human and Cynomolgus FcRn

The binding affinity (KD) of antibodies to human (TABLE 12) and cynomolgus FcRn (TABLE 13) at pH 5.8 and 7.4 was determined by surface plasmon resonance, as described in Example 3, using Biacore 8K+. Briefly, a CM5 Series S sensor chip functionalized with anti-human kappa antibody was used to capture antibodies at 5 μg/mL. Subsequently, concentrations of human FcRn ranging from 1.5 μM to 47 nM and cynomolgus FcRn from 1.0 μM to 31 nM were injected at 30 μL/min. The chip was regenerated with 10 mM glycine pH 1.5 and 10 mM NaOH between runs. The tested anti-OX40L antibodies bound human FcRn and cynomolgus FcRn with similar binding kinetics at pH 5.8.

TABLE 12

| | Human FcRn | | | | | |
|---|---|---|---|---|---|---|
| | pH 5.8 | | | pH 7.4 | | |
| Clone | kₒₙ (M⁻¹ × s⁻¹) | k_{off} (s⁻¹) | K_{D} (M) | kₒₙ (M⁻¹ × s⁻¹) | k_{off} (s⁻¹) | K_{D} (M) |
| 105 | 3.06 × 10⁵ | 4.42 × 10⁻² | 1.45 × 10⁻⁷ | n.b. | n.b. | n.b. |
| 114 | 3.33 × 10⁵ | 4.71 × 10⁻² | 1.41 × 10⁻⁷ | n.b. | n.b. | n.b. |
| 231 | 3.28 × 10⁵ | 4.62 × 10⁻² | 1.41 × 10⁻⁷ | n.b. | n.b. | n.b. |
| 328 | 3.36 × 10⁵ | 4.60 × 10⁻² | 1.37 × 10⁻⁷ | n.b. | n.b. | n.b. |
| Pos. | N/A | N/A | 1.11 × 10⁻⁶ | n.b. | n.b. | n.b. |
| Ctrl. | | | | | | |

n.b.: No binding.

TABLE 13

| | Cynomolgus FcRn | | | | | |
|---|---|---|---|---|---|---|
| | pH 5.8 | | | pH 7.4 | | |
| Clone | kₒₙ (M⁻¹ × s⁻¹) | k_{off} (s⁻¹) | K_{D} (M) | kₒₙ (M⁻¹ × s⁻¹) | k_{off} (s⁻¹) | K_{D} (M) |
| 105 | 3.74 × 10⁵ | 4.53 × 10⁻² | 1.21 × 10⁻⁷ | n.b. | n.b. | n.b. |
| 114 | 3.81 × 10⁵ | 4.45 × 10⁻² | 1.17 × 10⁻⁷ | n.b. | n.b. | n.b. |
| 231 | 3.98 × 10⁵ | 4.65 × 10⁻² | 1.17 × 10⁻⁷ | n.b. | n.b. | n.b. |
| 328 | 3.99 × 10⁵ | 4.62 × 10⁻² | 1.16 × 10⁻⁷ | n.b. | n.b. | n.b. |
| Pos. | N/A | N/A | 9.26 × 10⁻⁷ | n.b. | n.b. | n.b. |
| Ctrl. | | | | | | |

n.b.: No binding.

### Example 9. Determination of Antibody Affinity to Human Fcγ Receptors and C1q

Binding affinity (KD) of antibodies to human Fcγ receptors (CD64, CD32a 167H, CD32a 167R, CD32b, CD16a 176F, CD16a 176V, and CD16b) (TABLE 14) and mean response unit (RU) to C1q at 25 nM (TABLE 15) was determined by surface plasmon resonance, as described in Example 3, using Biacore 8K+. Briefly, a CM5 Series S sensor chip functionalized with anti-human IgG Fc antibody was used to capture antibodies at 5 μg/mL. Subsequently, concentrations of human CD64 ranging from 12.5 nM to 0.3906 nM, CD32a 167H and 167R from 8 μM to 250 nM, CD32b from 18 μM to 562.5 nM, CD16a 176F and 176V from 6 μM to 187.5 nM, CD16b from 10 μM to 312.5 nM, and Clq from 25 nM to 0.7813 nM were injected at 30 μL/min. The chip was regenerated with 10 mM glycine pH 1.5 and 10 mM NaOH between runs. Rituximab, an anti-CD20 antibody, was included for comparison. TABLE 14 shows that while Rituximab displayed measurable binding to the various human Fcγ receptors, the tested anti-OX40L antibodies displayed no significant binding to any of the human Fcγ receptors (TABLE 14). Further, while Rituximab displayed C1q binding, none of the tested anti-OX40L antibodies displayed C1q binding. (TABLE 15).

TABLE 14

| | K_{D} (M) | | | | | | |
|---|---|---|---|---|---|---|---|
| | | CD32a | CD32a | | CD16a | CD16a | |
| Clone | CD64 | 167H | 167R | CD32b | 176F | 176V | CD16b |
| Rituximab | 2.81 × 10⁻¹⁰ | 4.21 × 10⁻⁶ | 2.67 × 10⁻⁶ | 7.40 × 10⁻⁶ | 2.18 × 10⁻⁶ | 8.97 × 10⁻⁷ | 1.48 × 10⁻⁶ |
| 105 | n.b. | n.b. | n.b. | n.b. | n.b. | n.b. | n.b. |
| 114 | n.b. | n.b. | n.b. | n.b. | n.b. | n.b. | n.b. |
| 231 | n.b. | n.b. | n.b. | n.b. | n.b. | n.b. | n.b. |
| 328 | n.b. | n.b. | n.b. | n.b. | n.b. | n.b. | n.b. |

n.b.: No binding.

TABLE 15

| | RU at 25 nM |
|---|---|
| Clone | C1q |
| Rituximab | 386.1 |
| 105 | −3.6 |
| 114 | −4.8 |
| 231 | 1.8 |
| 328 | 0.2 |

### Example 10. Functional Activity of Anti-hOX40L Antibodies in Allogeneic PBMC/CD4+T Mixed Lymphocyte Reaction (MLR)

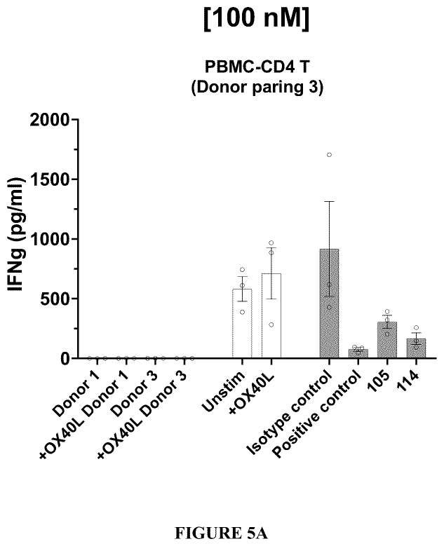

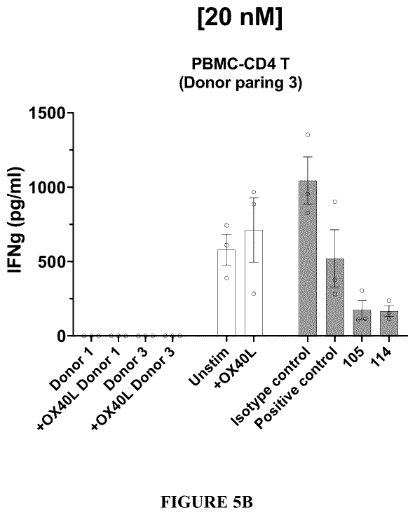

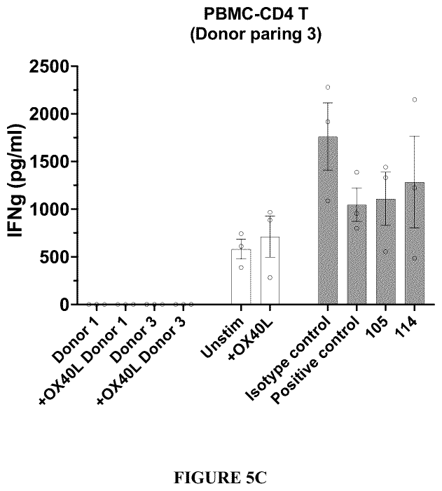

Anti-hOX40L antibodies were assessed for their ability to block the interaction between exogenous OX40L and OX40L expressed on activated CD4+ T cells from human donors, thereby inhibiting the release of interferon gamma (IFNY) in mixed lymphocyte reaction (MLR). PBMCs were preincubated with mitomycin C at 10 μg/mL for 37° C. for 1 h. Allogeneic CD4+ T cells were isolated from PBMCs using an isolation kit, such as EasySep™ Human CD4+ T Cell Isolation Kit. Antibodies (100 nM, 20 nM, or 4 nM), recombinant OX40L (final concentration 1000 ng/ml), mitomycin C-treated PBMCs (1×10⁵ cells/well), and CD4+ t cells (1×10⁵ cells/well) were added to microplates sequentially and co-cultured for 5 days at 37° C. Supernatant was collected and IFNγ was measured by an ELISA detection kit. Data was collected on 6 different donor pairs in triplicate (FIG. 5A-C, TABLE 16). As shown in FIGS. 5A-5C and TABLE 16, tested anti-OX40L antibodies inhibited IFNγ to a similar extent as the positive control at higher antibody concentrations, such as 100 nM.

TABLE 16
Measurement of IFNγ concentration

| | | | | | | | | | | MLR + OX40L | | | | | | | |
|---|---|---|---|---|---|---|---|---|---|---|---|---|---|---|---|---|---|
| | | PBMC Donor | | CD4-T Donor | | | | | | | | | | | | | |
| | TA | alone + | | alone + | | | | | | | | Positive | | | | | |
| Donor | conc | OX40L | | OX40L | | MLR | | MLR + OX40L | | Isotype | | control | | 105 | | 114 | |
| pairing | (nM) | Mean | SEM | Mean | SEM | Mean | SEM | Mean | SEM | Mean | SEM | Mean | SEM | Mean | SEM | Mean | SEM |
| 3 | 100 | 0 | 0 | 0 | 0 | 581 | 103.4 | 712 | 215.9 | 916.1 | 398.0 | 75.4 | 15.4 | 305.5 | 55.5 | 165.6 | 48.6 |
| | 20 | | | | | | | | | 1044.6 | 158.5 | 520.0 | 192.8 | 175.5 | 64.9 | 166.8 | 36.3 |
| | 4 | | | | | | | | | 1760.6 | 353.5 | 1046.4 | 176.1 | 1108.3 | 279.1 | 1284.7 | 482.0 |
| 4 | 100 | 0 | 0 | 2 | 2 | 487 | 149.2 | 1209 | 230.0 | 623.9 | 285.0 | 161.2 | 21.6 | 408.5 | 117.2 | 216.8 | 49.0 |
| | 20 | | | | | | | | | 754.6 | 91.2 | 423.6 | 90.1 | 256.8 | 68.2 | 128.5 | 72.1 |
| | 4 | | | | | | | | | 1060.8 | 158.8 | 1166.8 | 403.5 | 1114.5 | 271.6 | 628.5 | 80.7 |
| 1 | 100 | 0 | 0 | 0 | 0 | 162 | 85.1 | 210 | 48.8 | 362.2 | 189.9 | 145.2 | 38.7 | 18.4 | 4.9 | 32.0 | 7.1 |
| | 20 | | | | | | | | | 185.2 | 49.7 | 41.4 | 15.5 | 73.4 | 15.9 | 29.2 | 10.6 |
| | 4 | | | | | | | | | 249.7 | 163.0 | 201.2 | 88.7 | 238.5 | 181.7 | 250.8 | 88.3 |
| 5 | 100 | 2 | 1 | 1 | 1 | 1087 | 277.6 | 1822 | 357.3 | 1940.6 | 308.7 | 877.9 | 219.3 | 398.3 | 100.0 | 677.1 | 261.3 |
| | 20 | | | | | | | | | 1495.9 | 39.6 | 780.3 | 164.9 | 519.0 | 84.9 | 790.6 | 254.2 |
| | 4 | | | | | | | | | 1916.0 | 334.1 | 1347.8 | 242.6 | 1704.9 | 390.6 | 1514.9 | 280.7 |
| 6 | 100 | 0 | 0 | 0 | 0 | 1422 | 906.3 | 1814 | 446.1 | 689.4 | 115.0 | 202.3 | 18.4 | 224.9 | 122.9 | 260.2 | 44.8 |
| | 20 | | | | | | | | | 1603.7 | 675.3 | 279.0 | 81.0 | 353.2 | 291.6 | 771.7 | 440.1 |
| | 4 | | | | | | | | | 544.6 | 107.5 | 780.3 | 307.7 | 528.4 | 103.6 | 868.0 | 315.0 |
| 8 | 100 | 0 | 0 | 0 | 0 | 22 | 8.9 | 15 | 5.4 | 120.4 | 51.1 | 92.5 | 44.4 | 53.6 | 3.5 | 78.9 | 20.6 |
| | 20 | | | | | | | | | 335.5 | 37.7 | 10.8 | 6.5 | 24.0 | 9.6 | 20.8 | 7.2 |
| | 4 | | | | | | | | | 24.1 | 10.3 | 17.3 | 10.0 | 159.0 | 72.7 | 40.6 | 28.9 |

TA conc. = Test antibody concentration; nM
Mean = Mean IFNγ concentration, pg/ml
SEM = Standard error of the mean

### Example 11. Pharmacokinetic Profiles of Anti-hOX40L Antibodies in Cynomolgus Monkeys

Anti-hOX40L antibodies were evaluated for their pharmacokinetic profiles following single intravenous (IV) or subcutaneous (SC) administration to naïve male cynomolgus monkeys as shown in TABLE 17. An n of 4 was used for each group.

TABLE 17

| | | | | | Fc |
|---|---|---|---|---|---|
| Group # | Clone | Route | Dosage | Isotype | Mutations |
| Group 1 | 105 | IV | 50.29 mg/kg | IgG1 | LALA YTE |
| Group 2 | 105 | SC | 50.29 mg/kg | IgG1 | LALA YTE |
| Group 3 | 114 | IV | 49.99 mg/kg | IgG1 | LALA YTE |
| Group 4 | 114 | SC | 49.99 mg/kg | IgG1 | LALA YTE |
| Group 5 | 231 | IV | 50.79 mg/kg | IgG1 | LALA YTE |
| Group 6 | 231 | SC | 50.79 mg/kg | IgG1 | LALA YTE |
| Group 7 | 328 | IV | 51.54 mg/kg | IgG1 | LALA YTE |
| Group 8 | 328 | SC | 51.54 mg/kg | IgG1 | LALA YTE |
| Group 9 | Pos. Ctrl. | IV | 51.27 mg/kg | IgG4 | SPLE |
| Group 10 | Pos. Ctrl. | SC | 51.27 mg/kg | IgG4 | SPLE |

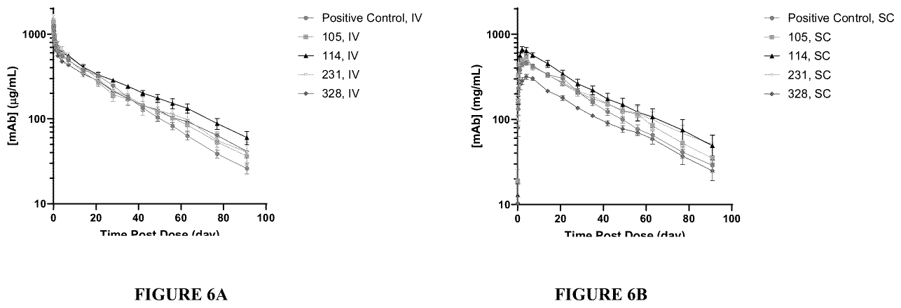

Serum samples were collected from individual animals pre-dose, and at the time points post dose of 10 min, 1 h, 4 h, 8 h, 24 h, and days 2, 4, 7, 14, 21, 28, 35, 42, 49, 56, 63, 77, and 91. Standard curves were generated for each antibody, which were diluted in 2-10% pooled monkey serum, by ELISA to calculate antibody concentration based on ELISA measurements. Three different antibody concentrations were tested by ELISA for quality control purposes of the accuracy of the standard curves. PK analyses were performed on serum concentration versus time data using linear trapezoidal rule for AUC calculations (TABLE 18, FIG. 6A, FIG. 6B). Nominal dose values and sampling times were used for calculations. Bioavailability was calculated by dividing the mean dose-normalized AUC_{0-inf} following subcutaneous administration by the mean dose-normalized AUC_{0-inf} following intravenous administration. The normalization of doses was done using doses based on measured formulation concentration and body weights of the subjects. For PK calculations and serum concentration descriptive statistics, any concentration reported as below the limit of quantification (BLQ) was set equal to zero. As shown in FIG. 6A and FIG. 6B, the tested antibodies exhibited a longer half-life than did the positive control.

TABLE 18

| | | | Mean (SEM) | | | | | | |
|---|---|---|---|---|---|---|---|---|---|
| Test | Dose | | tmax | Cmax | AUC0-inf | Cl | Vss | F | Half-life |
| Article | (mg/kg) | Route | (days) | (μg/mL) | (h × μg/mL) | (mL/day/kg) | (mL/kg) | (%) | (days) |
| Positive | 51.27 | IV | 0.06 (0.05) | 1249.60 (65.75) | 18276.89 (1480.79) | 2.77 (0.22) | 75.11 (4.33) | N/A | 19.77 (0.81) |
| Control | 51.27 | SC | 4.25 (1.03) | 465.36 (28.12) | 15958.16 (1225.03) | 3.47 (0.30) | 109.14 (5.18) | 92 | 22.19 (1.84) |
| 105 | 50.29 | IV | 0.007 (0) | 1363.16 (104.11) | 18925.43 (2197.26) | 2.74 (0.28) | 85.01 (6.36) | N/A | 23.50 (0.95) |
| | 50.29 | SC | 3.50 (0.50) | 549.63 (24.72) | 17299.22 (738.65) | 2.90 (0.11) | 98.47 (7.31) | 100 | 23.42 (1.09) |
| 114 | 49.99 | IV | 0.007 (0) | 1212.64 (45.60) | 24608.55 (1753.10) | 2.06 (0.14) | 77.21 (2.09) | N/A | 26.90 (0.92) |
| | 49.99 | SC | 3.00 (0.58) | 679.59 (48.45) | 22688.43 (3012.04) | 2.41 (0.27) | 85.45 (10.45) | 96 | 25.51 (3.86) |
| 231 | 50.79 | IV | 0.007 (0) | 1620.72 (47.87) | 21295.44 (2697.41) | 2.01 (0.76) | 55.01 (19.79) | N/A | 23.95 (4.78) |
| | 50.79 | SC | 2.75 (0.75) | 507.76 (16.98) | 18969.47 (2565.65) | 3.30 (0.60) | 125.89 (5.91) | 86 | 27.98 (2.97) |
| 328 | 51.54 | IV | 0.007 (0) | 1277.44 (51.40) | 18778.25 (366.43) | 2.67 (0.05) | 95.45 (4.09) | N/A | 26.57 (0.83) |
| | 51.54 | SC | 4.75 (0.75) | 320.61 (21.14) | 11486.47 (844.16) | 7.04 (0.52) | 256.21 (17.13) | 63 | 25.75 (3.03) |

Tmax = peak time
Cmax = peak concentration
AUC = area under the curve
F = fraction absorbed
CL = clearance
Vss = volume of distribution under steady state conditions

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

Informal sequence listing

| | | SEQ ID |
|---|---|---|
| Description | Sequence | NO |
| Kabat HCDR1 | NYAMT | SEQ ID |
| | | NO: 1 |
| Kabat HCDR1 | SYAMN | SEQ ID |
| | | NO: 2 |
| Kabat HCDR1 | SNYMS | SEQ ID |
| | | NO: 3 |
| Kabat HCDR1 | NYWMH | SEQ ID |
| | | NO: 4 |
| Kabat HCDR1 | NAWMS | SEQ ID |
| | | NO: 5 |
| Kabat HCDR1 | NAWMT | SEQ ID |
| | | NO: 6 |
| Kabat HCDR1 | YAWMS | SEQ ID |
| | | NO: 7 |
| Kabat HCDR1 | NYDIN | SEQ ID |
| | | NO: 8 |
| Kabat HCDR1 | SYAMY | SEQ ID |
| | | NO: 9 |
| Kabat HCDR1 | SYAMT | SEQ ID |
| | | NO: 10 |
| Kabat HCDR1 | SYAMS | SEQ ID |
| | | NO: 11 |
| Kabat HCDR1 | TYAMT | SEQ ID |
| | | NO: 12 |
| Kabat HCDR1 | SHAMT | SEQ ID |
| | | NO: 13 |
| Kabat HCDR1 | IYAMT | SEQ ID |
| | | NO: 14 |
| Kabat HCDR1 | SYWMH | SEQ ID |
| | | NO: 15 |
| Kabat HCDR1 | SFWMH | SEQ ID |
| | | NO: 16 |
| Kabat HCDR1 | SAWMS | SEQ ID |
| | | NO: 17 |
| Kabat HCDR1 | NAWMR | SEQ ID |
| | | NO: 18 |
| Kabat HCDR1 | YAWMT | SEQ ID |
| | | NO: 19 |
| Kabat HCDR1 | NAWMN | SEQ ID |
| | | NO: 20 |
| Kabat HCDR1 | TAWMS | SEQ ID |
| | | NO: 21 |
| Chothia HCDR1 | GFTFSNY | SEQ ID |
| | | NO: 22 |
| Chothia HCDR1 | GFRFSSY | SEQ ID |
| | | NO: 23 |
| Chothia HCDR1 | GFPVSSN | SEQ ID |
| | | NO: 24 |
| Chothia HCDR1 | GFTFSYA | SEQ ID |
| | | NO: 25 |
| Chothia HCDR1 | GYTFTNY | SEQ ID |
| | | NO: 26 |
| Chothia HCDR1 | GFTFSSY | SEQ ID |
| | | NO: 27 |
| Chothia HCDR1 | GFTFRNY | SEQ ID |
| | | NO: 28 |
| Chothia HCDR1 | GFTFNTY | SEQ ID |
| | | NO: 29 |
| Chothia HCDR1 | GFTFSSH | SEQ ID |
| | | NO: 30 |
| Chothia HCDR1 | GFTFGNY | SEQ ID |
| | | NO: 31 |
| Chothia HCDR1 | GFTFTNY | SEQ ID |
| | | NO: 32 |
| Chothia HCDR1 | GFTFSIY | SEQ ID |
| | | NO: 33 |
| Chothia HCDR1 | GFTFRSY | SEQ ID |
| | | NO: 34 |
| Chothia HCDR1 | GLTFSNY | SEQ ID |
| | | NO: 35 |
| Chothia HCDR1 | GFTFSSF | SEQ ID |
| | | NO: 36 |
| Chothia HCDR1 | GFTFHNA | SEQ ID |
| | | NO: 37 |
| Chothia HCDR1 | GFTFNSA | SEQ ID |
| | | NO: 38 |
| Chothia HCDR1 | GFIFNNA | SEQ ID |
| | | NO: 39 |
| Chothia HCDR1 | GFTFNYA | SEQ ID |
| | | NO: 40 |
| Chothia HCDR1 | GFTFINA | SEQ ID |
| | | NO: 41 |
| Chothia HCDR1 | GFTFSTA | SEQ ID |
| | | NO: 42 |
| Chothia HCDR1 | GFIFSNA | SEQ ID |
| | | NO: 43 |
| IMGT CDR1 | GFTFSNYA | SEQ ID |
| | | NO: 44 |
| IMGT CDR1 | GFRFSSYA | SEQ ID |
| | | NO: 45 |
| IMGT CDR1 | GFPVSSNY | SEQ ID |
| | | NO: 46 |
| IMGT CDR1 | GFTFSNYW | SEQ ID |
| | | NO: 47 |
| IMGT CDR1 | GFTFSNAW | SEQ ID |
| | | NO: 48 |
| IMGT CDR1 | GFTFNNAW | SEQ ID |
| | | NO: 49 |
| IMGT CDR1 | GFTFSYAW | SEQ ID |
| | | NO: 50 |
| IMGT CDR1 | GYTFTNYD | SEQ ID |
| | | NO: 51 |
| IMGT CDR1 | GFTFSSYA | SEQ ID |
| | | NO: 52 |
| IMGT CDR1 | GFTFRNYA | SEQ ID |
| | | NO: 53 |
| IMGT CDR1 | GFTFNTYA | SEQ ID |
| | | NO: 54 |
| IMGT CDR1 | GFTFSSHA | SEQ ID |
| | | NO: 55 |
| IMGT CDR1 | GFTFGNYA | SEQ ID |
| | | NO: 56 |
| IMGT CDR1 | GFTFTNYA | SEQ ID |
| | | NO: 57 |
| IMGT CDR1 | GFTFSIYA | SEQ ID |
| | | NO: 58 |
| IMGT CDR1 | GFTFRSYW | SEQ ID |
| | | NO: 59 |
| IMGT CDR1 | GLTFSNYW | SEQ ID |
| | | NO: 60 |
| IMGT CDR1 | GFTFSSFW | SEQ ID |
| | | NO: 61 |
| IMGT CDR1 | GFTFSSYW | SEQ ID |
| | | NO: 62 |
| IMGT CDR1 | GFTFHNAW | SEQ ID |
| | | NO: 63 |
| IMGT CDR1 | GFTFNSAW | SEQ ID |
| | | NO: 64 |
| IMGT CDR1 | GFIFNNAW | SEQ ID |
| | | NO: 65 |
| IMGT CDR1 | GFTFNYAW | SEQ ID |
| | | NO: 66 |
| IMGT CDR1 | GFTFINAW | SEQ ID |
| | | NO: 67 |
| IMGT CDR1 | GFTFSTAW | SEQ ID |
| | | NO: 68 |
| IMGT CDR1 | GFIFSNAW | SEQ ID |
| | | NO: 69 |
| Kabat HCDR2 | LISGSGGLTKYADSVKG | SEQ ID |
| | | NO: 70 |
| Kabat HCDR2 | IIYSRDSTHYADSVKG | SEQ ID |
| | | NO: 71 |
| Kabat HCDR2 | RINSDGISTRYADSVKG | SEQ ID |
| | | NO: 72 |
| Kabat HCDR2 | RIKSNSYGGTTDYAAPAIG | SEQ ID |
| | | NO: 73 |
| Kabat HCDR2 | RIKSISYGGTTDSAAPVKG | SEQ ID |
| | | NO: 74 |
| Kabat HCDR2 | RIKSETDGGTTDYAAPVKG | SEQ ID |
| | | NO: 75 |
| Kabat HCDR2 | WMNPHSGQTGYAQKFQG | SEQ ID |
| | | NO: 76 |
| Kabat HCDR2 | LITGSGGSTHYADSVKG | SEQ ID |
| | | NO: 77 |
| Kabat HCDR2 | LISGSGGITKYADSVKG | SEQ ID |
| | | NO: 78 |
| Kabat HCDR2 | LISGSGGITHYADSVKG | SEQ ID |
| | | NO: 79 |
| Kabat HCDR2 | LISGSGGVTHYADSVKG | SEQ ID |
| | | NO: 80 |
| Kabat HCDR2 | LISGSGAITHYADSVKG | SEQ ID |
| | | NO: 81 |
| Kabat HCDR2 | LISGSGGLTNYADSVKG | SEQ ID |
| | | NO: 82 |
| Kabat HCDR2 | LISGSGGSTHYADSVKG | SEQ ID |
| | | NO: 83 |
| Kabat HCDR2 | LISGSGGLTKYADSVRG | SEQ ID |
| | | NO: 84 |
| Kabat HCDR2 | LISGSGSLTKYADSVKG | SEQ ID |
| | | NO: 85 |
| Kabat HCDR2 | LISGGGGLTYYADSVKG | SEQ ID |
| | | NO: 86 |
| Kabat HCDR2 | LISGSGGLTYYADSVKG | SEQ ID |
| | | NO: 87 |
| Kabat HCDR2 | RIDNNGINTRYADSVKG | SEQ ID |
| | | NO: 88 |
| Kabat HCDR2 | RISSDGISTRYADSVKG | SEQ ID |
| | | NO: 89 |
| Kabat HCDR2 | RINNDGISTRYADSVKG | SEQ ID |
| | | NO: 90 |
| Kabat HCDR2 | RINSDGFSTRYADSVKG | SEQ ID |
| | | NO: 91 |
| Kabat HCDR2 | RIKGVTAGGTADYAAPVKG | SEQ ID |
| | | NO: 92 |
| Kabat HCDR2 | RVKSVTAGGTTDYAAPVKG | SEQ ID |
| | | NO: 93 |
| Kabat HCDR2 | RIKSKTDGGTTDYAAPVKG | SEQ ID |
| | | NO: 94 |
| Kabat HCDR2 | RIKSVTTGGTTDYAAPVKG | SEQ ID |
| | | NO: 95 |
| Kabat HCDR2 | RIKSVTAGGTTDYAAPVKG | SEQ ID |
| | | NO: 96 |
| Kabat HCDR2 | RIKSVTAGGTTDFPAPVKG | SEQ ID |
| | | NO: 97 |
| Kabat HCDR2 | HIKSETDGGTTDYAAPVKG | SEQ ID |
| | | NO: 98 |
| Kabat HCDR2 | RIKRKTDGGTTDYAAPVQG | SEQ ID |
| | | NO: 99 |
| Kabat HCDR2 | RIKSLTDGGTTDYAAPVKG | SEQ ID |
| | | NO: 100 |
| Kabat HCDR2 | RIKRKTDGGTREYAAPVKG | SEQ ID |
| | | NO: 101 |
| Kabat HCDR2 | RIKRNTDGGTTDYAAPVKG | SEQ ID |
| | | NO: 102 |
| Kabat HCDR2 | RIKTNTDGGTTDYAAPVKG | SEQ ID |
| | | NO: 103 |
| Kabat HCDR2 | RIKSKTYGGTTDYDAPVKG | SEQ ID |
| | | NO: 104 |
| Kabat HCDR2 | RIKSQTYGGTTDYAAPVKG | SEQ ID |
| | | NO: 105 |
| Kabat HCDR2 | RIKSKTYGGTTDYAAPVKG | SEQ ID |
| | | NO: 106 |
| Kabat HCDR2 | RIKRKTDGGTRDYAAPVKG | SEQ ID |
| | | NO: 107 |
| Kabat HCDR2 | RIKRETDGGTTDYASPVKG | SEQ ID |
| | | NO: 108 |
| Kabat HCDR2 | RIKRKSDGGTTDYAAPVKG | SEQ ID |
| | | NO: 109 |
| Kabat HCDR2 | RIKSEIDGGTTDYAAPVKG | SEQ ID |
| | | NO: 110 |
| Chothia HCDR2 | SGSGGL | SEQ ID |
| | | NO: 111 |
| Chothia HCDR2 | YSRDS | SEQ ID |
| | | NO: 112 |
| Chothia HCDR2 | NSDGIS | SEQ ID |
| | | NO: 113 |
| Chothia HCDR2 | KSNSYGGT | SEQ ID |
| | | NO: 114 |
| Chothia HCDR2 | KSISYGGT | SEQ ID |
| | | NO: 115 |
| Chothia HCDR2 | KSETDGGT | SEQ ID |
| | | NO: 116 |
| Chothia HCDR2 | NPHSGQ | SEQ ID |
| | | NO: 117 |
| Chothia HCDR2 | TGSGGS | SEQ ID |
| | | NO: 118 |
| Chothia HCDR2 | SGSGGI | SEQ ID |
| | | NO: 119 |
| Chothia HCDR2 | SGSGGV | SEQ ID |
| | | NO: 120 |
| Chothia HCDR2 | SGSGAI | SEQ ID |
| | | NO: 121 |
| Chothia HCDR2 | SGSGGS | SEQ ID |
| | | NO: 122 |
| Chothia HCDR2 | SGSGSL | SEQ ID |
| | | NO: 123 |
| Chothia HCDR2 | SGGGGL | SEQ ID |
| | | NO: 124 |
| Chothia HCDR2 | DNNGIN | SEQ ID |
| | | NO: 125 |
| Chothia HCDR2 | SSDGIS | SEQ ID |
| | | NO: 126 |
| Chothia HCDR2 | NNDGIS | SEQ ID |
| | | NO: 127 |
| Chothia HCDR2 | NSDGFS | SEQ ID |
| | | NO: 128 |
| Chothia HCDR2 | KGVTAGGT | SEQ ID |
| | | NO: 129 |
| Chothia HCDR2 | KSVTAGGT | SEQ ID |
| | | NO: 130 |
| Chothia HCDR2 | KSKTDGGT | SEQ ID |
| | | NO: 131 |
| Chothia HCDR2 | KSVTTGGT | SEQ ID |
| | | NO: 132 |
| Chothia HCDR2 | KRKTDGGT | SEQ ID |
| | | NO: 133 |
| Chothia HCDR2 | KSLTDGGT | SEQ ID |
| | | NO: 134 |
| Chothia HCDR2 | KRNTDGGT | SEQ ID |
| | | NO: 135 |
| Chothia HCDR2 | KRNTDGGT | SEQ ID |
| | | NO: 136 |
| Chothia HCDR2 | KSKTYGGT | SEQ ID |
| | | NO: 137 |
| Chothia HCDR2 | KSQTYGGT | SEQ ID |
| | | NO: 138 |
| Chothia HCDR2 | KRETDGGT | SEQ ID |
| | | NO: 139 |
| Chothia HCDR2 | KRKSDGGT | SEQ ID |
| | | NO: 140 |
| Chothia HCDR2 | KSEIDGGT | SEQ ID |
| | | NO: 141 |
| IMGT HCDR2 | ISGSGGLT | SEQ ID |
| | | NO: 142 |
| IMGT HCDR2 | IYSRDST | SEQ ID |
| | | NO: 143 |
| IMGT HCDR2 | INSDGIST | SEQ ID |
| | | NO: 144 |
| IMGT HCDR2 | IKSNSYGGTT | SEQ ID |
| | | NO: 145 |
| IMGT HCDR2 | IKSISYGGTT | SEQ ID |
| | | NO: 146 |
| IMGT HCDR2 | IKSETDGGTT | SEQ ID |
| | | NO: 147 |
| IMGT HCDR2 | MNPHSGQT | SEQ ID |
| | | NO: 148 |
| IMGT HCDR2 | ITGSGGST | SEQ ID |
| | | NO: 149 |
| IMGT HCDR2 | ISGSGGIT | SEQ ID |
| | | NO: 150 |
| IMGT HCDR2 | ISGSGGVT | SEQ ID |
| | | NO: 151 |
| IMGT HCDR2 | ISGSGAIT | SEQ ID |
| | | NO: 152 |
| IMGT HCDR2 | ISGSGGST | SEQ ID |
| | | NO: 153 |
| IMGT HCDR2 | ISGSGSLT | SEQ ID |
| | | NO: 154 |
| IMGT HCDR2 | ISGGGGLT | SEQ ID |
| | | NO: 155 |
| IMGT HCDR2 | IDNNGINT | SEQ ID |
| | | NO: 156 |
| IMGT HCDR2 | ISSDGIST | SEQ ID |
| | | NO: 157 |
| IMGT HCDR2 | INNDGIST | SEQ ID |
| | | NO: 158 |
| IMGT HCDR2 | INSDGFST | SEQ ID |
| | | NO: 159 |
| IMGT HCDR2 | IKGVTAGGTA | SEQ ID |
| | | NO: 160 |
| IMGT HCDR2 | VKSVTAGGTT | SEQ ID |
| | | NO: 161 |
| IMGT HCDR2 | IKSKTDGGTT | SEQ ID |
| | | NO: 162 |
| IMGT HCDR2 | IKSVTTGGTT | SEQ ID |
| | | NO: 163 |
| IMGT HCDR2 | IKSVTAGGTT | SEQ ID |
| | | NO: 164 |
| IMGT HCDR2 | IKRKTDGGTT | SEQ ID |
| | | NO: 165 |
| IMGT HCDR2 | IKSLTDGGTT | SEQ ID |
| | | NO: 166 |
| IMGT HCDR2 | IKRKTDGGTR | SEQ ID |
| | | NO: 167 |
| IMGT HCDR2 | IKRNTDGGTT | SEQ ID |
| | | NO: 168 |
| IMGT HCDR2 | IKTNTDGGTT | SEQ ID |
| | | NO: 169 |
| IMGT HCDR2 | IKSKTYGGTT | SEQ ID |
| | | NO: 170 |
| IMGT HCDR2 | IKSQTYGGTT | SEQ ID |
| | | NO: 171 |
| IMGT HCDR2 | IKRETDGGTT | SEQ ID |
| | | NO: 172 |
| IMGT HCDR2 | IKRKSDGGTT | SEQ ID |
| | | NO: 173 |
| IMGT HCDR2 | IKSEIDGGTT | SEQ ID |
| | | NO: 174 |
| Kabat HCDR3 | DEGLTTGEY | SEQ ID |
| | | NO: 175 |
| Kabat HCDR3 | ERWEPSFHY | SEQ ID |
| | | NO: 176 |
| Kabat HCDR3 | TLTYYYGAGSDY | SEQ ID |
| | | NO: 177 |
| Kabat HCDR3 | DFQLELPDY | SEQ ID |
| | | NO: 178 |
| Kabat HCDR3 | DHPKFGFDF | SEQ ID |
| | | NO: 179 |
| Kabat HCDR3 | GSVWKYGGSLDF | SEQ ID |
| | | NO: 180 |
| Kabat HCDR3 | GSSWNVYFFDF | SEQ ID |
| | | NO: 181 |
| Kabat HCDR3 | EGYDLWSGDSSNWLDP | SEQ ID |
| | | NO: 182 |
| Kabat HCDR3 | EGYDLWSGESSNWLDP | SEQ ID |
| | | NO: 183 |
| Kabat HCDR3 | EGYDLWSGHSSNWLDP | SEQ ID |
| | | NO: 184 |
| Kabat HCDR3 | EGYDLWSGYSSNWLDP | SEQ ID |
| | | NO: 185 |
| Kabat HCDR3 | DEGLTTGEF | SEQ ID |
| | | NO: 186 |
| Kabat HCDR3 | DEGLTTGES | SEQ ID |
| | | NO: 187 |
| Kabat HCDR3 | EEGLTTGEY | SEQ ID |
| | | NO: 188 |
| Kabat HCDR3 | DEGLTSGEY | SEQ ID |
| | | NO: 189 |
| Kabat HCDR3 | DFQLELRDY | SEQ ID |
| | | NO: 190 |
| Kabat HCDR3 | DFQLELRDF | SEQ ID |
| | | NO: 191 |
| Kabat HCDR3 | DHPKFGFDY | SEQ ID |
| | | NO: 192 |
| Kabat HCDR3 | GGVWNYGGSLDF | SEQ ID |
| | | NO: 193 |
| Kabat HCDR3 | GGVWKYGGSLDF | SEQ ID |
| | | NO: 194 |
| Kabat HCDR3 | GSVWNYGGSLDF | SEQ ID |
| | | NO: 195 |
| Kabat HCDR3 | GSVWNYGGSLDY | SEQ ID |
| | | NO: 196 |
| Kabat HCDR3 | GSVWKYGGSLDY | SEQ ID |
| | | NO: 197 |
| Kabat HCDR3 | GSVWRYGGSLDF | SEQ ID |
| | | NO: 198 |
| Kabat HCDR3 | GSSWHVYFFDF | SEQ ID |
| | | NO: 199 |
| Kabat HCDR3 | GSSWSLYYFDF | SEQ ID |
| | | NO: 200 |
| Kabat HCDR3 | GSSWYVYYFDY | SEQ ID |
| | | NO: 201 |
| Kabat HCDR3 | GSSWYVYYFDF | SEQ ID |
| | | NO: 202 |
| Chothia HCDR3 | EGLTTGE | SEQ ID |
| | | NO: 203 |
| Chothia HCDR3 | RWEPSFH | SEQ ID |
| | | NO: 204 |
| Chothia HCDR3 | LTYYYGAGSD | SEQ ID |
| | | NO: 205 |
| Chothia HCDR3 | FQLELPD | SEQ ID |
| | | NO: 206 |
| Chothia HCDR3 | HPKFGFD | SEQ ID |
| | | NO: 207 |
| Chothia HCDR3 | SVWKYGGSLD | SEQ ID |
| | | NO: 208 |
| Chothia HCDR3 | SSWNVYFFD | SEQ ID |
| | | NO: 209 |
| Chothia HCDR3 | GYDLWSGDSSNWLD | SEQ ID |
| | | NO: 210 |
| Chothia HCDR3 | GYDLWSGESSNWLD | SEQ ID |
| | | NO: 211 |
| Chothia HCDR3 | GYDLWSGHSSNWLD | SEQ ID |
| | | NO: 212 |
| Chothia HCDR3 | GYDLWSGYSSNWLD | SEQ ID |
| | | NO: 213 |
| Chothia HCDR3 | EGLTSGE | SEQ ID |
| | | NO: 214 |
| Chothia HCDR3 | FQLELRD | SEQ ID |
| | | NO: 215 |
| Chothia HCDR3 | GVWNYGGSLD | SEQ ID |
| | | NO: 216 |
| Chothia HCDR3 | GVWKYGGSLD | SEQ ID |
| | | NO: 217 |
| Chothia HCDR3 | SVWNYGGSLD | SEQ ID |
| | | NO: 218 |
| Chothia HCDR3 | SVWRYGGSLD | SEQ ID |
| | | NO: 219 |
| Chothia HCDR3 | SSWHVYFFD | SEQ ID |
| | | NO: 220 |
| Chothia HCDR3 | SSWSLYYFD | SEQ ID |
| | | NO: 221 |
| Chothia HCDR3 | SSWYVYYFD | SEQ ID |
| | | NO: 222 |
| IMGT HCDR3 | VKDEGLTTGEY | SEQ ID |
| | | NO: 223 |
| IMGT HCDR3 | AKERWEPSFHY | SEQ ID |
| | | NO: 224 |
| IMGT HCDR3 | AGTLTYYYGAGSDY | SEQ ID |
| | | NO: 225 |
| IMGT HCDR3 | VRDFQLELPDY | SEQ ID |
| | | NO: 226 |
| IMGT HCDR3 | TTDHPKFGFDF | SEQ ID |
| | | NO: 227 |
| IMGT HCDR3 | STGSVWKYGGSLDF | SEQ ID |
| | | NO: 228 |
| IMGT HCDR3 | TTGSSWNVYFFDF | SEQ ID |
| | | NO: 229 |
| IMGT HCDR3 | AREGYDLWSGDSSNWLDP | SEQ ID |
| | | NO: 230 |
| IMGT HCDR3 | AREGYDLWSGESSNWLDP | SEQ ID |
| | | NO: 231 |
| IMGT HCDR3 | AREGYDLWSGHSSNWLDP | SEQ ID |
| | | NO: 232 |
| IMGT HCDR3 | AREGYDLWSGYSSNWLDP | SEQ ID |
| | | NO: 233 |
| IMGT HCDR3 | AKDEGLTTGEY | SEQ ID |
| | | NO: 234 |
| IMGT HCDR3 | VKDEGLTTGEF | SEQ ID |
| | | NO: 235 |
| IMGT HCDR3 | VKDEGLTTGES | SEQ ID |
| | | NO: 236 |
| IMGT HCDR3 | AKEEGLTTGEY | SEQ ID |
| | | NO: 237 |
| IMGT HCDR3 | AKDEGLTSGEY | SEQ ID |
| | | NO: 238 |
| IMGT HCDR3 | VKEEGLTTGEY | SEQ ID |
| | | NO: 239 |
| IMGT HCDR3 | VRDFQLELRDY | SEQ ID |
| | | NO: 240 |
| IMGT HCDR3 | VSDFQLELPDY | SEQ ID |
| | | NO: 241 |
| IMGT HCDR3 | VRDFQLELRDF | SEQ ID |
| | | NO: 242 |
| IMGT HCDR3 | TTDHPKFGFDY | SEQ ID |
| | | NO: 243 |
| IMGT HCDR3 | TTGGVWNYGGSLDF | SEQ ID |
| | | NO: 244 |
| IMGT HCDR3 | ITGGVWNYGGSLDF | SEQ ID |
| | | NO: 245 |
| IMGT HCDR3 | TTGGVWKYGGSLDF | SEQ ID |
| | | NO: 246 |
| IMGT HCDR3 | ATGGVWKYGGSLDF | SEQ ID |
| | | NO: 247 |
| IMGT HCDR3 | TTGSVWKYGGSLDF | SEQ ID |
| | | NO: 248 |
| IMGT HCDR3 | STGSVWNYGGSLDF | SEQ ID |
| | | NO: 249 |
| IMGT HCDR3 | TTGSVWNYGGSLDY | SEQ ID |
| | | NO: 250 |
| IMGT HCDR3 | TTGSVWKYGGSLDY | SEQ ID |
| | | NO: 251 |
| IMGT HCDR3 | TTGSVWRYGGSLDF | SEQ ID |
| | | NO: 252 |
| IMGT HCDR3 | TTGSVWNYGGSLDF | SEQ ID |
| | | NO: 253 |
| IMGT HCDR3 | TTGSSWHVYFFDF | SEQ ID |
| | | NO: 254 |
| IMGT HCDR3 | TTGSSWSLYYFDF | SEQ ID |
| | | NO: 255 |
| IMGT HCDR3 | TTGSSWYVYYFDY | SEQ ID |
| | | NO: 256 |
| IMGT HCDR3 | TTGSSWYVYYFDF | SEQ ID |
| | | NO: 257 |
| Kabat LCDR1 | RASQDIRNDLA | SEQ ID |
| | | NO: 258 |
| Kabat LCDR1 | QASQGITNYLN | SEQ ID |
| | | NO: 259 |
| Kabat LCDR1 | RASQGIRNDLG | SEQ ID |
| | | NO: 260 |
| Kabat LCDR1 | RASQGIRDDLA | SEQ ID |
| | | NO: 261 |
| Kabat LCDR1 | KSSQSVFYSSNNKNYLA | SEQ ID |
| | | NO: 262 |
| Kabat LCDR1 | RASQGISRFLN | SEQ ID |
| | | NO: 263 |
| Kabat LCDR1 | RASQSISNYLN | SEQ ID |
| | | NO: 264 |
| Kabat LCDR1 | RASQGISSYLA | SEQ ID |
| | | NO: 265 |
| Kabat LCDR1 | RASQDIRNDLD | SEQ ID |
| | | NO: 266 |
| Kabat LCDR1 | RASQAIRNDLG | SEQ ID |
| | | NO: 267 |
| Kabat LCDR1 | RTSQGIRNDLA | SEQ ID |
| | | NO: 268 |
| Kabat LCDR1 | RASQGIRNDLA | SEQ ID |
| | | NO: 269 |
| Kabat LCDR1 | RASQGIRNNLG | SEQ ID |
| | | NO: 270 |
| Kabat LCDR1 | RASQAIRDDLA | SEQ ID |
| | | NO: 271 |
| Kabat LCDR1 | KSSQSVFYTSNSKHYLN | SEQ ID |
| | | NO: 272 |
| Kabat LCDR1 | KSSQSVLYSSNNKNYLA | SEQ ID |
| | | NO: 273 |
| Kabat LCDR1 | KSSQSVFYTSNNKNYLN | SEQ ID |
| | | NO: 274 |
| Kabat LCDR1 | KSSQSVFYTSSNKHYLN | SEQ ID |
| | | NO: 275 |
| Kabat LCDR1 | RASQSITRYLN | SEQ ID |
| | | NO: 276 |
| Kabat LCDR1 | RASQFITRYLN | SEQ ID |
| | | NO: 277 |
| Kabat LCDR1 | RASQGISRYLN | SEQ ID |
| | | NO: 278 |
| Kabat LCDR1 | RASQSITRSLN | SEQ ID |
| | | NO: 279 |
| Kabat LCDR1 | RASQSISRYLN | SEQ ID |
| | | NO: 280 |
| Kabat LCDR1 | RASQNISRYLN | SEQ ID |
| | | NO: 281 |
| Kabat LCDR1 | RASQIISRYLN | SEQ ID |
| | | NO: 282 |
| Kabat LCDR1 | RASQSMSSHLN | SEQ ID |
| | | NO: 283 |
| Kabat LCDR1 | RASQSISTYLN | SEQ ID |
| | | NO: 284 |
| Kabat LCDR1 | RASQSISSYLN | SEQ ID |
| | | NO: 285 |
| Chothia LCDR1 | SQDIRND | SEQ ID |
| | | NO: 286 |
| Chothia LCDR1 | SQGITNY | SEQ ID |
| | | NO: 287 |
| Chothia LCDR1 | SQGIRND | SEQ ID |
| | | NO: 288 |
| Chothia LCDR1 | SQGIRDD | SEQ ID |
| | | NO: 289 |
| Chothia LCDR1 | SQSVFYSSNNKNY | SEQ ID |
| | | NO: 290 |
| Chothia LCDR1 | SQGISRF | SEQ ID |
| | | NO: 291 |
| Chothia LCDR1 | SQSISNY | SEQ ID |
| | | NO: 292 |
| Chothia LCDR1 | SQGISSY | SEQ ID |
| | | NO: 293 |
| Chothia LCDR1 | SQAIRND | SEQ ID |
| | | NO: 294 |
| Chothia LCDR1 | SQGIRNN | SEQ ID |
| | | NO: 295 |
| Chothia LCDR1 | SQAIRDD | SEQ ID |
| | | NO: 296 |
| Chothia LCDR1 | SQSVFYTSNSKHY | SEQ ID |
| | | NO: 297 |
| Chothia LCDR1 | SQSVLYSSNNKNY | SEQ ID |
| | | NO: 298 |
| Chothia LCDR1 | SQSVFYTSNNKNY | SEQ ID |
| | | NO: 299 |
| Chothia LCDR1 | SQSVFYTSSNKHY | SEQ ID |
| | | NO: 300 |
| Chothia LCDR1 | SQSITRY | SEQ ID |
| | | NO: 301 |
| Chothia LCDR1 | SQFITRY | SEQ ID |
| | | NO: 302 |
| Chothia LCDR1 | SQGISRY | SEQ ID |
| | | NO: 303 |
| Chothia LCDR1 | SQSITRS | SEQ ID |
| | | NO: 304 |
| Chothia LCDR1 | SQSISRY | SEQ ID |
| | | NO: 305 |
| Chothia LCDR1 | SQNISRY | SEQ ID |
| | | NO: 306 |
| Chothia LCDR1 | SQIISRY | SEQ ID |
| | | NO: 307 |
| Chothia LCDR1 | SQSMSSH | SEQ ID |
| | | NO: 308 |
| Chothia LCDR1 | SQSISTY | SEQ ID |
| | | NO: 309 |
| Chothia LCDR1 | SQSISSY | SEQ ID |
| | | NO: 310 |
| IMGT LCDR1 | QDIRND | SEQ ID |
| | | NO: 311 |
| IMGT LCDR1 | QGITNY | SEQ ID |
| | | NO: 312 |
| IMGT LCDR1 | QGIRND | SEQ ID |
| | | NO: 313 |
| IMGT LCDR1 | QGIRDD | SEQ ID |
| | | NO: 314 |
| IMGT LCDR1 | QSVFYSSNNKNY | SEQ ID |
| | | NO: 315 |
| IMGT LCDR1 | QGISRF | SEQ ID |
| | | NO: 316 |
| IMGT LCDR1 | QSISNY | SEQ ID |
| | | NO: 317 |
| IMGT LCDR1 | QGISSY | SEQ ID |
| | | NO: 318 |
| IMGT LCDR1 | QAIRND | SEQ ID |
| | | NO: 319 |
| IMGT LCDR1 | QAIRDD | SEQ ID |
| | | NO: 320 |
| IMGT LCDR1 | QSVFYTSNSKHY | SEQ ID |
| | | NO: 321 |
| IMGT LCDR1 | QSVLYSSNNKNY | SEQ ID |
| | | NO: 322 |
| IMGT LCDR1 | QSVFYTSNNKNY | SEQ ID |
| | | NO: 323 |
| IMGT LCDR1 | QSVFYTSSNKHY | SEQ ID |
| | | NO: 324 |
| IMGT LCDR1 | QSITRY | SEQ ID |
| | | NO: 325 |
| IMGT LCDR1 | QFITRY | SEQ ID |
| | | NO: 326 |
| IMGT LCDR1 | QGISRY | SEQ ID |
| | | NO: 327 |
| IMGT LCDR1 | QSITRS | SEQ ID |
| | | NO: 328 |
| IMGT LCDR1 | QSISRY | SEQ ID |
| | | NO: 329 |
| IMGT LCDR1 | QNISRY | SEQ ID |
| | | NO: 330 |
| IMGT LCDR1 | QIISRY | SEQ ID |
| | | NO: 331 |
| IMGT LCDR1 | QSMSSH | SEQ ID |
| | | NO: 332 |
| IMGT LCDR1 | QSISTY | SEQ ID |
| | | NO: 333 |
| IMGT LCDR1 | QSISSY | SEQ ID |
| | | NO: 334 |
| Kabat LCDR2 | ASSSLQS | SEQ ID |
| | | NO: 335 |
| Kabat LCDR2 | DASNLET | SEQ ID |
| | | NO: 336 |
| Kabat LCDR2 | AASSLQS | SEQ ID |
| | | NO: 337 |
| Kabat LCDR2 | WASTRES | SEQ ID |
| | | NO: 338 |
| Kabat LCDR2 | AASTLQN | SEQ ID |
| | | NO: 339 |
| Kabat LCDR2 | AAYRLQT | SEQ ID |
| | | NO: 340 |
| Kabat LCDR2 | AASTLQS | SEQ ID |
| | | NO: 341 |
| Kabat LCDR2 | VVSSFQS | SEQ ID |
| | | NO: 342 |
| Kabat LCDR2 | ATSSLQS | SEQ ID |
| | | NO: 343 |
| Kabat LCDR2 | ATSNLQS | SEQ ID |
| | | NO: 344 |
| Kabat LCDR2 | AASSFQS | SEQ ID |
| | | NO: 345 |
| Kabat LCDR2 | AASSLKS | SEQ ID |
| | | NO: 346 |
| Kabat LCDR2 | ATSSFQS | SEQ ID |
| | | NO: 347 |
| Kabat LCDR2 | AVSSLQS | SEQ ID |
| | | NO: 348 |
| Kabat LCDR2 | ATSILQS | SEQ ID |
| | | NO: 349 |
| Kabat LCDR2 | AASSLQN | SEQ ID |
| | | NO: 350 |
| Kabat LCDR2 | AASILQS | SEQ ID |
| | | NO: 351 |
| Kabat LCDR2 | GASTLQS | SEQ ID |
| | | NO: 352 |
| Kabat LCDR2 | AASRLQS | SEQ ID |
| | | NO: 353 |
| Kabat LCDR2 | GASSLES | SEQ ID |
| | | NO: 354 |
| Kabat LCDR2 | GASSLQS | SEQ ID |
| | | NO: 355 |
| Kabat LCDR2 | AASRLES | SEQ ID |
| | | NO: 356 |
| Kabat LCDR2 | AASNLQS | SEQ ID |
| | | NO: 357 |
| Kabat LCDR2 | AASSLQS | SEQ ID |
| | | NO: 358 |
| Chothia and | ASS | SEQ ID |
| IMGT LCDR2 | | NO: 359 |
| Chothia and | DAS | SEQ ID |
| IMGT LCDR2 | | NO: 360 |
| Chothia and | AAS | SEQ ID |
| IMGT LCDR2 | | NO: 361 |
| Chothia and | WAS | SEQ ID |
| IMGT LCDR2 | | NO: 362 |
| Chothia and | AAY | SEQ ID |
| IMGT LCDR2 | | NO: 363 |
| Chothia and | VVS | SEQ ID |
| IMGT LCDR2 | | NO: 364 |
| Chothia and | ATS | SEQ ID |
| IMGT LCDR2 | | NO: 365 |
| Chothia and | AVS | SEQ ID |
| IMGT LCDR2 | | NO: 366 |
| Chothia and | GAS | SEQ ID |
| IMGT LCDR2 | | NO: 367 |
| Kabat LCDR3 | LQHNNYPFT | SEQ ID |
| | | NO: 368 |
| Kabat LCDR3 | QQYENLPYT | SEQ ID |
| | | NO: 369 |
| Kabat LCDR3 | LQHNSYPWS | SEQ ID |
| | | NO: 370 |
| Kabat LCDR3 | LQHKSNPFT | SEQ ID |
| | | NO: 371 |
| Kabat LCDR3 | QQHYDIPRT | SEQ ID |
| | | NO: 372 |
| Kabat LCDR3 | QQTYSTPLT | SEQ ID |
| | | NO: 373 |
| Kabat LCDR3 | QQSYSSPLT | SEQ ID |
| | | NO: 374 |
| Kabat LCDR3 | QQLNSYPRT | SEQ ID |
| | | NO: 375 |
| Kabat LCDR3 | QQLNNYPRT | SEQ ID |
| | | NO: 376 |
| Kabat LCDR3 | QQLQSYPRT | SEQ ID |
| | | NO: 377 |
| Kabat LCDR3 | LQHNSYPFT | SEQ ID |
| | | NO: 378 |
| Kabat LCDR3 | LQHNTYPFS | SEQ ID |
| | | NO: 379 |
| Kabat LCDR3 | LQHSSYPFT | SEQ ID |
| | | NO: 380 |
| Kabat LCDR3 | LQHNTYPFT | SEQ ID |
| | | NO: 381 |
| Kabat LCDR3 | LQHNSNPFT | SEQ ID |
| | | NO: 382 |
| Kabat LCDR3 | LQHYSNPFT | SEQ ID |
| | | NO: 383 |
| Kabat LCDR3 | QQYYSIPRT | SEQ ID |
| | | NO: 384 |
| Kabat LCDR3 | QQHYSTPRT | SEQ ID |
| | | NO: 385 |
| Kabat LCDR3 | QQYYSTPRT | SEQ ID |
| | | NO: 386 |
| Kabat LCDR3 | QQSYSTPLT | SEQ ID |
| | | NO: 387 |
| Kabat LCDR3 | QQSYNTPLT | SEQ ID |
| | | NO: 388 |
| Kabat LCDR3 | QQSYSTPVT | SEQ ID |
| | | NO: 389 |
| Kabat LCDR3 | QQSYRTSLT | SEQ ID |
| | | NO: 390 |
| Kabat LCDR3 | QQSYGTPLT | SEQ ID |
| | | NO: 391 |
| Kabat LCDR3 | QQSYSTPFT | SEQ ID |
| | | NO: 392 |
| Kabat LCDR3 | QQSYRSPFT | SEQ ID |
| | | NO: 393 |
| Chothia and | HNNYPF | SEQ ID |
| IMGT LCDR3 | | NO: 394 |
| Chothia and | YENLPY | SEQ ID |
| IMGT LCDR3 | | NO: 395 |
| Chothia and | HNSYPW | SEQ ID |
| IMGT LCDR3 | | NO: 396 |
| Chothia and | HKSNPF | SEQ ID |
| IMGT LCDR3 | | NO: 397 |
| Chothia and | HYDIPR | SEQ ID |
| IMGT LCDR3 | | NO: 398 |
| Chothia and | TYSTPL | SEQ ID |
| IMGT LCDR3 | | NO: 399 |
| Chothia and | SYSSPL | SEQ ID |
| IMGT LCDR3 | | NO: 400 |
| Chothia and | LNSYPR | SEQ ID |
| IMGT LCDR3 | | NO: 401 |
| Chothia and | LNNYPR | SEQ ID |
| IMGT LCDR3 | | NO: 402 |
| Chothia and | LQSYPR | SEQ ID |
| IMGT LCDR3 | | NO: 403 |
| Chothia and | HNSYPF | SEQ ID |
| IMGT LCDR3 | | NO: 404 |
| Chothia and | HNTYPF | SEQ ID |
| IMGT LCDR3 | | NO: 405 |
| Chothia and | HSSYPF | SEQ ID |
| IMGT LCDR3 | | NO: 406 |
| Chothia and | HNSNPF | SEQ ID |
| IMGT LCDR3 | | NO: 407 |
| Chothia and | HYSNPF | SEQ ID |
| IMGT LCDR3 | | NO: 408 |
| Chothia and | YYSIPR | SEQ ID |
| IMGT LCDR3 | | NO: 409 |
| Chothia and | HYSTPR | SEQ ID |
| IMGT LCDR3 | | NO: 410 |
| Chothia and | YYSTPR | SEQ ID |
| IMGT LCDR3 | | NO: 411 |
| Chothia and | SYSTPL | SEQ ID |
| IMGT LCDR3 | | NO: 412 |
| Chothia and | SYNTPL | SEQ ID |
| IMGT LCDR3 | | NO: 413 |
| Chothia and | SYSTPV | SEQ ID |
| IMGT LCDR3 | | NO: 414 |
| Chothia and | SYRTSL | SEQ ID |
| IMGT LCDR3 | | NO: 415 |
| Chothia and | SYGTPL | SEQ ID |
| IMGT LCDR3 | | NO: 416 |
| Chothia and | SYSTPF | SEQ ID |
| IMGT LCDR3 | | NO: 417 |
| Chothia and | SYRSPF | SEQ ID |
| IMGT LCDR3 | | NO: 418 |
| Heavy Chain | EVQVVESGGGLVQPGGSLRLSCAASGFTFSNYAMTWVRQSP | SEQ ID |
| Variable Domain | GKGLEWVSLISGSGGLTKYADSVKGRFTISRDNSKKTLFLQM | NO: 419 |
| 1 | NSLRAEDTAAYYCVKDEGLTTGEYWGQGTQVTVSS | |
| Heavy Chain | KVQLVESGGGLVQPGGSLRLSCAASGFRFSSYAMNWVRQAP | SEQ ID |
| Variable Domain | GKGLEWVSLISGSGGLTKYADSVKGRFTISRDNSRNTLYLQM | NO: 420 |
| 2 | ISLRAEDTAVYFCAKERWEPSFHYWGQGTLVTVSS | |
| Heavy Chain | EVQVVESGGGLIQPGGSLRISCAASGFPVSSNYMSWVRQAPG | SEQ ID |
| Variable Domain | KGLDWVSIIYSRDSTHYADSVKGRFTMSRDNSKNTVYLQMN | NO: 421 |
| 3 | SLRAEDTAVYYCAGTLTYYYGAGSDYWGQGTLVTVSS | |
| Heavy Chain | EVQLVESGGGLVQPGGSLRLSCAASGFTFSNYWMHWVRQA | SEQ ID |
| Variable Domain | PGKGLVWVSRINSDGISTRYADSVKGRFTISRDNAKNTLYLQ | NO: 422 |
| 4 | MKSLRAEDTAVYYCVRDFQLELPDYWGQGTLVTVSS | |
| Heavy Chain | EVQLVESGGGFVKPGGSLRLSCAASGFTFSNAWMSWVRQAP | SEQ ID |
| Variable Domain | GKGLEWVGRIKSNSYGGTTDYAAPAIGRFTISRDGSKSTLYL | NO: 423 |
| 5 | QMNSLKTEDTAVYYCTTDHPKFGFDFWGQGTLVTVSS | |
| Heavy Chain | EVQLVESGGGLVTPGGSLRISCAASGFTFNNAWMTWVRQAP | SEQ ID |
| Variable Domain | GKGLEWVGRIKSISYGGTTDSAAPVKGRFTISRDDSENTLYL | NO: 424 |
| 6 | QMSSLKTEDTAVYYCSTGSVWKYGGSLDFWGQGTLVTVSS | |
| Heavy Chain | EVQLVESGGGLVKPGGSLRLSCAASGFTFSYAWMSWVRQAP | SEQ ID |
| Variable Domain | GRGLEWVGRIKSETDGGTTDYAAPVKGRFTISRDHSENTLFL | NO: 425 |
| 7 | QMNSLKTEDTAVYYCTTGSSWNVYFFDFWGQGTLVTVSS | |
| Heavy Chain | QVQLVQSGAEVKKPGASVKVSCKASGYTFTNYDINWVRQA | SEQ ID |
| Variable Domain | TGQGLEWMGWMNPHSGQTGYAQKFQGRVTMTRNTAISTA | NO: 426 |
| 8 | YMELSSLRSEDTAVYYCAREGYDLWSGDSSNWLDPWGQGT | |
| | LVTVSA | |
| Heavy Chain | QVQLVQSGAEVKKPGASVKVSCKASGYTFTNYDINWVRQA | SEQ ID |
| Variable Domain | TGQGLEWMGWMNPHSGQTGYAQKFQGRVTMTRNTAISTA | NO: 427 |
| 9 | YMELSSLRSEDTAVYYCAREGYDLWSGESSNWLDPWGQGT | |
| | LVTVSA | |
| Heavy Chain | QVQLVQSGAEVKKPGASVKVSCKASGYTFTNYDINWVRQA | SEQ ID |
| Variable Domain | TGQGLEWMGWMNPHSGQTGYAQKFQGRVTMTRNTAISTA | NO: 428 |
| 10 | YMELSSLRSEDTAVYYCAREGYDLWSGHSSNWLDPWGQGT | |
| | LVTVSA | |
| Heavy Chain | QVQLVQSGAEVKKPGASVKVSCKASGYTFTNYDINWVRQA | SEQ ID |
| Variable Domain | TGQGLEWMGWMNPHSGQTGYAQKFQGRVTMTRNTAISTA | NO: 429 |
| 11 | YMELSSLRSEDTAVYYCAREGYDLWSGYSSNWLDPWGQGT | |
| | LVTVSA | |
| Heavy Chain | EVQVVESGGGLVQPGGSLRLSCAASGFTFSSYAMYWVRQSP | SEQ ID |
| Variable Domain | GKGLEWVSLITGSGGSTHYADSVKGRFTISRDNSKKTLFLQM | NO: 430 |
| 12 | NSLRAEDTAAYYCAKDEGLTTGEYWGQGTQVTVSS | |
| Heavy Chain | EVQVVESGGGLVQPGGSLRLSCAASGFTFRNYAMTWVRQSP | SEQ ID |
| Variable Domain | GKGLEWVSLISGSGGITKYADSVKGRFTISRDNSKKTLFLQM | NO: 431 |
| 13 | NSLRAEDTAAYYCVKDEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGITHYADSVKGRFTISRDNSKKTLFLQM | NO: 432 |
| 14 | NSLRAEDTAAYYCVKDEGLTTGEFWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGVTHYADSVKGRFTISRDNSKKTLFLQM | NO: 433 |
| 15 | NSLRAEDTAAYYCVKDEGLTTGEFWGQGTQVTVSS | |
| Heavy Chain | EVQVVESGGGLVQPGGSLRLSCAASGFTFSSYAMSWVRQSP | SEQ ID |
| Variable Domain | GKGLEWVSLISGSGGITHYADSVKGRFTISRDNSKKTLFLQM | NO: 434 |
| 16 | NSLRAEDTAAYYCVKDEGLTTGEYWGQGTQVTVSS | |
| Heavy Chain | EVQVVESGGGLVQPGGSLRLSCAASGFTFSSYAMSWVRQSP | SEQ ID |
| Variable Domain | GKGLEWVSLISGSGAITHYADSVKGRFTISRDNSKKTLFLQM | NO: 435 |
| 17 | NSLRAEDTAAYYCVKDEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGVTHYADSVKGRFTISRDNSKKTLFLQM | NO: 436 |
| 18 | NSLRAEDTAAYYCVKDEGLTTGESWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGITHYADSVKGRFTISRDNSKKTLFLQM | NO: 437 |
| 19 | NSLRAEDTAAYYCVKDEGLTTGESWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGLTKYADSVKGRFTISRDNSKKTLFLQM | NO: 438 |
| 20 | NSLRAEDTAAYYCAKEEGLTTGEYWGQGTQVTVSS | |
| Heavy Chain | EVQVVESGGGLVQPGGSLRLSCAASGFTFNTYAMTWVRQSP | SEQ ID |
| Variable Domain | GKGLEWVSLISGSGGLTKYADSVKGRFTISRDNSKKTLFLQM | NO: 439 |
| 21 | NSLRAEDTAAYYCAKEEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGLTNYADSVKGRFTISRDNSKKTLFLQM | NO: 440 |
| 22 | NSLRAEDTAAYYCAKDEGLTSGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGSTHYADSVKGRFTISRDNSKKTLFLQM | NO: 441 |
| 23 | NSLRAEDTAAYYCAKDEGLTTGEYWGQGTQVTVSS | |
| Heavy Chain | EVQVVESGGGLVQPGGSLRLSCAASGFTFSSHAMTWVRQSP | SEQ ID |
| Variable Domain | GKGLEWVSLISGSGGLTKYADSVRGRFTISRDNSKKTLFLQM | NO: 442 |
| 24 | NSLRAEDTAAYYCVKEEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGLTKYADSVKGRFTISRDNSKKTLFLQM | NO: 443 |
| 25 | NSLRAEDTAAYYCAKDEGLTTGEYWGQGTQVTVSS | |
| Heavy Chain | EVQVVESGGGLVQPGGSLRLSCAASGFTFSSYAMTWVRQSP | |
| Variable Domain | GKGLEWVSLISGSGSLTKYADSVKGRFTISRDNSKKTLFLQM | SEQ ID |
| 26 | NSLRAEDTAAYYCVKEEGLTTGEYWGQGTQVTVSS | NO: 444 |
| Heavy Chain | EVQVVESGGGLVQPGGSLRLSCAASGFTFSSYAMTWVRQSP | SEQ ID |
| Variable Domain | GKGLEWVSLISGSGGLTKYADSVKGRFTISRDNSKKTLFLQM | NO: 445 |
| 27 | NSLRAEDTAAYYCVKDEGLTTGEYWGQGTQVTVSS | |
| Heavy Chain | EVQVVESGGGLVQPGGSLRLSCAASGFTFGNYAMTWVRQSP | SEQ ID |
| Variable Domain | GKGLEWVSLISGSGGLTKYADSVKGRFTISRDNSKKTLFLQM | NO: 446 |
| 28 | NSLRAEDTAAYYCVKDEGLTTGEYWGQGTQVTVSS | |
| Heavy Chain | EVQVVESGGGLVQPGGSLRLSCAASGFTFTNYAMTWVRQSP | |
| Variable Domain | GKGLEWVSLISGGGGLTYYADSVKGRFTISRDNSKKTLFLQ | SEQ ID |
| 29 | MNSLRAEDTAAYYCVKDEGLTTGEYWGQGTQVTVSS | NO: 447 |
| Heavy Chain | EVQVVESGGGLVQPGGSLRLSCAASGFTFSIYAMTWVRQSP | SEQ ID |
| Variable Domain | GKGLEWVSLISGSGGLTKYADSVKGRFTISRDNSKKTLFLQM | NO: 448 |
| 30 | NSLRAEDTAAYYCVKDEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGLTYYADSVKGRFTISRDNSKKTLFLQM | NO: 449 |
| 31 | NSLRAEDTAAYYCVKDEGLTTGEYWGQGTQVTVSS | |
| Heavy Chain | EVQVVESGGGLVQPGGSLRLSCAASGFTFSSHAMTWVRQSP | |
| Variable Domain | GKGLEWVSLISGSGGLTYYADSVKGRFTISRDNSKKTLFLQM | SEQ ID |
| 32 | NSLRAEDTAAYYCVKDEGLTTGEYWGQGTQVTVSS | NO: 450 |
| Heavy Chain | EVQVVESGGGLVQPGGSLRLSCAASGFTFSNYAMTWVRQSP | SEQ ID |
| Variable Domain | GKGLEWVSLISGGGGLTYYADSVKGRFTISRDNSKKTLFLQ | NO: 451 |
| 33 | MNSLRAEDTAAYYCVKDEGLTTGEYWGQGTQVTVSS | |
| Heavy Chain | EVQVVESGGGLVQPGGSLRLSCAASGFTFSSYAMTWVRQSP | |
| Variable Domain | GKGLEWVSLISGSGGLTKYADSVKGRFTISRDNSKKTLFLQM | SEQ ID |
| 34 | NSLRAEDTAAYYCVKEEGLTTGEYWGQGTQVTVSS | NO: 452 |
| Heavy Chain | EVQVVESGGGLVQPGGSLRLSCAASGFTFSSYAMTWVRQSP | SEQ ID |
| Variable Domain | GKGLEWVSLISGSGGITKYADSVKGRFTISRDNSKKTLFLQM | NO: 453 |
| 35 | NSLRAEDTAAYYCAKEEGLTTGEYWGQGTQVTVSS | |
| Heavy Chain | EVQLVESGGGLVQPGGSLRLSCAASGFRFSSYAMNWVRQAP | |
| Variable Domain | GKGLEWVSLISGSGGLTKYADSVKGRFTISRDNSRNTLYLQM | SEQ ID |
| 36 | ISLRAEDTAVYFCAKERWEPSFHYWGQGTLVTVSS | NO: 454 |
| Heavy Chain | EVQLVESGGGLVQPGGSLRLSCAASGFTFRSYWMHWVRQA | SEQ ID |
| Variable Domain | PGKGLVWVSRIDNNGINTRYADSVKGRFTISRDNAKNTLYL | NO: 455 |
| 37 | QMKSLRAEDTAVYYCVRDFQLELRDYWGQGTLVTVSS | |
| Heavy Chain | EVQLVESGGGLVQPGGSLRLSCAASGFTFSNYWMHWVRQA | SEQ ID |
| Variable Domain | PGKGLVWVSRISSDGISTRYADSVKGRFTISRDNAKNTLYLQ | NO: 456 |
| 38 | MKSLRAEDTAVYYCVRDFQLELPDYWGQGTLVTVSS | |
| Heavy Chain | EVQLVESGGGLVQPGGSLRLSCAASGLTFSNYWMHWVRQA | SEQ ID |
| Variable Domain | PGKGLVWVSRINSDGISTRYADSVKGRFTISRDNAKNTLYLQ | NO: 457 |
| 39 | MKSLRAEDTAVYYCVSDFQLELPDYWGQGTLVTVSS | |
| Heavy Chain | EVQLVESGGGLVQPGGSLRLSCAASGFTFSNYWMHWVRQA | |
| Variable Domain | PGKGLVWVSRINNDGISTRYADSVKGRFTISRDNAKNTLYLQ | SEQ ID |
| 40 | MKSLRAEDTAVYYCVRDFQLELPDYWGQGTLVTVSS | NO: 458 |
| Heavy Chain | EVQLVESGGGLVQPGGSLRLSCAASGFTFSSFWMHWVRQAP | SEQ ID |
| Variable Domain | GKGLVWVSRINSDGFSTRYADSVKGRFTISRDNAKNTLYLQ | NO: 459 |
| 41 | MKSLRAEDTAVYYCVRDFQLELRDFWGQGTLVTVSS | |
| Heavy Chain | EVQLVESGGGLVQPGGSLRLSCAASGFTFSSFWMHWVRQAP | SEQ ID |
| Variable Domain | GKGLVWVSRINSDGFSTRYADSVKGRFTISRDNAKNTLYLQ | NO: 460 |
| 42 | MKSLRAEDTAVYYCVRDFQLELRDYWGQGTLVTVSS | |
| Heavy Chain | EVQLVESGGGLVQPGGSLRLSCAASGFTFSSYWMHWVRQAP | SEQ ID |
| Variable Domain | GKGLVWVSRINSDGISTRYADSVKGRFTISRDNAKNTLYLQ | NO: 461 |
| 43 | MKSLRAEDTAVYYCVRDFQLELRDYWGQGTLVTVSS | |
| Heavy Chain | EVQLVESGGGFVKPGGSLRLSCAASGFTFHNAWMSWVRQA | SEQ ID |
| Variable Domain | PGKGLEWVGRIKGVTAGGTADYAAPVKGRFTISRDGSKSTL | NO: 462 |
| 44 | YLQMNSLKTEDTAVYYCTTDHPKFGFDFWGQGTLVTVSS | |
| Heavy Chain | EVQLVESGGGFVKPGGSLRLSCAASGFTFNNAWMSWVRQA | SEQ ID |
| Variable Domain | PGKGLEWVGRVKSVTAGGTTDYAAPVKGRFTISRDGSKSTL | NO: 463 |
| 45 | YLQMNSLKTEDTAVYYCTTDHPKFGFDFWGQGTLVTVSS | |
| Heavy Chain | EVQLVESGGGFVKPGGSLRLSCAASGFTFSNAWMSWVRQAP | SEQ ID |
| Variable Domain | GKGLEWVGRIKSKTDGGTTDYAAPVKGRFTISRDGSKSTLYL | NO: 464 |
| 46 | QMNSLKTEDTAVYYCTTDHPKFGFDYWGQGTLVTVSS | |
| Heavy Chain | EVQLVESGGGFVKPGGSLRLSCAASGFTFNSAWMSWVRQAP | |
| Variable Domain | GKGLEWVGRIKSVTTGGTTDYAAPVKGRFTISRDGSKSTLYL | SEQ ID |
| 47 | QMNSLKTEDTAVYYCTTDHPKFGFDFWGQGTLVTVSS | NO: 465 |
| Heavy Chain | EVQLVESGGGFVKPGGSLRLSCAASGFTFNNAWMSWVRQA | SEQ ID |
| Variable Domain | PGKGLEWVGRIKSVTAGGTTDYAAPVKGRFTISRDGSKSTLY | NO: 466 |
| 48 | LQMNSLKTEDTAVYYCTTDHPKFGFDYWGQGTLVTVSS | |
| Heavy Chain | EVQLVESGGGFVKPGGSLRLSCAASGFTFNNAWMSWVRQA | SEQ ID |
| Variable Domain | PGKGLEWVGRIKSVTAGGTTDFPAPVKGRFTISRDGSKSTLY | NO: 467 |
| 49 | LQMNSLKTEDTAVYYCTTDHPKFGFDFWGQGTLVTVSS | |
| Heavy Chain | EVQLVESGGGLVTPGGSLRISCAASGFIFNNAWMRWVRQAP | |
| Variable Domain | GKGLEWVGHIKSETDGGTTDYAAPVKGRFTISRDDSENTLYL | SEQ ID |
| 50 | QMSSLKTEDTAVYYCTTGGVWNYGGSLDFWGQGTLVTVSS | NO: 468 |
| Heavy Chain | EVQLVESGGGLVTPGGSLRISCAASGFIFNNAWMRWVRQAP | |
| Variable Domain | GKGLEWVGHIKSETDGGTTDYAAPVKGRFTISRDDSENTLYL | SEQ ID |
| 51 | QMSSLKTEDTAVYYCITGGVWNYGGSLDFWGQGTLVTVSS | NO: 469 |
| Heavy Chain | EVQLVESGGGLVTPGGSLRISCAASGFIFNNAWMRWVRQAP | SEQ ID |
| Variable Domain | GKGLEWVGHIKSETDGGTTDYAAPVKGRFTISRDDSENTLYL | NO: 470 |
| 52 | QMSSLKTEDTAVYYCTTGGVWKYGGSLDFWGQGTLVTVSS | |
| Heavy Chain | EVQLVESGGGLVTPGGSLRISCAASGFIFNNAWMRWVRQAP | SEQ ID |
| Variable Domain | GKGLEWVGHIKSETDGGTTDYAAPVKGRFTISRDDSENTLYL | NO: 471 |
| 53 | QMSSLKTEDTAVYYCATGGVWKYGGSLDFWGQGTLVTVSS | |
| Heavy Chain | EVQLVESGGGLVTPGGSLRISCAASGFTFNYAWMTWVRQAP | SEQ ID |
| Variable Domain | GKGLEWVGRIKRKTDGGTTDYAAPVQGRFTISRDDSENTLY | NO: 472 |
| 54 | LQMSSLKTEDTAVYYCTTGSVWKYGGSLDFWGQGTLVTVS | |
| | S | |
| Heavy Chain | EVQLVESGGGLVTPGGSLRISCAASGFTFINAWMNWVRQAP | SEQ ID |
| Variable Domain | GKGLEWVGRIKSLTDGGTTDYAAPVKGRFTISRDDSENTLYL | NO: 473 |
| 55 | QMSSLKTEDTAVYYCSTGSVWNYGGSLDFWGQGTLVTVSS | |
| Heavy Chain | EVQLVESGGGLVTPGGSLRISCAASGFTFSNAWMNWVRQAP | SEQ ID |
| Variable Domain | GKGLEWVGRIKRKTDGGTREYAAPVKGRFTISRDDSENTLY | NO: 474 |
| 56 | LQMSSLKTEDTAVYYCTTGSVWKYGGSLDFWGQGTLVTVS | |
| | S | |
| Heavy Chain | EVQLVESGGGLVTPGGSLRISCAASGFTFSNAWMSWVRQAP | SEQ ID |
| Variable Domain | GKGLEWVGRIKRNTDGGTTDYAAPVKGRFTISRDDSENTLY | NO: 475 |
| 57 | LQMSSLKTEDTAVYYCTTGSVWKYGGSLDFWGQGTLVTVS | |
| | S | |
| Heavy Chain | EVQLVESGGGLVTPGGSLRISCAASGFTFSNAWMSWVRQAP | SEQ ID |
| Variable Domain | GKGLEWVGRIKTNTDGGTTDYAAPVKGRFTISRDDSENTLY | NO: 476 |
| 58 | LQMSSLKTEDTAVYYCTTGSVWNYGGSLDYWGQGTLVTVS | |
| | S | |
| Heavy Chain | EVQLVESGGGLVTPGGSLRISCAASGFTFSNAWMSWVRQAP | SEQ ID |
| Variable Domain | GKGLEWVGRIKSKTYGGTTDYDAPVKGRFTISRDDSENTLY | NO: 477 |
| 59 | LQMSSLKTEDTAVYYCTTGSVWKYGGSLDYWGQGTLVTVS | |
| | S | |
| Heavy Chain | EVQLVESGGGLVTPGGSLRISCAASGFTFSNAWMSWVRQAP | SEQ ID |
| Variable Domain | GKGLEWVGRIKSQTYGGTTDYAAPVKGRFTISRDDSENTLY | NO: 478 |
| 60 | LQMSSLKTEDTAVYYCTTGSVWKYGGSLDYWGQGTLVTVS | |
| | S | |
| Heavy Chain | EVQLVESGGGLVTPGGSLRISCAASGFTFSNAWMSWVRQAP | SEQ ID |
| Variable Domain | GKGLEWVGRIKSKTDGGTTDYAAPVKGRFTISRDDSENTLY | NO: 479 |
| 61 | LQMSSLKTEDTAVYYCTTGSVWRYGGSLDFWGQGTLVTVS | |
| | S | |
| Heavy Chain | EVQLVESGGGLVTPGGSLRISCAASGFTFSNAWMNWVRQAP | SEQ ID |
| Variable Domain | GKGLEWVGRIKSKTDGGTTDYAAPVKGRFTISRDDSENTLY | NO: 480 |
| 62 | LQMSSLKTEDTAVYYCTTGSVWNYGGSLDFWGQGTLVTVS | |
| | S | |
| Heavy Chain | EVQLVESGGGLVTPGGSLRISCAASGFTFSNAWMSWVRQAP | SEQ ID |
| Variable Domain | GKGLEWVGRIKSKTDGGTTDYAAPVKGRFTISRDDSENTLY | NO: 481 |
| 63 | LQMSSLKTEDTAVYYCTTGSVWKYGGSLDFWGQGTLVTVS | |
| | S | |
| Heavy Chain | EVQLVESGGGLVTPGGSLRISCAASGFTFSNAWMTWVRQAP | SEQ ID |
| Variable Domain | GKGLEWVGRIKSKTYGGTTDYAAPVKGRFTISRDDSENTLY | NO: 482 |
| 64 | LQMSSLKTEDTAVYYCTTGSVWNYGGSLDFWGQGTLVTVS | |
| | S | |
| Heavy Chain | EVQLVESGGGLVTPGGSLRISCAASGFTFINAWMNWVRQAP | SEQ ID |
| Variable Domain | GKGLEWVGRIKRKTDGGTRDYAAPVKGRFTISRDDSENTLY | NO: 483 |
| 65 | LQMSSLKTEDTAVYYCTTGSVWKYGGSLDFWGQGTLVTVS | |
| | S | |
| Heavy Chain | EVQLVESGGGLVKPGGSLRLSCAASGFTFSNAWMSWVRQAP | SEQ ID |
| Variable Domain | GRGLEWVGRIKRETDGGTTDYASPVKGRFTISRDHSENTLFL | NO: 484 |
| 66 | QMNSLKTEDTAVYYCTTGSSWNVYFFDFWGQGTLVTVSS | |
| Heavy Chain | EVQLVESGGGLVKPGGSLRLSCAASGFTFSTAWMSWVRQAP | SEQ ID |
| Variable Domain | GRGLEWVGRIKRKSDGGTTDYAAPVKGRFTISRDHSENTLFL | NO: 485 |
| 67 | QMNSLKTEDTAVYYCTTGSSWHVYFFDFWGQGTLVTVSS | |
| Heavy Chain | EVQLVESGGGLVKPGGSLRLSCAASGFIFSNAWMSWVRQAP | SEQ ID |
| Variable Domain | GRGLEWVGRIKSKTYGGTTDYAAPVKGRFTISRDHSENTLFL | NO: 486 |
| 68 | QMNSLKTEDTAVYYCTTGSSWSLYYFDFWGQGTLVTVSS | |
| Heavy Chain | EVQLVESGGGLVKPGGSLRLSCAASGFIFSNAWMSWVRQAP | SEQ ID |
| Variable Domain | GRGLEWVGRIKSKTDGGTTDYAAPVKGRFTISRDHSENTLFL | NO: 487 |
| 69 | QMNSLKTEDTAVYYCTTGSSWYVYYFDYWGQGTLVTVSS | |
| Heavy Chain | EVQLVESGGGLVKPGGSLRLSCAASGFIFSNAWMSWVRQAP | SEQ ID |
| Variable Domain | GRGLEWVGRIKSEIDGGTTDYAAPVKGRFTISRDHSENTLFL | NO: 488 |
| 70 | QMNSLKTEDTAVYYCTTGSSWYVYYFDFWGQGTLVTVSS | |
| Light Chain | DIQMTQSPSSLSASVGDRVTITCRASQDIRNDLAWYQQKPGK | SEQ ID |
| Variable Domain | APKRLIYASSSLQSGVPSRFSGSGSGTEFTLTISSLQPEDFATY | NO: 489 |
| 1 | YCLQHNNYPFTFGPGTKVDIK | |
| Light Chain | DIQMTQSPSSLSASVGDSVIITCQASQGITNYLNWYQQKPGK | SEQ ID |
| Variable Domain | APNLLIYDASNLETGVPSRFSGSGSGTYFTFTISSLQPEDIATY | NO: 490 |
| 2 | FCQQYENLPYTFGQGTKLEIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 491 |
| 3 | YCLQHNSYPWSFGQGTKVEIK | |
| Light Chain | DIQMTQSPSSLSASVGDRVTITCRASQGIRDDLAWYQQKAGK | SEQ ID |
| Variable Domain | APKRLIFAASSLQSGVPSRFSGSGSGTEFTLTISGLQPEDFANY | NO: 492 |
| 4 | YCLQHKSNPFTFGQGTKLEIK | |
| Light Chain | DIVMTQSPDSLAVSLGERATIDCKSSQSVFYSSNNKNYLAWF | SEQ ID |
| Variable Domain | QQKPGQPPKLLIYWASTRESGVPDRFSGSGSGTDFTLTISSLQ | NO: 493 |
| 5 | AEDVAVYYCQQHYDIPRTFGQGTKVEIK | |
| Light Chain | DIQMTQSPSSLSASVGDRVTFTCRASQGISRFLNWYQQKPGK | SEQ ID |
| Variable Domain | APKLLIYAASTLQNGVPSRFSGSGSGTDFTLTISSLQPEDFATY | NO: 494 |
| 6 | FCQQTYSTPLTFGGGTKVEIY | |
| Light Chain | DIQMTQSPSSLSASVGDSVTITCRASQSISNYLNWYHQKPGK | SEQ ID |
| Variable Domain | APKLLIYAAYRLQTGVPSRFSGSGSGTDFTLTISSLQPEDFAT | NO: 495 |
| 7 | YYCQQSYSSPLTFGGGTKVEIK | |
| Light Chain | DIQLTQSPSFLSASVGDRVTITCRASQGISSYLAWYQQKPGKA | SEQ ID |
| Variable Domain | PKLLIYAASTLQSGFRSRFSGSGSGTEFTLTISSLQPEDFASYY | NO: 496 |
| 8 | CQQLNSYPRTFGQGTKVEIK | |
| Light Chain | DIQLTQSPSFLSASVGDRVTITCRASQGISSYLAWYQQKPGKA | SEQ ID |
| Variable Domain | PKLLIYAASTLQSGFRSRFSGSGSGTEFTLTISSLQPEDFASYY | NO: 497 |
| 9 | CQQLNNYPRTFGQGTKVEIK | |
| Light Chain | DIQLTQSPSFLSASVGDRVTITCRASQGISSYLAWYQQKPGKA | SEQ ID |
| Variable Domain | PKLLIYAASTLQSGFRSRFSGSGSGTEFTLTISSLQPEDFASYY | NO: 498 |
| 10 | CQQLQSYPRTFGQGTKVEIK | |
| Light Chain | DIQMTQSPSSLSASVGDRVTITCRASQGIRNDLGWYQQKPGK | SEQ ID |
| Variable Domain | APKRLIYVVSSFQSGVPSRFSGSGSGTEFTLTISSLQPEDFATY | NO: 499 |
| 11 | YCLQHNNYPFTFGPGTKVDIK | |
| Light Chain | DIQMTQSPSSLSASVGDRVTITCRASQDIRNDLDWYQQKPGK | SEQ ID |
| Variable Domain | APKRLIYATSSLQSGVPSRFSGSGSGTEFTLTISSLQPEDFATY | NO: 500 |
| 12 | YCLQHNSYPFTFGPGTKVDIK | |
| Light Chain | DIQMTQSPSSLSASVGDRVTITCRASQAIRNDLGWYQQKPGK | SEQ ID |
| Variable Domain | APKRLIYATSNLQSGVPSRFSGSGSGTEFTLTISSLQPEDFATY | NO: 501 |
| 13 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYATSSLQSGVPSRFSGSGSGTEFTLTISSLQPEDFATY | NO: 502 |
| 14 | YCLQHNSYPFTFGPGTKVDIK | |
| Light Chain | DIQMTQSPSSLSASVGDRVTITCRTSQGIRNDLA WYQQKPGK | SEQ ID |
| Variable Domain | APKRLIYAASSLQSGVPSRFSGSGSGTEFTLTISSLQPEDFATY | NO: 503 |
| 15 | YCLQHNNYPFTFGPGTKVDIK | |
| Light Chain | DIQMTQSPSSLSASVGDRVTITCRASQGIRNDLAWYQQKPGK | SEQ ID |
| Variable Domain | APKRLIYAASSFQSGVPSRFSGSGSGTEFTLTISSLQPEDFATY | NO: 504 |
| 16 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSLKSGVPSRFSGSGSGTEFTLTISSLQPEDFATY | NO: 505 |
| 17 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 506 |
| 18 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYATSSFQSGVPSRFSGSGSGTEFTLTISSLQPEDFATY | NO: 507 |
| 19 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSFQSGVPSRFSGSGSGTEFTLTISSLQPEDFATY | NO: 508 |
| 20 | YCLQHNSYPFTFGPGTKVDIK | |
| Light Chain | DIQMTQSPSSLSASVGDRVTITCRASQGIRNNLGWYQQKPGK | SEQ ID |
| Variable Domain | APKRLIYAASSLQSGVPSRFSGSGSGTEFTLTISSLQPEDFATY | NO: 509 |
| 21 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 510 |
| 22 | YCLQHNTYPFSFGPGTKVDIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 511 |
| 23 | YCLQHSSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 512 |
| 24 | YCLQHNNYPFTFGPGTKVDIK | |
| Light Chain | DIQMTQSPSSLSASVGDRVTITCRASQGIRNDLGWYQQKPGK | SEQ ID |
| Variable Domain | APKRLIYAVSSLQSGVPSRFSGSGSGTEFTLTISSLQPEDFATY | NO: 513 |
| 25 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 514 |
| 26 | YCLQHNTYPFTFGPGTKVDIK | |
| Light Chain | DIQMTQSPSSLSASVGDRVTITCRASQGIRNDLGWYQQKPGK | SEQ ID |
| Variable Domain | APKRLIYASSSLQSGVPSRFSGSGSGTEFTLTISSLQPEDFATY | NO: 515 |
| 27 | YCLQHNNYPFTFGPGTKVDIK | |
| Light Chain | DIQMTQSPSSLSASVGDRVTITCRASQGIRNDLAWYQQKAGK | SEQ ID |
| Variable Domain | APKRLIFATSILQSGVPSRFSGSGSGTEFTLTISGLQPEDFANY | NO: 516 |
| 28 | YCLQHNSNPFTFGQGTKLEIK | |
| Light Chain | DIQMTQSPSSLSASVGDRVTITCRASQGIRNDLGWYQQKAGK | SEQ ID |
| Variable Domain | APKRLIFAASSLQSGVPSRFSGSGSGTEFTLTISGLQPEDFANY | NO: 517 |
| 29 | YCLQHKSNPFTFGQGTKLEIK | |
| Light Chain | DIQMTQSPSSLSASVGDRVTITCRASQAIRDDLAWYQQKAGK | SEQ ID |
| Variable Domain | APKRLIFAASSLQNGVPSRFSGSGSGTEFTLTISGLQPEDFANY | NO: 518 |
| 30 | YCLQHKSNPFTFGQGTKLEIK | |
| Light Chain | DIQMTQSPSSLSASVGDRVTITCRASQGIRNDLAWYQQKAGK | SEQ ID |
| Variable Domain | APKRLIFAASILQSGVPSRFSGSGSGTEFTLTISGLQPEDFANY | NO: 519 |
| 31 | YCLQHNSNPFTFGQGTKLEIK | |
| Light Chain | DIQMTQSPSSLSASVGDRVTITCRASQGIRNDLAWYQQKAGK | SEQ ID |
| Variable Domain | APKRLIFAASILQSGVPSRFSGSGSGTEFTLTISGLQPEDFANY | NO: 520 |
| 32 | YCLQHKSNPFTFGQGTKLEIK | |
| Light Chain | DIQMTQSPSSLSASVGDRVTITCRASQGIRNDLAWYQQKAGK | SEQ ID |
| Variable Domain | APKRLIFAASSLQSGVPSRFSGSGSGTEFTLTISGLQPEDFANY | NO: 521 |
| 33 | YCLQHNSNPFTFGQGTKLEIK | |
| Light Chain | DIQMTQSPSSLSASVGDRVTITCRASQGIRNDLAWYQQKAGK | SEQ ID |
| Variable Domain | APKRLIFAASILQSGVPSRFSGSGSGTEFTLTISGLQPEDFANY | NO: 522 |
| 34 | YCLQHYSNPFTFGQGTKLEIK | |
| Light Chain | DIVMTQSPDSLAVSLGERATIDCKSSQSVFYTSNSKHYLNWF | SEQ ID |
| Variable Domain | QQKPGQPPKLLIYWASTRESGVPDRFSGSGSGTDFTLTISSLQ | NO: 523 |
| 35 | AEDVAVYYCQQYYSIPRTFGQGTKVEIK | |
| Light Chain | DIVMTQSPDSLAVSLGERATIDCKSSQSVLYSSNNKNYLAWF | SEQ ID |
| Variable Domain | QQKPGQPPKLLIYWASTRESGVPDRFSGSGSGTDFTLTISSLQ | NO: 524 |
| 36 | AEDVAVYYCQQHYSTPRTFGQGTKVEIK | |
| Light Chain | DIVMTQSPDSLAVSLGERATIDCKSSQSVFYTSNSKHYLNWF | SEQ ID |
| Variable Domain | QQKPGQPPKLLIYWASTRESGVPDRFSGSGSGTDFTLTISSLQ | NO: 525 |
| 37 | AEDVAVYYCQQYYSTPRTFGQGTKVEIK | |
| Light Chain | DIVMTQSPDSLAVSLGERATIDCKSSQSVFYTSNNKNYLNWF | SEQ ID |
| Variable Domain | QQKPGQPPKLLIYWASTRESGVPDRFSGSGSGTDFTLTISSLQ | NO: 526 |
| 38 | AEDVAVYYCQQYYSTPRTFGQGTKVEIK | |
| Light Chain | DIVMTQSPDSLAVSLGERATIDCKSSQSVFYTSSNKHYLNWF | SEQ ID |
| Variable Domain | QQKPGQPPKLLIYWASTRESGVPDRFSGSGSGTDFTLTISSLQ | NO: 527 |
| 39 | AEDVAVYYCQQYYSTPRTFGQGTKVEIK | |
| Light Chain | DIQMTQSPSSLSASVGDRVTFTCRASQSITRYLNWYQQKPGK | SEQ ID |
| Variable Domain | APKLLIYAASSLQSGVPSRFSGSGSGTDFTLTISSLQPEDFATY | NO: 528 |
| 40 | FCQQSYSTPLTFGGGTKVEIY | |
| Light Chain | DIQMTQSPSSLSASVGDRVTFTCRASQFITRYLNWYQQKPGK | SEQ ID |
| Variable Domain | APKLLIYAASSLQSGVPSRFSGSGSGTDFTLTISSLQPEDFATY | NO: 529 |
| 41 | FCQQSYSTPLTFGGGTKVEIY | |
| Light Chain | DIQMTQSPSSLSASVGDRVTFTCRASQSITRYLNWYQQKPGK | SEQ ID |
| Variable Domain | APKLLIYAASSLQSGVPSRFSGSGSGTDFTLTISSLQPEDFATY | NO: 530 |
| 42 | FCQQSYNTPLTFGGGTKVEIY | |
| Light Chain | DIQMTQSPSSLSASVGDRVTFTCRASQGISRYLNWYQQKPGK | SEQ ID |
| Variable Domain | APKLLIYGASTLQSGVPSRFSGSGSGTDFTLTISSLQPEDFATY | NO: 531 |
| 43 | FCQQSYSTPLTFGGGTKVEIY | |
| Light Chain | DIQMTQSPSSLSASVGDRVTFTCRASQSITRSLNWYQQKPGK | SEQ ID |
| Variable Domain | APKLLIYAASRLQSGVPSRFSGSGSGTDFTLTISSLQPEDFATY | NO: 532 |
| 44 | FCQQSYSSPLTFGGGTKVEIY | |
| Light Chain | DIQMTQSPSSLSASVGDRVTFTCRASQSISRYLNWYQQKPGK | SEQ ID |
| Variable Domain | APKLLIYAASSLQSGVPSRFSGSGSGTDFTLTISSLQPEDFATY | NO: 533 |
| 45 | FCQQSYSTPVTFGGGTKVEIY | |
| Light Chain | DIQMTQSPSSLSASVGDRVTFTCRASQSISRYLNWYQQKPGK | SEQ ID |
| Variable Domain | APKLLIYAASSLQNGVPSRFSGSGSGTDFTLTISSLQPEDFATY | NO: 534 |
| 46 | FCQQSYSTPVTFGGGTKVEIY | |
| Light Chain | DIQMTQSPSSLSASVGDRVTFTCRASQSISRYLNWYQQKPGK | SEQ ID |
| Variable Domain | APKLLIYGASSLESGVPSRFSGSGSGTDFTLTISSLQPEDFATY | NO: 535 |
| 47 | FCQQSYRTSLTFGGGTKVEIY | |
| Light Chain | DIQMTQSPSSLSASVGDRVTFTCRASQNISRYLNWYQQKPGK | SEQ ID |
| Variable Domain | APKLLIYAASSLQSGVPSRFSGSGSGTDFTLTISSLQPEDFATY | NO: 536 |
| 48 | FCQQSYSTPLTFGGGTKVEIY | |
| Light Chain | DIQMTQSPSSLSASVGDRVTFTCRASQIISRYLNWYQQKPGK | SEQ ID |
| Variable Domain | APKLLIYAASSLQSGVPSRFSGSGSGTDFTLTISSLQPEDFATY | NO: 537 |
| 49 | FCQQSYGTPLTFGGGTKVEIY | |
| Light Chain | DIQMTQSPSSLSASVGDRVTFTCRASQSISRYLNWYQQKPGK | SEQ ID |
| Variable Domain | APKLLIYGASSLQSGVPSRFSGSGSGTDFTLTISSLQPEDFATY | NO: 538 |
| 50 | FCQQSYSTPLTFGGGTKVEIY | |
| Light Chain | DIQMTQSPSSLSASVGDRVTFTCRASQSISRYLNWYQQKPGK | SEQ ID |
| Variable Domain | APKLLIYAASSLQSGVPSRFSGSGSGTDFTLTISSLQPEDFATY | NO: 539 |
| 51 | FCQQSYSTPLTFGGGTKVEIY | |
| Light Chain | DIQMTQSPSSLSASVGDSVTITCRASQSMSSHLNWYHQKPGK | SEQ ID |
| Variable Domain | APKLLIYAASRLESGVPSRFSGSGSGTDFTLTISSLQPEDFATY | NO: 540 |
| 52 | YCQQSYSSPLTFGGGTKVEIK | |
| Light Chain | DIQMTQSPSSLSASVGDSVTITCRASQSISRYLNWYHQKPGK | SEQ ID |
| Variable Domain | APKLLIYAASSLQSGVPSRFSGSGSGTDFTLTISSLQPEDFATY | NO: 541 |
| 53 | YCQQSYSSPLTFGGGTKVEIK | |
| Light Chain | DIQMTQSPSSLSASVGDSVTITCRASQSISTYLNWYHQKPGK | SEQ ID |
| Variable Domain | APKLLIYAASSLQSGVPSRFSGSGSGTDFTLTISSLQPEDFATY | NO: 542 |
| 54 | YCQQSYSSPLTFGGGTKVEIK | |
| Light Chain | DIQMTQSPSSLSASVGDSVTITCRASQSISSYLNWYHQKPGKA | SEQ ID |
| Variable Domain | PKLLIYAASNLQSGVPSRFSGSGSGTDFTLTISSLQPEDFATYY | NO: 543 |
| 55 | CQQSYSTPFTFGGGTKVEIK | |
| Light Chain | DIQMTQSPSSLSASVGDSVTITCRASQSISSYLNWYHQKPGKA | |
| Variable Domain | PKLLIYAASSLQSGVPSRFSGSGSGTDFTLTISSLQPEDFATYY | SEQ ID |
| 56 | CQQSYRSPFTFGGGTKVEIK | NO: 544 |
| Consensus | EGYDLWSGX₁SSNWLDP, where X₁ = D, E, H, or Y | SEQ ID |
| CDRH3 (Kabat) | | NO: 545 |
| Consensus | GYDLWSGX₁SSNWLD, where X₁ = D, E, H, or Y | SEQ ID |
| CDRH3 | | NO: 546 |
| (Chothia) | | |
| Consensus | AREGYDLWSGX₁SSNWLDP, where X₁ = D, E, H, or Y | SEQ ID |
| CDRH3 (IMGT) | | NO: 547 |
| Consensus | QQLX₁X₂YPRT, where X₁ = N or Q, and X₂ = S or N | SEQ ID |
| CDRL3 (Kabat) | | NO: 548 |
| Consensus | LX₁X₂YPR, where X₁ = N or Q, and X₂ = S or N | SEQ ID |
| CDRL3 | | NO: 549 |
| (Chothia) | | |
| Consensus | QQLX₁X₂YPRT, where X₁ = N or Q, and X₂ = S or N | SEQ ID |
| CDRL3 (IMGT) | | NO: 550 |
| Consensus | X₁X₂AMX₃, where X₁ = N, S, T, or I, X₂ = Y or H, and X₃ = T, Y, | SEQ ID |
| CDRH1 (Kabat) | or S | NO: 551 |
| Consensus | LIX₁GX₂GX₃X₄TX YADSVX₆G, where X₁ = S or T, X₂ = S or G, | SEQ ID |
| CDRH2 (Kabat) | X₃ = G, A, or S, X₄ = L, S, I, or V, X₅ = K, H, N, or Y, and X₆ = K | NO: 552 |
| | or R | |
| Consensus | X₁EGLTX₂GEX₃, where X₁ = D or E, X₂ = T or S, and X₃ = Y, F, or | SEQ ID |
| CDRH3 (Kabat) | S | NO: 553 |
| Consensus | RX₁SQX₂IRNX₃LX₄, where X₁ = A or T, X₂ = D, G, or A, X₃ = D | SEQ ID |
| CDRL1 (Kabat) | or N, and X₄ = A, G, or D | NO: 554 |
| Consensus | X₁X₂SX₃X₄X₅S, where X₁ = A or V, X₂ = S, V, T, or A, X₃ = S or N, | SEQ ID |
| CDRL2 (Kabat) | X₄ = L or F, and X₅ = Q or K | NO: 555 |
| Consensus | LQHX₁X₂YPFX₃, where X₁ = N or S, X₂ = N, S, or T, and X₃ = T or | SEQ ID |
| CDRL3 (Kabat) | S | NO: 556 |
| Consensus | GFTFX₁X₂X₃, where X₁ = S, R, N, G, or T, X₂ = N, S, T, or I, and | SEQ ID |
| CDRH1 | X₃ = Y or H | NO: 557 |
| (Chothia) | | |
| Consensus | X₁GX₂GX₃X₄, where X₁ = S or T, X₂ = S or G, X₃ = G, A, or S, | SEQ ID |
| CDRH2 | and X₄ = L, S, I, or V | NO: 558 |
| (Chothia) | | |
| Consensus | EGLTX₁GE, where X₁ = T or S | SEQ ID |
| CDRH3 | | NO: 559 |
| (Chothia) | | |
| Consensus | SQX₁IRNX₂, where X₁ = D, G, or A, and X₂ = D or N | SEQ ID |
| CDRL1 | | NO: 560 |
| (Chothia) | | |
| Consensus | X₁X₂S, where X₁ = A or V, and X₂ = S, V, T, or A | SEQ ID |
| CDRL2 | | NO: 561 |
| (Chothia) | | |
| Consensus | HX₁X₂YPF, where X₁ = N or S, and X₂ = N, S, or T | SEQ ID |
| CDRL3 | | NO: 562 |
| (Chothia) | | |
| Consensus | GFTFX₁X₂X₃A, where X₁ = S, R, N, G, or T, X₂ = N, S, T, or I, and | SEQ ID |
| CDRH1 (IMGT) | X₃ = Y or H | NO: 563 |
| Consensus | IX₁GX₂GX₃X₄T, where X₁ = S or T, X₂ = S or G, X₃ = G, A, or S, | SEQ ID |
| CDRH2 (IMGT) | and X₄ = L, S, I, or V | NO: 564 |
| Consensus | X₁KX₂EGLTX₃GEX₄, where X₁ = V or A, X₂ = D or E, X₃ = T or S, | SEQ ID |
| CDRH3 (IMGT) | and X₄ = Y, F, or S | NO: 565 |
| Consensus | QX₁IRNX₂, where X₁ = D, G, or A, and X₂ = D or N | SEQ ID |
| CDRL1 (IMGT) | | NO: 566 |
| Consensus | X₁X₂S, where X₁ = A or V, and X₂ = S, V, T, or A | SEQ ID |
| CDRL2 (IMGT) | | NO: 567 |
| Consensus | LQHX₁X₂YPFX₃, where X₁ = N or S, X₂ = N, S, or T, and X₃ = T or | SEQ ID |
| CDRL3 (IMGT) | S | NO: 568 |
| Consensus | X₁X₂WMH, where X₁ = N or S, and X₂ = Y or F | SEQ ID |
| Variants of 122 | | NO: 569 |
| CDRH1 (Kabat) | | |
| Consensus | RIX₁X₂X₃GX₄X₅TRYADSVKG, where X₁ = N, D, or S, X₂ = S or | SEQ ID |
| CDRH2 (Kabat) | N, X₃ = D or N, X₄ = I or F, and X₅ = S or N | NO: 570 |
| Consensus | DFQLELX₁DX₂, where X₁ = P or R, and X₂ = Y or F | SEQ ID |
| CDRH3 (Kabat) | | NO: 571 |
| Consensus | RASQX?IRX₂DLX₃, where X₁ = G or A, X₂ = D or N, and X₃ = A | SEQ ID |
| CDRL1 (Kabat) | or G | NO: 572 |
| Consensus | AXISX₂LQX₃, where X₁ = A or T, X₂ = S or I, and X₃ = S or N | SEQ ID |
| CDRL2 (Kabat) | | NO: 573 |
| Consensus | LQHX₁SNPFT, where X₁ = K, N, or Y | SEQ ID |
| CDRL3 (Kabat) | | NO: 574 |
| Consensus | GX₁TFX₂X₃X₄, where X₁ = F or L, X₂ = S or R, and X₃ = N or S, | |
| CDRH1 | and X₄ = Y or F | SEQ ID |
| (Chothia) | | NO: 575 |
| Consensus | X₁X₂X₃GX₄X₅, where X₁ = N, D, or S, X₂ = S or N, X₃ = D or N, X₄ | SEQ ID |
| CDRH2 | | NO: 576 |
| (Chothia) | = I or F, and X₅ = S or N | |
| Consensus | FQLELX₁D, where X₁ = P or R | SEQ ID |
| CDRH3 | | NO: 577 |
| (Chothia) | | |
| Consensus | SQXIIRX₂D, where X₁ = G or A, and X₂ = D or N | SEQ ID |
| CDRL1 | | NO: 578 |
| (Chothia) | | |
| Consensus | AX₁S, where X₁ = A or T | SEQ ID |
| CDRL2 | | NO: 579 |
| (Chothia) | | |
| Consensus | HX₁SNPF, where X₁ = K, N, or Y | SEQ ID |
| CDRL3 | | NO: 580 |
| (Chothia) | | |
| Consensus | GX₁TFX₂X₃X₄W, where X₁ = F or L, X₂ =S or R, and X₃ = N or S, | SEQ ID |
| CDRH1 (IMGT) | and X₄ = Y or F | NO: 581 |
| Consensus | IX₁X₂X₃GX₄X₅T, where X₁ = N, D, or S, X₂ = S or N, X₃ = D or N, | SEQ ID |
| CDRH2 (IMGT) | X₄ = I or F, and X₅ = S or N | NO: 582 |
| Consensus | VX₁DFQLELX₂DX₃, where X₁ = R or S, X₂ = P or R, and X₃ = Y or | SEQ ID |
| CDRH3 (IMGT) | F | NO: 583 |
| Consensus | QX₁IRX₂D, where X₁ = G or A, X₂ = D or N | SEQ ID |
| CDRL1 (IMGT) | | NO: 584 |
| Consensus | AXIS, where X₁ = A or T | SEQ ID |
| CDRL2 (IMGT) | | NO: 585 |
| Consensus | LQHX₁SNPFT, where X₁ = K, N, or Y | SEQ ID |
| CDRL3 (IMGT) | | NO: 586 |
| Variants of 127 | | |
| Consensus | | SEQ ID |
| CDRH1 (Kabat) | X₁AWMS, where X₁ = N or S | NO: 587 |
| Consensus | RX₁KX₂X₃X₄X₅GGTX₆DX₇X₈APX₉X₁₀G, where X₁ = I or V, X₂ = | SEQ ID |
| CDRH2 (Kabat) | S or G, X₃ = N, V, or K, X₄ = S or T, X₅ = Y, A, D, or T, X₆ = T or | NO: 588 |
| | A, X₇ = Y or F, X₈ = A or P, X₉ = A or V, and X₁₀ = I or K | |
| Consensus | DHPKFGFDX₁, where X₁ = F or Y | SEQ ID |
| CDRH3 (Kabat) | | NO: 589 |
| Consensus | KSSQSVX₁YX₂SX₃X₄KX₅YLX₆, where X₁ = F or L, X₂ = S or T, | SEQ ID |
| CDRL1 (Kabat) | and X₃ = N or S, X₄ = N or S, X₅ = N or H, and X₆ = A or N | NO: 590 |
| Consensus | QQX₁YX₂X₃PRT, where X₁ = H or Y, X₂ = D or S, and X₃ = I or T | SEQ ID |
| CDRL3 (Kabat) | | NO: 591 |
| Consensus | GFTFX₁X₂A, where X₁ = S, H, or N, X₂ = N or S | SEQ ID |
| CDRH1 | | NO: 592 |
| (Chothia) | | |
| Consensus | KX₁X₂X₃X₄GGT, where X₁ = S or G, X₂ = N, V, or K, X₃ = S or T, | SEQ ID |
| CDRH2 | and X₄ = Y, A, D, or T | NO: 593 |
| (Chothia) | | |
| Consensus | SQSVX₁ YX₂SX₃X₄KX₅Y, where X₁ = F or L, X₂ = S or T, and X₃ = | SEQ ID |
| CDRL1 | N or S, X₄ = N or S, and X₅ = N or H | NO: 594 |
| (Chothia) | | |
| Consensus | X₁YX₂X₃PR, where X₁ = H or Y, X₂ = D or S, and X₃ = I or T | SEQ ID |
| CDRL3 | | NO: 595 |
| (Chothia) | | |
| Consensus | GFTFX₁X₂AW, where X₁ = S, H, or N, X₂ = N or S | SEQ ID |
| CDRH1 (IMGT) | | NO: 596 |
| Consensus | X₁KX₂X₃X₄X₅GGTX₆, where X₁ = I or V, X₂ = S or G, X₃ = N, V, | SEQ ID |
| CDRH2 (IMGT) | or K, X₄ = S or T, X₅ = Y, A, D, or T, X₆ = T or A | NO: 597 |
| Consensus | TTDHPKFGFDX₁, where X₁ = F or Y | SEQ ID |
| CDRH3 (IMGT) | | NO: 598 |
| Consensus | QSVX₁YX₂SX₃X₄KX,Y, where X₁ = F or L, X₂ = S or T, and X₃ = | SEQ ID |
| CDRL1 (IMGT) | N or S, X₄ = N or S, X₅ = N or H | NO: 599 |
| Consensus | QQX₁YX₂X₃PRT, where X₁ = H or Y, X₂ = D or S, and X₃ = I or T | SEQ ID |
| CDRL3 (IMGT | | NO: 600 |
| Variants of 128 | X₁AWMX₂, where X₁ = N or Y, and X₂ = T, R, N, or S | SEQ ID |
| Consensus | | NO: 601 |
| CDRH1 (Kabat) | | |
| Consensus | X₁IKX₂X₃X₄X₅GGTX₆X₇X₈X₉APVX₁₀G, where X₁ = R or H, X₂ = S, R, | SEQ ID |
| CDRH2 (Kabat) | or T, X₃ = I, E, K, L, N, or Q, X₄ = S or T, X₅ = Y or D, X₆ = T | NO: 602 |
| | or R, X₇ = D or E, X₈ = S or Y, X₉ = A or D, and X₁₀ = K or Q | |
| Consensus | GX₁VWX₂YGGSLDX₃, where X₁ = S or G, X₂ = K, N, or R, and X₃ | SEQ ID |
| CDRH3 (Kabat) | = F or Y | NO: 603 |
| Consensus | RASQXIX₂RX₃LN, where X₁ = G, S, F, N, or I, X₂ = S or T, and | SEQ ID |
| CDRL1 (Kabat) | X₃ = F, Y, or S | NO: 604 |
| Consensus | XIASX₂LX₃X₄, where X₁ = A or G, X₂ = T, S, or R, X₃ = Q or E, | SEQ ID |
| CDRL2 (Kabat) | and X₄ = N or S | NO: 605 |
| Consensus | QQX₁YX₂X₃X₄X₅T, where X₁ = T or S, X₂ = S, N, R, or G, X₃ = T | SEQ ID |
| CDRL3 (Kabat) | or S, X₄ = P or S, and X₅ = L or V | NO: 606 |
| Consensus | GFX₁FX₂X₃A, where X₁ = T or I, X₂ = N, I, or S, and X₃ = N, or Y | SEQ ID |
| CDRH1 | | NO: 607 |
| (Chothia) | | |
| Consensus | KX₁X₂X₃X₄GGT, where X₁ = S, R, or T, X₂ = I, E, K, L, N, or Q, X₃ | SEQ ID |
| CDRH2 | = S or T, and X₄ = Y or D | NO: 608 |
| (Chothia) | | |
| Consensus | X₁VWX₂YGGSLD, where X₁ = S or G, and X₂ = K, N, or R | SEQ ID |
| CDRH3 | | NO: 609 |
| (Chothia) | | |
| Consensus | SQX₁IX₂RX₃, where X₁ = G, S, F, N, or I, X₂ = S or T, and X₃ = F, | SEQ ID |
| CDRL1 | Y, or S | NO: 610 |
| (Chothia) | | |
| Consensus | X₁AS, where X₁ = A or G | SEQ ID |
| CDRL2 | | NO: 611 |
| (Chothia) | | |
| Consensus | X₁YX₂X₃X₄X₅, where X₁ = T or S, X₂ = S, N, R, or G, X₃ = T or S, | SEQ ID |
| CDRL3 | X₄ = P or S, and X₅ = L or V | NO: 612 |
| (Chothia) | | |
| Consensus | GFX₁FX₂X₃AW, where X₁ = T or I, X₂ = N, I, or S, and X₃ = N, or | SEQ ID |
| CDRH1 (IMGT) | Y | NO: 613 |
| Consensus | IKX₁X₂X₃X₄GGTX₅, where X₁ = S, R, or T, X₂ = I, E, K, L, N, or | SEQ ID |
| CDRH2 (IMGT) | Q, X₃ = S or T, X₄ = Y or D, and X₅ = T or R | NO: 614 |
| Consensus | XITGX₂VWX₃YGGSLDX₄, where X₁ = S, T, I, or A, X₂ = S or G, | SEQ ID |
| CDRH3 (IMGT) | X₃ = K, N, or R, and X₄ = F or Y | NO: 615 |
| Consensus | QX₁IX₂RX₃, where X₁ = G, S, F, N, or I, X₂ = S or T, and X₃ = F, Y, | SEQ ID |
| CDRL1 (IMGT) | or S | NO: 616 |
| Consensus | X₁AS, where X₁ = A or G | SEQ ID |
| CDRL2 (IMGT) | | NO: 617 |
| Consensus | QQX₁YX₂X₃X₄X₅T, where X₁ = T or S, X₂ = S, N, R, or G, X₃ = T | SEQ ID |
| CDRL3 (IMGT) | or S, X₄ = P or S, and X₅ = L or V | NO: 618 |
| Variants of 130 | X₁AWMS, where X₁ = Y, N, or T | SEQ ID |
| Consensus | | NO: 619 |
| CDRH1 (Kabat) | | |
| Consensus | RIKX₁X₂X₃X₄GGTTDYAX PVKG, where X₁ = S or R, X₂ = E or | SEQ ID |
| CDRH2 (Kabat) | K, X₃ = T, S, or I, X₄ = D or Y, and X₅ = A or S | NO: 620 |
| Consensus | GSSWX₁X₂YX₃FDX₄, where X₁ = N, H, S or Y, X₂ = V or L, X₃ = | SEQ ID |
| CDRH3 (Kabat) | For Y, and X₄ = F or Y | NO: 621 |
| Consensus | RASQSX₁SX₂X₃LN, where X₁ = I or M, X₂ = N, S, R or T, and X₃ = | SEQ ID |
| CDRL1 (Kabat) | Y or H | NO: 622 |
| Consensus | AAX₁X₂LX₃X₄, where X₁ = Y or S, X₂ = R, S, or N, X₃ = Q or E, | SEQ ID |
| CDRL2 (Kabat) | and X₄ = T or S | NO: 623 |
| Consensus | QQSYX₁X₂PX₃T, where X₁ = S or R, X₂ = S or T, and X₃ = L or F | SEQ ID |
| CDRL3 (Kabat) | | NO: 624 |
| Consensus | GFX₁FSX₂A, where X₁ = T or I, and X₂ = Y, N, or T | SEQ ID |
| CDRH1 | | NO: 625 |
| (Chothia) | | |
| Consensus | KX₁X₂X₃X₄GGT, where X₁ = S or R, X₂ = E or K, X₃ = T, S, or I, | SEQ ID |
| CDRH2 | X₄ = D or Y | NO: 626 |
| (Chothia) | | |
| Consensus | SSWX₁X₂YX₃FD, X₁ = N, H, S or Y, X₂ = V or L, and X₃ = F or Y | SEQ ID |
| CDRH3 | | NO: 627 |
| (Chothia) | | |
| Consensus | SQSX₁SX₂X₃, where X₁ = I or M, X₂ = N, S, R or T, and X₃ = Y or | SEQ ID |
| CDRL1 | H | NO: 628 |
| (Chothia) | | |
| Consensus | AAX₁, where X₁ = Y or S | SEQ ID |
| CDRL2 | | NO: 629 |
| (Chothia) | | |
| Consensus | SYX₁X₂PX₃, where X₁ = S or R, X₂ = S or T, and X₃ = L or F | |
| CDRL3 | | SEQ ID |
| (Chothia) | | NO: 630 |
| Consensus | GFX₁FSX₂AW, where X₁ = T or I, and X₂ = Y, N, or T | SEQ ID |
| CDRH1 (IMGT) | | NO: 631 |
| Consensus | IKX₁X₂X₃X₄GGTT, where X₁ = S or R, X₂ = E or K, X₃ = T, S, or I, | SEQ ID |
| CDRH2 (IMGT) | and X₄ = D or Y | NO: 632 |
| Consensus | TTGSSWX₁X₂YX₃FDX₄, where X₁ = N, H, S or Y, X₂ = V or L, X₃ | SEQ ID |
| CDRH3 (IMGT) | = F or Y, and X₄ = F or Y | NO: 633 |
| Consensus | QSX₁SX₂X₃, where X₁ = I or M, X₂ = N, S, R or T, and X₃ = Y or H | SEQ ID |
| CDRL1 (IMGT) | | NO: 634 |
| Consensus | AAX₁, where X₁ = Y or S | SEQ ID |
| CDRL2 (IMGT) | | NO: 635 |
| Consensus | QQSYX₁X₂PX₃T, where X₁ = S or R, X₂ = S or T, and X₃ = L or F | SEQ ID |
| CDRL3 (IMGT) | | NO: 636 |
| hIgG1* | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 637 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG4 | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 638 |
| | DHKPSNTKVDKRVESKYGPPCPSCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLGK | |
| IgG4-SP | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 639 |
| | DHKPSNTKVDKRVESKYGPPCPPCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLGK | |
| IgG4-SPLE | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 640 |
| | DHKPSNTKVDKRVESKYGPPCPPCPAPEELGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLGK | |
| IgG2 | ASTKGPSVFPLAPCSRSTSESTAALGCLVKDYFPEPVTVSWNS | SEQ ID |
| | GALTSGVHTFPAVLQSSGLYSLSSVVTVPSSNFGTQTYTCNV | NO: 641 |
| | DHKPSNTKVDKTVERKCCVECPPCPAPPVAGPSVFLFPPKPK | |
| | DTLMISRTPEVTCVVVDVSHEDPEVQFNWYVDGVEVHNAK | |
| | TKPREEQFNSTFRVVSVLTVVHQDWLNGKEYKCKVSNKGLP | |
| | APIEKTISKTKGQPREPQVYTLPPSREEMTKNQVSLTCLVKGF | |
| | YPSDIAVEWESNGQPENNYKTTPPMLDSDGSFFLYSKLTVDK | |
| | SRWQQGNVFSCSVMHEALHNHYTQKSLSLSP | |
| hIgG1-N297A | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 642 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1-D265A | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 643 |
| | NHKPSNTKVDKKVEPKSCDKTHTCPPCPAPELLGGPSVFLFP | |
| | PKPKDTLMISRTPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1-LALA | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 644 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1-LAGA | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 645 |
| | NHKPSNTKVDKKVEPKSCDKTHTCPPCPAPELAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAGA | SGALTSGVHTFPAVLQSSGLYSLSSVVTVPSSSLGTQTYICNV | NO: 646 |
| | NHKPSNTKVDKKVEPKSCDKTHTCPPCPAPEAAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1-LALAPG | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 647 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1-YTE | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 648 |
| | NHKPSNTKVDKKVEPKSCDKTHTCPPCPAPELLGGPSVFLFP | |
| | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| N297A/YTE | SGALTSGVHTFPAVLQSSGLYSLSSVVTVPSSSLGTQTYICNV | NO: 649 |
| | NHKPSNTKVDKKVEPKSCDKTHTCPPCPAPELLGGPSVFLFP | |
| | PKPKDTLYITREPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| D265A/YTE | SGALTSGVHTFPAVLQSSGLYSLSSVVTVPSSSLGTQTYICNV | NO: 650 |
| | NHKPSNTKVDKKVEPKSCDKTHTCPPCPAPELLGGPSVFLFP | |
| | PKPKDTLYITREPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALA/YTE | SGALTSGVHTFPAVLQSSGLYSLSSVVTVPSSSLGTQTYICNV | NO: 651 |
| | NHKPSNTKVDKKVEPKSCDKTHTCPPCPAPEAAGGPSVFLFP | |
| | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LAGA/YTE | SGALTSGVHTFPAVLQSSGLYSLSSVVTVPSSSLGTQTYICNV | NO: 652 |
| | NHKPSNTKVDKKVEPKSCDKTHTCPPCPAPELAGAPSVFLFP | |
| | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAGA/YTE | SGALTSGVHTFPAVLQSSGLYSLSSVVTVPSSSLGTQTYICNV | NO: 653 |
| | NHKPSNTKVDKKVEPKSCDKTHTCPPCPAPEAAGAPSVFLFP | |
| | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAPG/YTE | SGALTSGVHTFPAVLQSSGLYSLSSVVTVPSSSLGTQTYICNV | NO: 654 |
| | NHKPSNTKVDKKVEPKSCDKTHTCPPCPAPEAAGGPSVFLFP | |
| | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1-LS | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 655 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| hIgG1- | ASTKGPSVFPLAPSSKSTSGGTAALGCLVKDYFPEPVTVSWN | SEQ ID |
| N297A/LS | SGALTSGVHTFPAVLQSSGLYSLSSVVTVPSSSLGTQTYICNV | NO: 656 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| hIgG1- | ASTKGPSVFPLAPSSKSTSGGTAALGCLVKDYFPEPVTVSWN | SEQ ID |
| D265A/LS | SGALTSGVHTFPAVLQSSGLYSLSSVVTVPSSSLGTQTYICNV | NO: 657 |
| | NHKPSNTKVDKKVEPKSCDKTHTCPPCPAPELLGGPSVFLFP | |
| | PKPKDTLMISRTPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| hIgG1- | ASTKGPSVFPLAPSSKSTSGGTAALGCLVKDYFPEPVTVSWN | SEQ ID |
| LALA/LS | SGALTSGVHTFPAVLQSSGLYSLSSVVTVPSSSLGTQTYICNV | NO: 658 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| hIgG1- | ASTKGPSVFPLAPSSKSTSGGTAALGCLVKDYFPEPVTVSWN | SEQ ID |
| LAGA/LS | SGALTSGVHTFPAVLQSSGLYSLSSVVTVPSSSLGTQTYICNV | NO: 659 |
| | NHKPSNTKVDKKVEPKSCDKTHTCPPCPAPELAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| hIgG1- | ASTKGPSVFPLAPSSKSTSGGTAALGCLVKDYFPEPVTVSWN | SEQ ID |
| LALAGA/LS | SGALTSGVHTFPAVLQSSGLYSLSSVVTVPSSSLGTQTYICNV | NO: 660 |
| | NHKPSNTKVDKKVEPKSCDKTHTCPPCPAPEAAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| hIgG1- | ASTKGPSVFPLAPSSKSTSGGTAALGCLVKDYFPEPVTVSWN | SEQ ID |
| LALAPG/LS | SGALTSGVHTFPAVLQSSGLYSLSSVVTVPSSSLGTQTYICNV | NO: 661 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| hIgG1-DHS | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 662 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| hIgG1- | ASTKGPSVFPLAPSSKSTSGGTAALGCLVKDYFPEPVTVSWN | SEQ ID |
| N297A/DHS | SGALTSGVHTFPAVLQSSGLYSLSSVVTVPSSSLGTQTYICNV | NO: 663 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYASTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| hIgG1- | ASTKGPSVFPLAPSSKSTSGGTAALGCLVKDYFPEPVTVSWN | SEQ ID |
| D265A/DHS | SGALTSGVHTFPAVLQSSGLYSLSSVVTVPSSSLGTQTYICNV | NO: 664 |
| | NHKPSNTKVDKKVEPKSCDKTHTCPPCPAPELLGGPSVFLFP | |
| | PKPKDTLMISRTPEVTCVVVAVSHEDPEVKFNWYVDGVEVH | |
| | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| hIgG1- | ASTKGPSVFPLAPSSKSTSGGTAALGCLVKDYFPEPVTVSWN | SEQ ID |
| LALA/DHS | SGALTSGVHTFPAVLQSSGLYSLSSVVTVPSSSLGTQTYICNV | NO: 665 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| hIgG1- | ASTKGPSVFPLAPSSKSTSGGTAALGCLVKDYFPEPVTVSWN | SEQ ID |
| LAGA/DHS | SGALTSGVHTFPAVLQSSGLYSLSSVVTVPSSSLGTQTYICNV | NO: 666 |
| | NHKPSNTKVDKKVEPKSCDKTHTCPPCPAPELAGAPSVFLFP | |
| | PKPKDTLMISRTPEVTCVVVDVSHEDPEVKFNWYVDGVEVH | |
| | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| hIgG1- | ASTKGPSVFPLAPSSKSTSGGTAALGCLVKDYFPEPVTVSWN | SEQ ID |
| LALAGA/DHS | SGALTSGVHTFPAVLQSSGLYSLSSVVTVPSSSLGTQTYICNV | NO: 667 |
| | NHKPSNTKVDKKVEPKSCDKTHTCPPCPAPEAAGAPSVFLFP | |
| | PKPKDTLMISRTPEVTCVVVDVSHEDPEVKFNWYVDGVEVH | |
| | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| hIgG1- | NAKTKPREEQYNSTYRVVSVLTVDHHDWLNGKEYKCKVSN | SEQ ID |
| LALAPG/DHS | KALGAPIEKTISKAKGQPREPQVYTLPPSRDELTKNQVSLTCL | NO: 668 |
| | VKGFYPSDIAVEWESNGQPENNYKTTPPVLDSDGSFFLYSKL | |
| | TVDKSRWQQGNVFSCSVMHEALHSHYTQKSLSLSPG | |
| hIgG4-YTE | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 669 |
| | DHKPSNTKVDKRVESKYGPPCPSCPAPEFLGGPSVFLFPPKPK | |
| | DTLYITREPEVTCVVVDVSQEDPEVQFNWYVDGVEVHNAKT | |
| | KPREEQFNSTYRVVSVLTVLHQDWLNGKEYKCKVSNKGLPS | |
| | SIEKTISKAKGQPREPQVYTLPPSQEEMTKNQVSLTCLVKGFY | |
| | PSDIAVEWESNGQPENNYKTTPPVLDSDGSFFLYSRLTVDKS | |
| | RWQEGNVFSCSVMHEALHNHYTQKSLSLSLGK | |
| hIgG4-SP/YTE | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 670 |
| | DHKPSNTKVDKRVESKYGPPCPPCPAPEFLGGPSVFLFPPKPK | |
| | DTLYITREPEVTCVVVDVSQEDPEVQFNWYVDGVEVHNAKT | |
| | KPREEQFNSTYRVVSVLTVLHQDWLNGKEYKCKVSNKGLPS | |
| | SIEKTISKAKGQPREPQVYTLPPSQEEMTKNQVSLTCLVKGFY | |
| | PSDIAVEWESNGQPENNYKTTPPVLDSDGSFFLYSRLTVDKS | |
| | RWQEGNVFSCSVMHEALHNHYTQKSLSLSLGK | |
| hIgG4- | ASTKGPSVFPLAPCSRSTSESTAALGCLVKDYFPEPVTVSWNS | SEQ ID |
| SPLE/YTE | GALTSGVHTFPAVLQSSGLYSLSSVVTVPSSSLGTKTYTCNV | NO: 671 |
| | DHKPSNTKVDKRVESKYGPPCPPCPAPEELGGPSVFLFPPKPK | |
| | DTLYITREPEVTCVVVDVSQEDPEVQFNWYVDGVEVHNAKT | |
| | KPREEQFNSTYRVVSVLTVLHQDWLNGKEYKCKVSNKGLPS | |
| | SIEKTISKAKGQPREPQVYTLPPSQEEMTKNQVSLTCLVKGFY | |
| | PSDIAVEWESNGQPENNYKTTPPVLDSDGSFFLYSRLTVDKS | |
| | RWQEGNVFSCSVMHEALHNHYTQKSLSLSLGK | |
| hIgG4-LS | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 672 |
| | DHKPSNTKVDKRVESKYGPPCPSCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVLHEALHSYTQKSLSLSLGK | |
| hIgG4-SP/LS | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 673 |
| | DHKPSNTKVDKRVESKYGPPCPPCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVLHEALHSHYTQKSLSLSLGK | |
| hIgG4-SPLE/LS | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 674 |
| | DHKPSNTKVDKRVESKYGPPCPPCPAPEELGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVLHEALHSHYTQKSLSLSLGK | |
| hIgG4-DHS | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 675 |
| | DHKPSNTKVDKRVESKYGPPCPSCPAPEFLGGPSVFLFPPKPK | |
| | DTLMISRTPEVTCVVVDVSQEDPEVQFNWYVDGVEVHNAK | |
| | TKPREEQFNSTYRVVSVLTVDHHDWLNGKEYKCKVSNKGL | |
| | PSSIEKTISKAKGQPREPQVYTLPPSQEEMTKNQVSLTCLVKG | |
| | FYPSDIAVEWESNGQPENNYKTTPPVLDSDGSFFLYSRLTVD | |
| | KSRWQEGNVFSCSVMHEALHSHYTQKSLSLSLGK | |
| hIgG4-SP/DHS | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 676 |
| | DHKPSNTKVDKRVESKYGPPCPPCPAPEFLGGPSVFLFPPKPK | |
| | DTLMISRTPEVTCVVVDVSQEDPEVQFNWYVDGVEVHNAK | |
| | TKPREEQFNSTYRVVSVLTVDHHDWLNGKEYKCKVSNKGL | |
| | PSSIEKTISKAKGQPREPQVYTLPPSQEEMTKNQVSLTCLVKG | |
| | FYPSDIAVEWESNGQPENNYKTTPPVLDSDGSFFLYSRLTVD | |
| | KSRWQEGNVFSCSVMHEALHSHYTQKSLSLSLGK | |
| hIgG4- | ASTKGPSVFPLAPCSRSTSESTAALGCLVKDYFPEPVTVSWNS | SEQ ID |
| SPLE/DHS | GALTSGVHTFPAVLQSSGLYSLSSVVTVPSSSLGTKTYTCNV | NO: 677 |
| | DHKPSNTKVDKRVESKYGPPCPPCPAPEELGGPSVFLFPPKPK | |
| | DTLMISRTPEVTCVVVDVSQEDPEVQFNWYVDGVEVHNAK | |
| | TKPREEQFNSTYRVVSVLTVDHHDWLNGKEYKCKVSNKGL | |
| | PSSIEKTISKAKGQPREPQVYTLPPSQEEMTKNQVSLTCLVKG | |
| | FYPSDIAVEWESNGQPENNYKTTPPVLDSDGSFFLYSRLTVD | |
| | KSRWQEGNVFSCSVMHEALHSHYTQKSLSLSLGK | |
| hIgG2-YTE | ASTKGPSVFPLAPCSRSTSESTAALGCLVKDYFPEPVTVSWNS | SEQ ID |
| | GALTSGVHTFPAVLQSSGLYSLSSVVTVPSSNFGTQTYTCNV | NO: 678 |
| | DHKPSNTKVDKTVERKCCVECPPCPAPPVAGPSVFLFPPKPK | |
| | DTLYITREPEVTCVVVDVSHEDPEVQFNWYVDGVEVHNAKT | |
| | KPREEQFNSTFRVVSVLTVVHQDWLNGKEYKCKVSNKGLPA | |
| | PIEKTISKTKGQPREPQVYTLPPSREEMTKNQVSLTCLVKGFY | |
| | PSDIAVEWESNGQPENNYKTTPPMLDSDGSFFLYSKLTVDKS | |
| | RWQQGNVFSCSVLHEALHSHYTQKSLSLSP | |
| hIgG2-LS | ASTKGPSVFPLAPCSRSTSESTAALGCLVKDYFPEPVTVSWNS | SEQ ID |
| | GALTSGVHTFPAVLQSSGLYSLSSVVTVPSSNFGTQTYTCNV | NO: 679 |
| | DHKPSNTKVDKTVERKCCVECPPCPAPPVAGPSVFLFPPKPK | |
| | DTLMISRTPEVTCVVVDVSHEDPEVQFNWYVDGVEVHNAK | |
| | TKPREEQFNSTFRVVSVLTVVHQDWLNGKEYKCKVSNKGLP | |
| | APIEKTISKTKGQPREPQVYTLPPSREEMTKNQVSLTCLVKGF | |
| | YPSDIAVEWESNGQPENNYKTTPPMLDSDGSFFLYSKLTVDK | |
| | SRWQQGNVFSCSVMHEALHNHYTQKSLSLSP | |
| hIgG2-DHS | ASTKGPSVFPLAPCSRSTSESTAALGCLVKDYFPEPVTVSWNS | SEQ ID |
| | GALTSGVHTFPAVLQSSGLYSLSSVVTVPSSNFGTQTYTCNV | NO: 680 |
| | DHKPSNTKVDKTVERKCCVECPPCPAPPVAGPSVFLFPPKPK | |
| | DTLMISRTPEVTCVVVDVSHEDPEVQFNWYVDGVEVHNAK | |
| | TKPREEQFNSTFRVVSVLTVDHHDWLNGKEYKCKVSNKGLP | |
| | APIEKTISKTKGQPREPQVYTLPPSREEMTKNQVSLTCLVKGF | |
| | YPSDIAVEWESNGQPENNYKTTPPMLDSDGSFFLYSKLTVDK | |
| | SRWQQGNVFSCSVMHEALHSHYTQKSLSLSP | |
| IgG4-SP | ASTKGPSVFPLAPCSRSTSESTAALGCLVKDYFPEPVTVSWNS | SEQ ID |
| | GALTSGVHTFPAVLQSSGLYSLSSVVTVPSSSLGTKTYTCNV | NO: 681 |
| | DHKPSNTKVDKRVESKYGPPCPPCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLGK | |
| hIgG1-LA | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 682 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| hIgG1- | ASTKGPSVFPLAPSSKSTSGGTAALGCLVKDYFPEPVTVSWN | SEQ ID |
| N297A/LA | SGALTSGVHTFPAVLQSSGLYSLSSVVTVPSSSLGTQTYICNV | NO: 683 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| hIgG1- | ASTKGPSVFPLAPSSKSTSGGTAALGCLVKDYFPEPVTVSWN | SEQ ID |
| D265A/LA | SGALTSGVHTFPAVLQSSGLYSLSSVVTVPSSSLGTQTYICNV | NO: 684 |
| | NHKPSNTKVDKKVEPKSCDKTHTCPPCPAPELLGGPSVFLFP | |
| | PKPKDTLMISRTPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| hIgG1- | ASTKGPSVFPLAPSSKSTSGGTAALGCLVKDYFPEPVTVSWN | SEQ ID |
| LALA/LA | SGALTSGVHTFPAVLQSSGLYSLSSVVTVPSSSLGTQTYICNV | NO: 685 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| hIgG1- | ASTKGPSVFPLAPSSKSTSGGTAALGCLVKDYFPEPVTVSWN | SEQ ID |
| LAGA/LA | SGALTSGVHTFPAVLQSSGLYSLSSVVTVPSSSLGTQTYICNV | NO: 686 |
| | NHKPSNTKVDKKVEPKSCDKTHTCPPCPAPELAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| hIgG1- | ASTKGPSVFPLAPSSKSTSGGTAALGCLVKDYFPEPVTVSWN | SEQ ID |
| LALAGA/LA | SGALTSGVHTFPAVLQSSGLYSLSSVVTVPSSSLGTQTYICNV | NO: 687 |
| | NHKPSNTKVDKKVEPKSCDKTHTCPPCPAPEAAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| hIgG1- | ASTKGPSVFPLAPSSKSTSGGTAALGCLVKDYFPEPVTVSWN | SEQ ID |
| LALAPG/LA | SGALTSGVHTFPAVLQSSGLYSLSSVVTVPSSSLGTQTYICNV | NO: 688 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| hIgG1-N434A | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 689 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| hIgG1-N297A/ | ASTKGPSVFPLAPSSKSTSGGTAALGCLVKDYFPEPVTVSWN | SEQ ID |
| N434A | SGALTSGVHTFPAVLQSSGLYSLSSVVTVPSSSLGTQTYICNV | NO: 690 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| hIgG1-D265A/ | ASTKGPSVFPLAPSSKSTSGGTAALGCLVKDYFPEPVTVSWN | SEQ ID |
| N434A | SGALTSGVHTFPAVLQSSGLYSLSSVVTVPSSSLGTQTYICNV | NO: 691 |
| | NHKPSNTKVDKKVEPKSCDKTHTCPPCPAPELLGGPSVFLFP | |
| | PKPKDTLMISRTPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| hIgG1-LALA/ | ASTKGPSVFPLAPSSKSTSGGTAALGCLVKDYFPEPVTVSWN | SEQ ID |
| N434A | SGALTSGVHTFPAVLQSSGLYSLSSVVTVPSSSLGTQTYICNV | NO: 692 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| hIgG1-LAGA/ | ASTKGPSVFPLAPSSKSTSGGTAALGCLVKDYFPEPVTVSWN | SEQ ID |
| N434A | SGALTSGVHTFPAVLQSSGLYSLSSVVTVPSSSLGTQTYICNV | NO: 693 |
| | NHKPSNTKVDKKVEPKSCDKTHTCPPCPAPELAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| hIgG1- | ASTKGPSVFPLAPSSKSTSGGTAALGCLVKDYFPEPVTVSWN | SEQ ID |
| LALAGA/ | SGALTSGVHTFPAVLQSSGLYSLSSVVTVPSSSLGTQTYICNV | NO: 694 |
| N434A | NHKPSNTKVDKKVEPKSCDKTHTCPPCPAPEAAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| hIgG1- | ASTKGPSVFPLAPSSKSTSGGTAALGCLVKDYFPEPVTVSWN | SEQ ID |
| LALAPG/ | SGALTSGVHTFPAVLQSSGLYSLSSVVTVPSSSLGTQTYICNV | NO: 695 |
| N434A | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| hIgG1-N434W | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 696 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| hIgG1-N297A/ | ASTKGPSVFPLAPSSKSTSGGTAALGCLVKDYFPEPVTVSWN | SEQ ID |
| N434W | SGALTSGVHTFPAVLQSSGLYSLSSVVTVPSSSLGTQTYICNV | NO: 697 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| hIgG1-D265A/ | ASTKGPSVFPLAPSSKSTSGGTAALGCLVKDYFPEPVTVSWN | SEQ ID |
| N434W | SGALTSGVHTFPAVLQSSGLYSLSSVVTVPSSSLGTQTYICNV | NO: 698 |
| | NHKPSNTKVDKKVEPKSCDKTHTCPPCPAPELLGGPSVFLFP | |
| | PKPKDTLMISRTPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| hIgG1-LALA/ | ASTKGPSVFPLAPSSKSTSGGTAALGCLVKDYFPEPVTVSWN | SEQ ID |
| N434W | SGALTSGVHTFPAVLQSSGLYSLSSVVTVPSSSLGTQTYICNV | NO: 699 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| hIgG1-LAGA/ | ASTKGPSVFPLAPSSKSTSGGTAALGCLVKDYFPEPVTVSWN | SEQ ID |
| N434W | SGALTSGVHTFPAVLQSSGLYSLSSVVTVPSSSLGTQTYICNV | NO: 700 |
| | NHKPSNTKVDKKVEPKSCDKTHTCPPCPAPELAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| hIgG1- | ASTKGPSVFPLAPSSKSTSGGTAALGCLVKDYFPEPVTVSWN | SEQ ID |
| LALAGA/ | SGALTSGVHTFPAVLQSSGLYSLSSVVTVPSSSLGTQTYICNV | NO: 701 |
| N434W | NHKPSNTKVDKKVEPKSCDKTHTCPPCPAPEAAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| hIgG1- | ASTKGPSVFPLAPSSKSTSGGTAALGCLVKDYFPEPVTVSWN | SEQ ID |
| LALAPG/ | SGALTSGVHTFPAVLQSSGLYSLSSVVTVPSSSLGTQTYICNV | NO: 702 |
| N434W | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| hIgG1/DQ | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 703 |
| | NHKPSNTKVDKKVEPKSCDKTHTCPPCPAPELLGGPSVFLFP | |
| | PKPKDTLMISRDPEVTCVVVDVSHEDPEVKFNWYVDGVEVH | |
| | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| N297A/DQ | SGALTSGVHTFPAVLQSSGLYSLSSVVTVPSSSLGTQTYICNV | NO: 704 |
| | NHKPSNTKVDKKVEPKSCDKTHTCPPCPAPELLGGPSVFLFP | |
| | PKPKDTLMISRDPEVTCVVVDVSHEDPEVKFNWYVDGVEVH | |
| | NAKTKPREEQYASTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| D265A/DQ | SGALTSGVHTFPAVLQSSGLYSLSSVVTVPSSSLGTQTYICNV | NO: 705 |
| | NHKPSNTKVDKKVEPKSCDKTHTCPPCPAPELLGGPSVFLFP | |
| | PKPKDTLMISRDPEVTCVVVAVSHEDPEVKFNWYVDGVEVH | |
| | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALA/DQ | SGALTSGVHTFPAVLQSSGLYSLSSVVTVPSSSLGTQTYICNV | NO: 706 |
| | NHKPSNTKVDKKVEPKSCDKTHTCPPCPAPEAAGGPSVFLFP | |
| | PKPKDTLMISRDPEVTCVVVDVSHEDPEVKFNWYVDGVEVH | |
| | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LAGA/DQ | SGALTSGVHTFPAVLQSSGLYSLSSVVTVPSSSLGTQTYICNV | NO: 707 |
| | NHKPSNTKVDKKVEPKSCDKTHTCPPCPAPELAGAPSVFLFP | |
| | PKPKDTLMISRDPEVTCVVVDVSHEDPEVKFNWYVDGVEVH | |
| | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAGA/DQ | SGALTSGVHTFPAVLQSSGLYSLSSVVTVPSSSLGTQTYICNV | NO: 708 |
| | NHKPSNTKVDKKVEPKSCDKTHTCPPCPAPEAAGAPSVFLFP | |
| | PKPKDTLMISRDPEVTCVVVDVSHEDPEVKFNWYVDGVEVH | |
| | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAPG/DQ | SGALTSGVHTFPAVLQSSGLYSLSSVVTVPSSSLGTQTYICNV | NO: 709 |
| | NHKPSNTKVDKKVEPKSCDKTHTCPPCPAPEAAGGPSVFLFP | |
| | PKPKDTLMISRDPEVTCVVVDVSHEDPEVKFNWYVDGVEVH | |
| | NAKTKPREEQYNSTYRVVSVLQVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1/DW | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 710 |
| | NHKPSNTKVDKKVEPKSCDKTHTCPPCPAPELLGGPSVFLFP | |
| | PKPKDTLMISRDPEVTCVVVDVSHEDPEVKFNWYVDGVEVH | |
| | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| N297A/DW | SGALTSGVHTFPAVLQSSGLYSLSSVVTVPSSSLGTQTYICNV | NO: 711 |
| | NHKPSNTKVDKKVEPKSCDKTHTCPPCPAPELLGGPSVFLFP | |
| | PKPKDTLMISRDPEVTCVVVDVSHEDPEVKFNWYVDGVEVH | |
| | NAKTKPREEQYASTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| D265A/DW | SGALTSGVHTFPAVLQSSGLYSLSSVVTVPSSSLGTQTYICNV | NO: 712 |
| | NHKPSNTKVDKKVEPKSCDKTHTCPPCPAPELLGGPSVFLFP | |
| | PKPKDTLMISRDPEVTCVVVAVSHEDPEVKFNWYVDGVEVH | |
| | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALA/DW | SGALTSGVHTFPAVLQSSGLYSLSSVVTVPSSSLGTQTYICNV | NO: 713 |
| | NHKPSNTKVDKKVEPKSCDKTHTCPPCPAPEAAGGPSVFLFP | |
| | PKPKDTLMISRDPEVTCVVVDVSHEDPEVKFNWYVDGVEVH | |
| | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LAGA/DW | SGALTSGVHTFPAVLQSSGLYSLSSVVTVPSSSLGTQTYICNV | NO: 714 |
| | NHKPSNTKVDKKVEPKSCDKTHTCPPCPAPELAGAPSVFLFP | |
| | PKPKDTLMISRDPEVTCVVVDVSHEDPEVKFNWYVDGVEVH | |
| | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAGA/DW | SGALTSGVHTFPAVLQSSGLYSLSSVVTVPSSSLGTQTYICNV | NO: 715 |
| | NHKPSNTKVDKKVEPKSCDKTHTCPPCPAPEAAGAPSVFLFP | |
| | PKPKDTLMISRDPEVTCVVVDVSHEDPEVKFNWYVDGVEVH | |
| | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAPG/DW | SGALTSGVHTFPAVLQSSGLYSLSSVVTVPSSSLGTQTYICNV | NO: 716 |
| | NHKPSNTKVDKKVEPKSCDKTHTCPPCPAPEAAGGPSVFLFP | |
| | PKPKDTLMISRDPEVTCVVVDVSHEDPEVKFNWYVDGVEVH | |
| | NAKTKPREEQYNSTYRVVSVLWVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1/YD | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 717 |
| | NHKPSNTKVDKKVEPKSCDKTHTCPPCPAPELLGGPSVFLFP | |
| | PKPKDTLYISRDPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| N297A/YD | SGALTSGVHTFPAVLQSSGLYSLSSVVTVPSSSLGTQTYICNV | NO: 718 |
| | NHKPSNTKVDKKVEPKSCDKTHTCPPCPAPELLGGPSVFLFP | |
| | PKPKDTLYISRDPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| D265A/YD | SGALTSGVHTFPAVLQSSGLYSLSSVVTVPSSSLGTQTYICNV | NO: 719 |
| | NHKPSNTKVDKKVEPKSCDKTHTCPPCPAPELLGGPSVFLFP | |
| | PKPKDTLYISRDPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALA/YD | SGALTSGVHTFPAVLQSSGLYSLSSVVTVPSSSLGTQTYICNV | NO: 720 |
| | NHKPSNTKVDKKVEPKSCDKTHTCPPCPAPEAAGGPSVFLFP | |
| | PKPKDTLYISRDPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LAGA/YD | SGALTSGVHTFPAVLQSSGLYSLSSVVTVPSSSLGTQTYICNV | NO: 721 |
| | NHKPSNTKVDKKVEPKSCDKTHTCPPCPAPELAGAPSVFLFP | |
| | PKPKDTLYISRDPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAGA/YD | SGALTSGVHTFPAVLQSSGLYSLSSVVTVPSSSLGTQTYICNV | NO: 722 |
| | NHKPSNTKVDKKVEPKSCDKTHTCPPCPAPEAAGAPSVFLFP | |
| | PKPKDTLYISRDPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAPG/YD | SGALTSGVHTFPAVLQSSGLYSLSSVVTVPSSSLGTQTYICNV | NO: 723 |
| | NHKPSNTKVDKKVEPKSCDKTHTCPPCPAPEAAGGPSVFLFP | |
| | PKPKDTLYISRDPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| hIgG1/QVV | ASTKGPSVFPLAPSSKSTSGGTAALGCLVKDYFPEPVT | SEQ ID |
| | VSWNSGALTSGVHTFPAVLQSSGLYSLSSVVTVPSSSLGTQT | NO: 724 |
| | YICNVNHKPSNTKVDKKVEPKSCDKTHTCPPCPAPELLGGPS | |
| | VFLFPPKPKDTLMISRTPEVTCVVVDVSHEDPEVKFNWYVD | |
| | GVEVHNAKTKPREEQYNSTYRVVSVLQVLHVDWLNGKEYK | |
| | CKVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| N297A/QVV | SGALTSGVHTFPAVLQSSGLYSLSSVVTVPSSSLGTQTYICNV | NO: 725 |
| | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYASTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| D265A/QVV | SGALTSGVHTFPAVLQSSGLYSLSSVVTVPSSSLGTQTYICNV | NO: 726 |
| | NHKPSNTKVDKKVEPKSCDKTHTCPPCPAPELLGGPSVFLFP | |
| | PKPKDTLMISRTPEVTCVVVAVSHEDPEVKFNWYVDGVEVH | |
| | NAKTKPREEQYNSTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALA/QVV | SGALTSGVHTFPAVLQSSGLYSLSSVVTVPSSSLGTQTYICNV | NO: 727 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LAGA/QVV | SGALTSGVHTFPAVLQSSGLYSLSSVVTVPSSSLGTQTYICNV | NO: 728 |
| | NHKPSNTKVDKKVEPKSCDKTHTCPPCPAPELAGAPSVFLFP | |
| | PKPKDTLMISRTPEVTCVVVDVSHEDPEVKFNWYVDGVEVH | |
| | NAKTKPREEQYNSTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAGA/QVV | SGALTSGVHTFPAVLQSSGLYSLSSVVTVPSSSLGTQTYICNV | NO: 729 |
| | NHKPSNTKVDKKVEPKSCDKTHTCPPCPAPEAAGAPSVFLFP | |
| | PKPKDTLMISRTPEVTCVVVDVSHEDPEVKFNWYVDGVEVH | |
| | NAKTKPREEQYNSTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAPG/QVV | SGALTSGVHTFPAVLQSSGLYSLSSVVTVPSSSLGTQTYICNV | NO: 730 |
| | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLQVLHVDWLNGKEYKCKVSN | |
| | KALGAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1/DDRVV | ASTKGPSVFPLAPSSKSTSGGTAALGCLVKDYFPEPVTVSWN | SEQ ID |
| | SGALTSGVHTFPAVLQSSGLYSLSSVVTVPSSSLGTQTYICNV | NO: 731 |
| | NHKPSNTKVDKKVEPKSCDKTHTCPPCPAPELLGGPSVFLFP | |
| | PKPKDTLMISRDPEVTCVVVDVSHEDPEVKFNWYVDGVEVD | |
| | NAKTKPREEQYNSTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| N297A/DDRVV | SGALTSGVHTFPAVLQSSGLYSLSSVVTVPSSSLGTQTYICNV | NO: 732 |
| | NHKPSNTKVDKKVEPKSCDKTHTCPPCPAPELLGGPSVFLFP | |
| | PKPKDTLMISRDPEVTCVVVDVSHEDPEVKFNWYVDGVEVD | |
| | NAKTKPREEQYASTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| D265A/DDRVV | SGALTSGVHTFPAVLQSSGLYSLSSVVTVPSSSLGTQTYICNV | NO: 733 |
| | NHKPSNTKVDKKVEPKSCDKTHTCPPCPAPELLGGPSVFLFP | |
| | PKPKDTLMISRDPEVTCVVVAVSHEDPEVKFNWYVDGVEVD | |
| | NAKTKPREEQYNSTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALA/DDRVV | SGALTSGVHTFPAVLQSSGLYSLSSVVTVPSSSLGTQTYICNV | NO: 734 |
| | NHKPSNTKVDKKVEPKSCDKTHTCPPCPAPEAAGGPSVFLFP | |
| | PKPKDTLMISRDPEVTCVVVDVSHEDPEVKFNWYVDGVEVD | |
| | NAKTKPREEQYNSTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKKVEPKSCDKTHTCPPCPAPELAGAPSVFLFP | |
| hIgG1- | PKPKDTLMISRDPEVTCVVVDVSHEDPEVKFNWYVDGVEVD | SEQ ID |
| LAGA/DDRVV | NAKTKPREEQYNSTYRVVSVLRVLHVDWLNGKEYKCKVSN | NO: 735 |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAGA/DDR | SGALTSGVHTFPAVLQSSGLYSLSSVVTVPSSSLGTQTYICNV | NO: 736 |
| VV | NHKPSNTKVDKKVEPKSCDKTHTCPPCPAPEAAGAPSVFLFP | |
| | PKPKDTLMISRDPEVTCVVVDVSHEDPEVKFNWYVDGVEVD | |
| | NAKTKPREEQYNSTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| hIgG1- | ASTKGPSVFPLAPSSKSTSGGTAALGCLVKDYFPEPVTVSWN | SEQ ID |
| LALAPG/DDR | SGALTSGVHTFPAVLQSSGLYSLSSVVTVPSSSLGTQTYICNV | NO: 737 |
| VV | NHKPSNTKVDKKVEPKSCDKTHTCPPCPAPEAAGGPSVFLFP | |
| | PKPKDTLMISRDPEVTCVVVDVSHEDPEVKFNWYVDGVEVD | |
| | NAKTKPREEQYNSTYRVVSVLRVLHVDWLNGKEYKCKVSN | |
| | KALGAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Human kappa | RTVAAPSVFIFPPSDEQLKSGTASVVCLLNNFYPREAKVQWK | SEQ ID |
| LC | VDNALQSGNSQESVTEQDSKDSTYSLSSTLTLSKADYEKHKV | NO: 738 |
| | YACEVTHQGLSSPVTKSFNRGEC | |
| OX40L protein | MERVQPLEENVGNAARPRFERNKLLLVASVIQGLGLLLCFTY | SEQ ID |
| | ICLHFSALQVSHRYPRIQSIKVQFTEYKKEKGFILTSQKEDEIM | NO: 739 |
| | KVQNNSVIINCDGFYLISLKGYFSQEVNISLHYQKDEEPLFQL | |
| | KKVRSVNSLMVASLTYKDKVYLNVTTDNTSLDDFHVNGGE | |
| | LILIHQNPGEFCVL | |
| Kabat HCDR1 | SGNWWS | SEQ ID |
| | | NO: 740 |
| Kabat HCDR1 | SGGYYWS | SEQ ID |
| | | NO: 741 |
| Chothia HCDR1 | GGSISSGN | SEQ ID |
| | | NO: 742 |
| Chothia HCDR1 | GFTFDSA | SEQ ID |
| | | NO: 743 |
| Chothia HCDR1 | GGSISSGGY | SEQ ID |
| | | NO: 744 |
| IMGT CDR1 | GGSISSGNW | SEQ ID |
| | | NO: 745 |
| IMGT CDR1 | GFTFDSAW | SEQ ID |
| | | NO: 746 |
| IMGT CDR1 | GGSISSGGYY | SEQ ID |
| | | NO: 747 |
| Kabat HCDR2 | RFKSETDGGTKDFGAHVRG | SEQ ID |
| | | NO: 748 |
| Kabat HCDR2 | EIHHSGRTNYNPSLKS | SEQ ID |
| | | NO: 749 |
| Kabat HCDR2 | RIKRKTDGGTTDYAAPVKG | SEQ ID |
| | | NO: 750 |
| Kabat HCDR2 | RIKSKNEGGTIDYAAPVKG | SEQ ID |
| | | NO: 751 |
| Kabat HCDR2 | WIHPNTGKTGYAQKFQG | SEQ ID |
| | | NO: 752 |
| Kabat HCDR2 | HIYYSGSTKYNPSLKS | SEQ ID |
| | | NO: 753 |
| Chothia HCDR2 | HHSGR | SEQ ID |
| | | NO: 754 |
| Chothia HCDR2 | KSKNEGGT | SEQ ID |
| | | NO: 755 |
| Chothia HCDR2 | HPNTGK | SEQ ID |
| | | NO: 756 |
| Chothia HCDR2 | YYSGS | SEQ ID |
| | | NO: 757 |
| IMGT HCDR2 | FKSETDGGTK | SEQ ID |
| | | NO: 758 |
| IMGT HCDR2 | IHHSGRT | SEQ ID |
| | | NO: 759 |
| IMGT HCDR2 | IKSKNEGGTI | SEQ ID |
| | | NO: 760 |
| IMGT HCDR2 | IHPNTGKT | SEQ ID |
| | | NO: 761 |
| IMGT HCDR2 | IYYSGST | SEQ ID |
| | | NO: 762 |
| Kabat HCDR3 | GGTAVPFDF | SEQ ID |
| | | NO: 763 |
| Kabat HCDR3 | DROGSFAFDI | SEQ ID |
| | | NO: 764 |
| Kabat HCDR3 | DRPWGSFFDY | SEQ ID |
| | | NO: 765 |
| Kabat HCDR3 | DFLWFGELPFDY | SEQ ID |
| | | NO: 766 |
| Kabat HCDR3 | ESYDLWSGHSFNWFDP | SEQ ID |
| | | NO: 767 |
| Kabat HCDR3 | DNWGSGWYYMDV | SEQ ID |
| | | NO: 768 |
| Chothia HCDR3 | GTAVPFD | SEQ ID |
| | | NO: 769 |
| Chothia HCDR3 | RQGSFAFD | SEQ ID |
| | | NO: 770 |
| Chothia HCDR3 | RPWGSFFD | SEQ ID |
| | | NO: 771 |
| Chothia HCDR3 | FLWFGELPFD | SEQ ID |
| | | NO: 772 |
| Chothia HCDR3 | SYDLWSGHSFNWFD | SEQ ID |
| | | NO: 773 |
| Chothia HCDR3 | NWGSGWYYMD | SEQ ID |
| | | NO: 774 |
| IMGT HCDR3 | TTGGTAVPFDF | SEQ ID |
| | | NO: 775 |
| IMGT HCDR3 | ARDROGSFAFDI | SEQ ID |
| | | NO: 776 |
| IMGT HCDR3 | TLDRPWGSFFDY | SEQ ID |
| | | NO: 777 |
| IMGT HCDR3 | NTDFLWFGELPFDY | SEQ ID |
| | | NO: 778 |
| IMGT HCDR3 | ARESYDLWSGHSFNWFDP | SEQ ID |
| | | NO: 779 |
| IMGT HCDR3 | ARDNWGSGWYYMDV | SEQ ID |
| | | NO: 780 |
| Kabat LCDR1 | KSSQSLLYSSNNKNYLA | SEQ ID |
| | | NO: 781 |
| Kabat LCDR1 | RASQSISTWLV | SEQ ID |
| | | NO: 782 |
| Kabat LCDR1 | KSSQSVLYSSNNKNYFA | SEQ ID |
| | | NO: 783 |
| Kabat LCDR1 | RASQGISNHLA | SEQ ID |
| | | NO: 784 |
| Kabat LCDR1 | RASQGITSYLA | SEQ ID |
| | | NO: 785 |
| Kabat LCDR1 | RASQSVSNYFA | SEQ ID |
| | | NO: 786 |
| Chothia LCDR1 | SQSLLYSSNNKNY | SEQ ID |
| | | NO: 787 |
| Chothia LCDR1 | SQSISTW | SEQ ID |
| | | NO: 788 |
| Chothia LCDR1 | SQGISNH | SEQ ID |
| | | NO: 789 |
| Chothia LCDR1 | SQGITSY | SEQ ID |
| | | NO: 790 |
| Chothia LCDR1 | SQSVSNY | SEQ ID |
| | | NO: 791 |
| IMGT LCDR1 | QSLLYSSNNKNY | SEQ ID |
| | | NO: 792 |
| IMGT LCDR1 | QSISTW | SEQ ID |
| | | NO: 793 |
| IMGT LCDR1 | QGISNH | SEQ ID |
| | | NO: 794 |
| IMGT LCDR1 | QGITSY | SEQ ID |
| | | NO: 795 |
| IMGT LCDR1 | QSVSNY | SEQ ID |
| | | NO: 796 |
| Kabat LCDR2 | WTSTRES | SEQ ID |
| | | NO: 797 |
| Kabat LCDR2 | KASSLES | SEQ ID |
| | | NO: 798 |
| Kabat LCDR2 | RASTRES | SEQ ID |
| | | NO: 799 |
| Kabat LCDR2 | GATTLQS | SEQ ID |
| | | NO: 800 |
| Kabat LCDR2 | DASNRAT | SEQ ID |
| | | NO: 801 |
| Chothia and | WTS | SEQ ID |
| IMGT LCDR2 | | NO: 802 |
| Chothia and | KAS | SEQ ID |
| IMGT LCDR2 | | NO: 803 |
| Chothia and | RAS | SEQ ID |
| IMGT LCDR2 | | NO: 804 |
| Chothia and | GAT | SEQ ID |
| IMGT LCDR2 | | NO: 805 |
| Kabat LCDR3 | LQYSNTPYS | SEQ ID |
| | | NO: 806 |
| Kabat LCDR3 | QQHISYPYT | SEQ ID |
| | | NO: 807 |
| Kabat LCDR3 | QQYYTTPLT | SEQ ID |
| | | NO: 808 |
| Kabat LCDR3 | QKLNSAPLT | SEQ ID |
| | | NO: 809 |
| Kabat LCDR3 | QQLQSYPYT | SEQ ID |
| | | NO: 810 |
| Kabat LCDR3 | QHRSNWPLT | SEQ ID |
| | | NO: 811 |
| Chothia and | YSNTPY | SEQ ID |
| IMGT LCDR3 | | NO: 812 |
| Chothia and | HISYPY | SEQ ID |
| IMGT LCDR3 | | NO: 813 |
| Chothia and | YYTTPL | SEQ ID |
| IMGT LCDR3 | | NO: 814 |
| Chothia and | LNSAPL | SEQ ID |
| IMGT LCDR3 | | NO: 815 |
| Chothia and | LQSYPY | SEQ ID |
| IMGT LCDR3 | | NO: 816 |
| Chothia and | RSNWPL | SEQ ID |
| IMGT LCDR3 | | NO: 817 |
| Heavy Chain | EVQLVESGGGLVKPGGSLRLSCAASGFTFSNAWMSWVRQAP | SEQ ID |
| Variable Domain | GKGLEWVGRFKSETDGGTKDFGAHVRGRFIISKDASKNTLY | NO: 818 |
| 71 | LQMNRLKTEDTAVYYCTTGGTAVPFDFWGLGTLVTVSS | |
| Heavy Chain | QVQLQESGPGLVKPSGTLSLTCAVSGGSISSGNWWSWVRQP | SEQ ID |
| Variable Domain | PGSGLEWIGEIHHSGRTNYNPSLKSRVVISVDKSKNHFSLKLS | NO: 819 |
| 72 | SVTAADTAVYYCARDROGSFAFDIWGQGTMVTVSS | |
| Heavy Chain | EVQLVESGGGLVKPGGSLRLSCTASGFTFDSAWMSWVRQAP | SEQ ID |
| Variable Domain | GKGLEWVGRIKRKTDGGTTDYAAPVKGRFTISRDDSTKTLY | NO: 820 |
| 73 | LQMNSLKTEDTAVYYCTLDRPWGSFFDYWGQGTLVTVSS | |
| Heavy Chain | EVQLVESGGGLVKPGGSLRLSCAASGFTFSNAWMSWVRQAP | SEQ ID |
| Variable Domain | GKGLEWVGRIKSKNEGGTIDYAAPVKGRFTISRDDSKNTLYL | NO: 821 |
| 74 | QMNSLKTEDTAVYYCNTDFLWFGELPFDYWGQGTLVTVSS | |
| Heavy Chain | EVQVVESGGGLVQPGGSLRLSCAASGFTFSSYAMTWVRQAP | SEQ ID |
| Variable Domain | GKGLEWVSLISGSGGLTKYADSVKGRFTISRDNSKKTLYLQ | NO: 822 |
| 75 | MNSLRAEDTAAYYCVKDEGLTTGEYWGQGTLVTVSS | |
| Heavy Chain | QVQLVQSGTEVKKPGASVKVSCKASGYTFTNYDINWVRQA | SEQ ID |
| Variable Domain | TGQGLEWVGWIHPNTGKTGYAQKFQGRVTMTRDISIGTAY | NO: 823 |
| 76 | MELNSLRSEDTAVYYCARESYDLWSGHSFNWFDPWGQGTL | |
| | VTVSS | |
| Heavy Chain | QVQLQESGPGLVKPSQTLSLTCTVSGGSISSGGYYWSWIRQY | SEQ ID |
| Variable Domain | PGKGLEWIGHIYYSGSTKYNPSLKSRLTISVDTSKNQFSLKLS | NO: 824 |
| 77 | SVTAADTAVYYCARDNWGSGWYYMDVWGKGTTVTVSS | |
| Light Chain | DIVMTQSPDSLAVSLGERATINCKSSQSLLYSSNNKNYLAWY | SEQ ID |
| Variable Domain | QQKPGQPPKLLFYWTSTRESGVPDRFGASGSGTDFTLTISSLQ | NO: 825 |
| 57 | VEDVAIYFCLQYSNTPYSFGQGTKLEIK | |
| Light Chain | DIQMTQSPATLSASVGDRVTITCRASQSISTWLVWYQQKPGK | SEQ ID |
| Variable Domain | APKLLIYKASSLESGVPSRFSGSGSGTEFTLTISSLQPDDFATY | NO: 826 |
| 58 | YCQQHISYPYTFGQGTRLEIK | |
| Light Chain | DIVMTQSPDSLAVSLGERATINCKSSQSVLYSSNNKNYFAWY | SEQ ID |
| Variable Domain | QQKPGQPPRLLIYRASTRESGVPDRFSGSGSGTDFTLTISSLQA | NO: 827 |
| 59 | EDVAVYYCQQYYTTPLTFGGGTKVEIR | |
| Light Chain | DNQMTQSPSSLSASVGDRVTITCRASQGISNHLAWYQQKPG | SEQ ID |
| Variable Domain | KVPDLLIYAASTLQSGVPSRFSGSGSGTDFTLTISSLQPEDVA | NO: 828 |
| 60 | NYYCQKLNSAPLTFGGGTKVEIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 829 |
| 61 | YCLQHNNYPFTFGPGTKVDIK | |
| Light Chain | DIQLTQSPSFLSASVGDRVIITCRASQGITSYLAWYQRKPGKA | SEQ ID |
| Variable Domain | PKLLIYGATTLQSGVPSRFSGSGSGTEFTLTISSLQPEDFATYY | NO: 830 |
| 62 | CQQLQSYPYTFGQGTKLEIK | |
| Light Chain | EIVLTQSPATLSLSPGERATLSCRASQSVSNYFAWYRQKPGQ | SEQ ID |
| Variable Domain | APRLLIYDASNRATGIPARFSGSGSGTDFTLTISSLEPEDFAVY | NO: 831 |
| 63 | YCQHRSNWPLTFGGGTKVEIK | |
| Amlitelimab | EVQLVESGGGLVQPGGSLRLSCAASGFTFSNYAMNWV | SEQ ID |
| VH | RQAPGKGLEWVSTISGSGGATRYADSVKGRFTISRDNS | NO: 832 |
| | RNTVYLQMNSLRVEDTAVFYCTKDRLIMATVRGPYYY | |
| | GMDVWGQGTTVTVSS | |
| Amlitelimab | DIQMTQSPSSLSASVGDRVTITCRASQSISSYLNWYQQK | SEQ ID |
| VL | PGKAPNLLIYAASSLQSGVPSRFSGSGSETDFTLTISSLQP | NO: 833 |
| | EDFATYYCQQSHSVSFTFGPGTKVDIK | |
| LC.001 | EVQLLESGGGLVQPGGSLRLSCAASGFTFNSYAMSWVR | SEQ ID |
| (Oxelumab) | QAPGKGLEWVSIISGSGGFTYYADSVKGRFTISRDNSRT | NO: 834 |
| VH | TLYLQMNSLRAEDTAVYYCAKDRLVAPGTFDYWGQG | |
| | ALVTVSS | |
| LC.001 | DIQMTQSPSSLSASVGDRVTITCRASQGISSWLAWYQQ | SEQ ID |
| (Oxelumab) | KPEKAPKSLIYAASSLQSGVPSRFSGSGSGTDFTLTISSL | NO: 835 |
| VL | QPEDFATYYCQQYNSYPYTFGQGTKLEIK | |
| LC.005 VH | QVQLVESGGGVVQPGRSLRLSCAASGFTFSNFGMHWV | SEQ ID |
| | RQAPGKGLEWVAAIWYDGHDKYYSYYVKGRFTISRDN | NO: 836 |
| | SKNTLFLQMNSLRAEDTAVYYCARDSSSWYRYFDYWG | |
| | QGTLVTVSS | |
| LC.005 VL | EIVLTQSPGTLSLSPGERATLSCRASQSVSSNYLAWYQQ | SEQ ID |
| | KPGQAPRLLIYGASSRATGIPDRFSGSGSGTDFTLTISRL | NO: 837 |
| | EPEDFAVYYCQQYGSSFTFGPGTKVDIK | |
| LC.019 VH | QKQLVEFGGGVVQPGRSLRLSCAASGFTFSNYGMHWV | SEQ ID |
| | RQAPGKGLWVAVIWNDGSNKYYVDSVKGRFIISRDNS | NO: 838 |
| | KNTLYLQMNSLRAEDTAVYYCARDRMGIYYYGMDVW | |
| | GQGTTVTVSS | |
| LC.019 VL | EIVLTQSPATLSLSPGERATLSCRASQGVSRYLAWYQQK | SEQ ID |
| | PGQAPRLLIYDASNRATGIPARVSGSGPGTDFTLTISSLE | NO: 839 |
| | PEDFAVDYCQQRSNWQYTFGQGTKLEIK | |
| Chothia | GFTFSNA | SEQ ID |
| HCDR1 | | NO: 840 |
| Heavy Chain | EVRLVESGGGLVQPGGSLRLSCAASGFTFSSYAMTWVR | SEQ ID |
| Variable Domain | QAPGKGLEWVSLISGSGGLTKYADSVKGRFTISRDNSK | NO: 841 |
| 78 | KTLYLQMNSLRAEDTAVYYCAKDEGLTTGEYWGQGT | |
| | LVTVSS | |
| Light Chain | DIQMTQSPSSLSASVGDRVTITCRASQGIRNDLGWYQQ | SEQ ID |
| Variable Domain | KPGKAPKRLIYATSSFQSGVPSRFSGSGSGTEFTLTISSLQ | NO: 842 |
| 64 | PEDFATYYCLQHNSYPFTFGPGTKVDVK | |

1-262. (canceled)

263. An isolated antibody that binds OX40L, wherein the antibody comprises:
a CDR-H1 comprising the sequence set forth in SEQ ID NO: 1;
a CDR-H2 comprising the sequence set forth in SEQ ID NO: 70;
a CDR-H3 comprising the sequence set forth in SEQ ID NO: 175;
a CDR-L1 comprising the sequence set forth in SEQ ID NO: 258;
a CDR-L2 comprising the sequence set forth in SEQ ID NO: 335; and
a CDR-L3 comprising the sequence set forth in SEQ ID NO: 368.

264. An isolated antibody that binds OX40L, wherein the antibody comprises:
a CDR-H1 comprising the sequence set forth in SEQ ID NO: 1;
a CDR-H2 comprising the sequence set forth in SEQ ID NO: 70;
a CDR-H3 comprising the sequence set forth in SEQ ID NO: 175;
a CDR-L1 comprising the sequence set forth in SEQ ID NO: 258;
a CDR-L2 comprising the sequence set forth in SEQ ID NO: 335; and
a CDR-L3 comprising the sequence set forth in SEQ ID NO: 368;
and wherein the antibody comprises a human IgG1 Fc region with LALA and/or YTE mutations.

265. The isolated antibody of claim 263, wherein the antibody comprises an Fc region comprising one or more amino acid substitutions selected from the group consisting of S228P (SP), M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, N434W, M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA) L235A/G237A (LAGA), L234A/L235A/G237A (LALAGA), L234A/L235A/P329G (LALAPG), N297A, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

266. The isolated antibody of claim 264, wherein the LALA mutations are at positions 235 and 236 (L235A/L236A) and the YTE mutations are at positions 253, 255, and 257 (M253Y/S255T/T257E).

267. An isolated antibody that binds OX40L, wherein the antibody comprises a heavy chain variable region sequence set forth in SEQ ID NO: 419, a light chain variable region sequence set forth in SEQ ID NO: 489, a constant heavy chain sequence set forth in SEQ ID NO: 651, and a constant light chain sequence set forth in SEQ ID NO: 738.

268. The isolated antibody of claim 263, wherein the antibody comprises a heavy chain variable region sequence set forth in SEQ ID NO: 419 and a light chain variable region sequence set forth in SEQ ID NO: 489.

269. The isolated antibody of claim 263, wherein the antibody comprises a constant heavy chain sequence set forth in SEQ ID NO: 651 and a constant light chain sequence set forth in SEQ ID NO: 738.

270. An isolated antibody that binds OX40L, wherein the antibody comprises a heavy chain variable region sequence set forth in SEQ ID NO: 419 and a light chain variable region sequence set forth in SEQ ID NO: 489, wherein the antibody comprises a human IgG1 Fc region with LALA and/or YTE mutations.

271. The isolated antibody of claim 270, wherein the antibody comprises a human IgG1 Fc region with LALA and YTE mutations.

272. The isolated antibody of claim 263, wherein the antibody is a humanized, human, or chimeric antibody.

273. The isolated antibody of claim 272, wherein the antibody is a humanized antibody.

274. The isolated antibody of claim 272, wherein the antibody comprises a human IgG1 Fc region.

275. The isolated antibody of claim 263, wherein the antibody is a monoclonal antibody.

276. The isolated antibody of claim 263, wherein the antibody binds an OX40L sequence set forth in SEQ ID NO: 739.

277. The isolated antibody of claim 263, wherein the antibody binds to an OX40L sequence set forth in SEQ ID NO: 739 with a Kp of less than or equal to about 1×10⁻⁹M, as measured by surface plasmon resonance (SPR).

278. The isolated antibody of claim 263, wherein the antibody is formulated for subcutaneous injection.

279. The isolated antibody of claim 263, wherein the antibody is formulated for intravenous injection.

280. The isolated antibody of claim 264, wherein the antibody comprises a human IgG1 Fc region with LALA and YTE mutations.

281. A composition comprising the isolated antibody of claim 263 and a pharmaceutically acceptable excipient.

282. A method for treating an inflammatory disorder or disease in a mammalian subject in need thereof, the method comprising administering to the mammalian subject the isolated antibody of claim 263.

283. The method of claim 282, wherein the inflammatory disorder or disease is atopic dermatitis.

284. The method of claim 282, wherein the inflammatory disorder or disease is selected from the group consisting of asthma, idiopathic pulmonary fibrosis, alopecia areata, chronic sinusitis with nasal polyps, Chronic Rhinosinusitis without Nasal Polyps (CRSsNP), eosinophilic esophagitis (EoE), an Eosinophilic gastrointestinal disorder or disease (ENID), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), celiac disease, inflammatory bowel disease, psoriasis, lupus, rheumatoid arthritis, hidradenitis suppurativa, and systemic sclerosis.

285. The method of claim 284, wherein the ENID is selected from the group consisting of Eosinophilic Gastritis (EoG), Eosinophilic Enteritis (EoN), Eosinophilic Colitis (EoC), and Eosinophilic Gastroenteritis (EGE).

286. A kit comprising:
(a) the isolated antibody that binds OX40L of claim 263; and
(b) instructions for use.

287. The kit of claim 286, for use in treating an inflammatory disorder or disease selected from the group consisting of atopic dermatitis, asthma, idiopathic pulmonary fibrosis, alopecia areata, chronic sinusitis with nasal polyps, Chronic Rhinosinusitis without Nasal Polyps (CRSsNP), eosinophilic esophagitis (EoE), an Eosinophilic gastrointestinal disorder or disease (ENID), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), celiac disease, inflammatory bowel disease, psoriasis, lupus, rheumatoid arthritis, hidradenitis suppurativa, and systemic sclerosis.

288. An isolated polynucleotide or set of polynucleotides encoding the isolated antibody of claim 263.

289. A vector or set of vectors comprising the polynucleotide or set of polynucleotides of claim 288.

290. A host cell comprising the vector or set of vectors of claim 289.

291. The host cell of claim 290, wherein the host cell is a Chinese hamster ovary (CHO) cell.

292. A method of producing an antibody, the method comprising expressing the antibody with the host cell of claim 290 and isolating the expressed antibody.


SEQUENCE LISTING (842 sequences)

SEQ ID NO: 1 - protein, 5 aa, synthetic construct
  REGION 1..5: note = Synthetic: Kabat HCDR1
NYAMT

SEQ ID NO: 2 - protein, 5 aa, synthetic construct
  REGION 1..5: note = Synthetic: Kabat HCDR1
SYAMN

SEQ ID NO: 3 - protein, 5 aa, synthetic construct
  REGION 1..5: note = Synthetic: Kabat HCDR1
SNYMS

SEQ ID NO: 4 - protein, 5 aa, synthetic construct
  REGION 1..5: note = Synthetic: Kabat HCDR1
NYWMH

SEQ ID NO: 5 - protein, 5 aa, synthetic construct
  REGION 1..5: note = Synthetic: Kabat HCDR1
NAWMS

SEQ ID NO: 6 - protein, 5 aa, synthetic construct
  REGION 1..5: note = Synthetic: Kabat HCDR1
NAWMT

SEQ ID NO: 7 - protein, 5 aa, synthetic construct
  REGION 1..5: note = Synthetic: Kabat HCDR1
YAWMS

SEQ ID NO: 8 - protein, 5 aa, synthetic construct
  REGION 1..5: note = Synthetic: Kabat HCDR1
NYDIN

SEQ ID NO: 9 - protein, 5 aa, synthetic construct
  REGION 1..5: note = Synthetic: Kabat HCDR1
SYAMY

SEQ ID NO: 10 - protein, 5 aa, synthetic construct
  REGION 1..5: note = Synthetic: Kabat HCDR1
SYAMT

SEQ ID NO: 11 - protein, 5 aa, synthetic construct
  REGION 1..5: note = Synthetic: Kabat HCDR1
SYAMS

SEQ ID NO: 12 - protein, 5 aa, synthetic construct
  REGION 1..5: note = Synthetic: Kabat HCDR1
TYAMT

SEQ ID NO: 13 - protein, 5 aa, synthetic construct
  REGION 1..5: note = Synthetic: Kabat HCDR1
SHAMT

SEQ ID NO: 14 - protein, 5 aa, synthetic construct
  REGION 1..5: note = Synthetic: Kabat HCDR1
IYAMT

SEQ ID NO: 15 - protein, 5 aa, synthetic construct
  REGION 1..5: note = Synthetic: Kabat HCDR1
SYWMH

SEQ ID NO: 16 - protein, 5 aa, synthetic construct
  REGION 1..5: note = Synthetic: Kabat HCDR1
SFWMH

SEQ ID NO: 17 - protein, 5 aa, synthetic construct
  REGION 1..5: note = Synthetic: Kabat HCDR1
SAWMS

SEQ ID NO: 18 - protein, 5 aa, synthetic construct
  REGION 1..5: note = Synthetic: Kabat HCDR1
NAWMR

SEQ ID NO: 19 - protein, 5 aa, synthetic construct
  REGION 1..5: note = Synthetic: Kabat HCDR1
YAWMT

SEQ ID NO: 20 - protein, 5 aa, synthetic construct
  REGION 1..5: note = Synthetic: Kabat HCDR1
NAWMN

SEQ ID NO: 21 - protein, 5 aa, synthetic construct
  REGION 1..5: note = Synthetic: Kabat HCDR1
TAWMS

SEQ ID NO: 22 - protein, 7 aa, synthetic construct
  REGION 1..7: note = Synthetic: Chothia HCDR1
GFTFSNY

SEQ ID NO: 23 - protein, 7 aa, synthetic construct
  REGION 1..7: note = Synthetic: Chothia HCDR1
GFRFSSY

SEQ ID NO: 24 - protein, 7 aa, synthetic construct
  REGION 1..7: note = Synthetic: Chothia HCDR1
GFPVSSN

SEQ ID NO: 25 - protein, 7 aa, synthetic construct
  REGION 1..7: note = Synthetic: Chothia HCDR1
GFTFSYA

SEQ ID NO: 26 - protein, 7 aa, synthetic construct
  REGION 1..7: note = Synthetic: Chothia HCDR1
GYTFTNY

SEQ ID NO: 27 - protein, 7 aa, synthetic construct
  REGION 1..7: note = Synthetic: Chothia HCDR1
GFTFSSY

SEQ ID NO: 28 - protein, 7 aa, synthetic construct
  REGION 1..7: note = Synthetic: Chothia HCDR1
GFTFRNY

SEQ ID NO: 29 - protein, 7 aa, synthetic construct
  REGION 1..7: note = Synthetic: Chothia HCDR1
GFTFNTY

SEQ ID NO: 30 - protein, 7 aa, synthetic construct
  REGION 1..7: note = Synthetic: Chothia HCDR1
GFTFSSH

SEQ ID NO: 31 - protein, 7 aa, synthetic construct
  REGION 1..7: note = Synthetic: Chothia HCDR1
GFTFGNY

SEQ ID NO: 32 - protein, 7 aa, synthetic construct
  REGION 1..7: note = Synthetic: Chothia HCDR1
GFTFTNY

SEQ ID NO: 33 - protein, 7 aa, synthetic construct
  REGION 1..7: note = Synthetic: Chothia HCDR1
GFTFSIY

SEQ ID NO: 34 - protein, 7 aa, synthetic construct
  REGION 1..7: note = Synthetic: Chothia HCDR1
GFTFRSY

SEQ ID NO: 35 - protein, 7 aa, synthetic construct
  REGION 1..7: note = Synthetic: Chothia HCDR1
GLTFSNY

SEQ ID NO: 36 - protein, 7 aa, synthetic construct
  REGION 1..7: note = Synthetic: Chothia HCDR1
GFTFSSF

SEQ ID NO: 37 - protein, 7 aa, synthetic construct
  REGION 1..7: note = Synthetic: Chothia HCDR1
GFTFHNA

SEQ ID NO: 38 - protein, 7 aa, synthetic construct
  REGION 1..7: note = Synthetic: Chothia HCDR1
GFTFNSA

SEQ ID NO: 39 - protein, 7 aa, synthetic construct
  REGION 1..7: note = Synthetic: Chothia HCDR1
GFIFNNA

SEQ ID NO: 40 - protein, 7 aa, synthetic construct
  REGION 1..7: note = Synthetic: Chothia HCDR1
GFTFNYA

SEQ ID NO: 41 - protein, 7 aa, synthetic construct
  REGION 1..7: note = Synthetic: Chothia HCDR1
GFTFINA

SEQ ID NO: 42 - protein, 7 aa, synthetic construct
  REGION 1..7: note = Synthetic: Chothia HCDR1
GFTFSTA

SEQ ID NO: 43 - protein, 7 aa, synthetic construct
  REGION 1..7: note = Synthetic: Chothia HCDR1
GFIFSNA

SEQ ID NO: 44 - protein, 8 aa, synthetic construct
  REGION 1..8: note = Synthetic: IMGT CDR1
GFTFSNYA

SEQ ID NO: 45 - protein, 8 aa, synthetic construct
  REGION 1..8: note = Synthetic: IMGT CDR1
GFRFSSYA

SEQ ID NO: 46 - protein, 8 aa, synthetic construct
  REGION 1..8: note = Synthetic: IMGT CDR1
GFPVSSNY

SEQ ID NO: 47 - protein, 8 aa, synthetic construct
  REGION 1..8: note = Synthetic: IMGT CDR1
GFTFSNYW

SEQ ID NO: 48 - protein, 8 aa, synthetic construct
  REGION 1..8: note = Synthetic: IMGT CDR1
GFTFSNAW

SEQ ID NO: 49 - protein, 8 aa, synthetic construct
  REGION 1..8: note = Synthetic: IMGT CDR1
GFTFNNAW

SEQ ID NO: 50 - protein, 8 aa, synthetic construct
  REGION 1..8: note = Synthetic: IMGT CDR1
GFTFSYAW

SEQ ID NO: 51 - protein, 8 aa, synthetic construct
  REGION 1..8: note = Synthetic: IMGT CDR1
GYTFTNYD

SEQ ID NO: 52 - protein, 8 aa, synthetic construct
  REGION 1..8: note = Synthetic: IMGT CDR1
GFTFSSYA

SEQ ID NO: 53 - protein, 8 aa, synthetic construct
  REGION 1..8: note = Synthetic: IMGT CDR1
GFTFRNYA

SEQ ID NO: 54 - protein, 8 aa, synthetic construct
  REGION 1..8: note = Synthetic: IMGT CDR1
GFTFNTYA

SEQ ID NO: 55 - protein, 8 aa, synthetic construct
  REGION 1..8: note = Synthetic: IMGT CDR1
GFTFSSHA

SEQ ID NO: 56 - protein, 8 aa, synthetic construct
  REGION 1..8: note = Synthetic: IMGT CDR1
GFTFGNYA

SEQ ID NO: 57 - protein, 8 aa, synthetic construct
  REGION 1..8: note = Synthetic: IMGT CDR1
GFTFTNYA

SEQ ID NO: 58 - protein, 8 aa, synthetic construct
  REGION 1..8: note = Synthetic: IMGT CDR1
GFTFSIYA

SEQ ID NO: 59 - protein, 8 aa, synthetic construct
  REGION 1..8: note = Synthetic: IMGT CDR1
GFTFRSYW

SEQ ID NO: 60 - protein, 8 aa, synthetic construct
  REGION 1..8: note = Synthetic: IMGT CDR1
GLTFSNYW

SEQ ID NO: 61 - protein, 8 aa, synthetic construct
  REGION 1..8: note = Synthetic: IMGT CDR1
GFTFSSFW

SEQ ID NO: 62 - protein, 8 aa, synthetic construct
  REGION 1..8: note = Synthetic: IMGT CDR1
GFTFSSYW

SEQ ID NO: 63 - protein, 8 aa, synthetic construct
  REGION 1..8: note = Synthetic: IMGT CDR1
GFTFHNAW

SEQ ID NO: 64 - protein, 8 aa, synthetic construct
  REGION 1..8: note = Synthetic: IMGT CDR1
GFTFNSAW

SEQ ID NO: 65 - protein, 8 aa, synthetic construct
  REGION 1..8: note = Synthetic: IMGT CDR1
GFIFNNAW

SEQ ID NO: 66 - protein, 8 aa, synthetic construct
  REGION 1..8: note = Synthetic: IMGT CDR1
GFTFNYAW

SEQ ID NO: 67 - protein, 8 aa, synthetic construct
  REGION 1..8: note = Synthetic: IMGT CDR1
GFTFINAW

SEQ ID NO: 68 - protein, 8 aa, synthetic construct
  REGION 1..8: note = Synthetic: IMGT CDR1
GFTFSTAW

SEQ ID NO: 69 - protein, 8 aa, synthetic construct
  REGION 1..8: note = Synthetic: IMGT CDR1
GFIFSNAW

SEQ ID NO: 70 - protein, 17 aa, synthetic construct
  REGION 1..17: note = Synthetic: Kabat HCDR2
LISGSGGLTKYADSVKG

SEQ ID NO: 71 - protein, 16 aa, synthetic construct
  REGION 1..16: note = Synthetic: Kabat HCDR2
IIYSRDSTHYADSVKG

SEQ ID NO: 72 - protein, 17 aa, synthetic construct
  REGION 1..17: note = Synthetic: Kabat HCDR2
RINSDGISTRYADSVKG

SEQ ID NO: 73 - protein, 19 aa, synthetic construct
  REGION 1..19: note = Synthetic: Kabat HCDR2
RIKSNSYGGTTDYAAPAIG

SEQ ID NO: 74 - protein, 19 aa, synthetic construct
  REGION 1..19: note = Synthetic: Kabat HCDR2
RIKSISYGGTTDSAAPVKG

SEQ ID NO: 75 - protein, 19 aa, synthetic construct
  REGION 1..19: note = Synthetic: Kabat HCDR2
RIKSETDGGTTDYAAPVKG

SEQ ID NO: 76 - protein, 17 aa, synthetic construct
  REGION 1..17: note = Synthetic: Kabat HCDR2
WMNPHSGQTGYAQKFQG

SEQ ID NO: 77 - protein, 17 aa, synthetic construct
  REGION 1..17: note = Synthetic: Kabat HCDR2
LITGSGGSTHYADSVKG

SEQ ID NO: 78 - protein, 17 aa, synthetic construct
  REGION 1..17: note = Synthetic: Kabat HCDR2
LISGSGGITKYADSVKG

SEQ ID NO: 79 - protein, 17 aa, synthetic construct
  REGION 1..17: note = Synthetic: Kabat HCDR2
LISGSGGITHYADSVKG

SEQ ID NO: 80 - protein, 17 aa, synthetic construct
  REGION 1..17: note = Synthetic: Kabat HCDR2
LISGSGGVTHYADSVKG

SEQ ID NO: 81 - protein, 17 aa, synthetic construct
  REGION 1..17: note = Synthetic: Kabat HCDR2
LISGSGAITHYADSVKG

SEQ ID NO: 82 - protein, 17 aa, synthetic construct
  REGION 1..17: note = Synthetic: Kabat HCDR2
LISGSGGLTNYADSVKG

SEQ ID NO: 83 - protein, 17 aa, synthetic construct
  REGION 1..17: note = Synthetic: Kabat HCDR2
LISGSGGSTHYADSVKG

SEQ ID NO: 84 - protein, 17 aa, synthetic construct
  REGION 1..17: note = Synthetic: Kabat HCDR2
LISGSGGLTKYADSVRG

SEQ ID NO: 85 - protein, 17 aa, synthetic construct
  REGION 1..17: note = Synthetic: Kabat HCDR2
LISGSGSLTKYADSVKG

SEQ ID NO: 86 - protein, 17 aa, synthetic construct
  REGION 1..17: note = Synthetic: Kabat HCDR2
LISGGGGLTYYADSVKG

SEQ ID NO: 87 - protein, 17 aa, synthetic construct
  REGION 1..17: note = Synthetic: Kabat HCDR2
LISGSGGLTYYADSVKG

SEQ ID NO: 88 - protein, 17 aa, synthetic construct
  REGION 1..17: note = Synthetic: Kabat HCDR2
RIDNNGINTRYADSVKG

SEQ ID NO: 89 - protein, 17 aa, synthetic construct
  REGION 1..17: note = Synthetic: Kabat HCDR2
RISSDGISTRYADSVKG

SEQ ID NO: 90 - protein, 17 aa, synthetic construct
  REGION 1..17: note = Synthetic: Kabat HCDR2
RINNDGISTRYADSVKG

SEQ ID NO: 91 - protein, 17 aa, synthetic construct
  REGION 1..17: note = Synthetic: Kabat HCDR2
RINSDGFSTRYADSVKG

SEQ ID NO: 92 - protein, 19 aa, synthetic construct
  REGION 1..19: note = Synthetic: Kabat HCDR2
RIKGVTAGGTADYAAPVKG

SEQ ID NO: 93 - protein, 19 aa, synthetic construct
  REGION 1..19: note = Synthetic: Kabat HCDR2
RVKSVTAGGTTDYAAPVKG

SEQ ID NO: 94 - protein, 19 aa, synthetic construct
  REGION 1..19: note = Synthetic: Kabat HCDR2
RIKSKTDGGTTDYAAPVKG

SEQ ID NO: 95 - protein, 19 aa, synthetic construct
  REGION 1..19: note = Synthetic: Kabat HCDR2
RIKSVTTGGTTDYAAPVKG

SEQ ID NO: 96 - protein, 19 aa, synthetic construct
  REGION 1..19: note = Synthetic: Kabat HCDR2
RIKSVTAGGTTDYAAPVKG

SEQ ID NO: 97 - protein, 19 aa, synthetic construct
  REGION 1..19: note = Synthetic: Kabat HCDR2
RIKSVTAGGTTDFPAPVKG

SEQ ID NO: 98 - protein, 19 aa, synthetic construct
  REGION 1..19: note = Synthetic: Kabat HCDR2
HIKSETDGGTTDYAAPVKG

SEQ ID NO: 99 - protein, 19 aa, synthetic construct
  REGION 1..19: note = Synthetic: Kabat HCDR2
RIKRKTDGGTTDYAAPVQG

SEQ ID NO: 100 - protein, 19 aa, synthetic construct
  REGION 1..19: note = Synthetic: Kabat HCDR2
RIKSLTDGGTTDYAAPVKG

SEQ ID NO: 101 - protein, 19 aa, synthetic construct
  REGION 1..19: note = Synthetic: Kabat HCDR2
RIKRKTDGGTREYAAPVKG

SEQ ID NO: 102 - protein, 19 aa, synthetic construct
  REGION 1..19: note = Synthetic: Kabat HCDR2
RIKRNTDGGTTDYAAPVKG

SEQ ID NO: 103 - protein, 19 aa, synthetic construct
  REGION 1..19: note = Synthetic: Kabat HCDR2
RIKTNTDGGTTDYAAPVKG

SEQ ID NO: 104 - protein, 19 aa, synthetic construct
  REGION 1..19: note = Synthetic: Kabat HCDR2
RIKSKTYGGTTDYDAPVKG

SEQ ID NO: 105 - protein, 19 aa, synthetic construct
  REGION 1..19: note = Synthetic: Kabat HCDR2
RIKSQTYGGTTDYAAPVKG

SEQ ID NO: 106 - protein, 19 aa, synthetic construct
  REGION 1..19: note = Synthetic: Kabat HCDR2
RIKSKTYGGTTDYAAPVKG

SEQ ID NO: 107 - protein, 19 aa, synthetic construct
  REGION 1..19: note = Synthetic: Kabat HCDR2
RIKRKTDGGTRDYAAPVKG

SEQ ID NO: 108 - protein, 19 aa, synthetic construct
  REGION 1..19: note = Synthetic: Kabat HCDR2
RIKRETDGGTTDYASPVKG

SEQ ID NO: 109 - protein, 19 aa, synthetic construct
  REGION 1..19: note = Synthetic: Kabat HCDR2
RIKRKSDGGTTDYAAPVKG

SEQ ID NO: 110 - protein, 19 aa, synthetic construct
  REGION 1..19: note = Synthetic: Kabat HCDR2
RIKSEIDGGTTDYAAPVKG

SEQ ID NO: 111 - protein, 6 aa, synthetic construct
  REGION 1..6: note = Synthetic: Chothia HCDR2
SGSGGL

SEQ ID NO: 112 - protein, 5 aa, synthetic construct
  REGION 1..5: note = Synthetic: Chothia HCDR2
YSRDS

SEQ ID NO: 113 - protein, 6 aa, synthetic construct
  REGION 1..6: note = Synthetic: Chothia HCDR2
NSDGIS

SEQ ID NO: 114 - protein, 8 aa, synthetic construct
  REGION 1..8: note = Synthetic: Chothia HCDR2
KSNSYGGT

SEQ ID NO: 115 - protein, 8 aa, synthetic construct
  REGION 1..8: note = Synthetic: Chothia HCDR2
KSISYGGT

SEQ ID NO: 116 - protein, 8 aa, synthetic construct
  REGION 1..8: note = Synthetic: Chothia HCDR2
KSETDGGT

SEQ ID NO: 117 - protein, 6 aa, synthetic construct
  REGION 1..6: note = Synthetic: Chothia HCDR2
NPHSGQ

SEQ ID NO: 118 - protein, 6 aa, synthetic construct
  REGION 1..6: note = Synthetic: Chothia HCDR2
TGSGGS

SEQ ID NO: 119 - protein, 6 aa, synthetic construct
  REGION 1..6: note = Synthetic: Chothia HCDR2
SGSGGI

SEQ ID NO: 120 - protein, 6 aa, synthetic construct
  REGION 1..6: note = Synthetic: Chothia HCDR2
SGSGGV

SEQ ID NO: 121 - protein, 6 aa, synthetic construct
  REGION 1..6: note = Synthetic: Chothia HCDR2
SGSGAI

SEQ ID NO: 122 - protein, 6 aa, synthetic construct
  REGION 1..6: note = Synthetic: Chothia HCDR2
SGSGGS

SEQ ID NO: 123 - protein, 6 aa, synthetic construct
  REGION 1..6: note = Synthetic: Chothia HCDR2
SGSGSL

SEQ ID NO: 124 - protein, 6 aa, synthetic construct
  REGION 1..6: note = Synthetic: Chothia HCDR2
SGGGGL

SEQ ID NO: 125 - protein, 6 aa, synthetic construct
  REGION 1..6: note = Synthetic: Chothia HCDR2
DNNGIN

SEQ ID NO: 126 - protein, 6 aa, synthetic construct
  REGION 1..6: note = Synthetic: Chothia HCDR2
SSDGIS

SEQ ID NO: 127 - protein, 6 aa, synthetic construct
  REGION 1..6: note = Synthetic: Chothia HCDR2
NNDGIS

SEQ ID NO: 128 - protein, 6 aa, synthetic construct
  REGION 1..6: note = Synthetic: Chothia HCDR2
NSDGFS

SEQ ID NO: 129 - protein, 8 aa, synthetic construct
  REGION 1..8: note = Synthetic: Chothia HCDR2
KGVTAGGT

SEQ ID NO: 130 - protein, 8 aa, synthetic construct
  REGION 1..8: note = Synthetic: Chothia HCDR2
KSVTAGGT

SEQ ID NO: 131 - protein, 8 aa, synthetic construct
  REGION 1..8: note = Synthetic: Chothia HCDR2
KSKTDGGT

SEQ ID NO: 132 - protein, 8 aa, synthetic construct
  REGION 1..8: note = Synthetic: Chothia HCDR2
KSVTTGGT

SEQ ID NO: 133 - protein, 8 aa, synthetic construct
  REGION 1..8: note = Synthetic: Chothia HCDR2
KRKTDGGT

SEQ ID NO: 134 - protein, 8 aa, synthetic construct
  REGION 1..8: note = Synthetic: Chothia HCDR2
KSLTDGGT

SEQ ID NO: 135 - protein, 8 aa, synthetic construct
  REGION 1..8: note = Synthetic: Chothia HCDR2
KRNTDGGT

SEQ ID NO: 136 - protein, 8 aa, synthetic construct
  REGION 1..8: note = Synthetic: Chothia HCDR2
KRNTDGGT

SEQ ID NO: 137 - protein, 8 aa, synthetic construct
  REGION 1..8: note = Synthetic: Chothia HCDR2
KSKTYGGT

SEQ ID NO: 138 - protein, 8 aa, synthetic construct
  REGION 1..8: note = Synthetic: Chothia HCDR2
KSQTYGGT

SEQ ID NO: 139 - protein, 8 aa, synthetic construct
  REGION 1..8: note = Synthetic: Chothia HCDR2
KRETDGGT

SEQ ID NO: 140 - protein, 8 aa, synthetic construct
  REGION 1..8: note = Synthetic: Chothia HCDR2
KRKSDGGT

SEQ ID NO: 141 - protein, 8 aa, synthetic construct
  REGION 1..8: note = Synthetic: Chothia HCDR2
KSEIDGGT

SEQ ID NO: 142 - protein, 8 aa, synthetic construct
  REGION 1..8: note = Synthetic: IMGT HCDR2
ISGSGGLT

SEQ ID NO: 143 - protein, 7 aa, synthetic construct
  REGION 1..7: note = Synthetic: IMGT HCDR2
IYSRDST

SEQ ID NO: 144 - protein, 8 aa, synthetic construct
  REGION 1..8: note = Synthetic: IMGT HCDR2
INSDGIST

SEQ ID NO: 145 - protein, 10 aa, synthetic construct
  REGION 1..10: note = Synthetic: IMGT HCDR2
IKSNSYGGTT

SEQ ID NO: 146 - protein, 10 aa, synthetic construct
  REGION 1..10: note = Synthetic: IMGT HCDR2
IKSISYGGTT

SEQ ID NO: 147 - protein, 10 aa, synthetic construct
  REGION 1..10: note = Synthetic: IMGT HCDR2
IKSETDGGTT

SEQ ID NO: 148 - protein, 8 aa, synthetic construct
  REGION 1..8: note = Synthetic: IMGT HCDR2
MNPHSGQT

SEQ ID NO: 149 - protein, 8 aa, synthetic construct
  REGION 1..8: note = Synthetic: IMGT HCDR2
ITGSGGST

SEQ ID NO: 150 - protein, 8 aa, synthetic construct
  REGION 1..8: note = Synthetic: IMGT HCDR2
ISGSGGIT

SEQ ID NO: 151 - protein, 8 aa, synthetic construct
  REGION 1..8: note = Synthetic: IMGT HCDR2
ISGSGGVT

SEQ ID NO: 152 - protein, 8 aa, synthetic construct
  REGION 1..8: note = Synthetic: IMGT HCDR2
ISGSGAIT

SEQ ID NO: 153 - protein, 8 aa, synthetic construct
  REGION 1..8: note = Synthetic: IMGT HCDR2
ISGSGGST

SEQ ID NO: 154 - protein, 8 aa, synthetic construct
  REGION 1..8: note = Synthetic: IMGT HCDR2
ISGSGSLT

SEQ ID NO: 155 - protein, 8 aa, synthetic construct
  REGION 1..8: note = Synthetic: IMGT HCDR2
ISGGGGLT

SEQ ID NO: 156 - protein, 8 aa, synthetic construct
  REGION 1..8: note = Synthetic: IMGT HCDR2
IDNNGINT

SEQ ID NO: 157 - protein, 8 aa, synthetic construct
  REGION 1..8: note = Synthetic: IMGT HCDR2
ISSDGIST

SEQ ID NO: 158 - protein, 8 aa, synthetic construct
  REGION 1..8: note = Synthetic: IMGT HCDR2
INNDGIST

SEQ ID NO: 159 - protein, 8 aa, synthetic construct
  REGION 1..8: note = Synthetic: IMGT HCDR2
INSDGFST

SEQ ID NO: 160 - protein, 10 aa, synthetic construct
  REGION 1..10: note = Synthetic: IMGT HCDR2
IKGVTAGGTA

SEQ ID NO: 161 - protein, 10 aa, synthetic construct
  REGION 1..10: note = Synthetic: IMGT HCDR2
VKSVTAGGTT

SEQ ID NO: 162 - protein, 10 aa, synthetic construct
  REGION 1..10: note = Synthetic: IMGT HCDR2
IKSKTDGGTT

SEQ ID NO: 163 - protein, 10 aa, synthetic construct
  REGION 1..10: note = Synthetic: IMGT HCDR2
IKSVTTGGTT

SEQ ID NO: 164 - protein, 10 aa, synthetic construct
  REGION 1..10: note = Synthetic: IMGT HCDR2
IKSVTAGGTT

SEQ ID NO: 165 - protein, 10 aa, synthetic construct
  REGION 1..10: note = Synthetic: IMGT HCDR2
IKRKTDGGTT

SEQ ID NO: 166 - protein, 10 aa, synthetic construct
  REGION 1..10: note = Synthetic: IMGT HCDR2
IKSLTDGGTT

SEQ ID NO: 167 - protein, 10 aa, synthetic construct
  REGION 1..10: note = Synthetic: IMGT HCDR2
IKRKTDGGTR

SEQ ID NO: 168 - protein, 10 aa, synthetic construct
  REGION 1..10: note = Synthetic: IMGT HCDR2
IKRNTDGGTT

SEQ ID NO: 169 - protein, 10 aa, synthetic construct
  REGION 1..10: note = Synthetic: IMGT HCDR2
IKTNTDGGTT

SEQ ID NO: 170 - protein, 10 aa, synthetic construct
  REGION 1..10: note = Synthetic: IMGT HCDR2
IKSKTYGGTT

SEQ ID NO: 171 - protein, 10 aa, synthetic construct
  REGION 1..10: note = Synthetic: IMGT HCDR2
IKSQTYGGTT

SEQ ID NO: 172 - protein, 10 aa, synthetic construct
  REGION 1..10: note = Synthetic: IMGT HCDR2
IKRETDGGTT

SEQ ID NO: 173 - protein, 10 aa, synthetic construct
  REGION 1..10: note = Synthetic: IMGT HCDR2
IKRKSDGGTT

SEQ ID NO: 174 - protein, 10 aa, synthetic construct
  REGION 1..10: note = Synthetic: IMGT HCDR2
IKSEIDGGTT

SEQ ID NO: 175 - protein, 9 aa, synthetic construct
  REGION 1..9: note = Synthetic: Kabat HCDR3
DEGLTTGEY

SEQ ID NO: 176 - protein, 9 aa, synthetic construct
  REGION 1..9: note = Synthetic: Kabat HCDR3
ERWEPSFHY

SEQ ID NO: 177 - protein, 12 aa, synthetic construct
  REGION 1..12: note = Synthetic: Kabat HCDR3
TLTYYYGAGSDY

SEQ ID NO: 178 - protein, 9 aa, synthetic construct
  REGION 1..9: note = Synthetic: Kabat HCDR3
DFQLELPDY

SEQ ID NO: 179 - protein, 9 aa, synthetic construct
  REGION 1..9: note = Synthetic: Kabat HCDR3
DHPKFGFDF

SEQ ID NO: 180 - protein, 12 aa, synthetic construct
  REGION 1..12: note = Synthetic: Kabat HCDR3
GSVWKYGGSLDF

SEQ ID NO: 181 - protein, 11 aa, synthetic construct
  REGION 1..11: note = Synthetic: Kabat HCDR3
GSSWNVYFFDF

SEQ ID NO: 182 - protein, 16 aa, synthetic construct
  REGION 1..16: note = Synthetic: Kabat HCDR3
EGYDLWSGDSSNWLDP

SEQ ID NO: 183 - protein, 16 aa, synthetic construct
  REGION 1..16: note = Synthetic: Kabat HCDR3
EGYDLWSGESSNWLDP

SEQ ID NO: 184 - protein, 16 aa, synthetic construct
  REGION 1..16: note = Synthetic: Kabat HCDR3
EGYDLWSGHSSNWLDP

SEQ ID NO: 185 - protein, 16 aa, synthetic construct
  REGION 1..16: note = Synthetic: Kabat HCDR3
EGYDLWSGYSSNWLDP

SEQ ID NO: 186 - protein, 9 aa, synthetic construct
  REGION 1..9: note = Synthetic: Kabat HCDR3
DEGLTTGEF

SEQ ID NO: 187 - protein, 9 aa, synthetic construct
  REGION 1..9: note = Synthetic: Kabat HCDR3
DEGLTTGES

SEQ ID NO: 188 - protein, 9 aa, synthetic construct
  REGION 1..9: note = Synthetic: Kabat HCDR3
EEGLTTGEY

SEQ ID NO: 189 - protein, 9 aa, synthetic construct
  REGION 1..9: note = Synthetic: Kabat HCDR3
DEGLTSGEY

SEQ ID NO: 190 - protein, 9 aa, synthetic construct
  REGION 1..9: note = Synthetic: Kabat HCDR3
DFQLELRDY

SEQ ID NO: 191 - protein, 9 aa, synthetic construct
  REGION 1..9: note = Synthetic: Kabat HCDR3
DFQLELRDF

SEQ ID NO: 192 - protein, 9 aa, synthetic construct
  REGION 1..9: note = Synthetic: Kabat HCDR3
DHPKFGFDY

SEQ ID NO: 193 - protein, 12 aa, synthetic construct
  REGION 1..12: note = Synthetic: Kabat HCDR3
GGVWNYGGSLDF

SEQ ID NO: 194 - protein, 12 aa, synthetic construct
  REGION 1..12: note = Synthetic: Kabat HCDR3
GGVWKYGGSLDF

SEQ ID NO: 195 - protein, 12 aa, synthetic construct
  REGION 1..12: note = Synthetic: Kabat HCDR3
GSVWNYGGSLDF

SEQ ID NO: 196 - protein, 12 aa, synthetic construct
  REGION 1..12: note = Synthetic: Kabat HCDR3
GSVWNYGGSLDY

SEQ ID NO: 197 - protein, 12 aa, synthetic construct
  REGION 1..12: note = Synthetic: Kabat HCDR3
GSVWKYGGSLDY

SEQ ID NO: 198 - protein, 12 aa, synthetic construct
  REGION 1..12: note = Synthetic: Kabat HCDR3
GSVWRYGGSLDF

SEQ ID NO: 199 - protein, 11 aa, synthetic construct
  REGION 1..11: note = Synthetic: Kabat HCDR3
GSSWHVYFFDF

SEQ ID NO: 200 - protein, 11 aa, synthetic construct
  REGION 1..11: note = Synthetic: Kabat HCDR3
GSSWSLYYFDF

SEQ ID NO: 201 - protein, 11 aa, synthetic construct
  REGION 1..11: note = Synthetic: Kabat HCDR3
GSSWYVYYFDY

SEQ ID NO: 202 - protein, 11 aa, synthetic construct
  REGION 1..11: note = Synthetic: Kabat HCDR3
GSSWYVYYFDF

SEQ ID NO: 203 - protein, 7 aa, synthetic construct
  REGION 1..7: note = Synthetic: Chothia HCDR3
EGLTTGE

SEQ ID NO: 204 - protein, 7 aa, synthetic construct
  REGION 1..7: note = Synthetic: Chothia HCDR3
RWEPSFH

SEQ ID NO: 205 - protein, 10 aa, synthetic construct
  REGION 1..10: note = Synthetic: Chothia HCDR3
LTYYYGAGSD

SEQ ID NO: 206 - protein, 7 aa, synthetic construct
  REGION 1..7: note = Synthetic: Chothia HCDR3
FQLELPD

SEQ ID NO: 207 - protein, 7 aa, synthetic construct
  REGION 1..7: note = Synthetic: Chothia HCDR3
HPKFGFD

SEQ ID NO: 208 - protein, 10 aa, synthetic construct
  REGION 1..10: note = Synthetic: Chothia HCDR3
SVWKYGGSLD

SEQ ID NO: 209 - protein, 9 aa, synthetic construct
  REGION 1..9: note = Synthetic: Chothia HCDR3
SSWNVYFFD

SEQ ID NO: 210 - protein, 14 aa, synthetic construct
  REGION 1..14: note = Synthetic: Chothia HCDR3
GYDLWSGDSSNWLD

SEQ ID NO: 211 - protein, 14 aa, synthetic construct
  REGION 1..14: note = Synthetic: Chothia HCDR3
GYDLWSGESSNWLD

SEQ ID NO: 212 - protein, 14 aa, synthetic construct
  REGION 1..14: note = Synthetic: Chothia HCDR3
GYDLWSGHSSNWLD

SEQ ID NO: 213 - protein, 14 aa, synthetic construct
  REGION 1..14: note = Synthetic: Chothia HCDR3
GYDLWSGYSSNWLD

SEQ ID NO: 214 - protein, 7 aa, synthetic construct
  REGION 1..7: note = Synthetic: Chothia HCDR3
EGLTSGE

SEQ ID NO: 215 - protein, 7 aa, synthetic construct
  REGION 1..7: note = Synthetic: Chothia HCDR3
FQLELRD

SEQ ID NO: 216 - protein, 10 aa, synthetic construct
  REGION 1..10: note = Synthetic: Chothia HCDR3
GVWNYGGSLD

SEQ ID NO: 217 - protein, 10 aa, synthetic construct
  REGION 1..10: note = Synthetic: Chothia HCDR3
GVWKYGGSLD

SEQ ID NO: 218 - protein, 10 aa, synthetic construct
  REGION 1..10: note = Synthetic: Chothia HCDR3
SVWNYGGSLD

SEQ ID NO: 219 - protein, 10 aa, synthetic construct
  REGION 1..10: note = Synthetic: Chothia HCDR3
SVWRYGGSLD

SEQ ID NO: 220 - protein, 9 aa, synthetic construct
  REGION 1..9: note = Synthetic: Chothia HCDR3
SSWHVYFFD

SEQ ID NO: 221 - protein, 9 aa, synthetic construct
  REGION 1..9: note = Synthetic: Chothia HCDR3
SSWSLYYFD

SEQ ID NO: 222 - protein, 9 aa, synthetic construct
  REGION 1..9: note = Synthetic: Chothia HCDR3
SSWYVYYFD

SEQ ID NO: 223 - protein, 11 aa, synthetic construct
  REGION 1..11: note = Synthetic: IMGT HCDR3
VKDEGLTTGEY

SEQ ID NO: 224 - protein, 11 aa, synthetic construct
  REGION 1..11: note = Synthetic: IMGT HCDR3
AKERWEPSFHY

SEQ ID NO: 225 - protein, 14 aa, synthetic construct
  REGION 1..14: note = Synthetic: IMGT HCDR3
AGTLTYYYGAGSDY

SEQ ID NO: 226 - protein, 11 aa, synthetic construct
  REGION 1..11: note = Synthetic: IMGT HCDR3
VRDFQLELPDY

SEQ ID NO: 227 - protein, 11 aa, synthetic construct
  REGION 1..11: note = Synthetic: IMGT HCDR3
TTDHPKFGFDF

SEQ ID NO: 228 - protein, 14 aa, synthetic construct
  REGION 1..14: note = Synthetic: IMGT HCDR3
STGSVWKYGGSLDF

SEQ ID NO: 229 - protein, 13 aa, synthetic construct
  REGION 1..13: note = Synthetic: IMGT HCDR3
TTGSSWNVYFFDF

SEQ ID NO: 230 - protein, 18 aa, synthetic construct
  REGION 1..18: note = Synthetic: IMGT HCDR3
AREGYDLWSGDSSNWLDP

SEQ ID NO: 231 - protein, 18 aa, synthetic construct
  REGION 1..18: note = Synthetic: IMGT HCDR3
AREGYDLWSGESSNWLDP

SEQ ID NO: 232 - protein, 18 aa, synthetic construct
  REGION 1..18: note = Synthetic: IMGT HCDR3
AREGYDLWSGHSSNWLDP

SEQ ID NO: 233 - protein, 18 aa, synthetic construct
  REGION 1..18: note = Synthetic: IMGT HCDR3
AREGYDLWSGYSSNWLDP

SEQ ID NO: 234 - protein, 11 aa, synthetic construct
  REGION 1..11: note = Synthetic: IMGT HCDR3
AKDEGLTTGEY

SEQ ID NO: 235 - protein, 11 aa, synthetic construct
  REGION 1..11: note = Synthetic: IMGT HCDR3
VKDEGLTTGEF

SEQ ID NO: 236 - protein, 11 aa, synthetic construct
  REGION 1..11: note = Synthetic: IMGT HCDR3
VKDEGLTTGES

SEQ ID NO: 237 - protein, 11 aa, synthetic construct
  REGION 1..11: note = Synthetic: IMGT HCDR3
AKEEGLTTGEY

SEQ ID NO: 238 - protein, 11 aa, synthetic construct
  REGION 1..11: note = Synthetic: IMGT HCDR3
AKDEGLTSGEY

SEQ ID NO: 239 - protein, 11 aa, synthetic construct
  REGION 1..11: note = Synthetic: IMGT HCDR3
VKEEGLTTGEY

SEQ ID NO: 240 - protein, 11 aa, synthetic construct
  REGION 1..11: note = Synthetic: IMGT HCDR3
VRDFQLELRDY

SEQ ID NO: 241 - protein, 11 aa, synthetic construct
  REGION 1..11: note = Synthetic: IMGT HCDR3
VSDFQLELPDY

SEQ ID NO: 242 - protein, 11 aa, synthetic construct
  REGION 1..11: note = Synthetic: IMGT HCDR3
VRDFQLELRDF

SEQ ID NO: 243 - protein, 11 aa, synthetic construct
  REGION 1..11: note = Synthetic: IMGT HCDR3
TTDHPKFGFDY

SEQ ID NO: 244 - protein, 14 aa, synthetic construct
  REGION 1..14: note = Synthetic: IMGT HCDR3
TTGGVWNYGGSLDF

SEQ ID NO: 245 - protein, 14 aa, synthetic construct
  REGION 1..14: note = Synthetic: IMGT HCDR3
ITGGVWNYGGSLDF

SEQ ID NO: 246 - protein, 14 aa, synthetic construct
  REGION 1..14: note = Synthetic: IMGT HCDR3
TTGGVWKYGGSLDF

SEQ ID NO: 247 - protein, 14 aa, synthetic construct
  REGION 1..14: note = Synthetic: IMGT HCDR3
ATGGVWKYGGSLDF

SEQ ID NO: 248 - protein, 14 aa, synthetic construct
  REGION 1..14: note = Synthetic: IMGT HCDR3
TTGSVWKYGGSLDF

SEQ ID NO: 249 - protein, 14 aa, synthetic construct
  REGION 1..14: note = Synthetic: IMGT HCDR3
STGSVWNYGGSLDF

SEQ ID NO: 250 - protein, 14 aa, synthetic construct
  REGION 1..14: note = Synthetic: IMGT HCDR3
TTGSVWNYGGSLDY

SEQ ID NO: 251 - protein, 14 aa, synthetic construct
  REGION 1..14: note = Synthetic: IMGT HCDR3
TTGSVWKYGGSLDY

SEQ ID NO: 252 - protein, 14 aa, synthetic construct
  REGION 1..14: note = Synthetic: IMGT HCDR3
TTGSVWRYGGSLDF

SEQ ID NO: 253 - protein, 14 aa, synthetic construct
  REGION 1..14: note = Synthetic: IMGT HCDR3
TTGSVWNYGGSLDF

SEQ ID NO: 254 - protein, 13 aa, synthetic construct
  REGION 1..13: note = Synthetic: IMGT HCDR3
TTGSSWHVYFFDF

SEQ ID NO: 255 - protein, 13 aa, synthetic construct
  REGION 1..13: note = Synthetic: IMGT HCDR3
TTGSSWSLYYFDF

SEQ ID NO: 256 - protein, 13 aa, synthetic construct
  REGION 1..13: note = Synthetic: IMGT HCDR3
TTGSSWYVYYFDY

SEQ ID NO: 257 - protein, 13 aa, synthetic construct
  REGION 1..13: note = Synthetic: IMGT HCDR3
TTGSSWYVYYFDF

SEQ ID NO: 258 - protein, 11 aa, synthetic construct
  REGION 1..11: note = Synthetic: Kabat LCDR1
RASQDIRNDLA

SEQ ID NO: 259 - protein, 11 aa, synthetic construct
  REGION 1..11: note = Synthetic: Kabat LCDR1
QASQGITNYLN

SEQ ID NO: 260 - protein, 11 aa, synthetic construct
  REGION 1..11: note = Synthetic: Kabat LCDR1
RASQGIRNDLG

SEQ ID NO: 261 - protein, 11 aa, synthetic construct
  REGION 1..11: note = Synthetic: Kabat LCDR1
RASQGIRDDLA

SEQ ID NO: 262 - protein, 17 aa, synthetic construct
  REGION 1..17: note = Synthetic: Kabat LCDR1
KSSQSVFYSSNNKNYLA

SEQ ID NO: 263 - protein, 11 aa, synthetic construct
  REGION 1..11: note = Synthetic: Kabat LCDR1
RASQGISRFLN

SEQ ID NO: 264 - protein, 11 aa, synthetic construct
  REGION 1..11: note = Synthetic: Kabat LCDR1
RASQSISNYLN

SEQ ID NO: 265 - protein, 11 aa, synthetic construct
  REGION 1..11: note = Synthetic: Kabat LCDR1
RASQGISSYLA

SEQ ID NO: 266 - protein, 11 aa, synthetic construct
  REGION 1..11: note = Synthetic: Kabat LCDR1
RASQDIRNDLD

SEQ ID NO: 267 - protein, 11 aa, synthetic construct
  REGION 1..11: note = Synthetic: Kabat LCDR1
RASQAIRNDLG

SEQ ID NO: 268 - protein, 11 aa, synthetic construct
  REGION 1..11: note = Synthetic: Kabat LCDR1
RTSQGIRNDLA

SEQ ID NO: 269 - protein, 11 aa, synthetic construct
  REGION 1..11: note = Synthetic: Kabat LCDR1
RASQGIRNDLA

SEQ ID NO: 270 - protein, 11 aa, synthetic construct
  REGION 1..11: note = Synthetic: Kabat LCDR1
RASQGIRNNLG

SEQ ID NO: 271 - protein, 11 aa, synthetic construct
  REGION 1..11: note = Synthetic: Kabat LCDR1
RASQAIRDDLA

SEQ ID NO: 272 - protein, 17 aa, synthetic construct
  REGION 1..17: note = Synthetic: Kabat LCDR1
KSSQSVFYTSNSKHYLN

SEQ ID NO: 273 - protein, 17 aa, synthetic construct
  REGION 1..17: note = Synthetic: Kabat LCDR1
KSSQSVLYSSNNKNYLA

SEQ ID NO: 274 - protein, 17 aa, synthetic construct
  REGION 1..17: note = Synthetic: Kabat LCDR1
KSSQSVFYTSNNKNYLN

SEQ ID NO: 275 - protein, 17 aa, synthetic construct
  REGION 1..17: note = Synthetic: Kabat LCDR1
KSSQSVFYTSSNKHYLN

SEQ ID NO: 276 - protein, 11 aa, synthetic construct
  REGION 1..11: note = Synthetic: Kabat LCDR1
RASQSITRYLN

SEQ ID NO: 277 - protein, 11 aa, synthetic construct
  REGION 1..11: note = Synthetic: Kabat LCDR1
RASQFITRYLN

SEQ ID NO: 278 - protein, 11 aa, synthetic construct
  REGION 1..11: note = Synthetic: Kabat LCDR1
RASQGISRYLN

SEQ ID NO: 279 - protein, 11 aa, synthetic construct
  REGION 1..11: note = Synthetic: Kabat LCDR1
RASQSITRSLN

SEQ ID NO: 280 - protein, 11 aa, synthetic construct
  REGION 1..11: note = Synthetic: Kabat LCDR1
RASQSISRYLN

SEQ ID NO: 281 - protein, 11 aa, synthetic construct
  REGION 1..11: note = Synthetic: Kabat LCDR1
RASQNISRYLN

SEQ ID NO: 282 - protein, 11 aa, synthetic construct
  REGION 1..11: note = Synthetic: Kabat LCDR1
RASQIISRYLN

SEQ ID NO: 283 - protein, 11 aa, synthetic construct
  REGION 1..11: note = Synthetic: Kabat LCDR1
RASQSMSSHLN

SEQ ID NO: 284 - protein, 11 aa, synthetic construct
  REGION 1..11: note = Synthetic: Kabat LCDR1
RASQSISTYLN

SEQ ID NO: 285 - protein, 11 aa, synthetic construct
  REGION 1..11: note = Synthetic: Kabat LCDR1
RASQSISSYLN

SEQ ID NO: 286 - protein, 7 aa, synthetic construct
  REGION 1..7: note = Synthetic: Chothia LCDR1
SQDIRND

SEQ ID NO: 287 - protein, 7 aa, synthetic construct
  REGION 1..7: note = Synthetic: Chothia LCDR1
SQGITNY

SEQ ID NO: 288 - protein, 7 aa, synthetic construct
  REGION 1..7: note = Synthetic: Chothia LCDR1
SQGIRND

SEQ ID NO: 289 - protein, 7 aa, synthetic construct
  REGION 1..7: note = Synthetic: Chothia LCDR1
SQGIRDD

SEQ ID NO: 290 - protein, 13 aa, synthetic construct
  REGION 1..13: note = Synthetic: Chothia LCDR1
SQSVFYSSNNKNY

SEQ ID NO: 291 - protein, 7 aa, synthetic construct
  REGION 1..7: note = Synthetic: Chothia LCDR1
SQGISRF

SEQ ID NO: 292 - protein, 7 aa, synthetic construct
  REGION 1..7: note = Synthetic: Chothia LCDR1
SQSISNY

SEQ ID NO: 293 - protein, 7 aa, synthetic construct
  REGION 1..7: note = Synthetic: Chothia LCDR1
SQGISSY

SEQ ID NO: 294 - protein, 7 aa, synthetic construct
  REGION 1..7: note = Synthetic: Chothia LCDR1
SQAIRND

SEQ ID NO: 295 - protein, 7 aa, synthetic construct
  REGION 1..7: note = Synthetic: Chothia LCDR1
SQGIRNN

SEQ ID NO: 296 - protein, 7 aa, synthetic construct
  REGION 1..7: note = Synthetic: Chothia LCDR1
SQAIRDD

SEQ ID NO: 297 - protein, 13 aa, synthetic construct
  REGION 1..13: note = Synthetic: Chothia LCDR1
SQSVFYTSNSKHY

SEQ ID NO: 298 - protein, 13 aa, synthetic construct
  REGION 1..13: note = Synthetic: Chothia LCDR1
SQSVLYSSNNKNY

SEQ ID NO: 299 - protein, 13 aa, synthetic construct
  REGION 1..13: note = Synthetic: Chothia LCDR1
SQSVFYTSNNKNY

SEQ ID NO: 300 - protein, 13 aa, synthetic construct
  REGION 1..13: note = Synthetic: Chothia LCDR1
SQSVFYTSSNKHY

SEQ ID NO: 301 - protein, 7 aa, synthetic construct
  REGION 1..7: note = Synthetic: Chothia LCDR1
SQSITRY

SEQ ID NO: 302 - protein, 7 aa, synthetic construct
  REGION 1..7: note = Synthetic: Chothia LCDR1
SQFITRY

SEQ ID NO: 303 - protein, 7 aa, synthetic construct
  REGION 1..7: note = Synthetic: Chothia LCDR1
SQGISRY

SEQ ID NO: 304 - protein, 7 aa, synthetic construct
  REGION 1..7: note = Synthetic: Chothia LCDR1
SQSITRS

SEQ ID NO: 305 - protein, 7 aa, synthetic construct
  REGION 1..7: note = Synthetic: Chothia LCDR1
SQSISRY

SEQ ID NO: 306 - protein, 7 aa, synthetic construct
  REGION 1..7: note = Synthetic: Chothia LCDR1
SQNISRY

SEQ ID NO: 307 - protein, 7 aa, synthetic construct
  REGION 1..7: note = Synthetic: Chothia LCDR1
SQIISRY

SEQ ID NO: 308 - protein, 7 aa, synthetic construct
  REGION 1..7: note = Synthetic: Chothia LCDR1
SQSMSSH

SEQ ID NO: 309 - protein, 7 aa, synthetic construct
  REGION 1..7: note = Synthetic: Chothia LCDR1
SQSISTY

SEQ ID NO: 310 - protein, 7 aa, synthetic construct
  REGION 1..7: note = Synthetic: Chothia LCDR1
SQSISSY

SEQ ID NO: 311 - protein, 6 aa, synthetic construct
  REGION 1..6: note = Synthetic: IMGT LCDR1
QDIRND

SEQ ID NO: 312 - protein, 6 aa, synthetic construct
  REGION 1..6: note = Synthetic: IMGT LCDR1
QGITNY

SEQ ID NO: 313 - protein, 6 aa, synthetic construct
  REGION 1..6: note = Synthetic: IMGT LCDR1
QGIRND

SEQ ID NO: 314 - protein, 6 aa, synthetic construct
  REGION 1..6: note = Synthetic: IMGT LCDR1
QGIRDD

SEQ ID NO: 315 - protein, 12 aa, synthetic construct
  REGION 1..12: note = Synthetic: IMGT LCDR1
QSVFYSSNNKNY

SEQ ID NO: 316 - protein, 6 aa, synthetic construct
  REGION 1..6: note = Synthetic: IMGT LCDR1
QGISRF

SEQ ID NO: 317 - protein, 6 aa, synthetic construct
  REGION 1..6: note = Synthetic: IMGT LCDR1
QSISNY

SEQ ID NO: 318 - protein, 6 aa, synthetic construct
  REGION 1..6: note = Synthetic: IMGT LCDR1
QGISSY

SEQ ID NO: 319 - protein, 6 aa, synthetic construct
  REGION 1..6: note = Synthetic: IMGT LCDR1
QAIRND

SEQ ID NO: 320 - protein, 6 aa, synthetic construct
  REGION 1..6: note = Synthetic: IMGT LCDR1
QAIRDD

SEQ ID NO: 321 - protein, 12 aa, synthetic construct
  REGION 1..12: note = Synthetic: IMGT LCDR1
QSVFYTSNSKHY

SEQ ID NO: 322 - protein, 12 aa, synthetic construct
  REGION 1..12: note = Synthetic: IMGT LCDR1
QSVLYSSNNKNY

SEQ ID NO: 323 - protein, 12 aa, synthetic construct
  REGION 1..12: note = Synthetic: IMGT LCDR1
QSVFYTSNNKNY

SEQ ID NO: 324 - protein, 12 aa, synthetic construct
  REGION 1..12: note = Synthetic: IMGT LCDR1
QSVFYTSSNKHY

SEQ ID NO: 325 - protein, 6 aa, synthetic construct
  REGION 1..6: note = Synthetic: IMGT LCDR1
QSITRY

SEQ ID NO: 326 - protein, 6 aa, synthetic construct
  REGION 1..6: note = Synthetic: IMGT LCDR1
QFITRY

SEQ ID NO: 327 - protein, 6 aa, synthetic construct
  REGION 1..6: note = Synthetic: IMGT LCDR1
QGISRY

SEQ ID NO: 328 - protein, 6 aa, synthetic construct
  REGION 1..6: note = Synthetic: IMGT LCDR1
QSITRS

SEQ ID NO: 329 - protein, 6 aa, synthetic construct
  REGION 1..6: note = Synthetic: IMGT LCDR1
QSISRY

SEQ ID NO: 330 - protein, 6 aa, synthetic construct
  REGION 1..6: note = Synthetic: IMGT LCDR1
QNISRY

SEQ ID NO: 331 - protein, 6 aa, synthetic construct
  REGION 1..6: note = Synthetic: IMGT LCDR1
QIISRY

SEQ ID NO: 332 - protein, 6 aa, synthetic construct
  REGION 1..6: note = Synthetic: IMGT LCDR1
QSMSSH

SEQ ID NO: 333 - protein, 6 aa, synthetic construct
  REGION 1..6: note = Synthetic: IMGT LCDR1
QSISTY

SEQ ID NO: 334 - protein, 6 aa, synthetic construct
  REGION 1..6: note = Synthetic: IMGT LCDR1
QSISSY

SEQ ID NO: 335 - protein, 7 aa, synthetic construct
  REGION 1..7: note = Synthetic: Kabat LCDR2
ASSSLQS

SEQ ID NO: 336 - protein, 7 aa, synthetic construct
  REGION 1..7: note = Synthetic: Kabat LCDR2
DASNLET

SEQ ID NO: 337 - protein, 7 aa, synthetic construct
  REGION 1..7: note = Synthetic: Kabat LCDR2
AASSLQS

SEQ ID NO: 338 - protein, 7 aa, synthetic construct
  REGION 1..7: note = Synthetic: Kabat LCDR2
WASTRES

SEQ ID NO: 339 - protein, 7 aa, synthetic construct
  REGION 1..7: note = Synthetic: Kabat LCDR2
AASTLQN

SEQ ID NO: 340 - protein, 7 aa, synthetic construct
  REGION 1..7: note = Synthetic: Kabat LCDR2
AAYRLQT

SEQ ID NO: 341 - protein, 7 aa, synthetic construct
  REGION 1..7: note = Synthetic: Kabat LCDR2
AASTLQS

SEQ ID NO: 342 - protein, 7 aa, synthetic construct
  REGION 1..7: note = Synthetic: Kabat LCDR2
VVSSFQS

SEQ ID NO: 343 - protein, 7 aa, synthetic construct
  REGION 1..7: note = Synthetic: Kabat LCDR2
ATSSLQS

SEQ ID NO: 344 - protein, 7 aa, synthetic construct
  REGION 1..7: note = Synthetic: Kabat LCDR2
ATSNLQS

SEQ ID NO: 345 - protein, 7 aa, synthetic construct
  REGION 1..7: note = Synthetic: Kabat LCDR2
AASSFQS

SEQ ID NO: 346 - protein, 7 aa, synthetic construct
  REGION 1..7: note = Synthetic: Kabat LCDR2
AASSLKS

SEQ ID NO: 347 - protein, 7 aa, synthetic construct
  REGION 1..7: note = Synthetic: Kabat LCDR2
ATSSFQS

SEQ ID NO: 348 - protein, 7 aa, synthetic construct
  REGION 1..7: note = Synthetic: Kabat LCDR2
AVSSLQS

SEQ ID NO: 349 - protein, 7 aa, synthetic construct
  REGION 1..7: note = Synthetic: Kabat LCDR2
ATSILQS

SEQ ID NO: 350 - protein, 7 aa, synthetic construct
  REGION 1..7: note = Synthetic: Kabat LCDR2
AASSLQN

SEQ ID NO: 351 - protein, 7 aa, synthetic construct
  REGION 1..7: note = Synthetic: Kabat LCDR2
AASILQS

SEQ ID NO: 352 - protein, 7 aa, synthetic construct
  REGION 1..7: note = Synthetic: Kabat LCDR2
GASTLQS

SEQ ID NO: 353 - protein, 7 aa, synthetic construct
  REGION 1..7: note = Synthetic: Kabat LCDR2
AASRLQS

SEQ ID NO: 354 - protein, 7 aa, synthetic construct
  REGION 1..7: note = Synthetic: Kabat LCDR2
GASSLES

SEQ ID NO: 355 - protein, 7 aa, synthetic construct
  REGION 1..7: note = Synthetic: Kabat LCDR2
GASSLQS

SEQ ID NO: 356 - protein, 7 aa, synthetic construct
  REGION 1..7: note = Synthetic: Kabat LCDR2
AASRLES

SEQ ID NO: 357 - protein, 7 aa, synthetic construct
  REGION 1..7: note = Synthetic: Kabat LCDR2
AASNLQS

SEQ ID NO: 358 - protein, 7 aa, synthetic construct
  REGION 1..7: note = Synthetic: Kabat LCDR2
AASSLQS

SEQ ID NO: 359-367 - skipped (9 entries)

SEQ ID NO: 368 - protein, 9 aa, synthetic construct
  REGION 1..9: note = Synthetic: Kabat LCDR3
LQHNNYPFT

SEQ ID NO: 369 - protein, 9 aa, synthetic construct
  REGION 1..9: note = Synthetic: Kabat LCDR3
QQYENLPYT

SEQ ID NO: 370 - protein, 9 aa, synthetic construct
  REGION 1..9: note = Synthetic: Kabat LCDR3
LQHNSYPWS

SEQ ID NO: 371 - protein, 9 aa, synthetic construct
  REGION 1..9: note = Synthetic: Kabat LCDR3
LQHKSNPFT

SEQ ID NO: 372 - protein, 9 aa, synthetic construct
  REGION 1..9: note = Synthetic: Kabat LCDR3
QQHYDIPRT

SEQ ID NO: 373 - protein, 9 aa, synthetic construct
  REGION 1..9: note = Synthetic: Kabat LCDR3
QQTYSTPLT

SEQ ID NO: 374 - protein, 9 aa, synthetic construct
  REGION 1..9: note = Synthetic: Kabat LCDR3
QQSYSSPLT

SEQ ID NO: 375 - protein, 9 aa, synthetic construct
  REGION 1..9: note = Synthetic: Kabat LCDR3
QQLNSYPRT

SEQ ID NO: 376 - protein, 9 aa, synthetic construct
  REGION 1..9: note = Synthetic: Kabat LCDR3
QQLNNYPRT

SEQ ID NO: 377 - protein, 9 aa, synthetic construct
  REGION 1..9: note = Synthetic: Kabat LCDR3
QQLQSYPRT

SEQ ID NO: 378 - protein, 9 aa, synthetic construct
  REGION 1..9: note = Synthetic: Kabat LCDR3
LQHNSYPFT

SEQ ID NO: 379 - protein, 9 aa, synthetic construct
  REGION 1..9: note = Synthetic: Kabat LCDR3
LQHNTYPFS

SEQ ID NO: 380 - protein, 9 aa, synthetic construct
  REGION 1..9: note = Synthetic: Kabat LCDR3
LQHSSYPFT

SEQ ID NO: 381 - protein, 9 aa, synthetic construct
  REGION 1..9: note = Synthetic: Kabat LCDR3
LQHNTYPFT

SEQ ID NO: 382 - protein, 9 aa, synthetic construct
  REGION 1..9: note = Synthetic: Kabat LCDR3
LQHNSNPFT

SEQ ID NO: 383 - protein, 9 aa, synthetic construct
  REGION 1..9: note = Synthetic: Kabat LCDR3
LQHYSNPFT

SEQ ID NO: 384 - protein, 9 aa, synthetic construct
  REGION 1..9: note = Synthetic: Kabat LCDR3
QQYYSIPRT

SEQ ID NO: 385 - protein, 9 aa, synthetic construct
  REGION 1..9: note = Synthetic: Kabat LCDR3
QQHYSTPRT

SEQ ID NO: 386 - protein, 9 aa, synthetic construct
  REGION 1..9: note = Synthetic: Kabat LCDR3
QQYYSTPRT

SEQ ID NO: 387 - protein, 9 aa, synthetic construct
  REGION 1..9: note = Synthetic: Kabat LCDR3
QQSYSTPLT

SEQ ID NO: 388 - protein, 9 aa, synthetic construct
  REGION 1..9: note = Synthetic: Kabat LCDR3
QQSYNTPLT

SEQ ID NO: 389 - protein, 9 aa, synthetic construct
  REGION 1..9: note = Synthetic: Kabat LCDR3
QQSYSTPVT

SEQ ID NO: 390 - protein, 9 aa, synthetic construct
  REGION 1..9: note = Synthetic: Kabat LCDR3
QQSYRTSLT

SEQ ID NO: 391 - protein, 9 aa, synthetic construct
  REGION 1..9: note = Synthetic: Kabat LCDR3
QQSYGTPLT

SEQ ID NO: 392 - protein, 9 aa, synthetic construct
  REGION 1..9: note = Synthetic: Kabat LCDR3
QQSYSTPFT

SEQ ID NO: 393 - protein, 9 aa, synthetic construct
  REGION 1..9: note = Synthetic: Kabat LCDR3
QQSYRSPFT

SEQ ID NO: 394 - protein, 6 aa, synthetic construct
  REGION 1..6: note = Synthetic: Chothia and IMGT LCDR3
HNNYPF

SEQ ID NO: 395 - protein, 6 aa, synthetic construct
  REGION 1..6: note = Synthetic: Chothia and IMGT LCDR3
YENLPY

SEQ ID NO: 396 - protein, 6 aa, synthetic construct
  REGION 1..6: note = Synthetic: Chothia and IMGT LCDR3
HNSYPW

SEQ ID NO: 397 - protein, 6 aa, synthetic construct
  REGION 1..6: note = Synthetic: Chothia and IMGT LCDR3
HKSNPF

SEQ ID NO: 398 - protein, 6 aa, synthetic construct
  REGION 1..6: note = Synthetic: Chothia and IMGT LCDR3
HYDIPR

SEQ ID NO: 399 - protein, 6 aa, synthetic construct
  REGION 1..6: note = Synthetic: Chothia and IMGT LCDR3
TYSTPL

SEQ ID NO: 400 - protein, 6 aa, synthetic construct
  REGION 1..6: note = Synthetic: Chothia and IMGT LCDR3
SYSSPL

SEQ ID NO: 401 - protein, 6 aa, synthetic construct
  REGION 1..6: note = Synthetic: Chothia and IMGT LCDR3
LNSYPR

SEQ ID NO: 402 - protein, 6 aa, synthetic construct
  REGION 1..6: note = Synthetic: Chothia and IMGT LCDR3
LNNYPR

SEQ ID NO: 403 - protein, 6 aa, synthetic construct
  REGION 1..6: note = Synthetic: Chothia and IMGT LCDR3
LQSYPR

SEQ ID NO: 404 - protein, 6 aa, synthetic construct
  REGION 1..6: note = Synthetic: Chothia and IMGT LCDR3
HNSYPF

SEQ ID NO: 405 - protein, 6 aa, synthetic construct
  REGION 1..6: note = Synthetic: Chothia and IMGT LCDR3
HNTYPF

SEQ ID NO: 406 - protein, 6 aa, synthetic construct
  REGION 1..6: note = Synthetic: Chothia and IMGT LCDR3
HSSYPF

SEQ ID NO: 407 - protein, 6 aa, synthetic construct
  REGION 1..6: note = Synthetic: Chothia and IMGT LCDR3
HNSNPF

SEQ ID NO: 408 - protein, 6 aa, synthetic construct
  REGION 1..6: note = Synthetic: Chothia and IMGT LCDR3
HYSNPF

SEQ ID NO: 409 - protein, 6 aa, synthetic construct
  REGION 1..6: note = Synthetic: Chothia and IMGT LCDR3
YYSIPR

SEQ ID NO: 410 - protein, 6 aa, synthetic construct
  REGION 1..6: note = Synthetic: Chothia and IMGT LCDR3
HYSTPR

SEQ ID NO: 411 - protein, 6 aa, synthetic construct
  REGION 1..6: note = Synthetic: Chothia and IMGT LCDR3
YYSTPR

SEQ ID NO: 412 - protein, 6 aa, synthetic construct
  REGION 1..6: note = Synthetic: Chothia and IMGT LCDR3
SYSTPL

SEQ ID NO: 413 - protein, 6 aa, synthetic construct
  REGION 1..6: note = Synthetic: Chothia and IMGT LCDR3
SYNTPL

SEQ ID NO: 414 - protein, 6 aa, synthetic construct
  REGION 1..6: note = Synthetic: Chothia and IMGT LCDR3
SYSTPV

SEQ ID NO: 415 - protein, 6 aa, synthetic construct
  REGION 1..6: note = Synthetic: Chothia and IMGT LCDR3
SYRTSL

SEQ ID NO: 416 - protein, 6 aa, synthetic construct
  REGION 1..6: note = Synthetic: Chothia and IMGT LCDR3
SYGTPL

SEQ ID NO: 417 - protein, 6 aa, synthetic construct
  REGION 1..6: note = Synthetic: Chothia and IMGT LCDR3
SYSTPF

SEQ ID NO: 418 - protein, 6 aa, synthetic construct
  REGION 1..6: note = Synthetic: Chothia and IMGT LCDR3
SYRSPF

SEQ ID NO: 419 - protein, 118 aa, synthetic construct
  REGION 1..118: note = Synthetic: Heavy Chain Variable Domain 1
EVQVVESGGGLVQPGGSLRLSCAASGFTFSNYAMTWVRQSPGKGLEWVSLISGSGGLTKYADSVKGRFTISRDNSKKTLFLQMNSLRAEDTAAYYCVKDEGLTTGEYWGQGTQVTVSS

SEQ ID NO: 420 - protein, 118 aa, synthetic construct
  REGION 1..118: note = Synthetic: Heavy Chain Variable Domain 2
KVQLVESGGGLVQPGGSLRLSCAASGFRFSSYAMNWVRQAPGKGLEWVSLISGSGGLTKYADSVKGRFTISRDNSRNTLYLQMISLRAEDTAVYFCAKERWEPSFHYWGQGTLVTVSS

SEQ ID NO: 421 - protein, 120 aa, synthetic construct
  REGION 1..120: note = Synthetic: Heavy Chain Variable Domain 3
EVQVVESGGGLIQPGGSLRISCAASGFPVSSNYMSWVRQAPGKGLDWVSIIYSRDSTHYADSVKGRFTMSRDNSKNTVYLQMNSLRAEDTAVYYCAGTLTYYYGAGSDYWGQGTLVTVSS

SEQ ID NO: 422 - protein, 118 aa, synthetic construct
  REGION 1..118: note = Synthetic: Heavy Chain Variable Domain 4
EVQLVESGGGLVQPGGSLRLSCAASGFTFSNYWMHWVRQAPGKGLVWVSRINSDGISTRYADSVKGRFTISRDNAKNTLYLQMKSLRAEDTAVYYCVRDFQLELPDYWGQGTLVTVSS

SEQ ID NO: 423 - protein, 120 aa, synthetic construct
  REGION 1..120: note = Synthetic: Heavy Chain Variable Domain 5
EVQLVESGGGFVKPGGSLRLSCAASGFTFSNAWMSWVRQAPGKGLEWVGRIKSNSYGGTTDYAAPAIGRFTISRDGSKSTLYLQMNSLKTEDTAVYYCTTDHPKFGFDFWGQGTLVTVSS

SEQ ID NO: 424 - protein, 123 aa, synthetic construct
  REGION 1..123: note = Synthetic: Heavy Chain Variable Domain 6
EVQLVESGGGLVTPGGSLRISCAASGFTFNNAWMTWVRQAPGKGLEWVGRIKSISYGGTTDSAAPVKGRFTISRDDSENTLYLQMSSLKTEDTAVYYCSTGSVWKYGGSLDFWGQGTLVTVSS

SEQ ID NO: 425 - protein, 122 aa, synthetic construct
  REGION 1..122: note = Synthetic: Heavy Chain Variable Domain 7
EVQLVESGGGLVKPGGSLRLSCAASGFTFSYAWMSWVRQAPGRGLEWVGRIKSETDGGTTDYAAPVKGRFTISRDHSENTLFLQMNSLKTEDTAVYYCTTGSSWNVYFFDFWGQGTLVTVSS

SEQ ID NO: 426 - protein, 125 aa, synthetic construct
  REGION 1..125: note = Synthetic: Heavy Chain Variable Domain 8
QVQLVQSGAEVKKPGASVKVSCKASGYTFTNYDINWVRQATGQGLEWMGWMNPHSGQTGYAQKFQGRVTMTRNTAISTAYMELSSLRSEDTAVYYCAREGYDLWSGDSSNWLDPWGQGTLVTVSA

SEQ ID NO: 427 - protein, 125 aa, synthetic construct
  REGION 1..125: note = Synthetic: Heavy Chain Variable Domain 9
QVQLVQSGAEVKKPGASVKVSCKASGYTFTNYDINWVRQATGQGLEWMGWMNPHSGQTGYAQKFQGRVTMTRNTAISTAYMELSSLRSEDTAVYYCAREGYDLWSGESSNWLDPWGQGTLVTVSA

SEQ ID NO: 428 - protein, 125 aa, synthetic construct
  REGION 1..125: note = Synthetic: Heavy Chain Variable Domain 10
QVQLVQSGAEVKKPGASVKVSCKASGYTFTNYDINWVRQATGQGLEWMGWMNPHSGQTGYAQKFQGRVTMTRNTAISTAYMELSSLRSEDTAVYYCAREGYDLWSGHSSNWLDPWGQGTLVTVSA

SEQ ID NO: 429 - protein, 125 aa, synthetic construct
  REGION 1..125: note = Synthetic: Heavy Chain Variable Domain 11
QVQLVQSGAEVKKPGASVKVSCKASGYTFTNYDINWVRQATGQGLEWMGWMNPHSGQTGYAQKFQGRVTMTRNTAISTAYMELSSLRSEDTAVYYCAREGYDLWSGYSSNWLDPWGQGTLVTVSA

SEQ ID NO: 430 - protein, 118 aa, synthetic construct
  REGION 1..118: note = Synthetic: Heavy Chain Variable Domain 12
EVQVVESGGGLVQPGGSLRLSCAASGFTFSSYAMYWVRQSPGKGLEWVSLITGSGGSTHYADSVKGRFTISRDNSKKTLFLQMNSLRAEDTAAYYCAKDEGLTTGEYWGQGTQVTVSS

SEQ ID NO: 431 - protein, 118 aa, synthetic construct
  REGION 1..118: note = Synthetic: Heavy Chain Variable Domain 13
EVQVVESGGGLVQPGGSLRLSCAASGFTFRNYAMTWVRQSPGKGLEWVSLISGSGGITKYADSVKGRFTISRDNSKKTLFLQMNSLRAEDTAAYYCVKDEGLTTGEYWGQGTQVTVSS

SEQ ID NO: 432 - protein, 118 aa, synthetic construct
  REGION 1..118: note = Synthetic: Heavy Chain Variable Domain 14
EVQVVESGGGLVQPGGSLRLSCAASGFTFSSYAMTWVRQSPGKGLEWVSLISGSGGITHYADSVKGRFTISRDNSKKTLFLQMNSLRAEDTAAYYCVKDEGLTTGEFWGQGTQVTVSS

SEQ ID NO: 433 - protein, 118 aa, synthetic construct
  REGION 1..118: note = Synthetic: Heavy Chain Variable Domain 15
EVQVVESGGGLVQPGGSLRLSCAASGFTFSSYAMTWVRQSPGKGLEWVSLISGSGGVTHYADSVKGRFTISRDNSKKTLFLQMNSLRAEDTAAYYCVKDEGLTTGEFWGQGTQVTVSS

SEQ ID NO: 434 - protein, 118 aa, synthetic construct
  REGION 1..118: note = Synthetic: Heavy Chain Variable Domain 16
EVQVVESGGGLVQPGGSLRLSCAASGFTFSSYAMSWVRQSPGKGLEWVSLISGSGGITHYADSVKGRFTISRDNSKKTLFLQMNSLRAEDTAAYYCVKDEGLTTGEYWGQGTQVTVSS

SEQ ID NO: 435 - protein, 118 aa, synthetic construct
  REGION 1..118: note = Synthetic: Heavy Chain Variable Domain 17
EVQVVESGGGLVQPGGSLRLSCAASGFTFSSYAMSWVRQSPGKGLEWVSLISGSGAITHYADSVKGRFTISRDNSKKTLFLQMNSLRAEDTAAYYCVKDEGLTTGEYWGQGTQVTVSS

SEQ ID NO: 436 - protein, 118 aa, synthetic construct
  REGION 1..118: note = Synthetic: Heavy Chain Variable Domain 18
EVQVVESGGGLVQPGGSLRLSCAASGFTFSSYAMTWVRQSPGKGLEWVSLISGSGGVTHYADSVKGRFTISRDNSKKTLFLQMNSLRAEDTAAYYCVKDEGLTTGESWGQGTQVTVSS

SEQ ID NO: 437 - protein, 118 aa, synthetic construct
  REGION 1..118: note = Synthetic: Heavy Chain Variable Domain 19
EVQVVESGGGLVQPGGSLRLSCAASGFTFSSYAMTWVRQSPGKGLEWVSLISGSGGITHYADSVKGRFTISRDNSKKTLFLQMNSLRAEDTAAYYCVKDEGLTTGESWGQGTQVTVSS

SEQ ID NO: 438 - protein, 118 aa, synthetic construct
  REGION 1..118: note = Synthetic: Heavy Chain Variable Domain 20
EVQVVESGGGLVQPGGSLRLSCAASGFTFSSYAMTWVRQSPGKGLEWVSLISGSGGLTKYADSVKGRFTISRDNSKKTLFLQMNSLRAEDTAAYYCAKEEGLTTGEYWGQGTQVTVSS

SEQ ID NO: 439 - protein, 118 aa, synthetic construct
  REGION 1..118: note = Synthetic: Heavy Chain Variable Domain 21
EVQVVESGGGLVQPGGSLRLSCAASGFTFNTYAMTWVRQSPGKGLEWVSLISGSGGLTKYADSVKGRFTISRDNSKKTLFLQMNSLRAEDTAAYYCAKEEGLTTGEYWGQGTQVTVSS

SEQ ID NO: 440 - protein, 118 aa, synthetic construct
  REGION 1..118: note = Synthetic: Heavy Chain Variable Domain 22
EVQVVESGGGLVQPGGSLRLSCAASGFTFSSYAMTWVRQSPGKGLEWVSLISGSGGLTNYADSVKGRFTISRDNSKKTLFLQMNSLRAEDTAAYYCAKDEGLTSGEYWGQGTQVTVSS

SEQ ID NO: 441 - protein, 118 aa, synthetic construct
  REGION 1..118: note = Synthetic: Heavy Chain Variable Domain 23
EVQVVESGGGLVQPGGSLRLSCAASGFTFSSYAMTWVRQSPGKGLEWVSLISGSGGSTHYADSVKGRFTISRDNSKKTLFLQMNSLRAEDTAAYYCAKDEGLTTGEYWGQGTQVTVSS

SEQ ID NO: 442 - protein, 118 aa, synthetic construct
  REGION 1..118: note = Synthetic: Heavy Chain Variable Domain 24
EVQVVESGGGLVQPGGSLRLSCAASGFTFSSHAMTWVRQSPGKGLEWVSLISGSGGLTKYADSVRGRFTISRDNSKKTLFLQMNSLRAEDTAAYYCVKEEGLTTGEYWGQGTQVTVSS

SEQ ID NO: 443 - protein, 118 aa, synthetic construct
  REGION 1..118: note = Synthetic: Heavy Chain Variable Domain 25
EVQVVESGGGLVQPGGSLRLSCAASGFTFSSYAMTWVRQSPGKGLEWVSLISGSGGLTKYADSVKGRFTISRDNSKKTLFLQMNSLRAEDTAAYYCAKDEGLTTGEYWGQGTQVTVSS

SEQ ID NO: 444 - protein, 118 aa, synthetic construct
  REGION 1..118: note = Synthetic: Heavy Chain Variable Domain 26
EVQVVESGGGLVQPGGSLRLSCAASGFTFSSYAMTWVRQSPGKGLEWVSLISGSGSLTKYADSVKGRFTISRDNSKKTLFLQMNSLRAEDTAAYYCVKEEGLTTGEYWGQGTQVTVSS

SEQ ID NO: 445 - protein, 118 aa, synthetic construct
  REGION 1..118: note = Synthetic: Heavy Chain Variable Domain 27
EVQVVESGGGLVQPGGSLRLSCAASGFTFSSYAMTWVRQSPGKGLEWVSLISGSGGLTKYADSVKGRFTISRDNSKKTLFLQMNSLRAEDTAAYYCVKDEGLTTGEYWGQGTQVTVSS

SEQ ID NO: 446 - protein, 118 aa, synthetic construct
  REGION 1..118: note = Synthetic: Heavy Chain Variable Domain 28
EVQVVESGGGLVQPGGSLRLSCAASGFTFGNYAMTWVRQSPGKGLEWVSLISGSGGLTKYADSVKGRFTISRDNSKKTLFLQMNSLRAEDTAAYYCVKDEGLTTGEYWGQGTQVTVSS

SEQ ID NO: 447 - protein, 118 aa, synthetic construct
  REGION 1..118: note = Synthetic: Heavy Chain Variable Domain 29
EVQVVESGGGLVQPGGSLRLSCAASGFTFTNYAMTWVRQSPGKGLEWVSLISGGGGLTYYADSVKGRFTISRDNSKKTLFLQMNSLRAEDTAAYYCVKDEGLTTGEYWGQGTQVTVSS

SEQ ID NO: 448 - protein, 118 aa, synthetic construct
  REGION 1..118: note = Synthetic: Heavy Chain Variable Domain 30
EVQVVESGGGLVQPGGSLRLSCAASGFTFSIYAMTWVRQSPGKGLEWVSLISGSGGLTKYADSVKGRFTISRDNSKKTLFLQMNSLRAEDTAAYYCVKDEGLTTGEYWGQGTQVTVSS

SEQ ID NO: 449 - protein, 118 aa, synthetic construct
  REGION 1..118: note = Synthetic: Heavy Chain Variable Domain 31
EVQVVESGGGLVQPGGSLRLSCAASGFTFSSYAMTWVRQSPGKGLEWVSLISGSGGLTYYADSVKGRFTISRDNSKKTLFLQMNSLRAEDTAAYYCVKDEGLTTGEYWGQGTQVTVSS

SEQ ID NO: 450 - protein, 118 aa, synthetic construct
  REGION 1..118: note = Synthetic: Heavy Chain Variable Domain 32
EVQVVESGGGLVQPGGSLRLSCAASGFTFSSHAMTWVRQSPGKGLEWVSLISGSGGLTYYADSVKGRFTISRDNSKKTLFLQMNSLRAEDTAAYYCVKDEGLTTGEYWGQGTQVTVSS

SEQ ID NO: 451 - protein, 118 aa, synthetic construct
  REGION 1..118: note = Synthetic: Heavy Chain Variable Domain 33
EVQVVESGGGLVQPGGSLRLSCAASGFTFSNYAMTWVRQSPGKGLEWVSLISGGGGLTYYADSVKGRFTISRDNSKKTLFLQMNSLRAEDTAAYYCVKDEGLTTGEYWGQGTQVTVSS

SEQ ID NO: 452 - protein, 118 aa, synthetic construct
  REGION 1..118: note = Synthetic: Heavy Chain Variable Domain 34
EVQVVESGGGLVQPGGSLRLSCAASGFTFSSYAMTWVRQSPGKGLEWVSLISGSGGLTKYADSVKGRFTISRDNSKKTLFLQMNSLRAEDTAAYYCVKEEGLTTGEYWGQGTQVTVSS

SEQ ID NO: 453 - protein, 118 aa, synthetic construct
  REGION 1..118: note = Synthetic: Heavy Chain Variable Domain 35
EVQVVESGGGLVQPGGSLRLSCAASGFTFSSYAMTWVRQSPGKGLEWVSLISGSGGITKYADSVKGRFTISRDNSKKTLFLQMNSLRAEDTAAYYCAKEEGLTTGEYWGQGTQVTVSS

SEQ ID NO: 454 - protein, 118 aa, synthetic construct
  REGION 1..118: note = Synthetic: Heavy Chain Variable Domain 36
EVQLVESGGGLVQPGGSLRLSCAASGFRFSSYAMNWVRQAPGKGLEWVSLISGSGGLTKYADSVKGRFTISRDNSRNTLYLQMISLRAEDTAVYFCAKERWEPSFHYWGQGTLVTVSS

SEQ ID NO: 455 - protein, 118 aa, synthetic construct
  REGION 1..118: note = Synthetic: Heavy Chain Variable Domain 37
EVQLVESGGGLVQPGGSLRLSCAASGFTFRSYWMHWVRQAPGKGLVWVSRIDNNGINTRYADSVKGRFTISRDNAKNTLYLQMKSLRAEDTAVYYCVRDFQLELRDYWGQGTLVTVSS

SEQ ID NO: 456 - protein, 118 aa, synthetic construct
  REGION 1..118: note = Synthetic: Heavy Chain Variable Domain 38
EVQLVESGGGLVQPGGSLRLSCAASGFTFSNYWMHWVRQAPGKGLVWVSRISSDGISTRYADSVKGRFTISRDNAKNTLYLQMKSLRAEDTAVYYCVRDFQLELPDYWGQGTLVTVSS

SEQ ID NO: 457 - protein, 118 aa, synthetic construct
  REGION 1..118: note = Synthetic: Heavy Chain Variable Domain 39
EVQLVESGGGLVQPGGSLRLSCAASGLTFSNYWMHWVRQAPGKGLVWVSRINSDGISTRYADSVKGRFTISRDNAKNTLYLQMKSLRAEDTAVYYCVSDFQLELPDYWGQGTLVTVSS

SEQ ID NO: 458 - protein, 118 aa, synthetic construct
  REGION 1..118: note = Synthetic: Heavy Chain Variable Domain 40
EVQLVESGGGLVQPGGSLRLSCAASGFTFSNYWMHWVRQAPGKGLVWVSRINNDGISTRYADSVKGRFTISRDNAKNTLYLQMKSLRAEDTAVYYCVRDFQLELPDYWGQGTLVTVSS

SEQ ID NO: 459 - protein, 118 aa, synthetic construct
  REGION 1..118: note = Synthetic: Heavy Chain Variable Domain 41
EVQLVESGGGLVQPGGSLRLSCAASGFTFSSFWMHWVRQAPGKGLVWVSRINSDGFSTRYADSVKGRFTISRDNAKNTLYLQMKSLRAEDTAVYYCVRDFQLELRDFWGQGTLVTVSS

SEQ ID NO: 460 - protein, 118 aa, synthetic construct
  REGION 1..118: note = Synthetic: Heavy Chain Variable Domain 42
EVQLVESGGGLVQPGGSLRLSCAASGFTFSSFWMHWVRQAPGKGLVWVSRINSDGFSTRYADSVKGRFTISRDNAKNTLYLQMKSLRAEDTAVYYCVRDFQLELRDYWGQGTLVTVSS

SEQ ID NO: 461 - protein, 118 aa, synthetic construct
  REGION 1..118: note = Synthetic: Heavy Chain Variable Domain 43
EVQLVESGGGLVQPGGSLRLSCAASGFTFSSYWMHWVRQAPGKGLVWVSRINSDGISTRYADSVKGRFTISRDNAKNTLYLQMKSLRAEDTAVYYCVRDFQLELRDYWGQGTLVTVSS

SEQ ID NO: 462 - protein, 120 aa, synthetic construct
  REGION 1..120: note = Synthetic: Heavy Chain Variable Domain 44
EVQLVESGGGFVKPGGSLRLSCAASGFTFHNAWMSWVRQAPGKGLEWVGRIKGVTAGGTADYAAPVKGRFTISRDGSKSTLYLQMNSLKTEDTAVYYCTTDHPKFGFDFWGQGTLVTVSS

SEQ ID NO: 463 - protein, 120 aa, synthetic construct
  REGION 1..120: note = Synthetic: Heavy Chain Variable Domain 45
EVQLVESGGGFVKPGGSLRLSCAASGFTFNNAWMSWVRQAPGKGLEWVGRVKSVTAGGTTDYAAPVKGRFTISRDGSKSTLYLQMNSLKTEDTAVYYCTTDHPKFGFDFWGQGTLVTVSS

SEQ ID NO: 464 - protein, 120 aa, synthetic construct
  REGION 1..120: note = Synthetic: Heavy Chain Variable Domain 46
EVQLVESGGGFVKPGGSLRLSCAASGFTFSNAWMSWVRQAPGKGLEWVGRIKSKTDGGTTDYAAPVKGRFTISRDGSKSTLYLQMNSLKTEDTAVYYCTTDHPKFGFDYWGQGTLVTVSS

SEQ ID NO: 465 - protein, 120 aa, synthetic construct
  REGION 1..120: note = Synthetic: Heavy Chain Variable Domain 47
EVQLVESGGGFVKPGGSLRLSCAASGFTFNSAWMSWVRQAPGKGLEWVGRIKSVTTGGTTDYAAPVKGRFTISRDGSKSTLYLQMNSLKTEDTAVYYCTTDHPKFGFDFWGQGTLVTVSS

SEQ ID NO: 466 - protein, 120 aa, synthetic construct
  REGION 1..120: note = Synthetic: Heavy Chain Variable Domain 48
EVQLVESGGGFVKPGGSLRLSCAASGFTFNNAWMSWVRQAPGKGLEWVGRIKSVTAGGTTDYAAPVKGRFTISRDGSKSTLYLQMNSLKTEDTAVYYCTTDHPKFGFDYWGQGTLVTVSS

SEQ ID NO: 467 - protein, 120 aa, synthetic construct
  REGION 1..120: note = Synthetic: Heavy Chain Variable Domain 49
EVQLVESGGGFVKPGGSLRLSCAASGFTFNNAWMSWVRQAPGKGLEWVGRIKSVTAGGTTDFPAPVKGRFTISRDGSKSTLYLQMNSLKTEDTAVYYCTTDHPKFGFDFWGQGTLVTVSS

SEQ ID NO: 468 - protein, 123 aa, synthetic construct
  REGION 1..123: note = Synthetic: Heavy Chain Variable Domain 50
EVQLVESGGGLVTPGGSLRISCAASGFIFNNAWMRWVRQAPGKGLEWVGHIKSETDGGTTDYAAPVKGRFTISRDDSENTLYLQMSSLKTEDTAVYYCTTGGVWNYGGSLDFWGQGTLVTVSS

SEQ ID NO: 469 - protein, 123 aa, synthetic construct
  REGION 1..123: note = Synthetic: Heavy Chain Variable Domain 51
EVQLVESGGGLVTPGGSLRISCAASGFIFNNAWMRWVRQAPGKGLEWVGHIKSETDGGTTDYAAPVKGRFTISRDDSENTLYLQMSSLKTEDTAVYYCITGGVWNYGGSLDFWGQGTLVTVSS

SEQ ID NO: 470 - protein, 123 aa, synthetic construct
  REGION 1..123: note = Synthetic: Heavy Chain Variable Domain 52
EVQLVESGGGLVTPGGSLRISCAASGFIFNNAWMRWVRQAPGKGLEWVGHIKSETDGGTTDYAAPVKGRFTISRDDSENTLYLQMSSLKTEDTAVYYCTTGGVWKYGGSLDFWGQGTLVTVSS

SEQ ID NO: 471 - protein, 123 aa, synthetic construct
  REGION 1..123: note = Synthetic: Heavy Chain Variable Domain 53
EVQLVESGGGLVTPGGSLRISCAASGFIFNNAWMRWVRQAPGKGLEWVGHIKSETDGGTTDYAAPVKGRFTISRDDSENTLYLQMSSLKTEDTAVYYCATGGVWKYGGSLDFWGQGTLVTVSS

SEQ ID NO: 472 - protein, 123 aa, synthetic construct
  REGION 1..123: note = Synthetic: Heavy Chain Variable Domain 54
EVQLVESGGGLVTPGGSLRISCAASGFTFNYAWMTWVRQAPGKGLEWVGRIKRKTDGGTTDYAAPVQGRFTISRDDSENTLYLQMSSLKTEDTAVYYCTTGSVWKYGGSLDFWGQGTLVTVSS

SEQ ID NO: 473 - protein, 123 aa, synthetic construct
  REGION 1..123: note = Synthetic: Heavy Chain Variable Domain 55
EVQLVESGGGLVTPGGSLRISCAASGFTFINAWMNWVRQAPGKGLEWVGRIKSLTDGGTTDYAAPVKGRFTISRDDSENTLYLQMSSLKTEDTAVYYCSTGSVWNYGGSLDFWGQGTLVTVSS

SEQ ID NO: 474 - protein, 123 aa, synthetic construct
  REGION 1..123: note = Synthetic: Heavy Chain Variable Domain 56
EVQLVESGGGLVTPGGSLRISCAASGFTFSNAWMNWVRQAPGKGLEWVGRIKRKTDGGTREYAAPVKGRFTISRDDSENTLYLQMSSLKTEDTAVYYCTTGSVWKYGGSLDFWGQGTLVTVSS

SEQ ID NO: 475 - protein, 123 aa, synthetic construct
  REGION 1..123: note = Synthetic: Heavy Chain Variable Domain 57
EVQLVESGGGLVTPGGSLRISCAASGFTFSNAWMSWVRQAPGKGLEWVGRIKRNTDGGTTDYAAPVKGRFTISRDDSENTLYLQMSSLKTEDTAVYYCTTGSVWKYGGSLDFWGQGTLVTVSS

SEQ ID NO: 476 - protein, 123 aa, synthetic construct
  REGION 1..123: note = Synthetic: Heavy Chain Variable Domain 58
EVQLVESGGGLVTPGGSLRISCAASGFTFSNAWMSWVRQAPGKGLEWVGRIKTNTDGGTTDYAAPVKGRFTISRDDSENTLYLQMSSLKTEDTAVYYCTTGSVWNYGGSLDYWGQGTLVTVSS

SEQ ID NO: 477 - protein, 123 aa, synthetic construct
  REGION 1..123: note = Synthetic: Heavy Chain Variable Domain 59
EVQLVESGGGLVTPGGSLRISCAASGFTFSNAWMSWVRQAPGKGLEWVGRIKSKTYGGTTDYDAPVKGRFTISRDDSENTLYLQMSSLKTEDTAVYYCTTGSVWKYGGSLDYWGQGTLVTVSS

SEQ ID NO: 478 - protein, 123 aa, synthetic construct
  REGION 1..123: note = Synthetic: Heavy Chain Variable Domain 60
EVQLVESGGGLVTPGGSLRISCAASGFTFSNAWMSWVRQAPGKGLEWVGRIKSQTYGGTTDYAAPVKGRFTISRDDSENTLYLQMSSLKTEDTAVYYCTTGSVWKYGGSLDYWGQGTLVTVSS

SEQ ID NO: 479 - protein, 123 aa, synthetic construct
  REGION 1..123: note = Synthetic: Heavy Chain Variable Domain 61
EVQLVESGGGLVTPGGSLRISCAASGFTFSNAWMSWVRQAPGKGLEWVGRIKSKTDGGTTDYAAPVKGRFTISRDDSENTLYLQMSSLKTEDTAVYYCTTGSVWRYGGSLDFWGQGTLVTVSS

SEQ ID NO: 480 - protein, 123 aa, synthetic construct
  REGION 1..123: note = Synthetic: Heavy Chain Variable Domain 62
EVQLVESGGGLVTPGGSLRISCAASGFTFSNAWMNWVRQAPGKGLEWVGRIKSKTDGGTTDYAAPVKGRFTISRDDSENTLYLQMSSLKTEDTAVYYCTTGSVWNYGGSLDFWGQGTLVTVSS

SEQ ID NO: 481 - protein, 123 aa, synthetic construct
  REGION 1..123: note = Synthetic: Heavy Chain Variable Domain 63
EVQLVESGGGLVTPGGSLRISCAASGFTFSNAWMSWVRQAPGKGLEWVGRIKSKTDGGTTDYAAPVKGRFTISRDDSENTLYLQMSSLKTEDTAVYYCTTGSVWKYGGSLDFWGQGTLVTVSS

SEQ ID NO: 482 - protein, 123 aa, synthetic construct
  REGION 1..123: note = Synthetic: Heavy Chain Variable Domain 64
EVQLVESGGGLVTPGGSLRISCAASGFTFSNAWMTWVRQAPGKGLEWVGRIKSKTYGGTTDYAAPVKGRFTISRDDSENTLYLQMSSLKTEDTAVYYCTTGSVWNYGGSLDFWGQGTLVTVSS

SEQ ID NO: 483 - protein, 123 aa, synthetic construct
  REGION 1..123: note = Synthetic: Heavy Chain Variable Domain 65
EVQLVESGGGLVTPGGSLRISCAASGFTFINAWMNWVRQAPGKGLEWVGRIKRKTDGGTRDYAAPVKGRFTISRDDSENTLYLQMSSLKTEDTAVYYCTTGSVWKYGGSLDFWGQGTLVTVSS

SEQ ID NO: 484 - protein, 122 aa, synthetic construct
  REGION 1..122: note = Synthetic: Heavy Chain Variable Domain 66
EVQLVESGGGLVKPGGSLRLSCAASGFTFSNAWMSWVRQAPGRGLEWVGRIKRETDGGTTDYASPVKGRFTISRDHSENTLFLQMNSLKTEDTAVYYCTTGSSWNVYFFDFWGQGTLVTVSS

SEQ ID NO: 485 - protein, 122 aa, synthetic construct
  REGION 1..122: note = Synthetic: Heavy Chain Variable Domain 67
EVQLVESGGGLVKPGGSLRLSCAASGFTFSTAWMSWVRQAPGRGLEWVGRIKRKSDGGTTDYAAPVKGRFTISRDHSENTLFLQMNSLKTEDTAVYYCTTGSSWHVYFFDFWGQGTLVTVSS

SEQ ID NO: 486 - protein, 122 aa, synthetic construct
  REGION 1..122: note = Synthetic: Heavy Chain Variable Domain 68
EVQLVESGGGLVKPGGSLRLSCAASGFIFSNAWMSWVRQAPGRGLEWVGRIKSKTYGGTTDYAAPVKGRFTISRDHSENTLFLQMNSLKTEDTAVYYCTTGSSWSLYYFDFWGQGTLVTVSS

SEQ ID NO: 487 - protein, 122 aa, synthetic construct
  REGION 1..122: note = Synthetic: Heavy Chain Variable Domain 69
EVQLVESGGGLVKPGGSLRLSCAASGFIFSNAWMSWVRQAPGRGLEWVGRIKSKTDGGTTDYAAPVKGRFTISRDHSENTLFLQMNSLKTEDTAVYYCTTGSSWYVYYFDYWGQGTLVTVSS

SEQ ID NO: 488 - protein, 122 aa, synthetic construct
  REGION 1..122: note = Synthetic: Heavy Chain Variable Domain 70
EVQLVESGGGLVKPGGSLRLSCAASGFIFSNAWMSWVRQAPGRGLEWVGRIKSEIDGGTTDYAAPVKGRFTISRDHSENTLFLQMNSLKTEDTAVYYCTTGSSWYVYYFDFWGQGTLVTVSS

SEQ ID NO: 489 - protein, 107 aa, synthetic construct
  REGION 1..107: note = Synthetic: Light Chain Variable Domain 1
DIQMTQSPSSLSASVGDRVTITCRASQDIRNDLAWYQQKPGKAPKRLIYASSSLQSGVPSRFSGSGSGTEFTLTISSLQPEDFATYYCLQHNNYPFTFGPGTKVDIK

SEQ ID NO: 490 - protein, 107 aa, synthetic construct
  REGION 1..107: note = Synthetic: Light Chain Variable Domain 2
DIQMTQSPSSLSASVGDSVIITCQASQGITNYLNWYQQKPGKAPNLLIYDASNLETGVPSRFSGSGSGTYFTFTISSLQPEDIATYFCQQYENLPYTFGQGTKLEIK

SEQ ID NO: 491 - protein, 107 aa, synthetic construct
  REGION 1..107: note = Synthetic: Light Chain Variable Domain 3
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWSFGQGTKVEIK

SEQ ID NO: 492 - protein, 107 aa, synthetic construct
  REGION 1..107: note = Synthetic: Light Chain Variable Domain 4
DIQMTQSPSSLSASVGDRVTITCRASQGIRDDLAWYQQKAGKAPKRLIFAASSLQSGVPSRFSGSGSGTEFTLTISGLQPEDFANYYCLQHKSNPFTFGQGTKLEIK

SEQ ID NO: 493 - protein, 113 aa, synthetic construct
  REGION 1..113: note = Synthetic: Light Chain Variable Domain 5
DIVMTQSPDSLAVSLGERATIDCKSSQSVFYSSNNKNYLAWFQQKPGQPPKLLIYWASTRESGVPDRFSGSGSGTDFTLTISSLQAEDVAVYYCQQHYDIPRTFGQGTKVEIK

SEQ ID NO: 494 - protein, 107 aa, synthetic construct
  REGION 1..107: note = Synthetic: Light Chain Variable Domain 6
DIQMTQSPSSLSASVGDRVTFTCRASQGISRFLNWYQQKPGKAPKLLIYAASTLQNGVPSRFSGSGSGTDFTLTISSLQPEDFATYFCQQTYSTPLTFGGGTKVEIY

SEQ ID NO: 495 - protein, 107 aa, synthetic construct
  REGION 1..107: note = Synthetic: Light Chain Variable Domain 7
DIQMTQSPSSLSASVGDSVTITCRASQSISNYLNWYHQKPGKAPKLLIYAAYRLQTGVPSRFSGSGSGTDFTLTISSLQPEDFATYYCQQSYSSPLTFGGGTKVEIK

SEQ ID NO: 496 - protein, 107 aa, synthetic construct
  REGION 1..107: note = Synthetic: Light Chain Variable Domain 8
DIQLTQSPSFLSASVGDRVTITCRASQGISSYLAWYQQKPGKAPKLLIYAASTLQSGFRSRFSGSGSGTEFTLTISSLQPEDFASYYCQQLNSYPRTFGQGTKVEIK

SEQ ID NO: 497 - protein, 107 aa, synthetic construct
  REGION 1..107: note = Synthetic: Light Chain Variable Domain 9
DIQLTQSPSFLSASVGDRVTITCRASQGISSYLAWYQQKPGKAPKLLIYAASTLQSGFRSRFSGSGSGTEFTLTISSLQPEDFASYYCQQLNNYPRTFGQGTKVEIK

SEQ ID NO: 498 - protein, 107 aa, synthetic construct
  REGION 1..107: note = Synthetic: Light Chain Variable Domain 10
DIQLTQSPSFLSASVGDRVTITCRASQGISSYLAWYQQKPGKAPKLLIYAASTLQSGFRSRFSGSGSGTEFTLTISSLQPEDFASYYCQQLQSYPRTFGQGTKVEIK

SEQ ID NO: 499 - protein, 107 aa, synthetic construct
  REGION 1..107: note = Synthetic: Light Chain Variable Domain 11
DIQMTQSPSSLSASVGDRVTITCRASQGIRNDLGWYQQKPGKAPKRLIYVVSSFQSGVPSRFSGSGSGTEFTLTISSLQPEDFATYYCLQHNNYPFTFGPGTKVDIK

SEQ ID NO: 500 - protein, 107 aa, synthetic construct
  REGION 1..107: note = Synthetic: Light Chain Variable Domain 12
DIQMTQSPSSLSASVGDRVTITCRASQDIRNDLDWYQQKPGKAPKRLIYATSSLQSGVPSRFSGSGSGTEFTLTISSLQPEDFATYYCLQHNSYPFTFGPGTKVDIK

SEQ ID NO: 501 - protein, 107 aa, synthetic construct
  REGION 1..107: note = Synthetic: Light Chain Variable Domain 13
DIQMTQSPSSLSASVGDRVTITCRASQAIRNDLGWYQQKPGKAPKRLIYATSNLQSGVPSRFSGSGSGTEFTLTISSLQPEDFATYYCLQHNSYPFTFGPGTKVDIK

SEQ ID NO: 502 - protein, 107 aa, synthetic construct
  REGION 1..107: note = Synthetic: Light Chain Variable Domain 14
DIQMTQSPSSLSASVGDRVTITCRASQGIRNDLGWYQQKPGKAPKRLIYATSSLQSGVPSRFSGSGSGTEFTLTISSLQPEDFATYYCLQHNSYPFTFGPGTKVDIK

SEQ ID NO: 503 - protein, 107 aa, synthetic construct
  REGION 1..107: note = Synthetic: Light Chain Variable Domain 15
DIQMTQSPSSLSASVGDRVTITCRTSQGIRNDLAWYQQKPGKAPKRLIYAASSLQSGVPSRFSGSGSGTEFTLTISSLQPEDFATYYCLQHNNYPFTFGPGTKVDIK

SEQ ID NO: 504 - protein, 107 aa, synthetic construct
  REGION 1..107: note = Synthetic: Light Chain Variable Domain 16
DIQMTQSPSSLSASVGDRVTITCRASQGIRNDLAWYQQKPGKAPKRLIYAASSFQSGVPSRFSGSGSGTEFTLTISSLQPEDFATYYCLQHNSYPFTFGPGTKVDIK

SEQ ID NO: 505 - protein, 107 aa, synthetic construct
  REGION 1..107: note = Synthetic: Light Chain Variable Domain 17
DIQMTQSPSSLSASVGDRVTITCRASQGIRNDLGWYQQKPGKAPKRLIYAASSLKSGVPSRFSGSGSGTEFTLTISSLQPEDFATYYCLQHNSYPFTFGPGTKVDIK

SEQ ID NO: 506 - protein, 107 aa, synthetic construct
  REGION 1..107: note = Synthetic: Light Chain Variable Domain 18
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPFTFGPGTKVDIK

SEQ ID NO: 507 - protein, 107 aa, synthetic construct
  REGION 1..107: note = Synthetic: Light Chain Variable Domain 19
DIQMTQSPSSLSASVGDRVTITCRASQGIRNDLGWYQQKPGKAPKRLIYATSSFQSGVPSRFSGSGSGTEFTLTISSLQPEDFATYYCLQHNSYPFTFGPGTKVDIK

SEQ ID NO: 508 - protein, 107 aa, synthetic construct
  REGION 1..107: note = Synthetic: Light Chain Variable Domain 20
DIQMTQSPSSLSASVGDRVTITCRASQGIRNDLGWYQQKPGKAPKRLIYAASSFQSGVPSRFSGSGSGTEFTLTISSLQPEDFATYYCLQHNSYPFTFGPGTKVDIK

SEQ ID NO: 509 - protein, 107 aa, synthetic construct
  REGION 1..107: note = Synthetic: Light Chain Variable Domain 21
DIQMTQSPSSLSASVGDRVTITCRASQGIRNNLGWYQQKPGKAPKRLIYAASSLQSGVPSRFSGSGSGTEFTLTISSLQPEDFATYYCLQHNSYPFTFGPGTKVDIK

SEQ ID NO: 510 - protein, 107 aa, synthetic construct
  REGION 1..107: note = Synthetic: Light Chain Variable Domain 22
DIQMTQSPSSLSASVGDRVTITCRASQGIRNDLGWYQQKPGKAPKRLIYAASSLQSGVPSRFSGSGSGTEFTLTISSLQPEDFATYYCLQHNTYPFSFGPGTKVDIK

SEQ ID NO: 511 - protein, 107 aa, synthetic construct
  REGION 1..107: note = Synthetic: Light Chain Variable Domain 23
DIQMTQSPSSLSASVGDRVTITCRASQGIRNDLGWYQQKPGKAPKRLIYAASSLQSGVPSRFSGSGSGTEFTLTISSLQPEDFATYYCLQHSSYPFTFGPGTKVDIK

SEQ ID NO: 512 - protein, 107 aa, synthetic construct
  REGION 1..107: note = Synthetic: Light Chain Variable Domain 24
DIQMTQSPSSLSASVGDRVTITCRASQGIRNDLGWYQQKPGKAPKRLIYAASSLQSGVPSRFSGSGSGTEFTLTISSLQPEDFATYYCLQHNNYPFTFGPGTKVDIK

SEQ ID NO: 513 - protein, 107 aa, synthetic construct
  REGION 1..107: note = Synthetic: Light Chain Variable Domain 25
DIQMTQSPSSLSASVGDRVTITCRASQGIRNDLGWYQQKPGKAPKRLIYAVSSLQSGVPSRFSGSGSGTEFTLTISSLQPEDFATYYCLQHNSYPFTFGPGTKVDIK

SEQ ID NO: 514 - protein, 107 aa, synthetic construct
  REGION 1..107: note = Synthetic: Light Chain Variable Domain 26
DIQMTQSPSSLSASVGDRVTITCRASQGIRNDLGWYQQKPGKAPKRLIYAASSLQSGVPSRFSGSGSGTEFTLTISSLQPEDFATYYCLQHNTYPFTFGPGTKVDIK

SEQ ID NO: 515 - protein, 107 aa, synthetic construct
  REGION 1..107: note = Synthetic: Light Chain Variable Domain 27
DIQMTQSPSSLSASVGDRVTITCRASQGIRNDLGWYQQKPGKAPKRLIYASSSLQSGVPSRFSGSGSGTEFTLTISSLQPEDFATYYCLQHNNYPFTFGPGTKVDIK

SEQ ID NO: 516 - protein, 107 aa, synthetic construct
  REGION 1..107: note = Synthetic: Light Chain Variable Domain 28
DIQMTQSPSSLSASVGDRVTITCRASQGIRNDLAWYQQKAGKAPKRLIFATSILQSGVPSRFSGSGSGTEFTLTISGLQPEDFANYYCLQHNSNPFTFGQGTKLEIK

SEQ ID NO: 517 - protein, 107 aa, synthetic construct
  REGION 1..107: note = Synthetic: Light Chain Variable Domain 29
DIQMTQSPSSLSASVGDRVTITCRASQGIRNDLGWYQQKAGKAPKRLIFAASSLQSGVPSRFSGSGSGTEFTLTISGLQPEDFANYYCLQHKSNPFTFGQGTKLEIK

SEQ ID NO: 518 - protein, 107 aa, synthetic construct
  REGION 1..107: note = Synthetic: Light Chain Variable Domain 30
DIQMTQSPSSLSASVGDRVTITCRASQAIRDDLAWYQQKAGKAPKRLIFAASSLQNGVPSRFSGSGSGTEFTLTISGLQPEDFANYYCLQHKSNPFTFGQGTKLEIK

SEQ ID NO: 519 - protein, 107 aa, synthetic construct
  REGION 1..107: note = Synthetic: Light Chain Variable Domain 31
DIQMTQSPSSLSASVGDRVTITCRASQGIRNDLAWYQQKAGKAPKRLIFAASILQSGVPSRFSGSGSGTEFTLTISGLQPEDFANYYCLQHNSNPFTFGQGTKLEIK

SEQ ID NO: 520 - protein, 107 aa, synthetic construct
  REGION 1..107: note = Synthetic: Light Chain Variable Domain 32
DIQMTQSPSSLSASVGDRVTITCRASQGIRNDLAWYQQKAGKAPKRLIFAASILQSGVPSRFSGSGSGTEFTLTISGLQPEDFANYYCLQHKSNPFTFGQGTKLEIK

SEQ ID NO: 521 - protein, 107 aa, synthetic construct
  REGION 1..107: note = Synthetic: Light Chain Variable Domain 33
DIQMTQSPSSLSASVGDRVTITCRASQGIRNDLAWYQQKAGKAPKRLIFAASSLQSGVPSRFSGSGSGTEFTLTISGLQPEDFANYYCLQHNSNPFTFGQGTKLEIK

SEQ ID NO: 522 - protein, 107 aa, synthetic construct
  REGION 1..107: note = Synthetic: Light Chain Variable Domain 34
DIQMTQSPSSLSASVGDRVTITCRASQGIRNDLAWYQQKAGKAPKRLIFAASILQSGVPSRFSGSGSGTEFTLTISGLQPEDFANYYCLQHYSNPFTFGQGTKLEIK

SEQ ID NO: 523 - protein, 113 aa, synthetic construct
  REGION 1..113: note = Synthetic: Light Chain Variable Domain 35
DIVMTQSPDSLAVSLGERATIDCKSSQSVFYTSNSKHYLNWFQQKPGQPPKLLIYWASTRESGVPDRFSGSGSGTDFTLTISSLQAEDVAVYYCQQYYSIPRTFGQGTKVEIK

SEQ ID NO: 524 - protein, 113 aa, synthetic construct
  REGION 1..113: note = Synthetic: Light Chain Variable Domain 36
DIVMTQSPDSLAVSLGERATIDCKSSQSVLYSSNNKNYLAWFQQKPGQPPKLLIYWASTRESGVPDRFSGSGSGTDFTLTISSLQAEDVAVYYCQQHYSTPRTFGQGTKVEIK

SEQ ID NO: 525 - protein, 113 aa, synthetic construct
  REGION 1..113: note = Synthetic: Light Chain Variable Domain 37
DIVMTQSPDSLAVSLGERATIDCKSSQSVFYTSNSKHYLNWFQQKPGQPPKLLIYWASTRESGVPDRFSGSGSGTDFTLTISSLQAEDVAVYYCQQYYSTPRTFGQGTKVEIK

SEQ ID NO: 526 - protein, 113 aa, synthetic construct
  REGION 1..113: note = Synthetic: Light Chain Variable Domain 38
DIVMTQSPDSLAVSLGERATIDCKSSQSVFYTSNNKNYLNWFQQKPGQPPKLLIYWASTRESGVPDRFSGSGSGTDFTLTISSLQAEDVAVYYCQQYYSTPRTFGQGTKVEIK

SEQ ID NO: 527 - protein, 113 aa, synthetic construct
  REGION 1..113: note = Synthetic: Light Chain Variable Domain 39
DIVMTQSPDSLAVSLGERATIDCKSSQSVFYTSSNKHYLNWFQQKPGQPPKLLIYWASTRESGVPDRFSGSGSGTDFTLTISSLQAEDVAVYYCQQYYSTPRTFGQGTKVEIK

SEQ ID NO: 528 - protein, 107 aa, synthetic construct
  REGION 1..107: note = Synthetic: Light Chain Variable Domain 40
DIQMTQSPSSLSASVGDRVTFTCRASQSITRYLNWYQQKPGKAPKLLIYAASSLQSGVPSRFSGSGSGTDFTLTISSLQPEDFATYFCQQSYSTPLTFGGGTKVEIY

SEQ ID NO: 529 - protein, 107 aa, synthetic construct
  REGION 1..107: note = Synthetic: Light Chain Variable Domain 41
DIQMTQSPSSLSASVGDRVTFTCRASQFITRYLNWYQQKPGKAPKLLIYAASSLQSGVPSRFSGSGSGTDFTLTISSLQPEDFATYFCQQSYSTPLTFGGGTKVEIY

SEQ ID NO: 530 - protein, 107 aa, synthetic construct
  REGION 1..107: note = Synthetic: Light Chain Variable Domain 42
DIQMTQSPSSLSASVGDRVTFTCRASQSITRYLNWYQQKPGKAPKLLIYAASSLQSGVPSRFSGSGSGTDFTLTISSLQPEDFATYFCQQSYNTPLTFGGGTKVEIY

SEQ ID NO: 531 - protein, 107 aa, synthetic construct
  REGION 1..107: note = Synthetic: Light Chain Variable Domain 43
DIQMTQSPSSLSASVGDRVTFTCRASQGISRYLNWYQQKPGKAPKLLIYGASTLQSGVPSRFSGSGSGTDFTLTISSLQPEDFATYFCQQSYSTPLTFGGGTKVEIY

SEQ ID NO: 532 - protein, 107 aa, synthetic construct
  REGION 1..107: note = Synthetic: Light Chain Variable Domain 44
DIQMTQSPSSLSASVGDRVTFTCRASQSITRSLNWYQQKPGKAPKLLIYAASRLQSGVPSRFSGSGSGTDFTLTISSLQPEDFATYFCQQSYSSPLTFGGGTKVEIY

SEQ ID NO: 533 - protein, 107 aa, synthetic construct
  REGION 1..107: note = Synthetic: Light Chain Variable Domain 45
DIQMTQSPSSLSASVGDRVTFTCRASQSISRYLNWYQQKPGKAPKLLIYAASSLQSGVPSRFSGSGSGTDFTLTISSLQPEDFATYFCQQSYSTPVTFGGGTKVEIY

SEQ ID NO: 534 - protein, 107 aa, synthetic construct
  REGION 1..107: note = Synthetic: Light Chain Variable Domain 46
DIQMTQSPSSLSASVGDRVTFTCRASQSISRYLNWYQQKPGKAPKLLIYAASSLQNGVPSRFSGSGSGTDFTLTISSLQPEDFATYFCQQSYSTPVTFGGGTKVEIY

SEQ ID NO: 535 - protein, 107 aa, synthetic construct
  REGION 1..107: note = Synthetic: Light Chain Variable Domain 47
DIQMTQSPSSLSASVGDRVTFTCRASQSISRYLNWYQQKPGKAPKLLIYGASSLESGVPSRFSGSGSGTDFTLTISSLQPEDFATYFCQQSYRTSLTFGGGTKVEIY

SEQ ID NO: 536 - protein, 107 aa, synthetic construct
  REGION 1..107: note = Synthetic: Light Chain Variable Domain 48
DIQMTQSPSSLSASVGDRVTFTCRASQNISRYLNWYQQKPGKAPKLLIYAASSLQSGVPSRFSGSGSGTDFTLTISSLQPEDFATYFCQQSYSTPLTFGGGTKVEIY

SEQ ID NO: 537 - protein, 107 aa, synthetic construct
  REGION 1..107: note = Synthetic: Light Chain Variable Domain 49
DIQMTQSPSSLSASVGDRVTFTCRASQIISRYLNWYQQKPGKAPKLLIYAASSLQSGVPSRFSGSGSGTDFTLTISSLQPEDFATYFCQQSYGTPLTFGGGTKVEIY

SEQ ID NO: 538 - protein, 107 aa, synthetic construct
  REGION 1..107: note = Synthetic: Light Chain Variable Domain 50
DIQMTQSPSSLSASVGDRVTFTCRASQSISRYLNWYQQKPGKAPKLLIYGASSLQSGVPSRFSGSGSGTDFTLTISSLQPEDFATYFCQQSYSTPLTFGGGTKVEIY

SEQ ID NO: 539 - protein, 107 aa, synthetic construct
  REGION 1..107: note = Synthetic: Light Chain Variable Domain 51
DIQMTQSPSSLSASVGDRVTFTCRASQSISRYLNWYQQKPGKAPKLLIYAASSLQSGVPSRFSGSGSGTDFTLTISSLQPEDFATYFCQQSYSTPLTFGGGTKVEIY

SEQ ID NO: 540 - protein, 107 aa, synthetic construct
  REGION 1..107: note = Synthetic: Light Chain Variable Domain 52
DIQMTQSPSSLSASVGDSVTITCRASQSMSSHLNWYHQKPGKAPKLLIYAASRLESGVPSRFSGSGSGTDFTLTISSLQPEDFATYYCQQSYSSPLTFGGGTKVEIK

SEQ ID NO: 541 - protein, 107 aa, synthetic construct
  REGION 1..107: note = Synthetic: Light Chain Variable Domain 53
DIQMTQSPSSLSASVGDSVTITCRASQSISRYLNWYHQKPGKAPKLLIYAASSLQSGVPSRFSGSGSGTDFTLTISSLQPEDFATYYCQQSYSSPLTFGGGTKVEIK

SEQ ID NO: 542 - protein, 107 aa, synthetic construct
  REGION 1..107: note = Synthetic: Light Chain Variable Domain 54
DIQMTQSPSSLSASVGDSVTITCRASQSISTYLNWYHQKPGKAPKLLIYAASSLQSGVPSRFSGSGSGTDFTLTISSLQPEDFATYYCQQSYSSPLTFGGGTKVEIK

SEQ ID NO: 543 - protein, 107 aa, synthetic construct
  REGION 1..107: note = Synthetic: Light Chain Variable Domain 55
DIQMTQSPSSLSASVGDSVTITCRASQSISSYLNWYHQKPGKAPKLLIYAASNLQSGVPSRFSGSGSGTDFTLTISSLQPEDFATYYCQQSYSTPFTFGGGTKVEIK

SEQ ID NO: 544 - protein, 107 aa, synthetic construct
  REGION 1..107: note = Synthetic: Light Chain Variable Domain 56
DIQMTQSPSSLSASVGDSVTITCRASQSISSYLNWYHQKPGKAPKLLIYAASSLQSGVPSRFSGSGSGTDFTLTISSLQPEDFATYYCQQSYRSPFTFGGGTKVEIK

SEQ ID NO: 545 - protein, 16 aa, synthetic construct
  REGION 1..16: note = Synthetic: Consensus CDRH3 (Kabat)
  VARIANT 9: note = X = D, E, H, or Y
EGYDLWSGXSSNWLDP

SEQ ID NO: 546 - protein, 14 aa, synthetic construct
  REGION 1..14: note = Synthetic: Consensus CDRH3 (Chothia)
  VARIANT 8: note = X = D, E, H, or Y
GYDLWSGXSSNWLD

SEQ ID NO: 547 - protein, 18 aa, synthetic construct
  REGION 1..18: note = Synthetic: Consensus CDRH3 (IMGT)
  VARIANT 11: note = X = D, E, H, or Y
AREGYDLWSGXSSNWLDP

SEQ ID NO: 548 - protein, 9 aa, synthetic construct
  REGION 1..9: note = Synthetic: Consensus CDRL3 (Kabat)
  VARIANT 4: note = X = N or Q
  VARIANT 5: note = X = S or N
QQLXXYPRT

SEQ ID NO: 549 - protein, 6 aa, synthetic construct
  REGION 1..6: note = Synthetic: Consensus CDRL3 (Chothia)
  VARIANT 2: note = X = N or Q
  VARIANT 3: note = X = S or N
LXXYPR

SEQ ID NO: 550 - protein, 9 aa, synthetic construct
  REGION 1..9: note = Synthetic: Consensus CDRL3 (IMGT)
  VARIANT 4: note = X = N or Q
  VARIANT 5: note = X = S or N
QQLXXYPRT

SEQ ID NO: 551 - skipped

SEQ ID NO: 552 - protein, 17 aa, synthetic construct
  REGION 1..17: note = Synthetic: Consensus CDRH2 (Kabat)
  VARIANT 3: note = X = S or T
  VARIANT 5: note = X = S or G
  VARIANT 7: note = X = G, A, or S
  VARIANT 8: note = X = L, S, I, or V
  VARIANT 10: note = X = K, H, N, or Y
  VARIANT 16: note = X = K or R
LIXGXGXXTXYADSVXG

SEQ ID NO: 553 - protein, 9 aa, synthetic construct
  REGION 1..9: note = Synthetic: Consensus CDRH3 (Kabat)
  VARIANT 1: note = X = D or E
  VARIANT 6: note = X = T or S
  VARIANT 9: note = X = Y, F, or S
XEGLTXGEX

SEQ ID NO: 554 - protein, 11 aa, synthetic construct
  REGION 1..11: note = Synthetic: Consensus CDRL1 (Kabat)
  VARIANT 2: note = X = A or T
  VARIANT 5: note = X = D, G, or A
  VARIANT 9: note = X = D or N
  VARIANT 11: note = X = A, G, or D
RXSQXIRNXLX

SEQ ID NO: 555 - skipped

SEQ ID NO: 556 - protein, 9 aa, synthetic construct
  REGION 1..9: note = Synthetic: Consensus CDRL3 (Kabat)
  VARIANT 4: note = X = N or S
  VARIANT 5: note = X = N, S, or T
  VARIANT 9: note = X = T or S
LQHXXYPFX

SEQ ID NO: 557 - protein, 7 aa, synthetic construct
  REGION 1..7: note = Synthetic: Consensus CDRH1 (Chothia)
  VARIANT 5: note = X = S, R, N, G, or T
  VARIANT 6: note = X = N, S, T, or I
  VARIANT 7: note = X = Y or H
GFTFXXX

SEQ ID NO: 558 - skipped

SEQ ID NO: 559 - protein, 7 aa, synthetic construct
  REGION 1..7: note = Synthetic: Consensus CDRH3 (Chothia)
  VARIANT 5: note = X = T or S
EGLTXGE

SEQ ID NO: 560 - protein, 7 aa, synthetic construct
  REGION 1..7: note = Synthetic: Consensus CDRL1 (Chothia)
  VARIANT 3: note = X = D, G, or A
  VARIANT 7: note = X = D or N
SQXIRNX

SEQ ID NO: 561 - skipped

SEQ ID NO: 562 - protein, 6 aa, synthetic construct
  REGION 1..6: note = Synthetic: Consensus CDRL3 (Chothia)
  VARIANT 2: note = X = N or S
  VARIANT 3: note = X = N, S, or T
HXXYPF

SEQ ID NO: 563 - protein, 8 aa, synthetic construct
  REGION 1..8: note = Synthetic: Consensus CDRH1 (IMGT)
  VARIANT 5: note = X = S, R, N, G, or T
  VARIANT 6: note = X = N, S, T, or I
  VARIANT 7: note = X = Y or H
GFTFXXXA

SEQ ID NO: 564 - protein, 8 aa, synthetic construct
  REGION 1..8: note = Synthetic: Consensus CDRH2 (IMGT)
  VARIANT 2: note = X = S or T
  VARIANT 4: note = X = S or G
  VARIANT 6: note = X = G, A, or S
  VARIANT 7: note = X = L, S, I, or V
IXGXGXXT

SEQ ID NO: 565 - protein, 11 aa, synthetic construct
  REGION 1..11: note = Synthetic: Consensus CDRH3 (IMGT)
  VARIANT 1: note = X = V or A
  VARIANT 3: note = X = D or E
  VARIANT 8: note = X = T or S
  VARIANT 11: note = X = Y, F, or S
XKXEGLTXGEX

SEQ ID NO: 566 - protein, 6 aa, synthetic construct
  REGION 1..6: note = Synthetic: Consensus CDRL1 (IMGT)
  VARIANT 2: note = X = D, G, or A
  VARIANT 6: note = X = D or N
QXIRNX

SEQ ID NO: 567 - skipped

SEQ ID NO: 568 - protein, 9 aa, synthetic construct
  REGION 1..9: note = Synthetic: Consensus CDRL3 (IMGT)
  VARIANT 4: note = X = N or S
  VARIANT 5: note = X = N, S, or T
  VARIANT 9: note = X = T or S
LQHXXYPFX

SEQ ID NO: 569 - skipped

SEQ ID NO: 570 - protein, 17 aa, synthetic construct
  REGION 1..17: note = Synthetic: Consensus CDRH2 (Kabat)
  VARIANT 3: note = X = N, D, or S
  VARIANT 4: note = X = S or N
  VARIANT 5: note = X = D or N
  VARIANT 7: note = X = I or F
  VARIANT 8: note = X = S or N
RIXXXGXXTRYADSVKG

SEQ ID NO: 571 - protein, 9 aa, synthetic construct
  REGION 1..9: note = Synthetic: Consensus CDRH3 (Kabat)
  VARIANT 7: note = X = P or R
  VARIANT 9: note = X = Y or F
DFQLELXDX

SEQ ID NO: 572 - protein, 11 aa, synthetic construct
  REGION 1..11: note = Synthetic: Consensus CDRL1 (Kabat)
  VARIANT 5: note = X = G or A
  VARIANT 8: note = X = D or N
  VARIANT 11: note = X = A or G
RASQXIRXDLX

SEQ ID NO: 573 - protein, 7 aa, synthetic construct
  REGION 1..7: note = Synthetic: Consensus CDRL2 (Kabat)
  VARIANT 2: note = X = A or T
  VARIANT 4: note = X = S or I
  VARIANT 7: note = X = S or N
AXSXLQX

SEQ ID NO: 574 - protein, 9 aa, synthetic construct
  REGION 1..9: note = Synthetic: Consensus CDRL3 (Kabat)
  VARIANT 4: note = X = K, N, or Y
LQHXSNPFT

SEQ ID NO: 575-576 - skipped (2 entries)

SEQ ID NO: 577 - protein, 7 aa, synthetic construct
  REGION 1..7: note = Synthetic: Consensus CDRH3 (Chothia)
  VARIANT 6: note = X = P or R
FQLELXD

SEQ ID NO: 578 - protein, 7 aa, synthetic construct
  REGION 1..7: note = Synthetic: Consensus CDRL1 (Chothia)
  VARIANT 3: note = X = G or A
  VARIANT 6: note = X = D or N
SQXIRXD

SEQ ID NO: 579 - skipped

SEQ ID NO: 580 - protein, 6 aa, synthetic construct
  REGION 1..6: note = Synthetic: Consensus CDRL3 (Chothia)
  VARIANT 2: note = X = K, N, or Y
HXSNPF

SEQ ID NO: 581 - protein, 8 aa, synthetic construct
  REGION 1..8: note = Synthetic: Consensus CDRH1 (IMGT)
  VARIANT 2: note = X = F or L
  VARIANT 5: note = X = S or R
  VARIANT 6: note = X = N or S
  VARIANT 7: note = X = Y or F
GXTFXXXW

SEQ ID NO: 582 - skipped

SEQ ID NO: 583 - protein, 11 aa, synthetic construct
  REGION 1..11: note = Synthetic: Consensus CDRH3 (IMGT)
  VARIANT 2: note = X = R or S
  VARIANT 9: note = X = P or R
  VARIANT 11: note = X = Y or F
VXDFQLELXDX

SEQ ID NO: 584 - protein, 6 aa, synthetic construct
  REGION 1..6: note = Synthetic: Consensus CDRL1 (IMGT)
  VARIANT 2: note = X = G or A
  VARIANT 5: note = X = D or N
QXIRXD

SEQ ID NO: 585 - skipped

SEQ ID NO: 586 - protein, 9 aa, synthetic construct
  REGION 1..9: note = Synthetic: Consensus CDRL3 (IMGT)
  VARIANT 4: note = X = K, N, or Y
LQHXSNPFT

SEQ ID NO: 587 - protein, 5 aa, synthetic construct
  REGION 1..5: note = Synthetic: Variants of 127 Consensus CDRH1 (Kabat)
  VARIANT 1: note = X = N or S
XAWMS

SEQ ID NO: 588 - protein, 19 aa, synthetic construct
  REGION 1..19: note = Synthetic: Consensus CDRH2 (Kabat)
  VARIANT 2: note = X = I or V
  VARIANT 4: note = X = S or G
  VARIANT 5: note = X = N, V, or K
  VARIANT 6: note = X = S or T
  VARIANT 7: note = X = Y, A, D, or T
  VARIANT 11: note = X = T or A
  VARIANT 13: note = X = Y or F
  VARIANT 14: note = X = A or P
  VARIANT 17: note = X = A or V
  VARIANT 18: note = X = I or K
RXKXXXXGGTXDXXAPXXG

SEQ ID NO: 589 - protein, 9 aa, synthetic construct
  REGION 1..9: note = Synthetic: Consensus CDRH3 (Kabat)
  VARIANT 9: note = X = F or Y
DHPKFGFDX

SEQ ID NO: 590 - protein, 17 aa, synthetic construct
  REGION 1..17: note = Synthetic: Consensus CDRL1 (Kabat)
  VARIANT 7: note = X = F or L
  VARIANT 9: note = X = S or T
  VARIANT 11: note = X = N or S
  VARIANT 12: note = X = N or S
  VARIANT 14: note = X = N or H
  VARIANT 17: note = X = A or N
KSSQSVXYXSXXKXYLX

SEQ ID NO: 591 - protein, 9 aa, synthetic construct
  REGION 1..9: note = Synthetic: Consensus CDRL3 (Kabat)
  VARIANT 3: note = X = H or Y
  VARIANT 5: note = X = D or S
  VARIANT 6: note = X = I or T
QQXYXXPRT

SEQ ID NO: 592 - protein, 7 aa, synthetic construct
  REGION 1..7: note = Synthetic: Consensus CDRH1 (Chothia)
  VARIANT 5: note = X = S, H, or N
  VARIANT 6: note = X = N or S
GFTFXXA

SEQ ID NO: 593 - protein, 8 aa, synthetic construct
  REGION 1..8: note = Synthetic: Consensus CDRH2 (Chothia)
  VARIANT 2: note = X = S or G
  VARIANT 3: note = X = N, V, or K
  VARIANT 4: note = X = S or T
  VARIANT 5: note = X = Y, A, D, or T
KXXXXGGT

SEQ ID NO: 594 - protein, 13 aa, synthetic construct
  REGION 1..13: note = Synthetic: Consensus CDRL1 (Chothia)
  VARIANT 5: note = X = F or L
  VARIANT 7: note = X = S or T
  VARIANT 9: note = X = N or S
  VARIANT 10: note = X = N or S
  VARIANT 12: note = X = N or H
SQSVXYXSXXKXY

SEQ ID NO: 595 - skipped

SEQ ID NO: 596 - protein, 8 aa, synthetic construct
  REGION 1..8: note = Synthetic: Consensus CDRH1 (IMGT)
  VARIANT 5: note = X = S, H, or N
  VARIANT 6: note = X = N or S
GFTFXXAW

SEQ ID NO: 597 - protein, 10 aa, synthetic construct
  REGION 1..10: note = Synthetic: Consensus CDRH2 (IMGT)
  VARIANT 1: note = X = I or V
  VARIANT 3: note = X = S or G
  VARIANT 4: note = X = N, V, or K
  VARIANT 5: note = X = S or T
  VARIANT 6: note = X = Y, A, D, or T
  VARIANT 10: note = X = T or A
XKXXXXGGTX

SEQ ID NO: 598 - protein, 11 aa, synthetic construct
  REGION 1..11: note = Synthetic: Consensus CDRH3 (IMGT)
  VARIANT 11: note = X = F or Y
TTDHPKFGFDX

SEQ ID NO: 599 - protein, 12 aa, synthetic construct
  REGION 1..12: note = Synthetic: Consensus CDRL1 (IMGT)
  VARIANT 4: note = X = F or L
  VARIANT 6: note = X = S or T
  VARIANT 8: note = X = N or S
  VARIANT 9: note = X = N or S
  VARIANT 11: note = X = N or H
QSVXYXSXXKXY

SEQ ID NO: 600 - protein, 9 aa, synthetic construct
  REGION 1..9: note = Synthetic: Consensus CDRL3 (IMGT
  VARIANT 3: note = X = H or Y
  VARIANT 5: note = X = D or S
  VARIANT 6: note = X = I or T
QQXYXXPRT

SEQ ID NO: 601 - skipped

SEQ ID NO: 602 - protein, 19 aa, synthetic construct
  REGION 1..19: note = Synthetic: Consensus CDRH2 (Kabat)
  VARIANT 1: note = X = R or H
  VARIANT 4: note = X = S, R, or T
  VARIANT 5: note = X = I, E, K, L, N, or Q
  VARIANT 6: note = X = S or T
  VARIANT 7: note = X = Y or D
  VARIANT 1..19: note = X = T or R
  VARIANT 1..19: note = X = D or E
  VARIANT 1..19: note = X = S or Y
  VARIANT 1..19: note = X = A or D
  VARIANT 18: note = X = K or Q
XIKXXXXGGTXXXXAPVXG

SEQ ID NO: 603 - protein, 12 aa, synthetic construct
  REGION 1..12: note = Synthetic: Consensus CDRH3 (Kabat)
  VARIANT 2: note = X = S or G
  VARIANT 5: note = X = K, N, or R
  VARIANT 12: note = X = F or Y
GXVWXYGGSLDX

SEQ ID NO: 604 - protein, 11 aa, synthetic construct
  REGION 1..11: note = Synthetic: Consensus CDRL1 (Kabat)
  VARIANT 5: note = X = G, S, F, N, or I
  VARIANT 7: note = X = S or T
  VARIANT 9: note = X = F, Y, or S
RASQXIXRXLN

SEQ ID NO: 605 - skipped

SEQ ID NO: 606 - protein, 9 aa, synthetic construct
  REGION 1..9: note = Synthetic: Consensus CDRL3 (Kabat)
  VARIANT 3: note = X = T or S
  VARIANT 5: note = X = S, N, R, or G
  VARIANT 6: note = X = T or S
  VARIANT 7: note = X = P or S
  VARIANT 8: note = X = L or V
QQXYXXXXT

SEQ ID NO: 607 - protein, 7 aa, synthetic construct
  REGION 1..7: note = Synthetic: Consensus CDRH1 (Chothia)
  VARIANT 3: note = X = T or I
  VARIANT 5: note = X = N, I, or S
  VARIANT 6: note = X = N, or Y
GFXFXXA

SEQ ID NO: 608 - protein, 8 aa, synthetic construct
  REGION 1..8: note = Synthetic: Consensus CDRH2 (Chothia)
  VARIANT 2: note = X = S, R, or T
  VARIANT 3: note = X = I, E, K, L, N, or Q
  VARIANT 4: note = X = S or T
  VARIANT 5: note = X = Y or D
KXXXXGGT

SEQ ID NO: 609 - protein, 10 aa, synthetic construct
  REGION 1..10: note = Synthetic: Consensus CDRH3 (Chothia)
  VARIANT 1: note = X = S or G
  VARIANT 4: note = X = K, N, or R
XVWXYGGSLD

SEQ ID NO: 610 - protein, 7 aa, synthetic construct
  REGION 1..7: note = Synthetic: Consensus CDRL1 (Chothia)
  VARIANT 3: note = X = G, S, F, N, or I
  VARIANT 5: note = X = S or T
  VARIANT 7: note = X = F, Y, or S
SQXIXRX

SEQ ID NO: 611-612 - skipped (2 entries)

SEQ ID NO: 613 - protein, 8 aa, synthetic construct
  REGION 1..8: note = Synthetic: Consensus CDRH1 (IMGT)
  VARIANT 3: note = X = T or I
  VARIANT 5: note = X = N, I, or S
  VARIANT 6: note = X = N, or Y
GFXFXXAW

SEQ ID NO: 614 - protein, 10 aa, synthetic construct
  REGION 1..10: note = Synthetic: Consensus CDRH2 (IMGT)
  VARIANT 3: note = X = S, R, or T
  VARIANT 4: note = X = I, E, K, L, N, or Q
  VARIANT 5: note = X = S or T
  VARIANT 6: note = X = Y or D
  VARIANT 10: note = X = T or R
IKXXXXGGTX

SEQ ID NO: 615 - protein, 14 aa, synthetic construct
  REGION 1..14: note = Synthetic: Consensus CDRH3 (IMGT)
  VARIANT 1: note = X = S, T, I, or A
  VARIANT 4: note = X = S or G
  VARIANT 7: note = X = K, N, or R
  VARIANT 14: note = X = F or Y
XTGXVWXYGGSLDX

SEQ ID NO: 616-617 - skipped (2 entries)

SEQ ID NO: 618 - protein, 9 aa, synthetic construct
  REGION 1..9: note = Synthetic: Consensus CDRL3 (IMGT)
  VARIANT 3: note = X = T or S
  VARIANT 5: note = X = S, N, R, or G
  VARIANT 6: note = X = T or S
  VARIANT 7: note = X = P or S
  VARIANT 8: note = X = L or V
QQXYXXXXT

SEQ ID NO: 619 - protein, 5 aa, synthetic construct
  REGION 1..5: note = Synthetic: Variants of 130 Consensus CDRH1 (Kabat)
  VARIANT 1: note = X = Y, N, or T
XAWMS

SEQ ID NO: 620 - protein, 19 aa, synthetic construct
  REGION 1..19: note = Synthetic: Consensus CDRH2 (Kabat)
  VARIANT 4: note = X = S or R
  VARIANT 5: note = X = E or K
  VARIANT 6: note = X = T, S, or I
  VARIANT 7: note = X = D or Y
  VARIANT 15: note = X = A or S
RIKXXXXGGTTDYAXPVKG

SEQ ID NO: 621 - protein, 11 aa, synthetic construct
  REGION 1..11: note = Synthetic: Consensus CDRH3 (Kabat)
  VARIANT 5: note = X = N, H, S or Y
  VARIANT 6: note = X = V or L
  VARIANT 8: note = X = F or Y
  VARIANT 11: note = X = F or Y
GSSWXXYXFDX

SEQ ID NO: 622 - protein, 11 aa, synthetic construct
  REGION 1..11: note = Synthetic: Consensus CDRL1 (Kabat)
  VARIANT 6: note = X = I or M
  VARIANT 8: note = X = N, S, R or T
  VARIANT 9: note = X = Y or H
RASQSXSXXLN

SEQ ID NO: 623 - skipped

SEQ ID NO: 624 - protein, 9 aa, synthetic construct
  REGION 1..9: note = Synthetic: Consensus CDRL3 (Kabat)
  VARIANT 5: note = X = S or R
  VARIANT 6: note = X = S or T
  VARIANT 8: note = X = L or F
QQSYXXPXT

SEQ ID NO: 625 - protein, 7 aa, synthetic construct
  REGION 1..7: note = Synthetic: Consensus CDRH1 (Chothia)
  VARIANT 3: note = X = T or I
  VARIANT 6: note = X = Y, N, or T
GFXFSXA

SEQ ID NO: 626 - protein, 8 aa, synthetic construct
  REGION 1..8: note = Synthetic: Consensus CDRH2 (Chothia)
  VARIANT 2: note = X = S or R
  VARIANT 3: note = X = E or K
  VARIANT 4: note = X = T, S, or I
  VARIANT 5: note = X = D or Y
KXXXXGGT

SEQ ID NO: 627 - protein, 9 aa, synthetic construct
  REGION 1..9: note = Synthetic: Consensus CDRH3 (Chothia)
  VARIANT 4: note = X = N, H, S or Y
  VARIANT 5: note = X = V or L
  VARIANT 7: note = X = F or Y
SSWXXYXFD

SEQ ID NO: 628 - protein, 7 aa, synthetic construct
  REGION 1..7: note = Synthetic: Consensus CDRL1 (Chothia)
  VARIANT 4: note = X = I or M
  VARIANT 6: note = X = N, S, R or T
  VARIANT 7: note = X = Y or H
SQSXSXX

SEQ ID NO: 629-630 - skipped (2 entries)

SEQ ID NO: 631 - protein, 8 aa, synthetic construct
  REGION 1..8: note = Synthetic: Consensus CDRH1 (IMGT)
  VARIANT 3: note = X = T or I
  VARIANT 6: note = X = Y, N, or T
GFXFSXAW

SEQ ID NO: 632 - protein, 10 aa, synthetic construct
  REGION 1..10: note = Synthetic: Consensus CDRH2 (IMGT)
  VARIANT 3: note = X = S or R
  VARIANT 4: note = X = E or K
  VARIANT 5: note = X = T, S, or I
  VARIANT 6: note = X = D or Y
IKXXXXGGTT

SEQ ID NO: 633 - protein, 13 aa, synthetic construct
  REGION 1..13: note = Synthetic: Consensus CDRH3 (IMGT)
  VARIANT 7: note = X = N, H, S or Y
  VARIANT 8: note = X = V or L
  VARIANT 10: note = X = F or Y
  VARIANT 13: note = X = F or Y
TTGSSWXXYXFDX

SEQ ID NO: 634-635 - skipped (2 entries)

SEQ ID NO: 636 - protein, 9 aa, synthetic construct
  REGION 1..9: note = Synthetic: Consensus CDRL3 (IMGT)
  VARIANT 5: note = X = S or R
  VARIANT 6: note = X = S or T
  VARIANT 8: note = X = L or F
QQSYXXPXT

SEQ ID NO: 637 - protein, 329 aa, synthetic construct
  REGION 1..329: note = Synthetic: hIgG1*
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 638 - protein, 327 aa, synthetic construct
  REGION 1..327: note = Synthetic: hIgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 639 - protein, 327 aa, synthetic construct
  REGION 1..327: note = Synthetic: IgG4-SP
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 640 - protein, 327 aa, synthetic construct
  REGION 1..327: note = Synthetic: IgG4-SPLE
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 641 - protein, 324 aa, synthetic construct
  REGION 1..324: note = Synthetic: IgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 642 - protein, 329 aa, synthetic construct
  REGION 1..329: note = Synthetic: hIgG1-N297A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 643 - protein, 329 aa, synthetic construct
  REGION 1..329: note = Synthetic: hIgG1-D265A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 644 - protein, 329 aa, synthetic construct
  REGION 1..329: note = Synthetic: hIgG1-LALA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 645 - protein, 329 aa, synthetic construct
  REGION 1..329: note = Synthetic: hIgG1-LAGA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 646 - protein, 329 aa, synthetic construct
  REGION 1..329: note = Synthetic: hIgG1-LALAGA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 647 - protein, 329 aa, synthetic construct
  REGION 1..329: note = Synthetic: hIgG1-LALAPG
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 648 - protein, 329 aa, synthetic construct
  REGION 1..329: note = Synthetic: hIgG1-YTE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 649 - protein, 329 aa, synthetic construct
  REGION 1..329: note = Synthetic: hIgG1-N297A/YTE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 650 - protein, 329 aa, synthetic construct
  REGION 1..329: note = Synthetic: hIgG1-D265A/YTE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 651 - protein, 329 aa, synthetic construct
  REGION 1..329: note = Synthetic: hIgG1-LALA/YTE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 652 - protein, 329 aa, synthetic construct
  REGION 1..329: note = Synthetic: hIgG1-LAGA/YTE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 653 - protein, 329 aa, synthetic construct
  REGION 1..329: note = Synthetic: hIgG1-LALAGA/YTE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 654 - protein, 329 aa, synthetic construct
  REGION 1..329: note = Synthetic: hIgG1-LALAPG/YTE
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 655 - protein, 329 aa, synthetic construct
  REGION 1..329: note = Synthetic: hIgG1-LS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 656 - protein, 329 aa, synthetic construct
  REGION 1..329: note = Synthetic: hIgG1-N297A/LS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 657 - protein, 329 aa, synthetic construct
  REGION 1..329: note = Synthetic: hIgG1-D265A/LS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 658 - protein, 329 aa, synthetic construct
  REGION 1..329: note = Synthetic: hIgG1-LALA/LS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 659 - protein, 329 aa, synthetic construct
  REGION 1..329: note = Synthetic: hIgG1-LAGA/LS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 660 - protein, 329 aa, synthetic construct
  REGION 1..329: note = Synthetic: hIgG1-LALAGA/LS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 661 - protein, 329 aa, synthetic construct
  REGION 1..329: note = Synthetic: hIgG1-LALAPG/LS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 662 - protein, 329 aa, synthetic construct
  REGION 1..329: note = Synthetic: hIgG1-DHS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 663 - protein, 329 aa, synthetic construct
  REGION 1..329: note = Synthetic: hIgG1-N297A/DHS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 664 - protein, 329 aa, synthetic construct
  REGION 1..329: note = Synthetic: hIgG1-D265A/DHS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 665 - protein, 329 aa, synthetic construct
  REGION 1..329: note = Synthetic: hIgG1-LALA/DHS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 666 - protein, 329 aa, synthetic construct
  REGION 1..329: note = Synthetic: hIgG1-LAGA/DHS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 667 - protein, 329 aa, synthetic construct
  REGION 1..329: note = Synthetic: hIgG1-LALAGA/DHS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 668 - protein, 329 aa, synthetic construct
  REGION 1..329: note = Synthetic: hIgG1-LALAPG/DHS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 669 - protein, 327 aa, synthetic construct
  REGION 1..327: note = Synthetic: hIgG4-YTE
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 670 - protein, 327 aa, synthetic construct
  REGION 1..327: note = Synthetic: hIgG4-SP/YTE
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 671 - protein, 327 aa, synthetic construct
  REGION 1..327: note = Synthetic: hIgG4-SPLE/YTE
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 672 - protein, 326 aa, synthetic construct
  REGION 1..326: note = Synthetic: hIgG4-LS
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSYTQKSLSLSLGK

SEQ ID NO: 673 - protein, 327 aa, synthetic construct
  REGION 1..327: note = Synthetic: hIgG4-SP/LS
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 674 - protein, 327 aa, synthetic construct
  REGION 1..327: note = Synthetic: hIgG4-SPLE/LS
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 675 - protein, 327 aa, synthetic construct
  REGION 1..327: note = Synthetic: hIgG4-DHS
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 676 - protein, 327 aa, synthetic construct
  REGION 1..327: note = Synthetic: hIgG4-SP/DHS
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 677 - protein, 327 aa, synthetic construct
  REGION 1..327: note = Synthetic: hIgG4-SPLE/DHS
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 678 - protein, 324 aa, synthetic construct
  REGION 1..324: note = Synthetic: hIgG2-YTE
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSP

SEQ ID NO: 679 - protein, 324 aa, synthetic construct; identical to SEQ ID NO: 641
  REGION 1..324: note = Synthetic: hIgG2-LS

SEQ ID NO: 680 - protein, 324 aa, synthetic construct
  REGION 1..324: note = Synthetic: hIgG2-DHS
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSP

SEQ ID NO: 681 - protein, 327 aa, synthetic construct; identical to SEQ ID NO: 639
  REGION 1..327: note = Synthetic: IgG4-SP

SEQ ID NO: 682 - protein, 329 aa, synthetic construct
  REGION 1..329: note = Synthetic: hIgG1-LA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 683 - protein, 329 aa, synthetic construct
  REGION 1..329: note = Synthetic: hIgG1-N297A/LA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 684 - protein, 329 aa, synthetic construct
  REGION 1..329: note = Synthetic: hIgG1-D265A/LA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 685 - protein, 329 aa, synthetic construct
  REGION 1..329: note = Synthetic: hIgG1-LALA/LA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 686 - protein, 329 aa, synthetic construct
  REGION 1..329: note = Synthetic: hIgG1-LAGA/LA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 687 - protein, 329 aa, synthetic construct
  REGION 1..329: note = Synthetic: hIgG1-LALAGA/LA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 688 - protein, 329 aa, synthetic construct
  REGION 1..329: note = Synthetic: hIgG1-LALAPG/LA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 689 - protein, 329 aa, synthetic construct
  REGION 1..329: note = Synthetic: hIgG1-N434A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 690 - protein, 329 aa, synthetic construct
  REGION 1..329: note = Synthetic: hIgG1-N297A/ N434A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 691 - protein, 329 aa, synthetic construct
  REGION 1..329: note = Synthetic: hIgG1-D265A/ N434A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 692 - protein, 329 aa, synthetic construct
  REGION 1..329: note = Synthetic: hIgG1-LALA/ N434A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 693 - protein, 329 aa, synthetic construct
  REGION 1..329: note = Synthetic: hIgG1-LAGA/ N434A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 694 - protein, 329 aa, synthetic construct
  REGION 1..329: note = Synthetic: hIgG1-LALAGA/ N434A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 695 - protein, 329 aa, synthetic construct
  REGION 1..329: note = Synthetic: hIgG1-LALAPG/ N434A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 696 - protein, 329 aa, synthetic construct
  REGION 1..329: note = Synthetic: hIgG1-N434W
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 697 - protein, 329 aa, synthetic construct
  REGION 1..329: note = Synthetic: hIgG1-N297A/ N434W
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 698 - protein, 329 aa, synthetic construct
  REGION 1..329: note = Synthetic: hIgG1-D265A/ N434W
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 699 - protein, 329 aa, synthetic construct
  REGION 1..329: note = Synthetic: hIgG1-LALA/ N434W
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 700 - protein, 329 aa, synthetic construct
  REGION 1..329: note = Synthetic: hIgG1-LAGA/ N434W
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 701 - protein, 329 aa, synthetic construct
  REGION 1..329: note = Synthetic: hIgG1-LALAGA/ N434W
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 702 - protein, 329 aa, synthetic construct
  REGION 1..329: note = Synthetic: hIgG1-LALAPG/ N434W
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 703 - protein, 329 aa, synthetic construct
  REGION 1..329: note = Synthetic: hIgG1/DQ
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 704 - protein, 329 aa, synthetic construct
  REGION 1..329: note = Synthetic: hIgG1-N297A/DQ
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 705 - protein, 329 aa, synthetic construct
  REGION 1..329: note = Synthetic: hIgG1-D265A/DQ
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 706 - protein, 329 aa, synthetic construct
  REGION 1..329: note = Synthetic: hIgG1-LALA/DQ
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 707 - protein, 329 aa, synthetic construct
  REGION 1..329: note = Synthetic: hIgG1-LAGA/DQ
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 708 - protein, 329 aa, synthetic construct
  REGION 1..329: note = Synthetic: hIgG1-LALAGA/DQ
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 709 - protein, 329 aa, synthetic construct
  REGION 1..329: note = Synthetic: hIgG1-LALAPG/DQ
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 710 - protein, 329 aa, synthetic construct
  REGION 1..329: note = Synthetic: hIgG1/DW
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 711 - protein, 329 aa, synthetic construct
  REGION 1..329: note = Synthetic: hIgG1-N297A/DW
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 712 - protein, 329 aa, synthetic construct
  REGION 1..329: note = Synthetic: hIgG1-D265A/DW
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 713 - protein, 329 aa, synthetic construct
  REGION 1..329: note = Synthetic: hIgG1-LALA/DW
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 714 - protein, 329 aa, synthetic construct
  REGION 1..329: note = Synthetic: hIgG1-LAGA/DW
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 715 - protein, 329 aa, synthetic construct
  REGION 1..329: note = Synthetic: hIgG1-LALAGA/DW
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 716 - protein, 329 aa, synthetic construct
  REGION 1..329: note = Synthetic: hIgG1-LALAPG/DW
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 717 - protein, 329 aa, synthetic construct
  REGION 1..329: note = Synthetic: hIgG1/YD
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 718 - protein, 329 aa, synthetic construct
  REGION 1..329: note = Synthetic: hIgG1-N297A/YD
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 719 - protein, 329 aa, synthetic construct
  REGION 1..329: note = Synthetic: hIgG1-D265A/YD
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 720 - protein, 329 aa, synthetic construct
  REGION 1..329: note = Synthetic: hIgG1-LALA/YD
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 721 - protein, 329 aa, synthetic construct
  REGION 1..329: note = Synthetic: hIgG1-LAGA/YD
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 722 - protein, 329 aa, synthetic construct
  REGION 1..329: note = Synthetic: hIgG1-LALAGA/YD
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 723 - protein, 329 aa, synthetic construct
  REGION 1..329: note = Synthetic: hIgG1-LALAPG/YD
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 724 - protein, 329 aa, synthetic construct
  REGION 1..329: note = Synthetic: hIgG1/QVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 725 - protein, 329 aa, synthetic construct
  REGION 1..329: note = Synthetic: hIgG1-N297A/QVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 726 - protein, 329 aa, synthetic construct
  REGION 1..329: note = Synthetic: hIgG1-D265A/QVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 727 - protein, 329 aa, synthetic construct
  REGION 1..329: note = Synthetic: hIgG1-LALA/QVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 728 - protein, 329 aa, synthetic construct
  REGION 1..329: note = Synthetic: hIgG1-LAGA/QVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 729 - protein, 329 aa, synthetic construct
  REGION 1..329: note = Synthetic: hIgG1-LALAGA/QVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 730 - protein, 329 aa, synthetic construct
  REGION 1..329: note = Synthetic: hIgG1-LALAPG/QVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 731 - protein, 329 aa, synthetic construct
  REGION 1..329: note = Synthetic: hIgG1/DDRVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 732 - protein, 329 aa, synthetic construct
  REGION 1..329: note = Synthetic: hIgG1-N297A/DDRVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 733 - protein, 329 aa, synthetic construct
  REGION 1..329: note = Synthetic: hIgG1-D265A/DDRVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 734 - protein, 329 aa, synthetic construct
  REGION 1..329: note = Synthetic: hIgG1-LALA/DDRVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 735 - protein, 329 aa, synthetic construct
  REGION 1..329: note = Synthetic: hIgG1-LAGA/DDRVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 736 - protein, 329 aa, synthetic construct
  REGION 1..329: note = Synthetic: hIgG1-LALAGA/DDRVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 737 - protein, 329 aa, synthetic construct
  REGION 1..329: note = Synthetic: hIgG1-LALAPG/DDRVV
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 738 - protein, 107 aa, synthetic construct [sequence omitted]

SEQ ID NO: 739 - protein, 183 aa, synthetic construct
  REGION 1..183: note = Synthetic: OX40L protein
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 740 - protein, 6 aa, synthetic construct
  REGION 1..6: note = Synthetic: Kabat HCDR1
SGNWWS

SEQ ID NO: 741 - protein, 7 aa, synthetic construct
  REGION 1..7: note = Synthetic: Kabat HCDR1
SGGYYWS

SEQ ID NO: 742 - protein, 8 aa, synthetic construct
  REGION 1..8: note = Synthetic: Chothia HCDR1
GGSISSGN

SEQ ID NO: 743 - protein, 7 aa, synthetic construct
  REGION 1..7: note = Synthetic: Chothia HCDR1
GFTFDSA

SEQ ID NO: 744 - protein, 9 aa, synthetic construct
  REGION 1..9: note = Synthetic: Chothia HCDR1
GGSISSGGY

SEQ ID NO: 745 - protein, 9 aa, synthetic construct
  REGION 1..9: note = Synthetic: IMGT CDR1
GGSISSGNW

SEQ ID NO: 746 - protein, 8 aa, synthetic construct
  REGION 1..8: note = Synthetic: IMGT CDR1
GFTFDSAW

SEQ ID NO: 747 - protein, 10 aa, synthetic construct
  REGION 1..10: note = Synthetic: IMGT CDR1
GGSISSGGYY

SEQ ID NO: 748 - protein, 19 aa, synthetic construct
  REGION 1..19: note = Synthetic: Kabat HCDR2
RFKSETDGGTKDFGAHVRG

SEQ ID NO: 749 - protein, 16 aa, synthetic construct
  REGION 1..16: note = Synthetic: Kabat HCDR2
EIHHSGRTNYNPSLKS

SEQ ID NO: 750 - protein, 19 aa, synthetic construct
  REGION 1..19: note = Synthetic: Kabat HCDR2
RIKRKTDGGTTDYAAPVKG

SEQ ID NO: 751 - protein, 19 aa, synthetic construct
  REGION 1..19: note = Synthetic: Kabat HCDR2
RIKSKNEGGTIDYAAPVKG

SEQ ID NO: 752 - protein, 17 aa, synthetic construct
  REGION 1..17: note = Synthetic: Kabat HCDR2
WIHPNTGKTGYAQKFQG

SEQ ID NO: 753 - protein, 16 aa, synthetic construct
  REGION 1..16: note = Synthetic: Kabat HCDR2
HIYYSGSTKYNPSLKS

SEQ ID NO: 754 - protein, 5 aa, synthetic construct
  REGION 1..5: note = Synthetic: Chothia HCDR2
HHSGR

SEQ ID NO: 755 - protein, 8 aa, synthetic construct
  REGION 1..8: note = Synthetic: Chothia HCDR2
KSKNEGGT

SEQ ID NO: 756 - protein, 6 aa, synthetic construct
  REGION 1..6: note = Synthetic: Chothia HCDR2
HPNTGK

SEQ ID NO: 757 - protein, 5 aa, synthetic construct
  REGION 1..5: note = Synthetic: Chothia HCDR2
YYSGS

SEQ ID NO: 758 - protein, 10 aa, synthetic construct
  REGION 1..10: note = Synthetic: IMGT HCDR2
FKSETDGGTK

SEQ ID NO: 759 - protein, 7 aa, synthetic construct
  REGION 1..7: note = Synthetic: IMGT HCDR2
IHHSGRT

SEQ ID NO: 760 - protein, 10 aa, synthetic construct
  REGION 1..10: note = Synthetic: IMGT HCDR2
IKSKNEGGTI

SEQ ID NO: 761 - protein, 8 aa, synthetic construct
  REGION 1..8: note = Synthetic: IMGT HCDR2
IHPNTGKT

SEQ ID NO: 762 - protein, 7 aa, synthetic construct
  REGION 1..7: note = Synthetic: IMGT HCDR2
IYYSGST

SEQ ID NO: 763 - protein, 9 aa, synthetic construct
  REGION 1..9: note = Synthetic: Kabat HCDR3
GGTAVPFDF

SEQ ID NO: 764 - protein, 10 aa, synthetic construct
  REGION 1..10: note = Synthetic: Kabat HCDR3
DRQGSFAFDI

SEQ ID NO: 765 - protein, 10 aa, synthetic construct
  REGION 1..10: note = Synthetic: Kabat HCDR3
DRPWGSFFDY

SEQ ID NO: 766 - protein, 12 aa, synthetic construct
  REGION 1..12: note = Synthetic: Kabat HCDR3
DFLWFGELPFDY

SEQ ID NO: 767 - protein, 16 aa, synthetic construct
  REGION 1..16: note = Synthetic: Kabat HCDR3
ESYDLWSGHSFNWFDP

SEQ ID NO: 768 - protein, 12 aa, synthetic construct
  REGION 1..12: note = Synthetic: Kabat HCDR3
DNWGSGWYYMDV

SEQ ID NO: 769 - protein, 7 aa, synthetic construct
  REGION 1..7: note = Synthetic: Chothia HCDR3
GTAVPFD

SEQ ID NO: 770 - protein, 8 aa, synthetic construct
  REGION 1..8: note = Synthetic: Chothia HCDR3
RQGSFAFD

SEQ ID NO: 771 - protein, 8 aa, synthetic construct
  REGION 1..8: note = Synthetic: Chothia HCDR3
RPWGSFFD

SEQ ID NO: 772 - protein, 10 aa, synthetic construct
  REGION 1..10: note = Synthetic: Chothia HCDR3
FLWFGELPFD

SEQ ID NO: 773 - protein, 14 aa, synthetic construct
  REGION 1..14: note = Synthetic: Chothia HCDR3
SYDLWSGHSFNWFD

SEQ ID NO: 774 - protein, 10 aa, synthetic construct
  REGION 1..10: note = Synthetic: Chothia HCDR3
NWGSGWYYMD

SEQ ID NO: 775 - protein, 11 aa, synthetic construct
  REGION 1..11: note = Synthetic: IMGT HCDR3
TTGGTAVPFDF

SEQ ID NO: 776 - protein, 12 aa, synthetic construct
  REGION 1..12: note = Synthetic: IMGT HCDR3
ARDRQGSFAFDI

SEQ ID NO: 777 - protein, 12 aa, synthetic construct
  REGION 1..12: note = Synthetic: IMGT HCDR3
TLDRPWGSFFDY

SEQ ID NO: 778 - protein, 14 aa, synthetic construct
  REGION 1..14: note = Synthetic: IMGT HCDR3
NTDFLWFGELPFDY

SEQ ID NO: 779 - protein, 18 aa, synthetic construct
  REGION 1..18: note = Synthetic: IMGT HCDR3
ARESYDLWSGHSFNWFDP

SEQ ID NO: 780 - protein, 14 aa, synthetic construct
  REGION 1..14: note = Synthetic: IMGT HCDR3
ARDNWGSGWYYMDV

SEQ ID NO: 781 - protein, 17 aa, synthetic construct
  REGION 1..17: note = Synthetic: Kabat LCDR1
KSSQSLLYSSNNKNYLA

SEQ ID NO: 782 - protein, 11 aa, synthetic construct
  REGION 1..11: note = Synthetic: Kabat LCDR1
RASQSISTWLV

SEQ ID NO: 783 - protein, 17 aa, synthetic construct
  REGION 1..17: note = Synthetic: Kabat LCDR1
KSSQSVLYSSNNKNYFA

SEQ ID NO: 784 - protein, 11 aa, synthetic construct
  REGION 1..11: note = Synthetic: Kabat LCDR1
RASQGISNHLA

SEQ ID NO: 785 - protein, 11 aa, synthetic construct
  REGION 1..11: note = Synthetic: Kabat LCDR1
RASQGITSYLA

SEQ ID NO: 786 - protein, 11 aa, synthetic construct
  REGION 1..11: note = Synthetic: Kabat LCDR1
RASQSVSNYFA

SEQ ID NO: 787 - protein, 13 aa, synthetic construct
  REGION 1..13: note = Synthetic: Chothia LCDR1
SQSLLYSSNNKNY

SEQ ID NO: 788 - protein, 7 aa, synthetic construct
  REGION 1..7: note = Synthetic: Chothia LCDR1
SQSISTW

SEQ ID NO: 789 - protein, 7 aa, synthetic construct
  REGION 1..7: note = Synthetic: Chothia LCDR1
SQGISNH

SEQ ID NO: 790 - protein, 7 aa, synthetic construct
  REGION 1..7: note = Synthetic: Chothia LCDR1
SQGITSY

SEQ ID NO: 791 - protein, 7 aa, synthetic construct
  REGION 1..7: note = Synthetic: Chothia LCDR1
SQSVSNY

SEQ ID NO: 792 - protein, 12 aa, synthetic construct
  REGION 1..12: note = Synthetic: IMGT LCDR1
QSLLYSSNNKNY

SEQ ID NO: 793 - protein, 6 aa, synthetic construct
  REGION 1..6: note = Synthetic: IMGT LCDR1
QSISTW

SEQ ID NO: 794 - protein, 6 aa, synthetic construct
  REGION 1..6: note = Synthetic: IMGT LCDR1
QGISNH

SEQ ID NO: 795 - protein, 6 aa, synthetic construct
  REGION 1..6: note = Synthetic: IMGT LCDR1
QGITSY

SEQ ID NO: 796 - protein, 6 aa, synthetic construct
  REGION 1..6: note = Synthetic: IMGT LCDR1
QSVSNY

SEQ ID NO: 797 - protein, 7 aa, synthetic construct
  REGION 1..7: note = Synthetic: Kabat LCDR2
WTSTRES

SEQ ID NO: 798 - protein, 7 aa, synthetic construct
  REGION 1..7: note = Synthetic: Kabat LCDR2
KASSLES

SEQ ID NO: 799 - protein, 7 aa, synthetic construct
  REGION 1..7: note = Synthetic: Kabat LCDR2
RASTRES

SEQ ID NO: 800 - protein, 7 aa, synthetic construct
  REGION 1..7: note = Synthetic: Kabat LCDR2
GATTLQS

SEQ ID NO: 801 - protein, 7 aa, synthetic construct
  REGION 1..7: note = Synthetic: Kabat LCDR2
DASNRAT

SEQ ID NO: 802-805 - skipped (4 entries)

SEQ ID NO: 806 - protein, 9 aa, synthetic construct
  REGION 1..9: note = Synthetic: Kabat LCDR3
LQYSNTPYS

SEQ ID NO: 807 - protein, 9 aa, synthetic construct
  REGION 1..9: note = Synthetic: Kabat LCDR3
QQHISYPYT

SEQ ID NO: 808 - protein, 9 aa, synthetic construct
  REGION 1..9: note = Synthetic: Kabat LCDR3
QQYYTTPLT

SEQ ID NO: 809 - protein, 9 aa, synthetic construct
  REGION 1..9: note = Synthetic: Kabat LCDR3
QKLNSAPLT

SEQ ID NO: 810 - protein, 9 aa, synthetic construct
  REGION 1..9: note = Synthetic: Kabat LCDR3
QQLQSYPYT

SEQ ID NO: 811 - protein, 9 aa, synthetic construct
  REGION 1..9: note = Synthetic: Kabat LCDR3
QHRSNWPLT

SEQ ID NO: 812 - protein, 6 aa, synthetic construct
  REGION 1..6: note = Synthetic: Chothia and IMGT LCDR3
YSNTPY

SEQ ID NO: 813 - protein, 6 aa, synthetic construct
  REGION 1..6: note = Synthetic: Chothia and IMGT LCDR3
HISYPY

SEQ ID NO: 814 - protein, 6 aa, synthetic construct
  REGION 1..6: note = Synthetic: Chothia and IMGT LCDR3
YYTTPL

SEQ ID NO: 815 - protein, 6 aa, synthetic construct
  REGION 1..6: note = Synthetic: Chothia and IMGT LCDR3
LNSAPL

SEQ ID NO: 816 - protein, 6 aa, synthetic construct
  REGION 1..6: note = Synthetic: Chothia and IMGT LCDR3
LQSYPY

SEQ ID NO: 817 - protein, 6 aa, synthetic construct
  REGION 1..6: note = Synthetic: Chothia and IMGT LCDR3
RSNWPL

SEQ ID NO: 818 - protein, 120 aa, synthetic construct
  REGION 1..120: note = Synthetic: Heavy Chain Variable Domain 71
EVQLVESGGGLVKPGGSLRLSCAASGFTFSNAWMSWVRQAPGKGLEWVGRFKSETDGGTKDFGAHVRGRFIISKDASKNTLYLQMNRLKTEDTAVYYCTTGGTAVPFDFWGLGTLVTVSS

SEQ ID NO: 819 - protein, 119 aa, synthetic construct
  REGION 1..119: note = Synthetic: Heavy Chain Variable Domain 72
QVQLQESGPGLVKPSGTLSLTCAVSGGSISSGNWWSWVRQPPGSGLEWIGEIHHSGRTNYNPSLKSRVVISVDKSKNHFSLKLSSVTAADTAVYYCARDRQGSFAFDIWGQGTMVTVSS

SEQ ID NO: 820 - protein, 121 aa, synthetic construct
  REGION 1..121: note = Synthetic: Heavy Chain Variable Domain 73
EVQLVESGGGLVKPGGSLRLSCTASGFTFDSAWMSWVRQAPGKGLEWVGRIKRKTDGGTTDYAAPVKGRFTISRDDSTKTLYLQMNSLKTEDTAVYYCTLDRPWGSFFDYWGQGTLVTVSS

SEQ ID NO: 821 - protein, 123 aa, synthetic construct
  REGION 1..123: note = Synthetic: Heavy Chain Variable Domain 74
EVQLVESGGGLVKPGGSLRLSCAASGFTFSNAWMSWVRQAPGKGLEWVGRIKSKNEGGTIDYAAPVKGRFTISRDDSKNTLYLQMNSLKTEDTAVYYCNTDFLWFGELPFDYWGQGTLVTVSS

SEQ ID NO: 822 - protein, 118 aa, synthetic construct
  REGION 1..118: note = Synthetic: Heavy Chain Variable Domain 75
EVQVVESGGGLVQPGGSLRLSCAASGFTFSSYAMTWVRQAPGKGLEWVSLISGSGGLTKYADSVKGRFTISRDNSKKTLYLQMNSLRAEDTAAYYCVKDEGLTTGEYWGQGTLVTVSS

SEQ ID NO: 823 - protein, 125 aa, synthetic construct
  REGION 1..125: note = Synthetic: Heavy Chain Variable Domain 76
QVQLVQSGTEVKKPGASVKVSCKASGYTFTNYDINWVRQATGQGLEWVGWIHPNTGKTGYAQKFQGRVTMTRDISIGTAYMELNSLRSEDTAVYYCARESYDLWSGHSFNWFDPWGQGTLVTVSS

SEQ ID NO: 824 - protein, 122 aa, synthetic construct
  REGION 1..122: note = Synthetic: Heavy Chain Variable Domain 77
QVQLQESGPGLVKPSQTLSLTCTVSGGSISSGGYYWSWIRQYPGKGLEWIGHIYYSGSTKYNPSLKSRLTISVDTSKNQFSLKLSSVTAADTAVYYCARDNWGSGWYYMDVWGKGTTVTVSS

SEQ ID NO: 825 - protein, 113 aa, synthetic construct
  REGION 1..113: note = Synthetic: Light Chain Variable Domain 57
DIVMTQSPDSLAVSLGERATINCKSSQSLLYSSNNKNYLAWYQQKPGQPPKLLFYWTSTRESGVPDRFGASGSGTDFTLTISSLQVEDVAIYFCLQYSNTPYSFGQGTKLEIK

SEQ ID NO: 826 - protein, 107 aa, synthetic construct
  REGION 1..107: note = Synthetic: Light Chain Variable Domain 58
DIQMTQSPATLSASVGDRVTITCRASQSISTWLVWYQQKPGKAPKLLIYKASSLESGVPSRFSGSGSGTEFTLTISSLQPDDFATYYCQQHISYPYTFGQGTRLEIK

SEQ ID NO: 827 - protein, 113 aa, synthetic construct
  REGION 1..113: note = Synthetic: Light Chain Variable Domain 59
DIVMTQSPDSLAVSLGERATINCKSSQSVLYSSNNKNYFAWYQQKPGQPPRLLIYRASTRESGVPDRFSGSGSGTDFTLTISSLQAEDVAVYYCQQYYTTPLTFGGGTKVEIR

SEQ ID NO: 828 - protein, 107 aa, synthetic construct
  REGION 1..107: note = Synthetic: Light Chain Variable Domain 60
DNQMTQSPSSLSASVGDRVTITCRASQGISNHLAWYQQKPGKVPDLLIYAASTLQSGVPSRFSGSGSGTDFTLTISSLQPEDVANYYCQKLNSAPLTFGGGTKVEIK

SEQ ID NO: 829 - protein, 107 aa, synthetic construct; identical to SEQ ID NO: 512
  REGION 1..107: note = Synthetic: Light Chain Variable Domain 61

SEQ ID NO: 830 - protein, 107 aa, synthetic construct
  REGION 1..107: note = Synthetic: Light Chain Variable Domain 62
DIQLTQSPSFLSASVGDRVIITCRASQGITSYLAWYQRKPGKAPKLLIYGATTLQSGVPSRFSGSGSGTEFTLTISSLQPEDFATYYCQQLQSYPYTFGQGTKLEIK

SEQ ID NO: 831 - protein, 107 aa, synthetic construct
  REGION 1..107: note = Synthetic: Light Chain Variable Domain 63
EIVLTQSPATLSLSPGERATLSCRASQSVSNYFAWYRQKPGQAPRLLIYDASNRATGIPARFSGSGSGTDFTLTISSLEPEDFAVYYCQHRSNWPLTFGGGTKVEIK

SEQ ID NO: 832 - protein, 127 aa, synthetic construct
  REGION 1..127: note = Synthetic: Amlitelimab VH
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSS

SEQ ID NO: 833 - protein, 107 aa, synthetic construct
  REGION 1..107: note = Synthetic: Amlitelimab VL
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIK

SEQ ID NO: 834 - protein, 120 aa, synthetic construct
  REGION 1..120: note = Synthetic: LC.001 (Oxelumab) VH
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSS

SEQ ID NO: 835 - protein, 107 aa, synthetic construct
  REGION 1..107: note = Synthetic: LC.001 (Oxelumab) VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 836 - protein, 120 aa, synthetic construct
  REGION 1..120: note = Synthetic: LC.005 VH
QVQLVESGGGVVQPGRSLRLSCAASGFTFSNFGMHWVRQAPGKGLEWVAAIWYDGHDKYYSYYVKGRFTISRDNSKNTLFLQMNSLRAEDTAVYYCARDSSSWYRYFDYWGQGTLVTVSS

SEQ ID NO: 837 - protein, 107 aa, synthetic construct
  REGION 1..107: note = Synthetic: LC.005 VL
EIVLTQSPGTLSLSPGERATLSCRASQSVSSNYLAWYQQKPGQAPRLLIYGASSRATGIPDRFSGSGSGTDFTLTISRLEPEDFAVYYCQQYGSSFTFGPGTKVDIK

SEQ ID NO: 838 - protein, 120 aa, synthetic construct
  REGION 1..120: note = Synthetic: LC.019 VH
QKQLVEFGGGVVQPGRSLRLSCAASGFTFSNYGMHWVRQAPGKGLWVAVIWNDGSNKYYVDSVKGRFIISRDNSKNTLYLQMNSLRAEDTAVYYCARDRMGIYYYGMDVWGQGTTVTVSS

SEQ ID NO: 839 - protein, 107 aa, synthetic construct
  REGION 1..107: note = Synthetic: LC.019 VL
EIVLTQSPATLSLSPGERATLSCRASQGVSRYLAWYQQKPGQAPRLLIYDASNRATGIPARVSGSGPGTDFTLTISSLEPEDFAVDYCQQRSNWQYTFGQGTKLEIK

SEQ ID NO: 840 - protein, 7 aa, synthetic construct
  REGION 1..7: note = Synthetic: Chothia HCDR1
GFTFSNA

SEQ ID NO: 841 - protein, 118 aa, synthetic construct
  REGION 1..118: note = Synthetic: Heavy Chain Variable Domain 78
EVRLVESGGGLVQPGGSLRLSCAASGFTFSSYAMTWVRQAPGKGLEWVSLISGSGGLTKYADSVKGRFTISRDNSKKTLYLQMNSLRAEDTAVYYCAKDEGLTTGEYWGQGTLVTVSS

SEQ ID NO: 842 - protein, 107 aa, synthetic construct
  REGION 1..107: note = Synthetic: Light Chain Variable Domain 64
DIQMTQSPSSLSASVGDRVTITCRASQGIRNDLGWYQQKPGKAPKRLIYATSSFQSGVPSRFSGSGSGTEFTLTISSLQPEDFATYYCLQHNSYPFTFGPGTKVDVK